SEQUENCE LISTING (8 sequences)

SEQ ID NO: 1 - protein, 799 aa, synthetic construct
GFKDNSPISDTSFSLQLSQDGLQLTPASSSSESLSIIDVASDQNLFQTFIKEWRCKKRFSISLACEKIRSLTSSKTATIGSRFKQASSPQEIPIRDDGFPIKGCDDTLVVGLAVCWGGRDAYYFSLQKEQKHSEISASLVPPSLDPSLTLKDRMWYLQSCLRKESDKECSVVIYDFIQSYKILLLSCGISLEQSYEDPKVACWLLDPDSQEPTLHSIVTSFLPHELPLLEGMETSQGIQSLGLNAGSEHSGRYRASVESILIFNSMNQLNSLLQKENLQDVFRKVEMPSQYCLALLELNGIGFSTAECESQKHIMQAKLDAIETQAYQLAGHSFSFTSSDDIAEVLFLELKLPPNREMKNQGSKKTLGSTRRGIDNGRKLRLGRQFSTSKDVLNKLKALHPLPGLILEWRRITNAITKVVFPLQREKCLNPFLGMERIYPVSQSHTATGRITFTEPNIQNVPRDFEIKMPTLVGESPPSQAVGKGLLPMGRGKYKKGFSVNPRCQAQMEERAADRGMPFSISMRHAFVPFPGGSILAADYSQLELRILAHLSHDRRLIQVLNTGADVFRSIAAEWKMIEPESVGDDLRQQAKQICYGIIYGMGAKSLGEQMGIKENDAACYIDSFKSRYTGINQFMTETVKNCKRDGFVQTILGRRRYLPGIKDNNPYRKAHAERQAINTIVQGSAADIVKIATVNIQKQLETFHSTFKSHGHREGMLQSDQTGLSRKRKLQGMFCPIRGGFFILQLHDELLYEVAEEDVVQVAQIVKNEMESAVKLSVKLKVKVKIGASWGELKDFDV

SEQ ID NO: 2-5 - skipped (4 entries)

SEQ ID NO: 6 - DNA, 37 bp, synthetic construct
  source 1..37: mol_type = other DNA
  modified_base 1: mod_base = OTHER; note = Conjugated to Cy5 at 5 position
cactgtgagcttagtcacatttcatcatgcaggacag

SEQ ID NO: 7 - DNA, 14 bp, synthetic construct
  source 1..14: mol_type = other DNA
  modified_base 14: mod_base = OTHER; note = Conjugated to Blackhole Quencher at 3' position
ctaagctcacagtg

SEQ ID NO: 8 - DNA, 15 bp, synthetic construct
  source 1..15: mol_type = other DNA
ctgtcctgcatgatg

(19) United States
(12) Patent Application Publication
(10) Pub. No.: US 2026/0317313 A1
(43) Pub. Date: Oct. 15, 2026

(54) DNA Polymerase Theta Inhibitors Containing Non-Saturated 5-Membered Heterocyclic Rings and Use Thereof

(71) Applicants: Thomas Jefferson University, Philadelphia, PA (US); Recombination Therapeutics, LLC, Doylestown, PA (US)

(72) Inventors: Richard T Pomerantz, Philadelphia, PA (US); Mercy Ramanjulu, King of Prussia, PA (US)

(21) Appl. No.: 19/144,147

(22) PCT Filed: Dec. 28, 2023
(86) PCT No.: PCT/US2023/086134

§ 371 Date: Jun. 27, 2025

Related U.S. Application Data
(60) Provisional application No. 63/477,472, filed on Dec. 28, 2022.

(51) Int. Cl. C07D 413/14; A61K 31/415; A61K 31/4166; A61K 31/4192; A61K 31/4196; A61K 31/4245; A61K 31/427; A61K 31/454; C07B 59/00; C07D 231/12; C07D 233/70; C07D 249/06; C07D 249/12; C07D 257/04; C07D 271/10; C07D 401/14; C07D 403/04; C07D 403/06; C07D 409/04; C07D 409/06

(52) U.S. Cl. CPC C07D 413/14; A61K 31/415; A61K 31/4166; A61K 31/4192; A61K 31/4196; A61K 31/4245; A61K 31/427; A61K 31/454; C07B 59/002; C07D 231/12; C07D 233/70; C07D 249/06; C07D 249/12; C07D 257/04; C07D 271/10; C07D 401/14; C07D 403/04; C07D 403/06; C07D 409/04; C07D 409/06; C07B 2200/05

(57) ABSTRACT

The invention relates to chemical compounds containing 5-membered heterocyclic non-saturated rings and derivatives thereof and their use in the treatment and prophylaxis of cancer, and to compositions containing said derivatives and processes for their preparation.

### CROSS-REFERENCE TO RELATED APPLICATIONS

The present application claims priority to U.S. Provisional Application No. 63/477,472, filed Dec. 28, 2022, which is incorporated by reference herein in its entirety.

### STATEMENT REGARDING FEDERALLY SPONSORED RESEARCH OR DEVELOPMENT

This invention was made with government support under 1R41CA239983-01A1 and 1R41CA265430-01 awarded to Recombination Therapeutics, LLC by the National Cancer Institute. The government has certain rights in the invention.

### FIELD OF INVENTION

The invention relates to chemical compounds containing a non-saturated 5-membered heterocyclic ring and their use in the treatment and prophylaxis of cancer, and to compositions containing said derivatives and processes for their preparation.

### BACKGROUND OF THE INVENTION

Mutations in BRCA1/2 (BRCA) and other homologous recombination (HR) genes predispose women to breast and ovarian cancer, and somatic BRCA2 mutations are frequently found advanced prostate cancers (Turner, N., et al., Curr Opin Pharmacol 5, 388-393 (2005); Konstantinopoulos, P. A., et al., Cancer Discov 5, 1137-1154 (2015); Caulfield, S. E., et al., J Adv Pract Oncol 10, 167-174 (2019); Pan. Z. & Xie, X. Oncotarget 8, 97657-97670 (2017); Munroe. M. & Kolesar, J. Am J Health Syst Pharm 73, 1037-1041 (2016)). Because BRCA mutated cancer cells are defective in HR, they are strongly susceptible to DNA damaging agents compared to normal cells (Lord, C. J. & Ashworth, A. Science 355, 1152-1158 (2017); McCabe, N. et al. Cancer Res 66, 8109-8115 (2006); Tutt, A. N. et al. Cold Spring Harb Symp Quant Biol 70, 139-148 (2005)). Drugs designed to induce DNA damage and/or suppress DNA repair (i.e. Poly (ADP-ribose) polymerase 1 (PARP1) inhibitors) therefore preferentially kill in HR-deficient cells (Sonnenblick, A., et al., Nat Rev Clin Oncol 12, 27-41 (2015); Farmer, H. et al. Nature 434, 917-921 (2005); Bryant. H. E. et al. Nature 434, 913-917 (2005)). Although PARPi showed initial clinical success, not all patients respond to PARPi and drug resistance is a major problem. Therefore, the development of additional DNA damage response (DDR) drug targets to help reduce drug resistance and improve patient outcomes is urgently needed.

In 2015, DNA polymerase theta (Polq) was identified as a potential cancer drug target with high potential for inducing synthetic lethality in BRCA-deficient cancers (Mateos-Gomez, P. A. et al. Nature 518, 254-257 (2015); Ceccaldi, R. L., Nature 517, 258-262 (2015)). Polq is a multi-functional enzyme possessing an amino-terminal super family 2 (SF2) helicase (Polq-hel), a central domain of unknown function, and a carboxy-terminal polymerase (Polq-pol) which belongs to the A-family of polymerases. Polq was found to be upregulated in the majority (70%) of breast tumors as well as ovarian cancers (Lemee, F. et al. Proc Natl Acad Sci USA 107, 13390-13395 (2010); Higgins, G. S. et al. Oncotarget 1, 175-184, (2010); Arana, M. E., et al., Nucleic Acids Res 36, 3847-3856 (2008); Seki, M. et al. EMBO J 23, 4484-4494, (2004)), and Polq overexpression correlates with HR deficiency and a poor outcome for patients with breast cancer (Begg, A. Oncotarget 1, 161-162, (2010)). Polq expression was also shown to confer cellular resistance to ionizing radiation and other DNA damaging agents (i.e. topoisomerase inhibitors, cisplatin), as well as PARPi (Chandramouly, G. et al. Cell Rep 34, 108820 (2021); Yousefzadeh, M. J. et al. PLoS Genet 10, e1004654 (2014); Higgins, G. S. et al. Cancer Res 70, 2984-2993 (2010); Dai, C. H. et al. Oncotarget 7, 65157-65170 (2016)).

Polq promotes double-strand break (DSB) repair by performing microhomology-mediated end-joining (MMEJ), which is also known as theta-mediated end-joining (TMEJ) and alternative end-joining (alt-EJ) (Kent. T., et al., Nature structural & molecular biology 22, 230-237 (2015)). For instance, Polq-pol facilitates MMEJ of DNA possessing 3′ ssDNA overhangs with short tracts (2-6 bp) of microhomologous DNA in vitro, and Polq expression was shown to be essential for cellular MMEJ (Black. S. J. et al. Nat Commun 10, 4423 (2019); Wyatt, D. W. et al. Mol Cell 63, 662-673 (2016)). Polq-hel is thought to play an auxiliary role in MMEJ by stripping RPA from ssDNA overhangs (Mateos-Gomez, P. A. et al. Nat Struct Mol Biol 24, 1116-1123 (2017)). Polq-hel also exhibits relatively weak ATP-dependent DNA unwinding activity (Ozdemir. A. Y., et al., J Biol Chem 293, 5259-5269 (2018)), and is reported to dissociate RAD51 from ssDNA.

In S and G2 cell cycle phases, DSB repair is mainly performed by HR which depends on BRCA1, BRCA2, PALB2 and many auxiliary factors that function with RAD51 recombinase and the Mre11-Nbs1-Rad50-CtIP DNA resection complex (Moynahan, M. E. & Jasin, M. Nat Rev Mol Cell Biol 11, 196-207 (2010); San Filippo, J., et al., Annual review of biochemistry 77, 229-257 (2008); Li, X. & Heyer, W. D. Cell research 18, 99-113 (2008); Truong, L. N. et al. Proc Natl Acad Sci USA 110, 7720-7725 (2013)). Because MMEJ acts as a backup DSB repair pathway, inactivation of Polq in HR deficient cells induces synthetic lethality, with little or no effect on HR proficient cells (Cho, N. W. & Greenberg, R. A. Nature 518, 174-176 (2015)); Mengwasser, K. E. et al. Mol Cell 73, 885-899 e886 (2019)). For instance, knockdown or knockout of Polq in BRCA-mutant cells causes a significant reduction in survival (Feng, W. et al. Nat Commun 10, 4286 (2019)). In contrast, knockdown or knockout of Polq in BRCA wild-type (WT) cells shows little or no effect. Furthermore, Polq inactivation appears to potentiate the effects of PARPi in BRCA-deficient cells (Zatreanu, D. et al. Nat Commun 12, 3636 (2021)). Thus, Polq inhibitors (Polqi) are expected to potentiate the effects of PARPi, possibly by reducing cellular resistance to PARPi. Finally, synthetic lethality between Polq and the HR factor FANCD2 was additionally shown in mice¹³.

Both the polymerase and helicase enzymatic domains have been shown to promote the survival of BRCA1-mutant mouse embryonic stem cells (mESCs), however, the cells were slightly more dependent on Polq-pol. Additional studies showed that only the polymerase activity of Polq (Polq-pol) is important for MMEJ and cellular resistance to the DSB inducing agent bleomycin. Based on these studies, and additional findings that Polq-pol enzymatic activity is essential for MMEJ, inhibitors of Polq-pol are expected to exhibit selective killing of HR-deficient cancer cells.

Polq was also shown to be synthetic lethal with other DDR factors, including Ku70/80, RAD54, FANCJ, and DNA-PKcs in the presence of DNA damaging agents (Kumar, R. J. et al. NAR Cancer 2, zcaa038, (2020)). Polq is also semi-synthetic lethal with the DDR kinases ATM and ATR (Shima, N., Munroe, R. J. & Schimenti, J. C. Mol Cell Biol 24, 10381-10389 (2004); Wang, Z. et al. J Biol Chem 294, 3909-3919 (2019)). Hence, Polq is an important drug target in cancers that are deficient in DDR pathways.

There is a need in the art for compositions and methods for inhibiting Polq for preventing or treating various diseases or disorders, such as cancer. The present invention satisfies this unmet need.

### SUMMARY OF THE INVENTION

In one aspect, the present invention relates to a compound having the structure of Formula (I), or a tautomeric or a stereochemically isomeric form, a pharmaceutically acceptable salt thereof, or a solvate thereof:

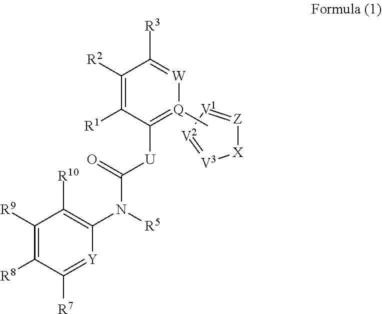

wherein:
W represents C(R⁴) or N;
U represents CH₂, O, S, or NR^{U};
Y represents C(R⁶) or N;
ring A represents an unsaturated 5-membered ring;
Q represents a carbon atom directly bonded to ring A;
R¹, R², R³, R⁴, R⁷, R⁸, R⁹, R¹⁰, and Ru independently represent hydrogen, deuterium, C₁₋₆ alkyl, C₂₋₆ alkenyl, alkynyl, hydroxy, thiol, C₁₋₆ alkoxy, halogen, haloC₁₋₆ alkyl, haloC₁₋₆ alkoxy, C₃₋₈ cycloalkyl, nitrile, NR^{X}R^{Y}, and combinations thereof; wherein two adjacent groups R¹ to R⁴ or R⁶ to R¹⁰ optionally join to form a 5- to 7-membered saturated or unsaturated ring optionally containing one or more heteroatoms selected from O, N or S;
R⁵ and R⁶ independently represent hydrogen, deuterium, C₁₋₆ alkyl, C₂₋₆ alkenyl, alkynyl, hydroxy, thiol, C₁₋₆ alkoxy, halogen, haloC₁₋₆ alkyl, haloC₁₋₆ alkoxy, C₃₋₈ cycloalkyl, nitrile, —NR^{X}R^{Y}, aryl, heteroaryl, heterocyclyl, amide, and combinations thereof;
Z represents CR^{Z} or N, V¹ is C, and the bond between V¹ and Z is a double bond; or
Z represents NR^{Z}, CR^{Z}R^{Z′}, C═S, or C═O, V¹ is N, and the bond between V¹ and Z is a single bond;
V² represents N or CR^{V2}
V³ represents N or CR^{V3}
X represents C(R¹⁵)(R¹⁶), N(R¹⁷), S, or O;
R¹⁵, R¹⁶, and R¹⁷ independently represent hydrogen, deuterium, C₁₋₂₀ alkyl, haloC₁₋₂₀ alkyl, —OR^{15a}, —SR^{15a}, nitrile, —COC₁₋₂₀ alkyl, —COOC₁₋₂₀ alkyl, hydroxy, C₁₋₂₀ alkoxy, C₁₋₂₀ alkanol, C₃₋₈ cycloalkyl, halogen, carbonyl, —NR^{V}R^{W}, —CH₂—NR^{V}R^{W}, —OSO₂NH₂, —P(O)OH₂, aryl, heteroaryl, heterocyclyl, and combinations thereof; wherein R¹⁵, R¹⁶, and R¹⁷ may further comprise one or more divalent linkers L selected from the group consisting alkylene, cycloalkylene, heteroalkylene, heterocycloalkylene, alkenylene, alkynylene, arylene, heteroarylene, silyl, amine, amide, ester, ether, carbonyl, carbamate, carbonate, sulfamate, sulfonic ester, sulfoximine, sulfonamide, thioether, thioester, disulfide, hydrazine, urea, thiourea, phosphate, phosphonate ester, poly(alkyl ether), heteroatom, and combinations thereof;
R^{Z}, R^{Z′}, R^{V2}, and R^{V3} independently represent hydrogen, deuterium, C₁₋₆ alkyl, hydroxy, C₁₋₆ alkoxy, C₁₋₆ alkanol, halogen, —OR^{15b}, CO₂H, CO₂R^{15b}, haloC₁₋₆ alkyl, and combinations thereof;
provided one of R^{Z}, R^{Z′}, R^{V2}, R^{V3}, R¹⁵, R¹⁶, and R¹⁷ represents a direct bond to Q;
each R^{15a} and R^{15b} independently represents hydrogen, deuterium, or C₁₋₆ alkyl;
R^{V}, R^{W}, R^{X} and R^{Y} independently represent hydrogen, deuterium, C₁₋₆ alkyl, haloC₁₋₆ alkyl, C₃₋₈ cycloalkyl, —COC₁₋₆ alkyl or heterocyclyl; wherein said alkyl groups may be optionally substituted with or more deuterium, hydroxy, amino or sulfone groups; and said heterocyclyl ring may be optionally substituted by one or more deuterium, oxo, hydroxy, C₁₋₆ alkanol or —COC₁₋₆ alkyl groups.

In one aspect, the present invention relates to a method of treating cancer in a subject in need thereof, the method comprising administering to the subject a compound of Formula (I) or a tautomeric or a stereochemically isomeric form, a pharmaceutically acceptable salt or a solvate thereof.

In one aspect, the present invention relates to a method of inhibiting the expression or stability of DNA polymerase theta (Polq) in a subject, the method comprising administering to the subject a Formula (I) or a tautomeric or a stereochemically isomeric form, a pharmaceutically acceptable salt or a solvate thereof.

In one aspect, the present invention relates to a method of inhibiting the activity of DNA polymerase theta (Polq), the method comprising the step of contacting Polq with a compound of Formula (I) or a tautomeric or a stereochemically isomeric form, a pharmaceutically acceptable salt or a solvate thereof.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 is a scatter plots showing that the survival of BRCA2-null HCT116 cells is significantly reduced by treatment with the indicated Polqi (Example 26) as compared to BRCA2-WT HCT116 cells, which are mostly resistant to Polqi. Data are represented as mean. n=3, +/−s.d.

FIG. 2 is a scatter plot showing that the survival of BRCA2-null DLD1 cells is significantly reduced by treatment with the indicated Polqi (Example 26) as compared to BRCA2-WT DLD1 cells, which are mostly resistant to Polqi. Data are represented as mean. n=3, +/−s.d.

FIG. 3 is a scatter plot showing that the survival of BRCA2-null HCT116 cells is significantly reduced by treatment with the indicated Polqi (Example 27) as compared to BRCA2-WT HCT116 cells, which are mostly resistant to Polqi. Data are represented as mean. n=3, +/−s.d.

FIG. 4 is a scatter plot showing that the survival of BRCA2-null DLD1 cells is significantly reduced by treatment with the indicated Polqi (Example 27) as compared to BRCA2-WT DLD1 cells, which are mostly resistant to Polqi. Data are represented as mean. n=3, +/−s.d.

### DETAILED DESCRIPTION

The present invention is based, in part, on the discovery that novel compounds containing non-saturated heterocyclic 5-member rings inhibited Polq DNA synthesis activity. Thus, the present invention is directed, in part, to compositions comprising said compounds containing non-saturated heterocyclic 5-member rings and methods for inhibiting Polq in vitro and in vivo. In various embodiments, the Polq (e.g., the activity of Polq, the expression level of Polq, etc.) is essential for the proliferation of cancer cells, such as those defective in HDR or other DNA repair pathways. Thus, the present invention also provides, in part, compounds and methods for preventing or treating cancer with compounds containing non-saturated heterocyclic 5-member rings. The invention also provides a kit for modifying or inhibiting Polq (e.g., the activity of Polq, the expression level of Polq, etc.).

### Definitions

Unless defined otherwise, all technical and scientific terms used herein have the same meaning as commonly understood by one of ordinary skill in the art to which this invention belongs. As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

As used herein, the term “alkyl,” by itself or as part of another substituent means, unless otherwise stated, a straight or branched chain hydrocarbon having the number of carbon atoms designated (i.e., C₁₋₆ means one to six carbon atoms) and includes straight, branched chain, or cyclic substituent groups. Examples include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. The term “alkyl,” unless otherwise noted, is also meant to include those derivatives of alkyl defined in more detail below, such as “heteroalkyl”, “haloalkyl” and “homoalkyl”.

The term “haloC₁₋₆ alkyl” as used herein as a group or part of a group refers to a C₁₋₆ alkyl group as defined herein wherein one or more than one hydrogen atom is replaced with a halogen. The term ‘haloC₁₋₆ alkyl’ therefore includes monohaloC₁₋₆ alkyl and also polyhaloC₁₋₆ alkyl. There may be one, two, three or more hydrogen atoms replaced with a halogen, so the haloC₁₋₆ alkyl may have one, two, three or more halogens. Examples of such groups include fluoroethyl, fluoromethyl, difluoromethyl, trifluoromethyl or trifluoroethyl and the like.

Similarly, the term “haloC₁₋₆ alkoxy” as used herein as a group or part of a group refers to a C₁₋₆ alkoxy group as defined herein wherein one or more than one hydrogen atom is replaced with a halogen. The term ‘haloC₁₋₆ alkoxy therefore includes monohaloC₁₋₆ alkoxy and also polyhaloC₁₋₆ alkoxy. There may be one, two, three or more hydrogen atoms replaced with a halogen, so the haloC₁₋₆ alkyl may have one, two, three or more halogens. Examples of such groups include fluoroethoxy, fluoromethoxy, difluoromethoxy, trifluoromethoxy or trifluoroethoxy and the like.

The term “C₃₋₈ cycloalkyl” as used herein refers to a saturated monocyclic hydrocarbon ring of 3 to 8 carbon atoms. Examples of such groups include cyclopropyl, cyclobutyl, cyclopentyl, cyclohexyl, cycloheptyl, cyclooctyl and the like.

As used herein, the term “substituted alkyl” means alkyl, as defined above, substituted by one, two or three substituents selected from the group consisting of halogen, —OH, alkoxy, —NH₂, —N(CH₃)₂, —C(═O)OH, trifluoromethyl, —C≡N, —C(═O)O(C₁-C₄)alkyl, —C(═O)NH₂, —SO₂NH₂, —C(═NH)NH₂, and —NO₂, preferably containing one or two substituents selected from halogen, —OH, alkoxy, —NH₂, trifluoromethyl. —N(CH₃)₂, and —C(═O)OH, more preferably selected from halogen, alkoxy and —OH. Examples of substituted alkyls include, but are not limited to, 2,2-difluoropropyl, 2-carboxycyclopentyl and 3-chloropropyl.

As used herein, the term “alkylene” by itself or as part of another molecule means a divalent radical derived from an alkane, as exemplified by (—CH₂—)ₙ. By way of example only, such groups include, but are not limited to, groups having 24 or fewer carbon atoms such as the structures —CH₂CH₂— and —CH₂CH₂CH₂CH₂—. The term “alkylene,” unless otherwise noted, is also meant to include those groups described below as “heteroalkylene.”

As used herein, the terms “alkoxy,” “alkylamino” and “alkylthio” are used in their conventional sense, and refer to alkyl groups linked to molecules via an oxygen atom, an amino group, a sulfur atom, respectively. As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred are (C₁-C₃) alkoxy, particularly ethoxy and methoxy.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N. Si, P, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃.

As used herein, the term “aromatic” refers to a carbocycle or heterocycle with one or more polyunsaturated rings and having aromatic character, i.e., having (4n+2) delocalized (pi) electrons, where n is an integer.

As used herein, the term “aryl,” employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl, and naphthyl. Preferred are phenyl and naphthyl, most preferred is phenyl.

As used herein, the term “heterocycle” or “heterocyclyl” or “heterocyclic” by itself or as part of another substituent means, unless otherwise stated, an unsubstituted or substituted, stable, mono- or multi-cyclic heterocyclic ring system that consists of carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In one embodiment, the heterocycle is a heteroaryl.

As used herein, the term “heteroaryl” or “heteroaromatic” refers to aryl groups which contain at least one heteroatom selected from N, O, Si, P, and S; wherein the nitrogen and sulfur atoms may be optionally oxidized, and the nitrogen atom(s) may be optionally quaternized. Heteroaryl groups may be substituted or unsubstituted. A heteroaryl group may be attached to the remainder of the molecule through a heteroatom. A polycyclic heteroaryl may include one or more rings that are partially saturated. Examples include tetrahydroquinoline, 2,3-dihydrobenzofuryl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl.

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide.

Examples of heteroaryl groups include pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As used herein, the term “amino aryl” refers to an aryl moiety which contains an amino moiety. Such amino moieties may include, but are not limited to primary amines, secondary amines, tertiary amines, masked amines, or protected amines. Such tertiary amines, masked amines, or protected amines may be converted to primary amine or secondary amine moieties. Additionally, the amine moiety may include an amine-like moiety which has similar chemical characteristics as amine moieties, including but not limited to chemical reactivity.

As used herein, the term “substituted” means that an atom or group of atoms has replaced hydrogen as the substituent attached to another group. For aryl, aryl-(C₁-C₃)alkyl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two. In yet another embodiment, the substituents are independently selected from the group consisting of C₁₋₆ alkyl, —OH, C₁₋₆ alkoxy, halo, amino, acetamido and nitro. In yet another embodiment, the substituents are independently selected from the group consisting of C₁₋₆ alkyl, C₁₋₆ alkoxy, halo, acetamido, and nitro. In another embodiment, the substituents are selected from the group consisting of hydrogen, deuterium, C₁₋₆ alkyl, C₂₋₆ alkenyl, hydroxy, C₁₋₆ alkoxy, halogen, haloC₁₋₆ alkyl, haloC₁₋₆ alkoxy, C₃₋₈ cycloalkyl, nitrile, amino, and combinations thereof. As used herein, where a substituent is an alkyl or alkoxy group, the carbon chain may be branched, straight or cyclic, with straight being preferred.

As used herein, “combinations thereof” refers to any combination of any two or more of the preceding substituents, without limit.

Several references to integers and R, R¹, R², R³, R⁴, R⁵, R⁶, etc. are made in chemical structures and moieties disclosed and described herein. Any description of integers and R, R¹, R², R³, R⁴, R⁵, R⁶, etc. in the specification is applicable to any structure or moiety reciting integers and R, R¹, R², R³, R⁴, R⁵, R⁶, etc. respectively.

As used herein, the term “protected,” as used herein, refers to the presence of a “protecting group” or moiety that prevents reaction of the chemically reactive functional group under certain reaction conditions. The protecting group will vary depending on the type of chemically reactive group being protected. By way of example only, (i) if the chemically reactive group is an amine or a hydrazide, the protecting group may be selected from tert-butyloxycarbonyl (t-Boc) and 9-fluorenylmethoxycarbonyl (Fmoc); (ii) if the chemically reactive group is a thiol, the protecting group may be orthopyridyldisulfide; and (iii) if the chemically reactive group is a carboxylic acid, such as butanoic or propionic acid, or a hydroxyl group, the protecting group may be benzyl or an alkyl group such as methyl, ethyl, or tert-butyl. Additionally, protecting groups include, but are not limited to, photolabile groups, such as Nvoc and MeNvoc, and other protecting groups known in the art. Other protecting groups are described in Greene and Wuts, Protective Groups in Organic Synthesis, 3rd Ed., John Wiley & Sons, New York, N.Y., 1999.

The term “derivative” refers to a small molecule that differs in structure from the reference molecule, but retains the essential properties of the reference molecule. A derivative may change its interaction with certain other molecules relative to the reference molecule. A derivative molecule may also include a salt, an adduct, tautomer, isomer, or other variant of the reference molecule.

The term “tautomers” are constitutional isomers of organic compounds that readily interconvert by a chemical process (tautomerization).

The term “isomers” or “stereoisomers” refer to compounds, which have identical chemical constitution, but differ with regard to the arrangement of the atoms or groups in space.

“Pharmaceutically acceptable” refers to those properties and/or substances which are acceptable to the subject from a pharmacological/toxicological point of view and to the manufacturing pharmaceutical chemist from a physical/chemical point of view regarding composition, formulation, stability, subject acceptance and bioavailability, “Pharmaceutically acceptable carrier” refers to a medium that does not interfere with the effectiveness of the biological activity of the active ingredient(s) and is not toxic to the host to which it is administered.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the subject. Some examples of materials that may serve as pharmaceutically acceptable carriers include: sugars, such as lactose, glucose, and sucrose; starches, such as corn starch and potato starch; cellulose, and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the subject. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art.

The term “pharmaceutically acceptable salt” refers to any pharmaceutically acceptable salt, which upon administration to the subject is capable of providing (directly or indirectly) a compound as described herein. Such salts preferably are acid addition salts with physiologically acceptable organic or inorganic acids. Examples of the acid addition salts include mineral acid addition salts such as, for example, hydrochloride, hydrobromide, hydroiodide, sulphate, nitrate, phosphate, and organic acid addition salts such as, for example, acetate, trifluoroacetate, maleate, fumarate, citrate, oxalate, succinate, tartrate, malate, mandelate, methane sulphonate, and p-toluenesulphonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine, and basic amino acids salts. However, it will be appreciated that non-pharmaceutically acceptable salts also fall within the scope of the invention since those may be useful in the preparation of pharmaceutically acceptable salts. Procedures for salt formation are conventional in the art.

The term “solvate” in accordance with this invention should be understood as meaning any form of the active compound in accordance with the invention in which the said compound is bonded by a non-covalent bond to another molecule (normally a polar solvent), including especially hydrates and alcoholates.

As used herein, the term “pharmaceutical composition” refers to a mixture of at least one compound of the invention with other chemical components and entities, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, topical, intraperitoneal, intramuscular, oral, aerosol, parenteral, ophthalmic, pulmonary, and topical administration.

As used herein, the terms “therapeutic compound”, “therapeutic agent”, “drug”, “active pharmaceutical”, and “active pharmaceutical ingredient” are used interchangeably to refer to chemical entities that display certain pharmacological effects in a body and are administered for such purpose. Non-limiting examples of therapeutic agents include, but are not limited to, antibiotics, analgesics, vaccines, anticonvulsants; anti-diabetic agents, antifungal agents, antineoplastic agents, anti-parkinsonian agents, anti-rheumatic agents, appetite suppressants, biological response modifiers, cardiovascular agents, central nervous system stimulants, contraceptive agents, dietary supplements, vitamins, minerals, lipids, saccharides, metals, metabolites, amino acids (and precursors), nucleic acids and precursors, contrast agents, diagnostic agents, dopamine receptor agonists, erectile dysfunction agents, fertility agents, gastrointestinal agents, hormones, immunomodulators, antihypercalcemia agents, mast cell stabilizers, muscle relaxants, nutritional agents, ophthalmic agents, osteoporosis agents, psychotherapeutic agents, parasympathomimetic agents, parasympatholytic agents, respiratory agents, sedative hypnotic agents, skin and mucous membrane agents, smoking cessation agents, steroids, sympatholytic agents, urinary tract agents, uterine relaxants, vaginal agents, vasodilator, anti-hypertensive, hyperthyroids, anti-hyperthyroids, anti-asthmatics and vertigo agents. In certain embodiments, the one or more therapeutic agents are water-soluble, poorly water-soluble drug or a drug with a low, medium or high melting point. The therapeutic agents may be provided with or without a stabilizing salt or salts.

Some examples of active ingredients suitable for use in the pharmaceutical formulations and methods of the present invention include: hydrophilic, lipophilic, amphiphilic or hydrophobic, and that can be solubilized, dispersed, or partially solubilized and dispersed, on or about the compounds or compositions of the present invention. Alternatively, an active ingredient may also be provided separately from the solid pharmaceutical composition, such as for co-administration. Such active ingredients can be any compound or mixture of compounds having therapeutic or other value when administered to an animal, particularly to a mammal, such as drugs, nutrients, cosmeceuticals, nutraceuticals, diagnostic agents, nutritional agents, and the like. The active agents described herein may be found in their native state, however, they will generally be provided in the form of a salt. The active agents described herein include their isomers, analogs and derivatives.

A “therapeutic” treatment is a treatment administered to a subject who exhibits signs or symptoms of a disease or disorder, for the purpose of diminishing or eliminating those signs or symptoms.

The terms “effective amount” and “pharmaceutically effective amount” refer to a sufficient amount of an agent to provide the desired biological result. That result can be reduction and/or alleviation of a sign, symptom, or cause of a disease or disorder, or any other desired alteration of a biological system. An appropriate effective amount in any individual case may be determined by one of ordinary skill in the art using routine experimentation.

A “therapeutically effective amount” refers to that amount which provides a therapeutic effect for a given condition and administration regimen. In particular, “therapeutically effective amount” means an amount that is effective to prevent, alleviate or ameliorate symptoms of the disease or prolong the survival of the subject being treated, which may be a human or non-human animal. Determination of a therapeutically effective amount is within the skill of the person skilled in the art.

As used herein, the term “stabilizers” refers to either, or both, primary particle and/or secondary stabilizers, which may be polymers or other small molecules. Non-limiting examples of primary particle and/or secondary stabilizers for use with the present invention include, e.g., starch, modified starch, and starch derivatives, gums, including but not limited to polymers, polypeptides, albumin, amino acids, thiols, amines, carboxylic acid and combinations or derivatives thereof. Other examples include xanthan gum, alginic acid, other alginates, benitoniite, veegum, agar, guar, locust bean gum, gum arabic, quince psyllium, flax seed, okra gum, arabinoglactin, pectin, tragacanth, scleroglucan, dextran, amylose, amylopectin, dextrin, etc., cross-linked polyvinylpyrrolidone, ion-exchange resins, potassium polymethacrylate, carrageenan (and derivatives), gum karaya and biosynthetic gum. Other examples of useful primary particle and/or secondary stabilizers include polymers such as: polycarbonates (linear polyesters of carbonic acid); microporous materials (bisphenol, a microporous poly(vinylchloride), microporous polyamides, microporous modacrylic copolymers, microporous styrene-acrylic and its copolymers); porous polysulfones, halogenated poly(vinylidene), polychloroethers, acetal polymers, polyesters prepared by esterification of a dicarboxylic acid or anhydride with an alkylene polyol, poly(alkylenesulfides), phenolics, polyesters, asymmetric porous polymers, cross-linked olefin polymers, hydrophilic microporous homopolymers, copolymers or interpolymers having a reduced bulk density, and other similar materials, poly(urethane), cross-linked chain-extended poly(urethane), poly(mides), poly(benzimidazoles), collodion, regenerated proteins, semi-solid cross-linked poly(vinylpyrrolidone).

As used herein, the terms “targeting domain”, “targeting moiety”, or “targeting group” are used interchangeably and refer to all molecules capable of specifically binding to a particular target molecule and forming a bound complex as described above. Thus, the ligand and its corresponding target molecule form a specific binding pair.

As used herein, the term “specific binding” refers to that binding which occurs between such paired species as enzyme/substrate, receptor/agonist, antibody/antigen, and lectin/carbohydrate which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions.

Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs. Thus, for example, an antibody preferably binds to a single epitope and to no other epitope within the family of proteins.

As used herein, the terms “peptide”, “polypeptide”, and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types, “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or any combination thereof.

The terms “patient,” “subject,” “individual,” and the like are used interchangeably herein, and refer to any animal, or cells thereof whether in vitro or in situ, amenable to the methods described herein.

In certain non-limiting embodiments, the patient, subject or individual is a human. In various embodiments, the subject is a human subject, and may be of any race, ethnicity, sex, and age.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The terms “cancer” or “neoplasm” as used herein, include, but are not limited to, benign and malignant cancers of the oral cavity (e.g., mouth, tongue, pharynx, etc.), digestive system (e.g., esophagus, stomach, small intestine, colon, rectum, liver, bile duct, gall bladder, pancreas, etc.), respiratory system (e.g., larynx, lung, bronchus, etc.), bones, joints, skin (e.g., basal cell, squamous cell, melanoma, etc.), breast, genital system, (e.g., uterus, ovary, prostate, testis, etc.), urinary system (e.g, bladder, kidney, ureter, etc.), eye, nervous system (e.g., brain, etc.), endocrine system (e.g., thyroid, etc.), and hematopoietic system (e.g., lymphoma, myeloma, leukemia, acute lymphocytic leukemia, chronic lymphocytic leukemia, acute myeloid leukemia, chronic myeloid leukemia, etc.).

As used herein, “treating a disease or disorder” means reducing the severity and/or frequency with which a sign or symptom of the disease or disorder is experienced by a subject.

A disease or disorder is “alleviated” if the severity of a sign or symptom of the disease or disorder, the frequency with which such a sign or symptom is experienced by a subject, or both, is reduced.

As used herein, the terms “therapy” or “therapeutic regimen” refer to those activities taken to alleviate or alter a disorder or disease state, e.g., a course of treatment intended to reduce or eliminate at least one sign or symptom of a disease or disorder using pharmacological, surgical, dietary and/or other techniques. A therapeutic regimen may include a prescribed dosage of one or more drugs or surgery. Therapies will most often be beneficial and reduce or eliminate at least one sign or symptom of the disorder or disease state, but in some instances the effect of a therapy will have non-desirable or side-effects. The effect of therapy will also be impacted by the physiological state of the subject, e.g., age, gender, genetics, weight, other disease conditions, etc.

By the term “modulating,” as used herein, is meant mediating a detectable increase or decrease in the level of a mRNA, polypeptide, or a response in a subject compared with the level of a mRNA, polypeptide or a response in the subject in the absence of a treatment or compound, and/or compared with the level of a mRNA, polypeptide, or a response in an otherwise identical but untreated subject. The term encompasses perturbing and/or affecting a native signal or response thereby mediating a beneficial therapeutic response in a subject, preferably, a human.

As used herein the terms “alteration,” “defect,” “variation,” or “mutation” refer to a mutation in a gene in a cell that affects the function, activity, expression (transcription or translation) or conformation of the polypeptide that it encodes. Mutations encompassed by the present invention can be any mutation of a gene in a cell that results in the enhancement or disruption of the function, activity, expression, or conformation of the encoded polypeptide, including the complete absence of expression of the encoded protein and can include, for example, missense and nonsense mutations, insertions, deletions, frameshifts, and premature terminations. Without being so limited, mutations encompassed by the present invention may alter splicing the mRNA (splice site mutation) or cause a shift in the reading frame (frameshift).

“Gene expression,” as used herein, encompasses the transcription of genomic DNA into mRNA and the translation of mRNA into protein.

A “genome” is all the genetic material of an organism. In some instances, the term genome may refer to the chromosomal DNA. Genome may be multichromosomal such that the DNA is cellularly distributed among a plurality of individual chromosomes. For example, in human there are 22 pairs of chromosomes plus a gender associated XX or XY pair. DNA derived from the genetic material in the chromosomes of a particular organism is genomic DNA. The term genome may also refer to genetic materials from organisms that do not have chromosomal structure. In addition, the term genome may refer to mitochondria DNA. A genomic library is a collection of DNA fragments representing the whole or a portion of a genome. Frequently, a genomic library is a collection of clones made from a set of randomly generated, sometimes overlapping DNA fragments representing the entire genome or a portion of the genome of an organism.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Homologous, homology” or “identical, identity” as used herein, refer to comparisons among amino acid and nucleic acid sequences. When referring to nucleic acid molecules, “homology,” “identity,” or “percent identical” refers to the percent of the nucleotides of the subject nucleic acid sequence that have been matched to identical nucleotides by a sequence analysis program. Homology can be readily calculated by known methods. Nucleic acid sequences and amino acid sequences can be compared using computer programs that align the similar sequences of the nucleic or amino acids and thus define the differences. In preferred methodologies, the BLAST programs (NCBI) and parameters used therein are employed, and the ExPaSy is used to align sequence fragments of genomic DNA sequences. However, equivalent alignment assessments can be obtained through the use of any standard alignment software.

As used herein, “homologous” refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 5′ATTGCC 3′ and 5′TATGGC 3′ share 50% homology.

As used herein, the term “fragment,” as applied to a nucleic acid, refers to a subsequence of a larger nucleic acid. A “fragment” of a nucleic acid can be at least about 15 nucleotides in length; for example, at least about 50 nucleotides to about 100 nucleotides; at least about 100 to about 500 nucleotides, at least about 500 to about 1000 nucleotides, at least about 1000 nucleotides to about 1500 nucleotides.

In one embodiment, about 1500 nucleotides to about 2500 nucleotides.

In one embodiment, about 2500 nucleotides (and any integer value in between).

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

As used herein, the term “purified” or “to purify” refers to the removal of components (e.g., contaminants) from a sample. For example, nucleic acids are purified by removal of contaminating cellular proteins or other undesired nucleic acid species. The removal of contaminants results in an increase in the percentage of desired nucleic acid in the sample.

The term “label” when used herein refers to a detectable compound or composition that is conjugated directly or indirectly to a probe to generate a “labeled” probe. The label may be detectable by itself (e.g, radioisotope labels or fluorescent labels) or, in the case of an enzymatic label, may catalyze chemical alteration of a substrate compound or composition that is detectable (e.g., avidin-biotin). In some instances, primers can be labeled to detect a PCR product.

As used herein, the term “nucleic acid” refers to both naturally-occurring molecules such as DNA and RNA, but also various derivatives and analogs. Generally, the probes, hairpin linkers, and target polynucleotides of the present teachings are nucleic acids, and typically comprise DNA. Additional derivatives and analogs can be employed as will be appreciated by one having ordinary skill in the art.

The term “nucleotide base”, as used herein, refers to a substituted or unsubstituted aromatic ring or rings. In certain embodiments, the aromatic ring or rings contain at least one nitrogen atom. In certain embodiments, the nucleotide base is capable of forming Watson-Crick and/or Hoogsteen hydrogen bonds with an appropriately complementary nucleotide base. Exemplary nucleotide bases and analogs thereof include, but are not limited to, naturally occurring nucleotide bases adenine, guanine, cytosine, 6 methyl-cytosine, uracil, thymine, and analogs of the naturally occurring nucleotide bases, e.g., 7-deazaadenine, 7-deazaguanine, 7-deaza-8-azaguanine, 7-deaza-8-azaadenine, N6 delta 2-isopentenyladenine (6iA), N6-delta 2-isopentenyl-2-methylthioadenine (2 ms6iA), N2-dimethylguanine (dmG), 7methylguanine (7mG), inosine, nebularine, 2-aminopurine, 2-amino-6-chloropurine, 2,6-diaminopurine, hypoxanthine, pseudouridine, pseudocytosine, pseudoisocytosine, 5-propynylcytosine, isocytosine, isoguanine, 7-deazaguanine, 2-thiopyrimidine, 6-thioguanine, 4-thiothymine, 4-thiouracil, 06-methylguanine, N6-methyladenine, 04-methylthymine, 5,6-dihydrothymine, 5,6-dihydrouracil, pyrazolo(3,4-D)pyrimidines (see, e.g., U.S. Pat. Nos. 6,143,877 and 6,127,121 and PCT published application WO 01/38584), ethenoadenine, indoles such as nitroindole and 4-methylindole, and pyrroles such as nitropyrrole. Certain exemplary nucleotide bases can be found, e.g., in Fasman, 1989, Practical Handbook of Biochemistry and Molecular Biology, pp. 385-394, CRC Press, Boca Raton, Fla., and the references cited therein.

The term “nucleotide”, as used herein, refers to a compound comprising a nucleotide base linked to the C-1′ carbon of a sugar, such as ribose, arabinose, xylose, and pyranose, and sugar analogs thereof. The term nucleotide also encompasses nucleotide analogs. The sugar may be substituted or unsubstituted. Substituted ribose sugars include, but are not limited to, those riboses in which one or more of the carbon atoms, for example the 2′-carbon atom, is substituted with one or more of the same or different Cl, F, —R, —OR, —NR2 or halogen groups, where each R is independently H, C1-C6 alkyl or C5-C14 aryl. Exemplary riboses include, but are not limited to, 2′-(C1-C6)alkoxyribose, 2′-(C5-C14)aryloxyribose, 2′,3′-didehydroribose, 2′-deoxy-3′-haloribose, 2′-deoxy-3′-fluororibose, 2′-deoxy-3′-chlororibose, 2′-deoxy-3′-aminoribose, 2′-deoxy-3′-(C1-C6)alkylribose, 2′-deoxy-3′-(C1-C6)alkoxyribose and 2′-deoxy-3′-(C5-C14)aryloxyribose, ribose, 2′-deoxyribose, 2′,3′-dideoxyribose, 2′-haloribose, 2′-fluororibose, 2′-chlororibose, and 2′-alkylribose, e.g., 2′-O-methyl, 4′-anomeric nucleotides, 1′-anomeric nucleotides, 2′-4′- and 3′-4′-linked and other “locked” or “LNA”, bicyclic sugar modifications (see, e.g., PCT published application nos. WO 98/22489, WO 98/39352; and WO 99/14226). The term “nucleic acid” typically refers to large polynucleotides.

The term “nucleotide analogs” as used herein refers to modified or non-naturally occurring nucleotides including, but not limited to, analogs that have altered stacking interactions such as 7-deaza purines (i.e., 7-deaza-dATP and 7-deaza-dGTP); base analogs with alternative hydrogen bonding configurations (e.g., such as Iso-C and Iso-G and other non-standard base pairs described in U.S. Pat. No. 6,001,983 to S. Benner and herein incorporated by reference); non-hydrogen bonding analogs (e.g., non-polar, aromatic nucleoside analogs such as 2,4-difluorotoluene, described by B. A. Schweitzer and E. T. Kool, J. Org. Chem., 1994, 59, 7238-7242; B. A. Schweitzer and E. T. Kool, J. Am. Chem. Soc., 1995, 117, 1863-1872); “universal” bases such as 5-nitroindole and 3-nitropyrrole; and universal purines and pyrimidines (such as “K” and “P” nucleotides, respectively; P. Kong, et al., Nucleic Acids Res., 1989, 17, 10373-10383, P. Kong et al., Nucleic Acids Res., 1992, 20, 5149-5152). Nucleotide analogs include nucleotides having one or more modification son the phosphate moiety, base moiety or sugar moiety, such as dideoxy nucleotides and 2′-O-methyl nucleotides. Nucleotide analogs include modified forms of deoxyribo-nucleotides as well as ribonucleotides.

Conventional notation is used herein to describe polynucleotide sequences: the left-hand end of a single-stranded polynucleotide sequence is the 5′-end. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.” In the sequences described herein:
A=adenine,
G=guanine,
T=thymine,
C=cytosine,
U=uracil,
H=A, C or T/U,
R=A or G,
M=A or C,
K=G or T/U,
S=G or C,
Y=C or T/U,
W=A or T/U,
B=G or C or T/U,
D=A or G, or T/U,
V=A or G or C,
N=A or G or C or T/U.

The skilled artisan will understand that all nucleic acid sequences set forth herein throughout in their forward orientation, are also useful in the compositions and methods of the invention in their reverse orientation, as well as in their forward and reverse complementary orientation, and are described herein as well as if they were explicitly set forth herein.

“Instructional material”, as that term is used herein, includes a publication, a recording, a diagram, or any other medium of expression which can be used to communicate the usefulness of the nucleic acid, peptide, and/or compound of the invention in the kit for identifying, diagnosing or alleviating or treating the various diseases or disorders recited herein. Optionally, or alternately, the instructional material may describe one or more methods of identifying, diagnosing or alleviating the diseases or disorders in a cell or a tissue of a subject. The instructional material of the kit may, for example, be affixed to a container that contains one or more components of the invention or be shipped together with a container that contains the one or more components of the invention. Alternatively, the instructional material may be shipped separately from the container with the intention that the recipient uses the instructional material and the components cooperatively.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range, such as from 1 to 6, should be considered to have specifically disclosed subranges, such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention relates, in part, to the discovery that various novel compounds containing non-saturated heterocyclic 5-member rings selectively inhibited the polymerase domain of the POLQ gene product DNA polymerase theta (Polq). The present invention also relates, in part, to pharmaceutical formulations comprising said derivatives containing non-saturated heterocyclic 5-member rings as well as methods of treating diseases or disorders, such as cancers defective in homology directed repair (HDR)(or homologous recombination), non-homologous end-joining, or other DNA damage response pathways by inhibiting Polq (e.g., the activity of Polq, the level of Polq, etc.) with disclosed derivatives containing non-saturated heterocyclic 5-member rings and analogs thereof.

The present invention is based, in part, on the discovery that that novel compounds containing non-saturated heterocyclic 5-member rings presented herein inhibit Polq DNA synthesis activity. For example, compounds containing aryl motifs flanking each end of an amide or carbamate motif that contain a non-saturated heterocyclic 5-member ring attached to one of the aryl motifs inhibit Polq DNA synthesis activity. Accordingly, the present invention provides methods and compositions for inhibiting Polq in vitro and in vivo. The present invention also demonstrates that compounds containing non-saturated heterocyclic 5-member rings and derivatives thereof that inhibit Polq activity additionally preferentially inhibit the proliferation of BRCA-deficient cancer cells or HDR-deficient cancer cells. The present invention also demonstrates that compounds containing non-saturated heterocyclic 5-member rings and derivatives thereof that inhibit Polq activity additionally inhibit the proliferation of BRCA-deficient cancer cells or HDR-deficient cancer cells in combination with PARP inhibitor (PARPi) treatment.

Polq is highly expressed in many types of cancer cells, confers resistance to ionizing radiation and various chemotherapy agents including etoposide, camptothecin and cisplatin, and promotes the survival of cancer cells, such as those deficient in HDR or other DNA repair or DDR pathways. High expression levels of Polq corresponds to a poor clinical outcome for cancer patients. Accordingly, another aspect of the invention provides a method of treating cancer in a subject by administering a composition of the invention.

In one embodiment, the method comprises administering a composition containing a non-saturated heterocyclic 5-member ring, an analog of a compound containing a non-saturated heterocyclic 5-member ring, a prodrug version of compound containing a non-saturated heterocyclic 5-member ring or derivative thereof, or a combination thereof. In some embodiments, the cancer is resistant to at least one type of chemotherapy agent. In some embodiments, the cancer is breast cancer. In some embodiments, the cancer is ovarian cancer. In some embodiments, the cancer is leukemia. In some embodiments, the cancer is prostate cancer. In some embodiments, the cancer is pancreatic cancer.

### DNA Polymerase Theta (Polq) Inhibitors

In one aspect, the present invention provides, in part, novel A family polymerase inhibitors. In one embodiment, the A family polymerase is DNA polymerase theta (Polq). In some embodiments, the A family polymerase is a fragment of Polq. In some embodiments, the fragment of Polq is Polq₁₇₉₂₋₂₅₉₀ (SEQ ID NO 1) or a fragment thereof. In some embodiments, the fragment of Polq is a fragment of Polq₁₋₂₅₉₀ (SEQ ID NO 3). In some embodiments, Polq is encoded by the human POLQ gene. In other embodiments, Polq is encoded by the Mus musculus Polq gene. In other embodiments, Polq is encoded by the C. elegans polq−1 gene.

Thus, in various embodiments, the present invention provides compounds that modulate or inhibit the level or activity of at least one A family polymerase (e.g., Polq). In another aspect, the present invention provides compounds useful for preventing or treating a disease or disorder (e.g., cancer). In various aspects, the compound of the present invention is a functionalized pyrrolidine or imidazolidine based compound or derivative thereof.

### Compounds

In one aspect, the present invention relates to a functionalized compound containing a non-saturated heterocyclic 5-member ring or derivative such as compounds having the structure of Formula (I), or a tautomeric or a stereochemically isomeric form, a pharmaceutically acceptable salt or a solvate thereof:

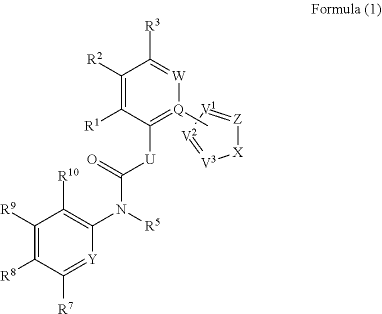

wherein:
W represents C(R⁴) or N;
U represents CH₂, O, S, or NR^{U};
Y represents C(R⁶) or N;
ring A represents an unsaturated 5-membered ring;
Q represents a carbon atom directly bonded to ring A;
R¹, R², R³, R⁴, R⁷, R⁸, R⁹, R¹⁰, and R^{U} independently represent hydrogen, deuterium, C₁₋₆ alkyl, C₂₋₆ alkenyl, alkynyl, hydroxy, thiol, C₁₋₆ alkoxy, halogen, haloC₁₋₆ alkyl, haloC₁₋₆ alkoxy, C₃₋₈ cycloalkyl, nitrile, NR^{X}R^{Y}, and combinations thereof; wherein two adjacent groups R¹ to R⁴ or R⁶ to R¹⁰ optionally join to form a 5- to 7-membered saturated or unsaturated ring optionally containing one or more heteroatoms selected from O, N or S;
R⁵ and R⁶ independently represent hydrogen, deuterium, C₁₋₆ alkyl, C₂₋₆ alkenyl, alkynyl, hydroxy, thiol, C₁₋₆ alkoxy, halogen, haloC₁₋₆ alkyl, haloC₁₋₆ alkoxy, C₃₋₈ cycloalkyl, nitrile, —NR^{X}R^{Y}, aryl, heteroaryl, heterocyclyl, amide, ester, sulfone, sulfonamide, sulfoxide, and combinations thereof;
Z represents CR^{Z} or N, V¹ is C, and the bond between V¹ and Z is a double bond; or
Z represents NR^{Z}, CR^{Z}R^{Z′}, C═S, or C═O, V¹ is N, and the bond between V¹ and Z is a single bond;
V² represents N or CR^{V2}
V³ represents N or CR^{V3}
X represents C(R¹⁵)(R¹⁶), N(R¹⁷), S, or O;
R¹⁵, R¹⁶, and R¹⁷ independently represent hydrogen, deuterium, C₁₋₂₀ alkyl, haloC₁₋₂₀ alkyl, —OR^{15a}, —SR^{15a}, nitrile, —COC₁₋₂₀ alkyl, —COOC₁₋₂₀ alkyl, hydroxy, C₁₋₂₀ alkoxy, C₁₋₂₀ alkanol, C₃₋₈ cycloalkyl, halogen, carbonyl, —NR^{V}R^{W}, —CH₂—NR^{V}R^{W}, —OSO₂NH₂, —P(O)OH₂, aryl, heteroaryl, heterocyclyl, and combinations thereof; wherein R¹⁵, R¹⁶, and R¹⁷ may further comprise one or more divalent linkers L selected from the group consisting alkylene, cycloalkylene, heteroalkylene, heterocycloalkylene, alkenylene, alkynylene, arylene, heteroarylene, silyl, amine, amide, ester, ether, carbonyl, carbamate, carbonate, sulfamate, sulfonic ester, sulfoximine, sulfonamide, thioether, thioester, disulfide, hydrazine, urea, thiourea, phosphate, phosphonate ester, poly(alkyl ether), heteroatom, and combinations thereof;
R^{Z}, R^{Z′}, R^{V2}, and R^{V3} independently represent hydrogen, deuterium, C₁₋₆ alkyl, hydroxy, C₁₋₆ alkoxy, C₁₋₆ alkanol, halogen, —OR^{15b}, CO₂H, CO₂R^{15b}, haloC₁₋₆ alkyl, and combinations thereof; provided one of R^{Z}, R^{Z′}, R^{V2}, R^{V3}, R¹⁵, R¹⁶, and R¹⁷ represents a direct bond to Q;
each R^{15a} and R^{15b} independently represents hydrogen, deuterium, C₁₋₂₀ alkyl, or aryl;
R^{V}, R^{W}, R^{X} and R^{Y} independently represent hydrogen, deuterium, C₁₋₂₀ alkyl, haloC₁₋₂₀ alkyl, C₃₋₈ cycloalkyl, —COC₁₋₂₀ alkyl or heterocyclyl; wherein said alkyl groups may be optionally substituted with or more deuterium, hydroxy, amino or sulfone groups; and said heterocyclyl ring may be optionally substituted by one or more deuterium, oxo, hydroxy, C₁₋₂₀ alkanol or —COC₁₋₂₀ alkyl groups.

In one embodiment, the compound of Formula (I) is represented by Formula (I′):

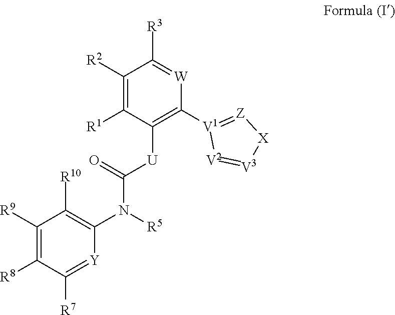

In one embodiment, the compound of Formula (I) is represented by Formula (Ia):

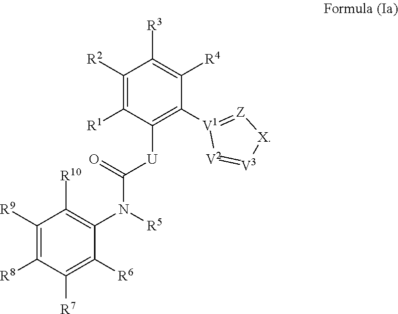

In one embodiment, the compound of Formula (I) is represented by Formula (Ib):

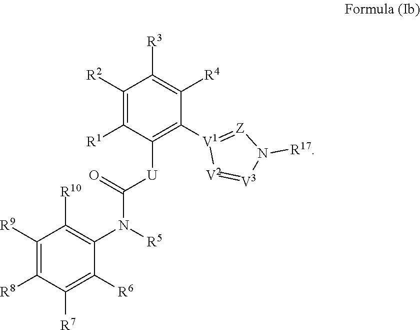

In one embodiment, the compound of Formula (I) is represented by Formula (Ic) or Formula (Ic′):

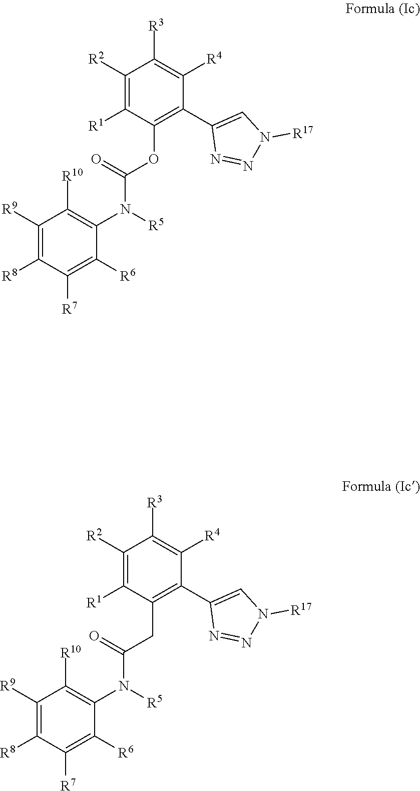

In one embodiment, the compound of Formula (I) is represented by Formula (Id) or Formula (Id′):

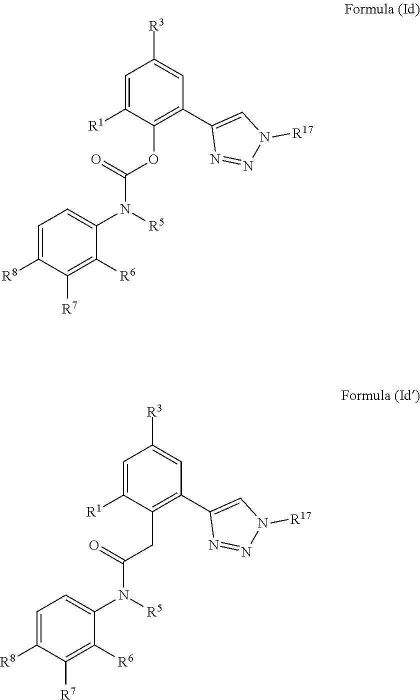

In one embodiment, the compound of Formula (I) is represented by Formula (Ie) or Formula (Ie′):

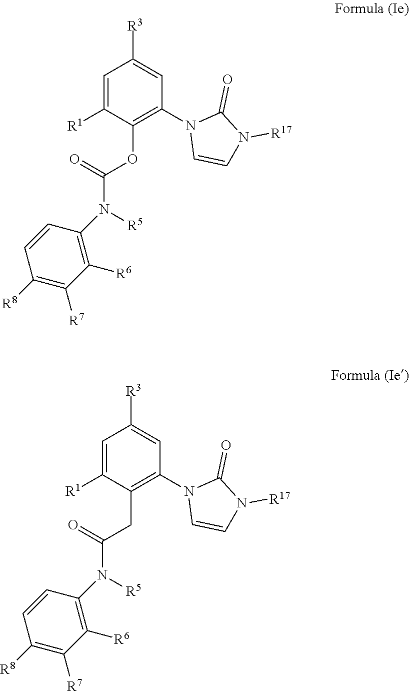

In one embodiment, the compound of Formula (I) is represented by Formula (If) or Formula (If′):

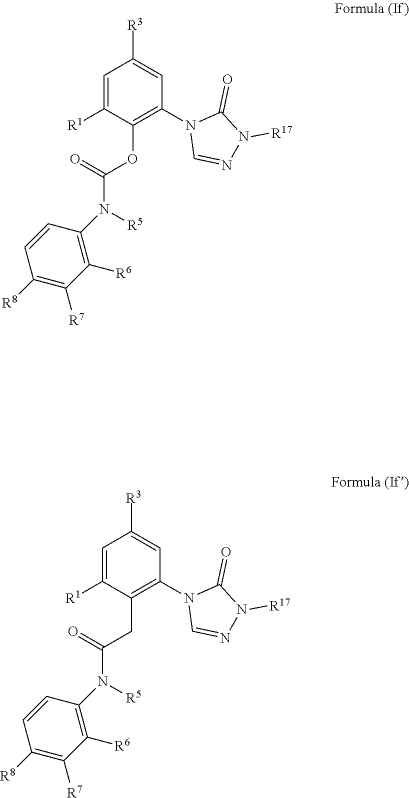

In one embodiment, the compound of Formula (I) is represented by Formula (Ig) or Formula (Ig′):

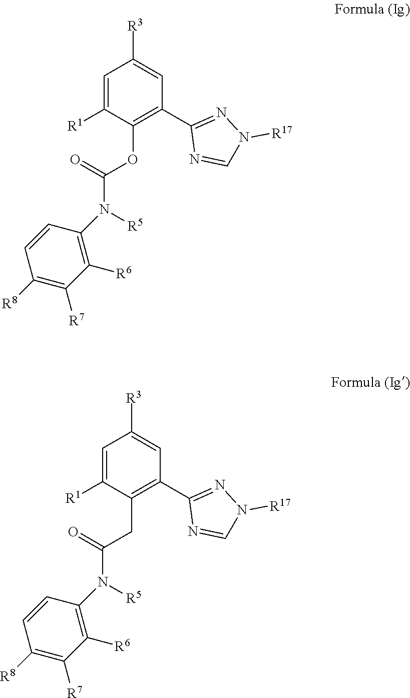

In one embodiment, the compound is represented by Formula (Ih) or Formula (Ih′):

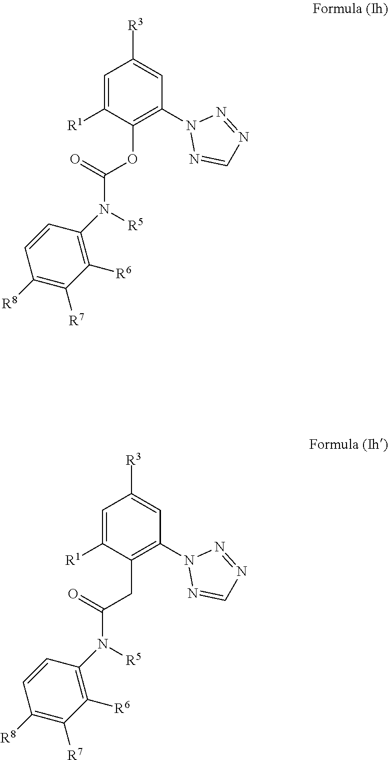

In one embodiment, the compound of Formula (I) is represented by Formula (Ii) or Formula (Ii′):

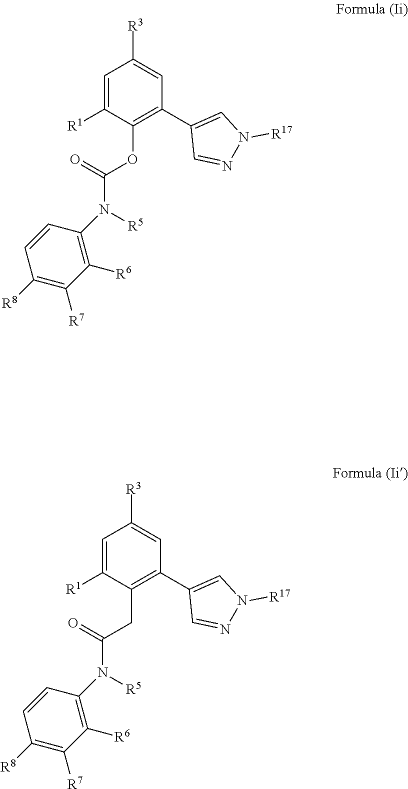

In one embodiment, the compound of Formula (I) is represented by Formula (Ij) or Formula (Ij′):

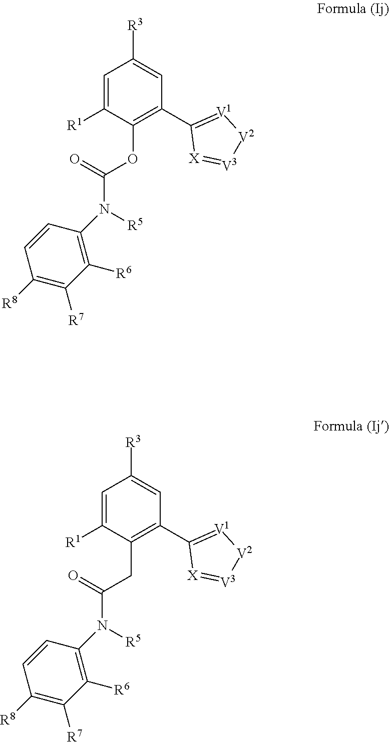

In one embodiment, U is O. In one embodiment, U is CH₂. In one embodiment, U is S.

In one embodiment, R⁷ and R⁸ or R⁸ and R⁹ join to form a pyrrolyl ring which is optionally substituted. In one embodiment, Y is N. In one embodiment, Z represents C═O. In one embodiment, Z represents C═S. In one embodiment, Z represents CR^{Z}R^{Z′}. In one embodiment, R^{Z} and R^{Z′} each represent hydrogen or deuterium.

In one embodiment, R¹⁷ represents hydrogen, deuterium, C₁₋₂₀ alkyl, haloC₁₋₂₀ alkyl, —OR^{15a}, —SR^{15a}, nitrile, —COC₁₋₂₀ alkyl, —COOC₁₋₂₀ alkyl, hydroxy, C₁₋₂₀ alkoxy, C₁₋₂₀ alkanol, C₃₋₈ cycloalkyl, halogen, carbonyl, —NR^{V}R^{W}, —CH₂—NR^{V}R^{W}, —OSO₂NH₂, —P(O)OH₂, aryl, heteroaryl, heterocyclyl, and combinations thereof. In one embodiment, R¹⁷ further comprises a divalent linker selected from the group consisting alkylene, cycloalkylene, heteroalkylene, heterocycloalkylene, alkenylene, alkynylene, arylene, heteroarylene, silyl, amine, amide, ester, ether, carbonyl, carbamate, sulfamate, thioether, thioester, disulfide, hydrazine, urea, thiourea, phosphate, phosphonate ester, poly(alkyl ether), heteroatom, and combinations thereof. In one embodiment, R¹⁷ comprises one or more divalent linkers. In one embodiment, R¹⁷ has the structure —CH₂CH₂O-L-R^{17′}, wherein R^{17′} represents hydrogen, deuterium, C₁₋₂₀ alkyl, haloC₁₋₂₀ alkyl, —OR^{15a}, —SR^{15a}, nitrile, —COC₁₋₂₀ alkyl, —COOC₁₋₂₀ alkyl, hydroxy, C₁₋₂₀ alkoxy, C₁₋₂₀ alkanol, C₃₋₈ cycloalkyl, halogen, carbonyl, —NR^{V}R^{W}, —CH₂—NR^{V}R^{W}, —OSO₂NH₂, aryl, heteroaryl, heterocyclyl, and combinations thereof.

In one embodiment, R¹⁷ represents one of the following substituents:

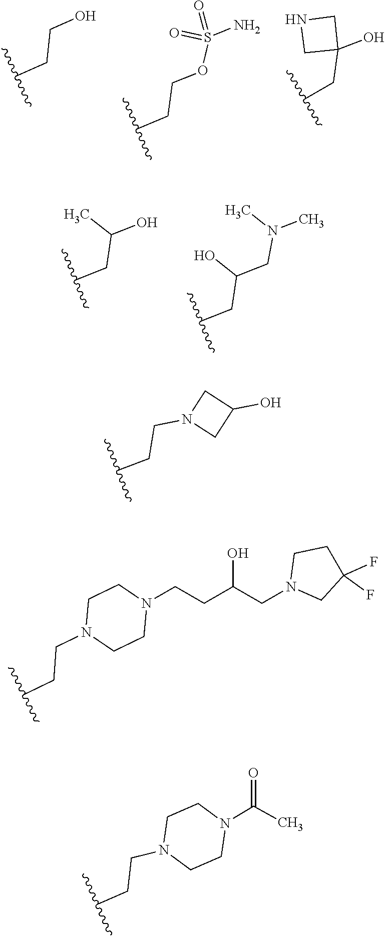

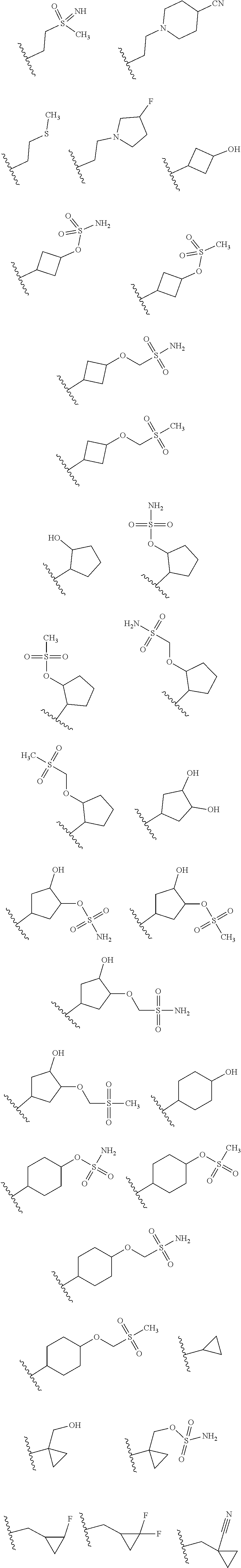

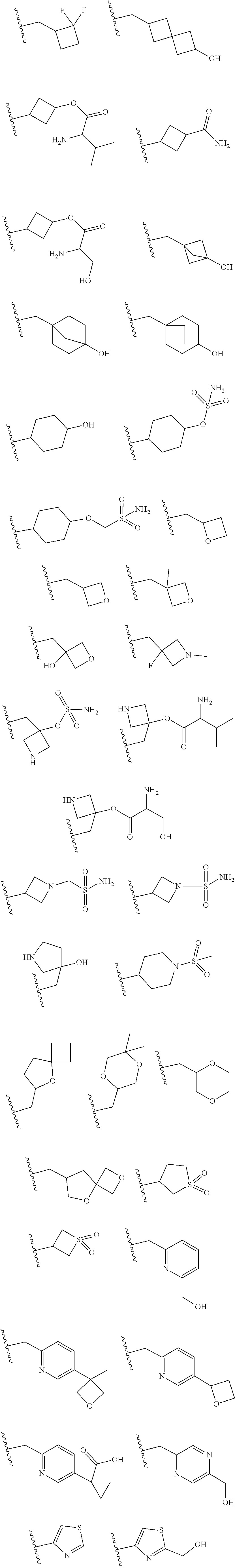

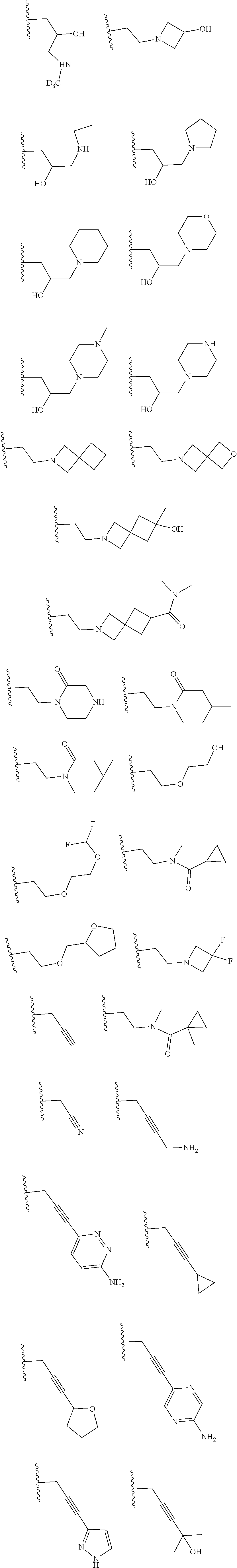

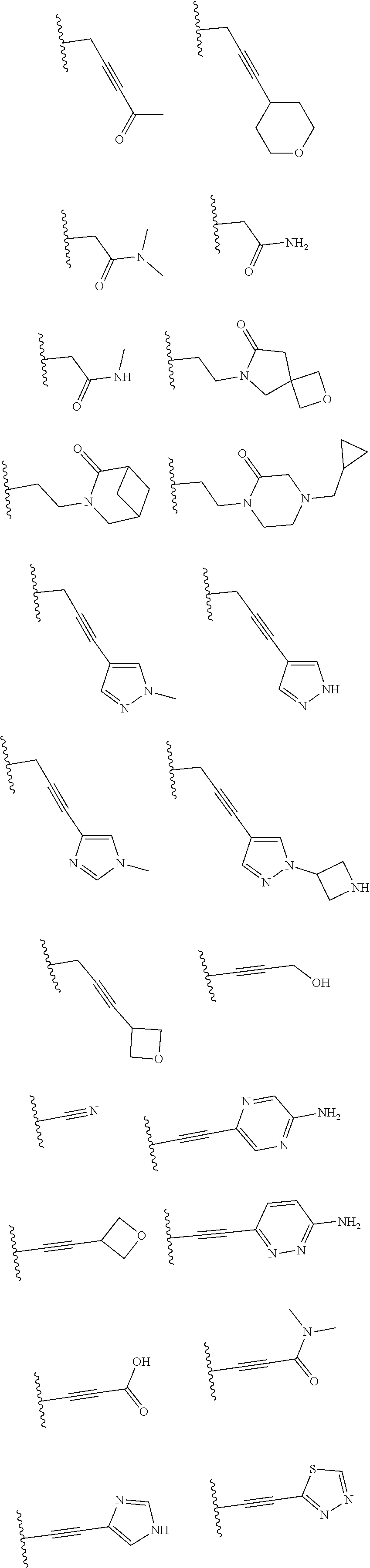

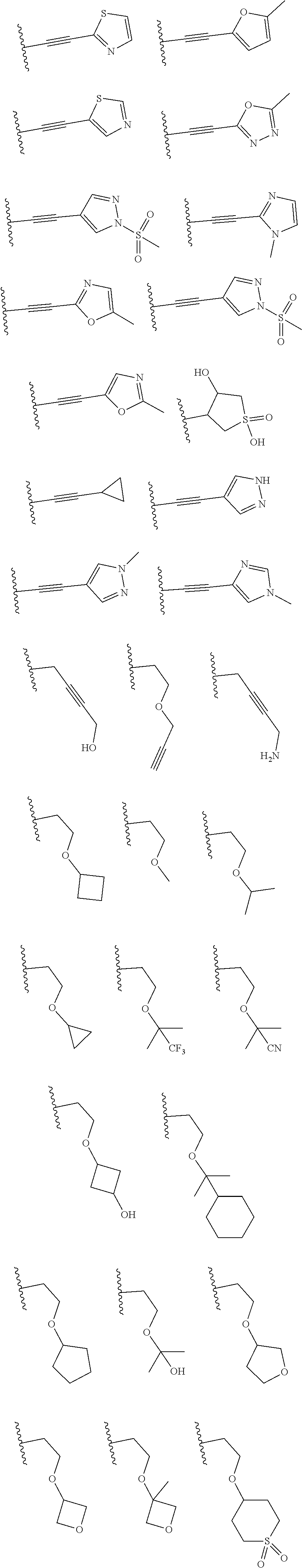

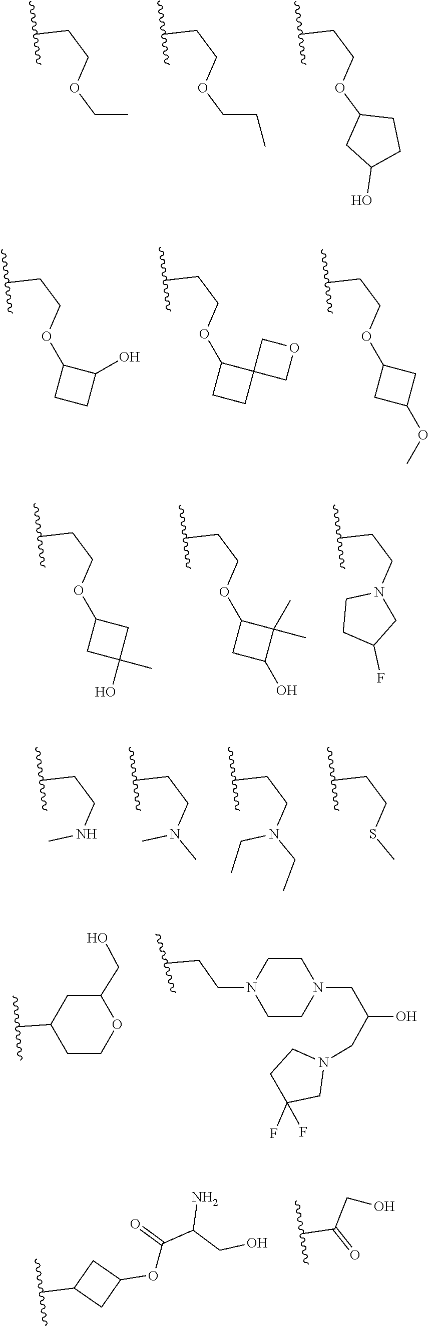

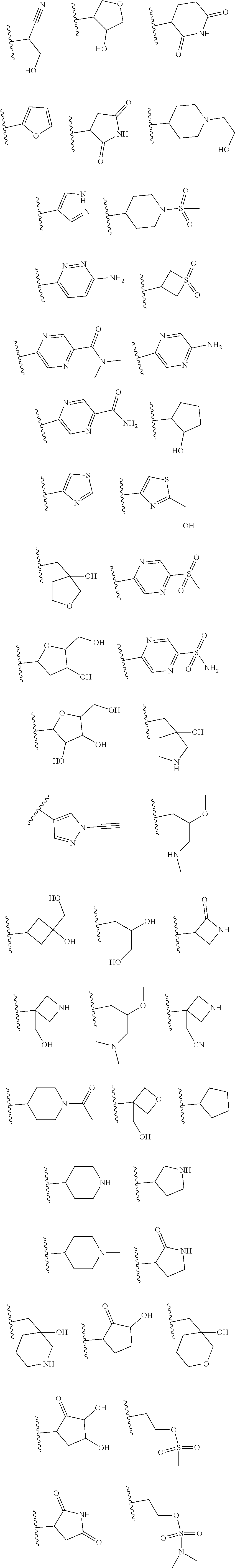

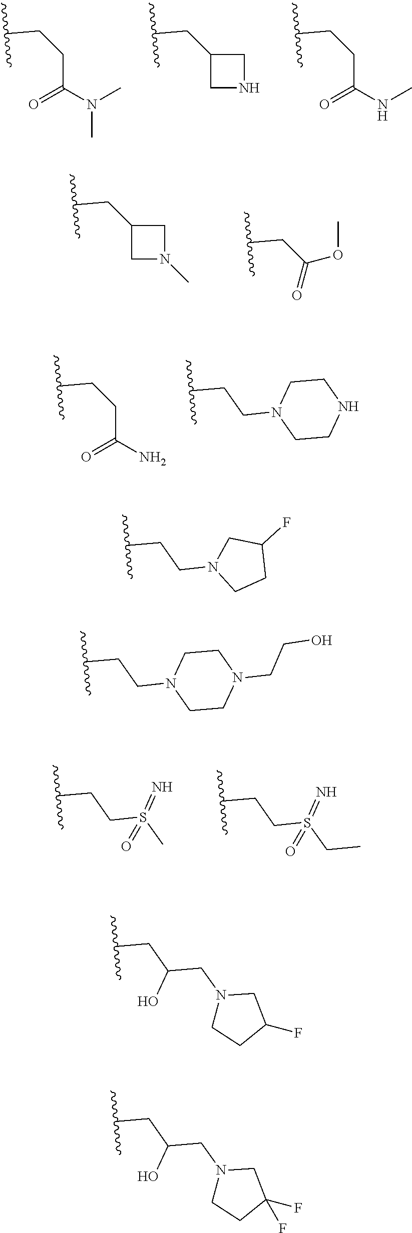

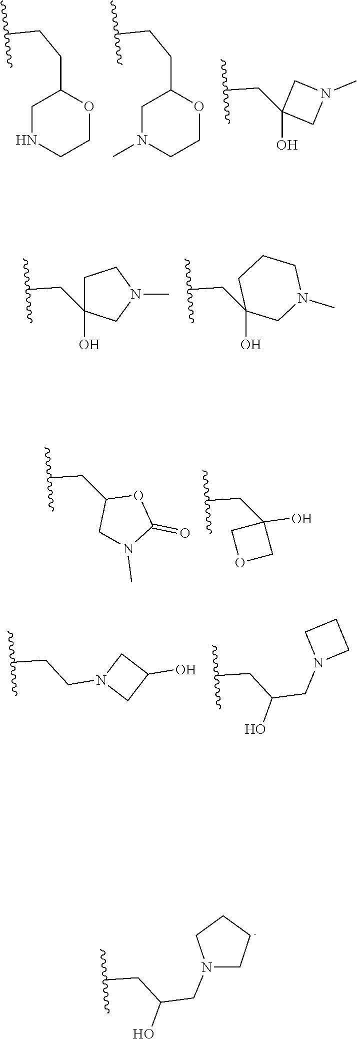

In one embodiment, R¹⁷ is represented by:

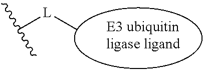

wherein L represents a divalent organic linker; and E3 ubiquitin ligase ligand represents any known E3 ubiquitin ligase ligand known in the art, including but not limited to pomalidomide, Von Hippel-Lindau (VHL), thalidomide, lenalidomide, iberdomide, and apremilast. In one embodiment, the E3 ubiquitin ligase ligand is an inhibitor of apoptosis (IAP) ligands, such as bestatin and MV1 derivative. In one embodiment, the E3 ubiquitin ligase ligand is Mouse double minute 2 homolog (MDM2).

In one embodiment, L is selected from the group consisting of alkylene, cycloalkylene, heteroalkylene, heterocycloalkylene, alkenylene, alkynylene, arylene, heteroarylene, silyl, amine, amide, ester, ether, carbonyl, carbamate, sulfamate, thioether, thioester, disulfide, hydrazine, urea, thiourea, phosphate, phosphonate ester, poly(alkyl ether), heteroatom, and combinations thereof.

In one embodiment, L comprises one or more of the following groups: amides, carbamates, carbomate, ester bonds, branched alkyl, cyclic alkyls, various heteroatoms, piperazine type of motifs, cyclobutane, cyclopropal, cyclohexane, aromatic rings, saturated rings, non-saturated rings, and heterocyclic rings saturated and non-saturated.

In one embodiment, R¹⁷ is represented by one of the following structures:

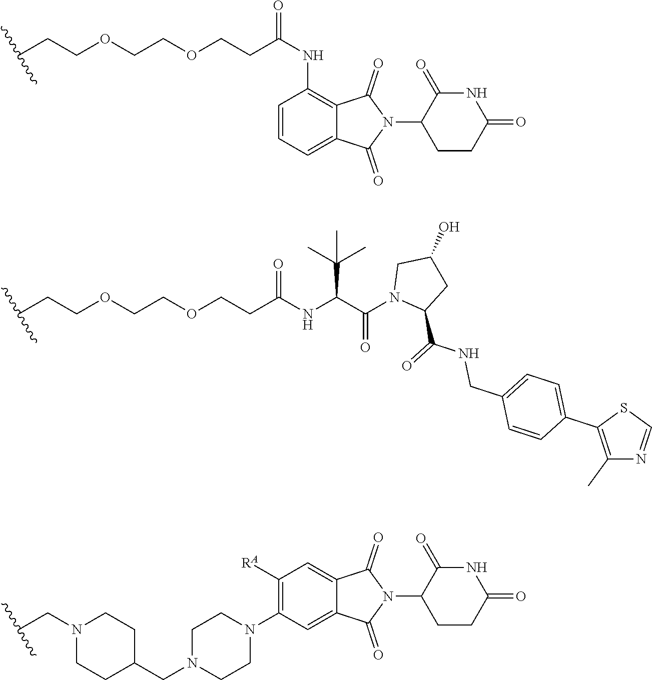

wherein R^{A} is H or halogen.

In one embodiment, W represents CR⁴. In an alternative embodiment, W represents N. In one embodiment, W represents CH, C(CN), or N. In one embodiment, W represents C(Cl) or CF.

In one embodiment, at least one of R¹ and R³ represents halogen or haloC₁₋₆ alkyl. In one embodiment, R¹ and R³ independently represent halogen or haloC₁₋₆ alkyl. In one embodiment, at least one of R¹ and R³ represents haloC₁₋₆ alkyl. In one embodiment, at least one of R¹ and R³ represents halogen. In one embodiment, R¹ and R³ each represent haloC₁₋₆ alkyl. In one embodiment, R¹ and R³ each independently represent halogen.

In one embodiment, R¹ represents hydrogen, deuterium, methyl, CD₃, haloC₁₋₆ alkyl (such as CF₃, CHF₂, or CH₂F), haloC₁₋₆ alkoxyl (such as OCF₃, OCHF₂, or OCH₂F), or halogen (such as fluorine or chlorine).

In one embodiment, R² represents hydrogen; deuterium C₁₋₆ alkyl (such as methyl or ethyl); C₂₋₆ alkenyl (such as ethenyl); C₁₋₆ alkoxy (such as methoxy); halogen (such as chlorine). In one embodiment, R² represents NR^{X}R^{Y} (such as N(Me)₂ or N(Me)(Et)). In one embodiment, R² represents: hydrogen; C₁₋₆ alkyl (such as methyl); C₁₋₆ alkoxy (such as methoxy). In one embodiment, R² represents —NR^{X}R^{Y} (such as —N(Me)₂ or —N(Me)(Et)). In one embodiment, R² represents C₁₋₆ alkyl (such as methyl). In one embodiment, R² represents: hydrogen; halogen (such as chlorine). In one embodiment, R² represents C₁₋₆ alkyl (such as methyl). In one embodiment, R² represents hydrogen.

In one embodiment, R³ represents hydrogen, deuterium, methyl, CD₃, haloC₁₋₆ alkyl (such as CF₃, CHF₂, or CH₂F), haloC₁₋₆ alkoxy (such as OCF₃, OCHF₂, or OCH₂F), or halogen (such as fluorine or chlorine). In one embodiment, R³ represents C₁₋₆ alkyl (such as methyl, ethyl or isopropyl); C₂₋₆ alkenyl (such as —C(═CH₂)(Me)); halogen (such as bromine); haloC₁₋₆ alkyl (such as trifluoromethyl or —C(H)(Me)-CF3).

In one embodiment, R³ represents haloC₁₋₆ alkoxy (such as difluoromethoxy). In one embodiment, R³ represents C₁₋₆ alkyl (such as methyl, ethyl or isopropyl).

In one embodiment, R³ represents haloC₁₋₆ alkyl (such as trifluoromethyl). In one embodiment, R³ represents haloC₁₋₆ alkyl (such as trifluoromethyl).

In one embodiment, R⁴ represents hydrogen, deuterium, C₁₋₆ alkyl (such as methyl, ethyl or isopropyl), CD₃, C₂ alkynyl, or nitrile. In one embodiment, R⁴ represents hydrogen.

In one embodiment, R⁵ represents CH₃ or CD₃. In one embodiment, R⁵ represents cyclopropyl or —CH₂CN. In one embodiment, R⁵ represents the following structure:

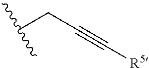

wherein R^{5′} is selected from the group consisting of hydrogen, deuterium, C₁₋₆ alkyl, C₂₋₆ alkenyl, alkynyl, hydroxy, thiol, C₁₋₆alkoxy, halogen, haloC₁₋₆ alkyl, haloC₁₋₆ alkoxy, C₃₋₈ cycloalkyl, nitrile, —NR^{X}R^{Y}, aryl, heteroaryl, heterocyclyl, amide, and combinations thereof.

In one embodiment, R⁵ represents one of the following substituents:

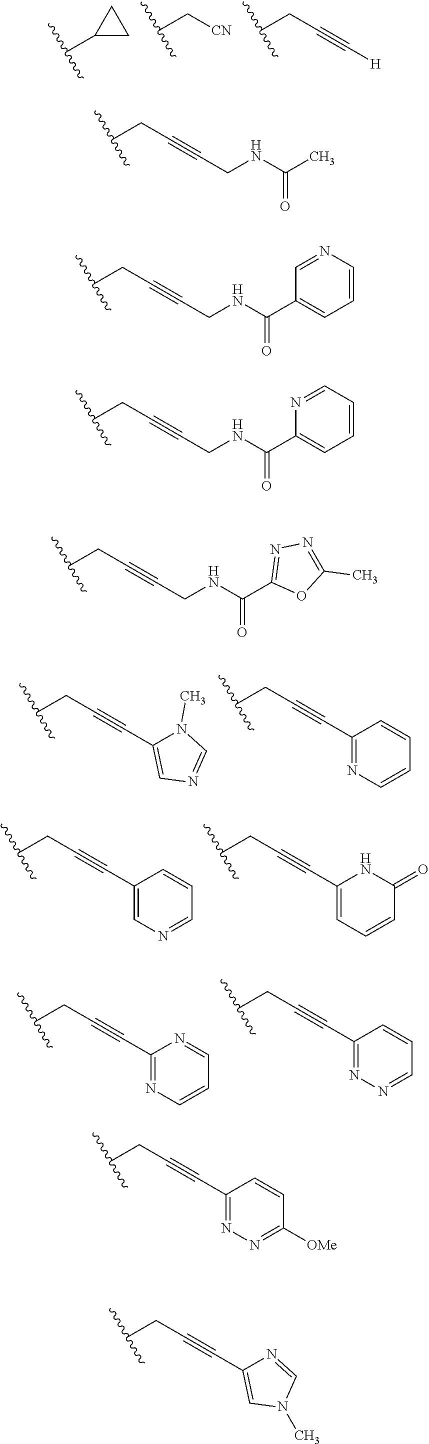

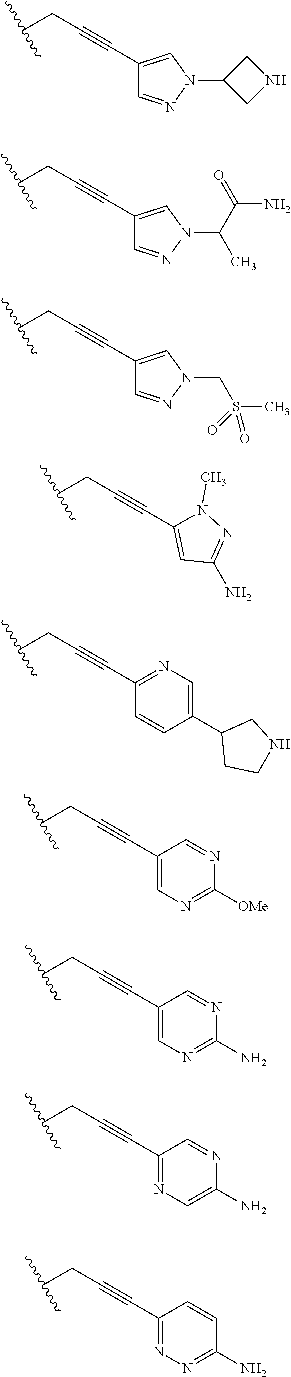

In one embodiment, R⁶ represents CH₃ or CD₃. In one embodiment, R⁶ represents cyclopropyl or CH₂CN. In one embodiment, R⁶ represents the following structure:

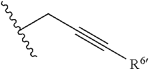

wherein R^{6′} is selected from the group consisting of hydrogen, deuterium, C₁₋₆ alkyl, C₂₋₆ alkenyl, alkynyl, hydroxy, thiol, C₁₋₆ alkoxy, halogen, haloC₁₋₆ alkyl, haloC₁₋₆ alkoxy, C₃₋₈ cycloalkyl, nitrile, —NR^{X}R^{Y}, aryl, heteroaryl, heterocyclyl, amide, and combinations thereof.

In one embodiment, R⁶ represents one of the following substituents:

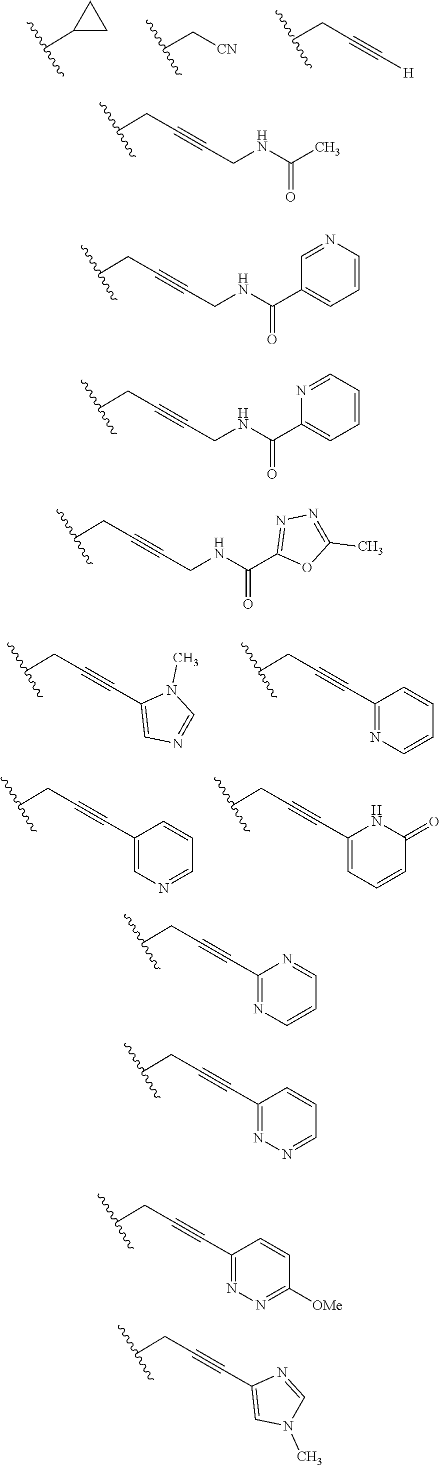

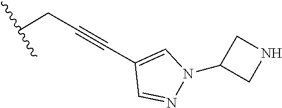 [SMILES: *CC#Cc1cnn(C2CNC2)c1]

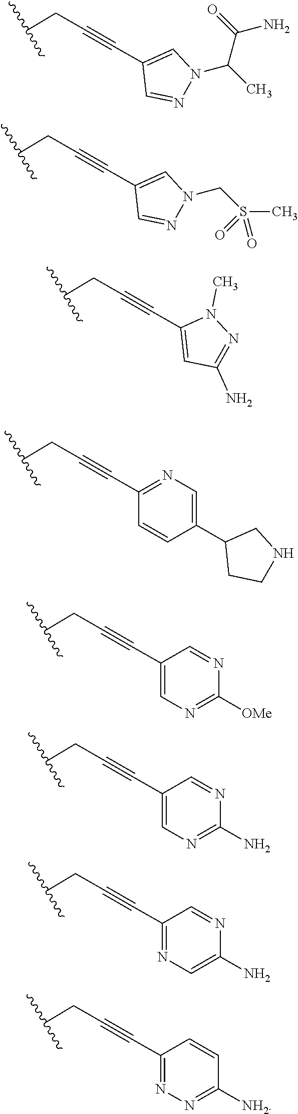

In one embodiment, R^{X} and R^{Y} represent C₁₋₆ alkyl (such as methyl or ethyl). In one embodiment, R^{X} and R^{Y} both represent C₁₋₆ alkyl (such as methyl or ethyl). In one embodiment, R^{X} and R^{Y} both represent methyl or one represents methyl and the other represents ethyl.

In one embodiment, W represents C(R⁴); R¹ represents C₁₋₆ alkyl (such as methyl), R² represents hydrogen, R³ represents haloC₁₋₆ alkyl (such as trifluoromethyl) and R⁴ represents nitrile.

In one embodiment, R¹ and R³ each independently represents halogen, haloC₁₋₆ alkyl, or CF₃.

In one embodiment, R¹ represents C₁₋₆ alkyl (such as methyl), R² represents hydrogen, R³ represents C₁₋₆ alkyl (such as methyl or isopropyl) and R represents nitrile.

In one embodiment, R¹ represents C₁₋₆ alkyl (such as methyl or ethyl), R² represents hydrogen, R³ represents haloC₁₋₆ alkyl (such as trifluoromethyl or —CH(Me)-CF3) and R⁴ represents hydrogen.

In one embodiment, R¹ represents C₁₋₆ alkyl (such as methyl), R² represents hydrogen, R³ represents C₁₋₆ alkyl (such as isopropyl) and R⁴ represents hydrogen.

In one embodiment, R¹ represents C₁₋₆ alkyl (such as methyl), R² represents halogen (such as chlorine), R³ represents C₁₋₆ alkyl (such as methyl) and R⁴ represents hydrogen.

In one embodiment, R¹ represents C₁₋₆ alkoxy (such as methoxy), R² represents hydrogen, R³ represents haloC₁₋₆ alkyl (such as trifluoromethyl) and R⁴ represents nitrile.

In one embodiment, R¹ represents —NR^{X}R^{Y} (such as —N(Me)2 or —N(Me)(Et)). R² represents hydrogen. R³ represents haloC₁₋₆ alkyl (such as trifluoromethyl), and R⁴ represents nitrile.

In one embodiment, R¹ represents hydrogen, R² represents hydrogen, R³ represents haloC₁₋₆ alkyl (such as trifluoromethyl) and R⁴ represents nitrile.

In one embodiment, R¹ represents hydrogen, R² represents C₁₋₆ alkyl (such as methyl), R³ represents C₁₋₆ alkyl (such as ethyl) and R⁴ represents hydrogen.

In one embodiment, R¹ represents halogen (such as chlorine), R² represents hydrogen. R³ represents haloC₁₋₆ alkyl (such as trifluoromethyl) and R⁴ represents hydrogen.

In one embodiment, R¹ represents halogen (such as chlorine), R² represents hydrogen. R³ represents haloC₁₋₆ alkoxy (such as difluoromethoxy) and R⁴ represents hydrogen.

In one embodiment, R¹ represents C₁₋₆ alkenyl (such as ethenyl), R² represents hydrogen, R³ represents haloC₁₋₆ alkyl (such as trifluoromethyl) and R⁴ represents hydrogen.

In one embodiment, R¹ represents C₁₋₆ alkyl (such as methyl), R² represents hydrogen, R³ represents haloC₁₋₆ alkoxy (such as difluoromethoxy) and R⁴ represents hydrogen.

In one embodiment, R¹ represents C₁₋₆ alkyl (such as methyl), R² represents hydrogen, R³ represents C₂₋₆ alkenyl (such as —C(Me)(═CH₂)) and R⁴ represents hydrogen.

In one embodiment, R¹ represents C₁₋₆ alkyl (such as methyl), R² represents hydrogen, R³ represents halogen (such as bromine) and R⁴ represents hydrogen.

In one embodiment, R¹ represents C₁₋₆ alkyl (such as methyl), R² represents hydrogen, R³ represents haloC₁₋₆ alkyl (such as trifluoromethyl) and R⁴ represents nitrile;

In one embodiment, R¹ represents C₁₋₆ alkyl (such as methyl), R² represents hydrogen, R³ represents C₁₋₆ alkyl (such as methyl or isopropyl) and R⁴ represents nitrile.

In one embodiment, R¹ represents C₁₋₆ alkyl (such as methyl), R² represents hydrogen, R³ represents haloC₁₋₆ alkyl (such as trifluoromethyl) and R⁴ represents hydrogen.

In one embodiment, R¹ represents C₁₋₆ alkyl (such as methyl), R² represents hydrogen, R³ represents C₁₋₆ alkyl (such as isopropyl) and R⁴ represents hydrogen.

In one embodiment, R¹ represents C₁₋₆ alkyl (such as methyl), R² represents halogen (such as chlorine), R³ represents C₁₋₆ alkyl (such as methyl) and R⁴ represents hydrogen.

In one embodiment, R¹ represents C₁₋₆ alkoxy (such as methoxy), R² represents hydrogen. R³ represents haloC₁₋₆ alkyl (such as trifluoromethyl) and R⁴ represents nitrile.

In one embodiment, R¹ represents —NR^{x}R^{y} (such as —N(Me)₂ or —N(Me)(Et)), R² represents hydrogen, R³ represents haloC₁₋₆ alkyl (such as trifluoromethyl) and R⁴ represents nitrile.

In one embodiment, R¹ represents hydrogen, R² represents hydrogen, R³ represents haloC₁₋₆ alkyl (such as trifluoromethyl) and R⁴ represents nitrile.

In one embodiment, R¹ represents hydrogen, R² represents C₁₋₆ alkyl (such as methyl), R³ represents C₁₋₆ alkyl (such as ethyl) and R⁴ represents hydrogen.

In one embodiment, R¹ represents C₁₋₆ alkyl (such as methyl), R² represents hydrogen, R³ represents haloC₁₋₆ alkyl (such as trifluoromethyl) and R⁴ represents hydrogen.

In one embodiment, R¹ represents N, R¹ represents C₁₋₆ alkyl (such as methyl), R² represents hydrogen and R³ represents haloC₁₋₆ alkyl (such as trifluoromethyl).

In one embodiment, X represents C(R¹⁵)(R¹⁶) or O. In one embodiment, X represents C(R¹⁵)(R¹⁶). In one embodiment, X represents O. In one embodiment, X represents —N(R¹⁷)—.

In one embodiment, Z represents CH₂ or C═O. In one embodiment, Z represents CH₂. In one embodiment, Z represents C═O. In one embodiment, Z represents C(R^{Z})(H). In one embodiment, Z represents C(CH₃)(H).

In one embodiment, R¹⁵ and R¹⁶ independently represent hydrogen; —OR^{15a} (such as hydroxy); halogen (such as fluorine); C₁₋₆ alkanol (such as CH₂OH); C₁₋₆ alkoxy (such as methoxy); —NR^{V}R^{W} (such as —NH₂, —NMe₂, —N(H)(Me), —N(H)(COMe), —N(H)((CH₂)₂OH), —N(H)((CH₂)₂SO₂Me), N(Me)((CH₂)₂SO₂Me), —N(H)(pyrrolidinyl), —N(Me)(pyrrolidinyl), —N(H)(azetidinyl), —N(H)(oxetanyl), —N(Me)(oxetanyl), —N(H)(cyclopentyl), —N(Me)(cyclopentyl), —N(H)(tetrahydropyranyl), —N(Me)(tetrahydropyranyl) or —N(H)((CH₂)₂NH₂), wherein said pyrrolidinyl, tetrahydropyranyl or cyclopentyl rings may be optionally substituted by one or more oxo, hydroxy, —COC₁₋₆ alkyl (such as —COMe) or —COOC₁₋₆ alkyl (such as —COOtBu) groups; —CH₂—NR^{V}R^{W} (such as —CH₂—N(Me)₂); L-aryl (such as —CH₂—O—CH₂-phenyl).

In one embodiment, heterocyclyl (such as azetidinyl, pyrrolidinyl, morpholinyl or piperazinyl) optionally substituted by one or more hydroxy or C₁₋₆ alkanol (such as CH₂OH) groups.

In one embodiment, R¹⁵ and R¹⁶ independently represent hydrogen; hydroxy; halogen (such as fluorine); C₁₋₂₀ alkoxy (such as methoxy); —NR^{V}R^{W} (such as —NH₂, —NMe₂, —N(H)(Me), —N(H)(COMe), —N(H)((CH₂)₂OH), —N(H)((CH₂)₂SO₂Me), N(Mex(CH₂)₂SO₂Me), —N(H)(pyrrolidinyl), —N(Me)(pyrrolidinyl), —N(H)(azetidinyl), —N(H)((CH₂)₂NH₂), wherein said pyrrolidinyl ring may be optionally substituted by an oxo or —COC₁₋₂₀ alkyl (such as —COMe) group.

In one embodiment, heterocyclyl (such as morpholinyl or piperazinyl).

In one embodiment, X represents C(H)(R¹⁶). In one embodiment, R¹⁶ represents halogen (such as fluorine). In one embodiment, R¹⁶ represents C₁₋₂₀ alkoxy (such as methoxy). In one embodiment, R¹⁶ represents —NR^{V}R^{W} (such as —NH₂, —NMe₂, —N(H)(Me), —N(H)((CH₂)₂OH), —N(H)((CH₂)₂SO₂Me), N(Me)((CH₂)₂SO₂Me), —N(H)(pyrrolidinyl), —N(Me)(pyrrolidinyl), —N(H)(azetidinyl), —N(H)(oxetanyl), —N(Me)(oxetanyl), —N(H)(cyclopentyl), —N(Me)(cyclopentyl), —N(H)(tetrahydropyranyl), —N(Me)(tetrahydropyranyl) or —N(H)((CH₂)₂NH₂), wherein said pyrrolidinyl, tetrahydropyranyl or cyclopentyl rings may be optionally substituted by one or more oxo, hydroxy, —COC₁₋₂₀ alkyl (such as —COMe) or —COOC₁₋₂₀ alkyl (such as —COOtBu) groups. In one embodiment, R¹⁶ represents heterocyclyl (such as azetidinyl, pyrrolidinyl, morpholinyl or piperazinyl optionally substituted by one or more hydroxy or C₁₋₂₀ alkanol (i.e. CH₂OH) groups).

In one embodiment, Z represents C═O, X represents CR¹⁵R¹⁶, and R¹⁵ and R¹⁶, each represent hydrogen. In one embodiment, Z represents C═O, X represents CHR¹⁶, and R¹⁶ represents —NR^{V}R^{W} (such as —NH₂ or —N(H)(COMe)). In one embodiment, Z represents C═O, X represents CR¹⁵R¹⁶, R¹⁵ and R¹⁶ each represent hydrogen, and R¹⁶ represents heterocyclyl (such as morpholinyl). In one embodiment, Z represents C═O, X represents CR¹⁵R¹⁶, R¹⁵ represents hydrogen, and R¹⁶ represents —OR^{15a} (such as hydroxy). In one embodiment, Z represents C═O, X represents CR¹⁵R¹⁶, R¹⁵ represents hydrogen, and R¹⁶ represents —OR^{15a} (such as hydroxy). In one embodiment, Z represents C═O, X represents CR¹⁵R¹⁶, R¹⁵ represents hydrogen, and R¹⁶ represents -L-aryl (such as —CH₂—O—CH₂-phenyl). In one embodiment, Z represents C═O, X represents CR¹⁵R¹⁶, R¹⁵ represents hydrogen, and R¹⁶ represents C₁₋₂₀ alkanol (such as CH₂OH). In one embodiment, represents C═O, X represents CR¹⁵R¹⁶, and R¹⁵ and R¹⁶ each represent hydrogen. In one embodiment, Z represents C═O, X represents CR¹⁵R¹⁶, X represents CR¹⁵R¹⁶, R¹⁵ represents hydrogen, and R¹⁶ represents CH₂—NR^{V}R^{W} (such as —CH₂—N(Me)₂).

In one embodiment, R^{Z} represents methyl. In one embodiment, X represents CH₂. In one embodiment, R¹⁶ represents —OR^{15a} (such as hydroxy). In one embodiment, R¹⁶ represents halogen (such as fluorine). In one embodiment, R¹⁶ represents C₁₋₂₀ alkoxy (such as methoxy). In one embodiment, R¹⁶ represents —NR^{V}R^{W} (such as —NH₂, —NMe₂, —N(H)(Me), —N(H)((CH₂)₂OH), —N(H)((CH₂)₂SO₂Me), N(Me)((CH₂)₂SO₂Me), —N(H)(pyrrolidinyl), —N(Me)(pyrrolidinyl), —N(H)(azetidinyl), —N(H)(oxetanyl), —N(Me)(oxetanyl), —N(H)(cyclopentyl), —N(Me(cyclopentyl), —N(H)(tetrahydropyranyl), —N(Me)(tetrahydropyranyl) or —N(HX(CH₂)₂NH₂), wherein said pyrrolidinyl, tetrahydropyranyl or cyclopentyl rings may be optionally substituted by one or more oxo, hydroxy, —COC₁₋₂₀ alkyl (such as —COMe) or —COOC₁₋₂₀ alkyl (such as —COOtBu) groups. In one embodiment, R¹⁶ represents heterocyclyl (such as azetidinyl, pyrrolidinyl, morpholinyl or piperazinyl optionally substituted by one or more hydroxy or C₁₋₂₀ alkanol (i.e. CH₂OH) groups). In one embodiment, R¹⁶ represents —OR^{15a} (such as hydroxy).

In one embodiment, Z represents C═O; X represents —C(H)(R¹⁶)—; and R¹⁶, R^{A}, R^{A′}, R^{B}, and R^{B′} each represent hydrogen. In one embodiment, Z represents C═O; X represents —C(H)(R¹⁶), and R¹⁶ represents —NR^{V}R^{W} (such as —IMH₂ or —N(H)(COMe)). In one embodiment, Z represents C═O; X represents —C(H)(R¹⁶)—; and R¹⁶ represents heterocyclyl (such as morpholinyl). In one embodiment, Z represents C═O; X represents —C(H)(R¹⁶)—; and R¹⁶ represents —OR^{15a} (such as hydroxy). In one embodiment, Z represents C═O; X represents —C(H)(R¹⁶)—; and R¹⁶ represents -L-aryl (such as —CH₂—O—CH₂-phenyl). In one embodiment, Z represents C═O; X represents —C(H)(R¹⁶)—; and R¹⁶ represents C₁₋₂₀ alkanol (such as CH₂OH). In one embodiment, Z represents C═O; X represents —C(H)(R¹⁶)—; and R¹⁶ represents —CH₂—NR^{V}R^{W} (such as —CH₂—N(Me)₂).

In one embodiment, X represents —C(H)(R¹⁶)— and R¹⁶ represents hydroxyl. In one embodiment, R¹⁶ represents halogen (such as fluorine). In one embodiment, R¹⁶ represents C₁₋₂₀ alkoxy (such as methoxy). In one embodiment, R¹⁶ represents —NR^{V}R^{W} (such as —NH₂, —NMe₂, —N(H)(Me), —N(H)((CH₂)₂OH), —N(HX(CH₂)₂SO₂Me), N(Mex(CH₂)₂SO₂Me), —N(H)(pyrrolidinyl), —N(Me)(pyrrolidinyl), —N(H)(azetidinyl), —N(H)((CH₂)₂NH₂), wherein said pyrrolidinyl ring may be optionally substituted by an oxo or —COC₁₋₂₀ alkyl (such as —COMe) group. In one embodiment, R¹⁶ represents heterocyclyl (such as morpholinyl or piperazinyl). In one embodiment, R¹⁶ represents hydroxy.

In one embodiment, R¹⁶ represents —NR^{V}R^{W} (such as —NH₂ or —N(H)(COMe)). In one embodiment, R¹⁶ represents heterocyclyl (such as morpholinyl). In one embodiment, R¹⁶ represents hydroxy.

In one embodiment, Z represents C═O and X represents O.

In one embodiment, Z represents C═O; X represents —N(R¹⁷)—, and R¹⁷ represents hydrogen. In one embodiment, Z represents C═O; X represents —N(R¹⁷)—, and R¹⁷ represents C₁₋₂₀ alkanol (such as —CH₂—CH(OH)Me, —(CH₂)₂—OH, —CH₂—CHOH—CH₂OH or —(CH₂)₂—CHOH—CH₂OH); -L-SO₂—C₁₋₂₀ alkyl (such as —SO₂-Me or —(CH₂)₂—SO₂-Me); -L-SO₂—NR^{V}R^{W} (such as —(CH₂)₂—SO₂—N(Me)₂); -L-NR^{V}R^{W} (such as —(CH₂)₂—N(Me)₂, —(CH₂)₃—N(Me)₂ or —CH₂—CHOH—CH₂—NMe₂); -L-CO—NR^{V}R^{W} (such as —CH₂—CONH₂, —CH₂—CON(Me)₂, —(CH₂)₂—CON(Me)₂ or —(CH₂)₂—CON(H)(Me)); -L-NH—SO₂—C₁₋₂₀ alkyl (such as —(CH₂)₂—NH—SO₂-Me); -L-S(═NH)(═O)(C₁₋₂₀ alkyl) (such as —(CH₂)₂—S(═NH)(═O)(Me)); -L-O—SO₂—NR^{V}R^{W} (such as —(CH₂)₂—O—SO₂—NH₂); -L-N═S(═O)(C₁₋₂₀ alkyl), (such as —(CH₂)₂—N═S(═O)(Me)₂).

In one embodiment, R¹⁷ represents -L-heterocyclyl (such as —CH₂-oxetanyl, —CH₂-azetidinyl, —(CH₂)-azetidinyl, —CH₂-oxazolidinyl, —(CH₂)₂-piperidinyl, —(CH₂)₂-piperazinyl, —(CH₂)3-piperazinyl, —CH₂-morpholinyl, —(CH₂)₂-morpholinyl, —CH₂—CHOH—CH₂-morpholinyl, —(CH₂)₂-thiomorpholinyl, —CH₂-pyrrolidinyl, —(CH₂)₂-pyrrolidinyl or —CH₂—CHOH—CH₂-pyrrolidinyl), wherein said heterocyclyl ring may be optionally substituted by one or more oxo, hydroxy, halogen (such as fluorine), nitrile, C₁₋₂₀ alkyl (such as methyl), —COC₁₋₂₀ alkyl (such as —COMe), —NR^{V}—COC₁₋₂₀ alkyl (such as —NMe-COMe) or C₁₋₂₀ alkanol (such as —CH₂OH or —(CH₂)₂—OH) groups.

In one embodiment, Z represents C═O; X represents —N(R¹⁷)—; and R¹⁷ represents hydrogen. In one embodiment, R¹⁷ represents C₁₋₂₀ alkanol (such as —CH₂—CH(OH)Me). In one embodiment, R¹⁷ represents —SO₂—C₁₋₂₀ alkyl (such as —SO₂-Me).

In one embodiment, R^{V} and R^{W} represent hydrogen, C₁₋₆ alkyl (such as methyl), —COC₁₋₆ alkyl (such as —COMe), C₃₋₈ cycloalkyl (such as cyclopentyl) or heterocyclyl (such as oxetanyl, azetidinyl, tetrahydropyranyl or pyrrolidinyl), wherein said alkyl groups may be optionally substituted with or more hydroxy (such as (Chb\hat{ }OH), amino (such as (Chb\hat{ }Nhb) or sulfone (such as (CH₂)₂SO₂Me) groups and said heterocyclyl ring may be optionally substituted by one or more oxo or —COC₁₋₆ alkyl (such as —COMe) groups. In one embodiment, R^{V} and R^{W} represent hydrogen, C₁₋₆ alkyl (such as methyl), —COC₁₋₆ alkyl (such as —COMe) or heterocyclyl (such as azetidinyl or pyrrolidinyl), wherein said alkyl groups may be optionally substituted with or more hydroxy (such as (Chb\hat{ }OH), amino (such as (CH₂)₂NH₂) or sulfone (such as (CH₂)₂SO₂Me) groups and said heterocyclyl ring may be optionally substituted by one or more oxo or —COC₁₋₆ alkyl (such as —COMe) groups. In one embodiment, R^{V} and R^{W} both represent hydrogen or C₁₋₆ alkyl (such as methyl) or one represents hydrogen and the other represents C₁₋₆ alkyl (such as methyl) or one represents hydrogen or C₁₋₆ alkyl (such as methyl) and the other represents —COC₁₋₆ alkyl (such as —COMe), C₃₋₈ cycloalkyl (such as cyclopentyl) or heterocyclyl (such as azetidinyl, tetrahydropyranyl or pyrrolidinyl), wherein said alkyl groups may be optionally substituted with or more hydroxy (such as (Chb\hat{ }OH), amino (such as (Chb\hat{ }Nhb) or sulfone (such as (CH₂)₂SO₂Mc) groups and said heterocyclyl ring may be optionally substituted by one or more oxo or —COC₁₋₆ alkyl (such as —COMe) groups. In one embodiment, R^{V} and R^{W} both represent hydrogen or C₁₋₆ alkyl (such as methyl) or one represents hydrogen and the other represents C₁₋₆ alkyl (such as methyl) or one represents hydrogen or C₁₋₆ alkyl (such as methyl) and the other represents —COC₁₋₆ alkyl (such as —COMe) or heterocyclyl (such as azetidinyl or pyrrolidinyl), wherein said alkyl groups may be optionally substituted with or more hydroxy, amino, or sulfone (such as (CH₂)₂SO₂Me) groups and said heterocyclyl ring may be optionally substituted by one or more oxo or —COC₁₋₆ alkyl (such as —COMe) groups.

In one embodiment, R⁵ represents C₁₋₆ alkyl (such as CH₃, CD₃, ethyl or isopropyl) or C₃₋₈ cycloalkyl (such as cyclopropyl). In one embodiment, R⁵ represents C₁₋₆ alkyl (such as methyl, ethyl or isopropyl). In one embodiment, R⁵ represents C₁₋₆ alkyl (such as CH₃, CD₃ or ethyl). In one embodiment, R⁵ represents C₁₋₆ alkyl (such as methyl or ethyl). In a yet further embodiment, R⁵ represents C₁₋₆ alkyl (such as CHs or CDs).

In one embodiment, Y represents —C(R⁶)═. In an alternative embodiment, Y represents N. In one embodiment, R⁶ represents: hydrogen; halogen (such as fluorine or chlorine).

In one embodiment, R⁶ represents C₁₋₆ alkoxy (such as methoxy). In one embodiment, R⁶ represents: hydrogen; halogen (such as fluorine). In one embodiment, R⁶ represents C₁₋₆ alkoxy (such as methoxy). In one embodiment, R⁶ represents: hydrogen. In one embodiment, R⁶ represents halogen (such as fluorine). In one embodiment, R⁶ represents hydrogen.

In one embodiment, R⁷ represents: hydrogen; halogen (such as fluorine, bromine or chlorine); C₁₋₆ alkyl (such as methyl or ethyl); C₂₋₆ alkenyl (such as ethenyl); hydroxy; C₁₋₆ alkoxy (such as methoxy); —NR^{X}R^{Y} (such as —NH₂, —NHMe or —NMe₂). In one embodiment, R⁷ represents: hydrogen; halogen (such as fluorine, bromine or chlorine); C₁₋₆ alkyl (such as methyl or ethyl); C₂₋₆ alkenyl (such as ethenyl); hydroxy; C₁₋₆ alkoxy (such as methoxy). In one embodiment, R⁷ represents: hydrogen; halogen (such as fluorine or chlorine).

In one embodiment, R^{X} and R^{Y} represent C₁₋₆ alkyl (such as methyl). In one embodiment, R⁷ represents halogen (such as chlorine). In one embodiment, R^{X} and R^{Y} independently represent hydrogen or methyl. In one embodiment, both of R^{X} and R^{Y} represent hydrogen or both of R^{X} and R^{Y} represent methyl or one of R^{X} and R^{Y} represents hydrogen and the other represents methyl.

In one embodiment, R⁸, R⁹, and R¹⁰ each represent halogen. In one embodiment, R⁷, R⁸, and R¹⁰ each represent halogen. In one embodiment, R⁸ and R⁹ each represent halogen. In one embodiment, R⁸ represents fluorine. In one embodiment, R⁹ represents chlorine. In one embodiment, R¹⁰ represents fluorine.

In one embodiment, R⁸ represents: hydrogen; halogen (such as fluorine, bromine or chlorine); C₁₋₆ alkyl (such as methyl or ethyl); C₂₋₆ alkenyl (such as ethenyl); C₃₋₈ cycloalkyl (such as cyclopropyl); haloC₁₋₆ alkyl (such as trifluoromethyl). In one embodiment, R⁸ represents: hydrogen; halogen (such as fluorine, bromine or chlorine); C₁₋₆ alkyl (such as methyl or ethyl); C₂₋₆ alkenyl (such as ethenyl); haloC₁₋₆ alkyl (such as trifluoromethyl). In one embodiment, R⁸ and R⁷ join to form a 5 to 7 membered saturated or unsaturated ring optionally containing one or more heteroatoms selected from O, N or S (such as a pyrrolinyl or tetrahydropyranyl ring). In one embodiment, R⁸ represents: hydrogen; halogen (such as fluorine or chlorine).

In one embodiment, R⁸ represents haloC₁₋₆ alkyl (such as trifluoromethyl). In one embodiment, R⁸ represents halogen (such as fluorine).

In one embodiment, R⁷ and R⁸ join to form a 5 to 7 membered saturated or unsaturated ring optionally containing one or more heteroatoms selected from O, N or S (such as a benzyl, pyridinyl, purinyl, pyrimidinyl, diazinyl, pyrrolyl, pyrrolinyl, tetrahydropyranyl, pyrazolyl, morpholinyl, pyridyl, furanyl or thiophenyl ring optionally substituted by one or more methyl or fluorine groups). In one embodiment, R⁷ and R⁸ join to form a 5 to 7 membered saturated or unsaturated ring optionally containing one or more heteroatoms selected from O, N or S (such as a pyrrolinyl or tetrahydropyranyl ring). In one embodiment, R⁸ and R⁷ join to form a 5 to 7 membered saturated or unsaturated ring optionally containing one or more heteroatoms selected from O, N or S (such as a benzyl, pyridinyl, purinyl, pyrimidinyl, diazinyl, pyrrolyl, pyrrolinyl, tetrahydropyranyl, pyrazolyl, morpholinyl, pyridyl, furanyl or thiophenyl ring optionally substituted by one or more methyl or fluorine groups).

In one embodiment, R⁹ represents: hydrogen; halogen (such as fluorine or chlorine); C₁₋₆ alkyl (such as methyl); haloC₁₋₆ alkyl (such as fluoromethyl, difluoromethyl or trifluoromethyl). In one embodiment, R⁹ represents C₁₋₆ alkoxy (such as methoxy). In one embodiment, R⁹ represents hydrogen.

In one embodiment, R¹⁰ represents: hydrogen.

In one embodiment, R¹⁰ represents halogen (such as fluorine). In one embodiment, R¹⁰ represents hydrogen.

In one embodiment, Y represents —C(R⁶)═; and each of R⁶, R⁷, R⁸, R⁹ and R¹⁰ represent hydrogen.

In one embodiment, each of R⁶, R⁷, R⁸ and R¹⁰ represent hydrogen and R⁹ represents C₁₋₆ alkyl (such as methyl).

In one embodiment, each of R⁶, R⁷ and R¹⁰ represent hydrogen and R⁸ and R⁹ both represents halogen (such as fluorine or chlorine).

In one embodiment, each of R⁶, R⁷ and R¹⁰ represent hydrogen, R⁸ represents halogen (such as fluorine) and R⁹ represents C₁₋₆ alkyl (such as methyl).

In one embodiment, each of R⁶, R⁷ and R¹⁰ represent hydrogen, R⁸ represents halogen (such as fluorine) and R⁹ represents haloC₁₋₆ alkyl (such as fluoromethyl or trifluoromethyl).

In one embodiment, each of R⁶, R⁷, R⁸ and R¹⁰ represent hydrogen and R⁹ represents haloC₁₋₆ alkyl (such as fluoromethyl or difluoromethyl).

In one embodiment, each of R⁶, R⁷, R⁸ and R¹⁰ represent hydrogen and R⁹ represents halogen (such as chlorine).

In one embodiment, each of R⁶, R⁷ and R¹⁰ represent hydrogen, R⁸ represents halogen (such as fluorine) and R⁹ represents C₁₋₆ alkoxy (such as methoxy).

In one embodiment, each of R⁶, R⁸ and R¹⁰ represent hydrogen and R⁷ and R⁹ both represent halogen (such as fluorine or chlorine).

In one embodiment, each of R⁶, R⁷ and R¹⁰ represent hydrogen, R⁸ represents haloC₁₋₆ alkyl (such as trifluoromethyl) and R⁹ represents halogen (such as fluorine).

In one embodiment, each of R⁶, R⁸, R⁹ and R¹⁰ represent hydrogen and R⁷ represents C₁₋₆ alkyl (such as methyl); each of R⁶, R⁸, R⁹ and R¹⁰ represent hydrogen and R⁷ represents halogen (such as chlorine); each of R⁶, R⁹ and R¹⁰ represent hydrogen and R⁷ and R⁸ both represent halogen (such as fluorine, bromine or chlorine); each of R⁶, R⁷ and R¹⁰ represent hydrogen, R⁸ represents C₁₋₆ alkyl (such as methyl) and R⁹ represents halogen (such as chlorine); each of R⁶, R⁹ and R¹⁰ represent hydrogen, R⁷ represents C₁₋₆ alkyl (such as methyl or ethyl) and R⁸ represents halogen (such as chlorine or fluorine); each of R⁶, R⁹ and R¹⁰ represent hydrogen, R⁷ represents C₁₋₆ alkoxy (such as methoxy) and R⁸ represents halogen (such as fluorine); each of R⁶, R⁹ and R¹⁰ represent hydrogen, R⁷ represents C₁₋₆ alkoxy (such as methoxy) and R⁸ represents C₁₋₆ alkyl (such as methyl); each of R⁶, R⁹ and R¹⁰ represent hydrogen, R⁷ represents halogen (such as chlorine) and R⁸ represents C₁₋₆ alkyl (such as methyl); both of R⁹ and R¹⁰ represent hydrogen, both of R⁶ and R⁸ represent halogen (such as fluorine) and R⁷ represents C₁₋₆ alkyl (such as methyl or ethyl); each of R⁷, R⁹ and R¹⁰ represent hydrogen and both of R⁶ and R⁸ represent halogen (such as chlorine, bromine or fluorine); both of R⁶ and R⁹ represent hydrogen and each of R⁷, R⁸ and R¹⁰ represent halogen (such as chlorine or fluorine); each of R⁸, R⁹ and R¹⁰ represent hydrogen and both of R⁶ and R⁷ represents halogen (such as chlorine or fluorine); each of R⁶, R⁹ and R¹⁰ represent hydrogen, R⁷ represents hydroxy and R⁸ represents halogen (such as fluorine); each of R⁶, R⁹ and R¹⁰ represent hydrogen, R⁷ represents C₂₋₆ alkenyl (such as ethenyl) and R⁸ represents halogen (such as fluorine) each of R⁶, R⁹ and R¹⁰ represent hydrogen, R⁷ represents hydroxy and R⁸ represents C₁₋₆ alkyl (such as methyl); each of R⁷, R⁸ and R¹⁰ represent hydrogen and both of R⁶ and R⁹ represent halogen (such as fluorine or chlorine); both of R⁷ and R¹⁰ represent hydrogen, both of R⁸ and R⁹ represent halogen (such as fluorine or chlorine) and R⁶ represents C₁₋₆ alkoxy (such as methoxy); both of R⁹ and R¹⁰ represent hydrogen and each of R⁶, R⁷ and R⁸ represent halogen (such as fluorine or chlorine); both of R⁹ and R¹⁰ represent hydrogen, both of R⁶ and R⁸ represent halogen (such as fluorine) and R⁷ represents C₂₋₆ alkenyl (such as ethenyl); each of R⁶, R⁹ and R¹⁰ represent hydrogen and R⁷ and R⁸ join to form a pyrrolinyl, tetrahydropyranyl, pyrazolyl or pyridyl ring optionally substituted by a methyl group; both of R⁷ and R¹⁰ represent hydrogen and each of R⁶, R⁸ and R⁹ represent halogen (such as chlorine or fluorine).

In one embodiment, each of R⁶, R⁹ and R¹⁰ represent hydrogen, R⁷ represents halogen (such as chlorine) and R⁸ represents C₃₋₈ cycloalkyl (such as cyclopropyl).

In one embodiment, Y represents —C(R⁶)═; and each of R⁶, R⁷, R⁸, R⁹ and R¹⁰ represent hydrogen.

In one embodiment, each of R⁶, R⁷, R⁸ and R¹⁰ represent hydrogen and R⁹ represents C₁₋₆ alkyl (such as methyl).

In one embodiment, each of R⁶, R⁷ and R¹⁰ represent hydrogen and R⁸ and R⁹ both represents halogen (such as fluorine or chlorine).

In one embodiment, each of R⁶, R⁷ and R¹⁰ represent hydrogen, R⁸ represents halogen (such as fluorine) and R⁹ represents C₁₋₆ alkyl (such as methyl).

In one embodiment, each of R⁶, R⁷ and R¹⁰ represent hydrogen, R⁸ represents halogen (such as fluorine) and R⁹ represents haloC₁₋₆ alkyl (such as fluoromethyl or trifluoromethyl).

In one embodiment, each of R⁶, R⁷, R⁸ and R¹⁰ represent hydrogen and R⁹ represents haloC₁₋₆ alkyl (such as fluoromethyl or difluoromethyl).

In one embodiment, each of R⁶, R⁷, R⁸ and R¹⁰ represent hydrogen and R⁹ represents halogen (such as chlorine).

In one embodiment, each of R⁶, R⁷ and R¹⁰ represent hydrogen, R⁸ represents halogen (such as fluorine) and R⁹ represents C₁₋₆ alkoxy (such as methoxy).

In one embodiment, each of R⁶, R⁸ and R¹⁰ represent hydrogen and R⁷ and R⁹ both represent halogen (such as fluorine or chlorine).

In one embodiment, each of R⁶, R⁷ and R¹⁰ represent hydrogen, R⁸ represents haloC₁₋₆ alkyl (such as trifluoromethyl) and R⁹ represents halogen (such as fluorine).

In one embodiment, each of R⁶, R⁸, R⁹ and R¹⁰ represent hydrogen and R⁷ represents C₁₋₆ alkyl (such as methyl); each of R⁶, R⁸, R⁹ and R¹⁰ represent hydrogen and R⁷ represents halogen (such as chlorine); each of R⁶, R⁹ and R¹⁰ represent hydrogen and R⁷ and R⁸ both represent halogen (such as fluorine, bromine or chlorine); each of R⁶, R⁷ and R¹⁰ represent hydrogen, R⁸ represents C₁₋₆ alkyl (such as methyl) and R⁹ represents halogen (such as chlorine); each of R⁶, R⁹ and R¹⁰ represent hydrogen, R⁷ represents C₁₋₆ alkyl (such as methyl or ethyl) and R⁸ represents halogen (such as chlorine or fluorine); each of R⁶, R⁹ and R¹⁰ represent hydrogen, R⁷ represents C₁₋₆ alkoxy (such as methoxy) and R⁸ represents halogen (such as fluorine); each of R⁶, R⁹ and R¹⁰ represent hydrogen, R⁷ represents C₁₋₆ alkoxy (such as methoxy) and R⁸ represents C₁₋₆ alkyl (such as methyl); each of R⁶, R⁹ and R¹⁰ represent hydrogen, R⁷ represents halogen (such as chlorine) and R⁸ represents C₁₋₆ alkyl (such as methyl); both of R⁹ and R¹⁰ represent hydrogen, both of R⁶ and R⁸ represent halogen (such as fluorine) and R⁷ represents C₁₋₆ alkyl (such as methyl or ethyl); each of R⁷, R⁹ and R¹⁰ represent hydrogen and both of R⁶ and R⁸ represent halogen (such as chlorine, bromine or fluorine); both of R⁶ and R⁹ represent hydrogen and each of R⁷, R⁸ and R¹⁰ represent halogen (such as chlorine or fluorine); each of R⁸, R⁹ and R¹⁰ represent hydrogen and both of R⁶ and R⁷ represents halogen (such as chlorine or fluorine); each of R⁶, R⁹ and R¹⁰ represent hydrogen, R⁷ represents hydroxy and R⁸ represents halogen (such as fluorine); each of R⁶, R⁹ and R¹⁰ represent hydrogen, R⁷ represents C₂₋₆ alkenyl (such as ethenyl) and R⁸ represents halogen (such as fluorine) each of R⁶, R⁹ and R¹⁰ represent hydrogen, R⁷ represents hydroxy and R⁸ represents C₁₋₆ alkyl (such as methyl); each of R⁷, R⁸ and R¹⁰ represent hydrogen and both of R⁶ and R⁹ represent halogen (such as fluorine or chlorine); both of R⁷ and R¹⁰ represent hydrogen, both of R8 and R⁹ represent halogen (such as fluorine or chlorine) and R⁶ represents C₁₋₆ alkoxy (such as methoxy); both of R⁹ and R¹⁰ represent hydrogen and each of R⁶, R⁷ and R⁸ represent halogen (such as fluorine or chlorine); both of R⁹ and R¹⁰ represent hydrogen, both of R⁶ and R⁸ represent halogen (such as fluorine) and R⁷ represents C₂₋₆ alkenyl (such as ethenyl); each of R⁶, R⁹ and R¹⁰ represent hydrogen and R⁷ and R⁸ join to form a pyrrolinyl or tetrahydropyranyl ring.

In one embodiment, both of R⁷ and R¹⁰ represent hydrogen and each of R⁶, R⁸ and R⁹ represent halogen (such as chlorine or fluorine).

In one embodiment, Y represents —C(R⁶)═; and each of R⁶, R⁷, R⁸, R⁹ and R¹⁰ represent hydrogen.

In one embodiment, each of R⁶, R⁷, R⁸ and R¹⁰ represent hydrogen and R⁹ represents C₁₋₆ alkyl (such as methyl).

In one embodiment, each of R⁶, R⁷ and R¹⁰ represent hydrogen and R⁸ and R⁹ both represents halogen (such as fluorine or chlorine).

In one embodiment, each of R⁶, R⁷ and R¹⁰ represent hydrogen, R⁸ represents halogen (such as fluorine) and R⁹ represents C₁₋₆ alkyl (such as methyl).

In one embodiment, each of R⁶, R⁷ and R¹⁰ represent hydrogen, R⁸ represents halogen (such as fluorine) and R⁹ represents haloC₁₋₆ alkyl (such as fluoromethyl or trifluoromethyl).

In one embodiment, each of R⁶, R⁷, R⁸ and R¹⁰ represent hydrogen and R⁹ represents haloC₁₋₆ alkyl (such as fluoromethyl or difluoromethyl).

In one embodiment, each of R⁶, R⁷, R⁸ and R¹⁰ represent hydrogen and R⁹ represents halogen (such as chlorine).

In one embodiment, each of R⁶, R⁷ and R¹⁰ represent hydrogen, R⁸ represents halogen (such as fluorine) and R⁹ represents C₁₋₆ alkoxy (such as methoxy).

In one embodiment, each of R⁶, R⁸ and R¹⁰ represent hydrogen and R⁷ and R⁹ both represent halogen (such as fluorine).

In one embodiment, each of R⁶, R⁷ and R¹⁰ represent hydrogen, R⁸ represents haloC₁₋₆ alkyl (such as trifluoromethyl) and R⁹ represents halogen (such as fluorine).

In one embodiment, each of R⁶, R⁸, R⁹ and R¹⁰ represent hydrogen and R⁷ represents C₁₋₆ alkyl (such as methyl).

In one embodiment, each of R⁶, R⁹ and R¹⁰ represent hydrogen and R⁷ and R⁸ both represent halogen (such as fluorine or chlorine).

In one embodiment, Y represents —C(R⁶)═; and each of R⁶, R⁹ and R¹⁰ represent hydrogen and R⁷ and R⁸ both represent halogen (such as fluorine or chlorine), such as each of R⁶, R⁹ and R¹⁰ represent hydrogen, R⁷ represents halogen (such as chlorine) and R⁸ represents halogen (such as fluorine).

In an alternative embodiment, Y represents —N═; and R⁸ and R¹⁰ both represent hydrogen, R⁷ represents C₁₋₆ alkyl (such as methyl) and R⁹ represents haloC₁₋₆ alkyl (such as trifluoromethyl).

R⁷ and R⁸ join to form a pyrrolinyl ring and R⁹ and R¹⁰ both represent hydrogen.

R⁹ and R¹⁰ both represent hydrogen, R⁷ represents C₁₋₆ alkyl (such as methyl) and R⁸ represents halogen (such as fluorine); each of R⁸, R⁹ and R¹⁰ represent hydrogen and R⁷ represents —NR^{m}Rⁿ (such as —NH₂, —NHMe or —NMe₂).

R⁹ and R¹⁰ both represent hydrogen and R⁷ and R⁸ join to form a pyrrolinyl, morpholinyl, furanyl or thiophenyl ring optionally substituted by a methyl, fluorine or chlorine group.

In one embodiment, R¹⁰ represents hydrogen, R⁹ represents halogen (such as chlorine) and R⁷ and R⁸ join to form a pyrrolinyl ring optionally substituted by a methyl group.

In one embodiment, Y represents —N═; and R⁸ and R¹⁰ both represent hydrogen, R⁷ represents C₁₋₆ alkyl (such as methyl) and R⁹ represents haloC₁₋₆ alkyl (such as trifluoromethyl).

In one embodiment, R⁷ and R⁸ join to form a pyrrolinyl ring and R⁹ and R¹⁰ both represent hydrogen.

In one embodiment, Y represents —N═; and R⁸ and R¹⁰ both represent hydrogen, R⁷ represents C₁₋₆ alkyl (such as methyl) and R⁹ represents haloC₁₋₆ alkyl (such as trifluoromethyl).

In one embodiment, the compound of Formula (I) is represented by one of the following compounds:

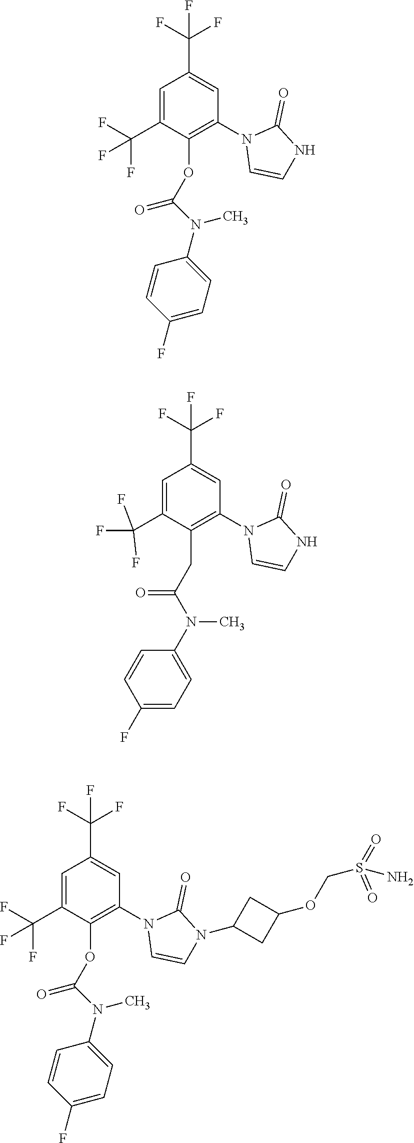 [SMILES: CN(C(=O)Oc1c(-n2cc[nH]c2=O)cc(C(F)(F)F)cc1C(F)(F)F)c1ccc(F)cc1 ; CN(C(=O)Cc1c(-n2cc[nH]c2=O)cc(C(F)(F)F)cc1C(F)(F)F)c1ccc(F)cc1 ; CN(C(=O)Oc1c(-n2ccn(C3CC(OCS(N)(=O)=O)C3)c2=O)cc(C(F)(F)F)cc1C(F)(F)F)c1ccc(F)cc1]

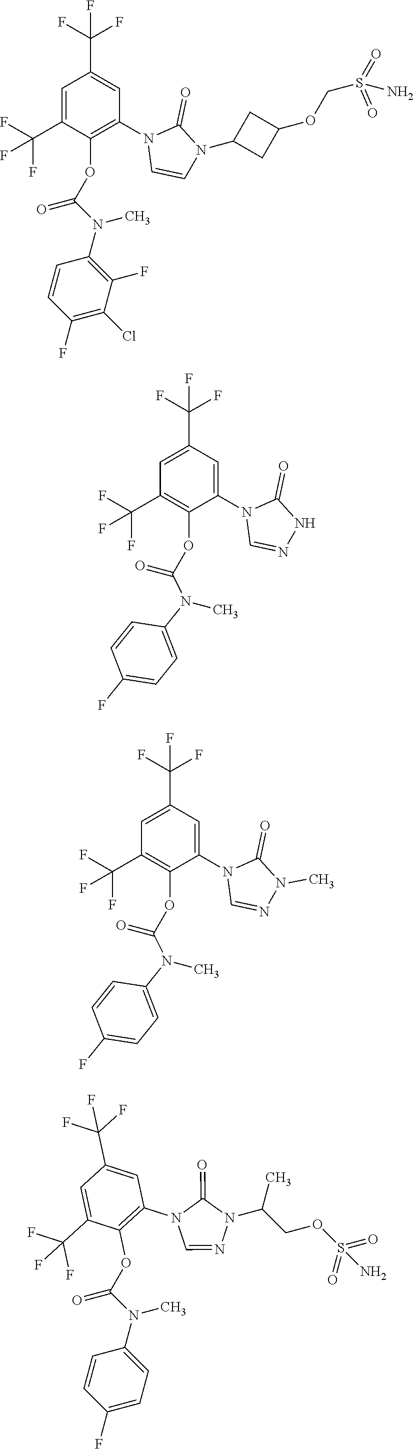

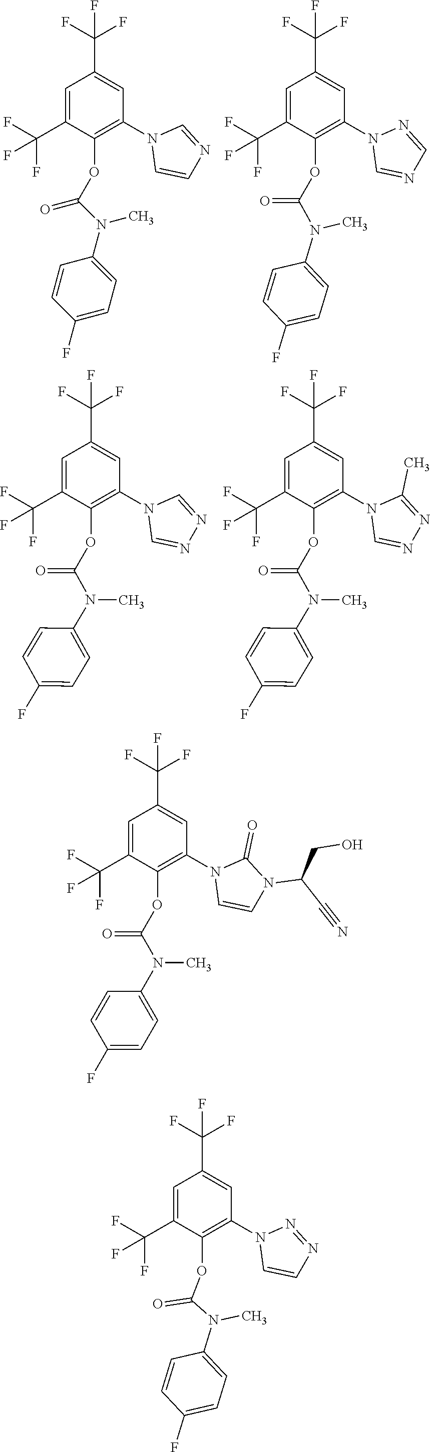

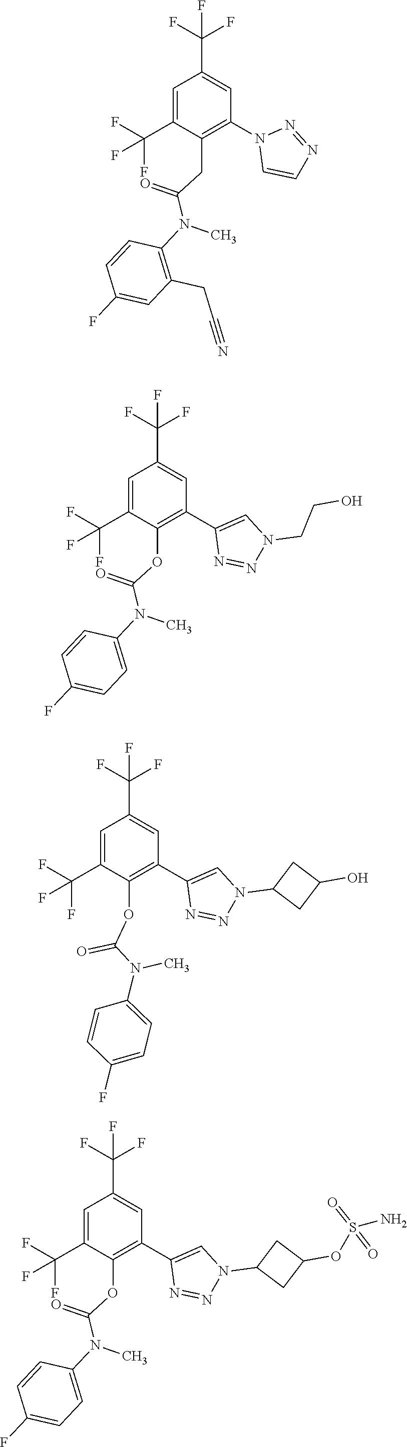

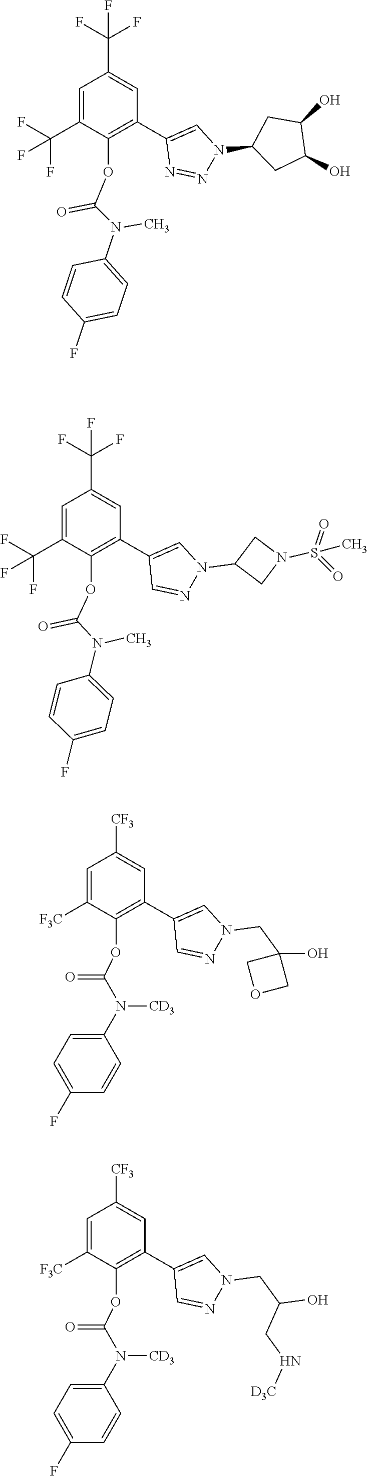 [SMILES: CN(C(=O)Oc1c(-c2cn([C@H]3C[C@@H](O)[C@@H](O)C3)nn2)cc(C(F)(F)F)cc1C(F)(F)F)c1ccc(F)cc1 ; CN(C(=O)Oc1c(-c2cnn(C3CN(S(C)(=O)=O)C3)c2)cc(C(F)(F)F)cc1C(F)(F)F)c1ccc(F)cc1 ; [2H]C([2H])([2H])N(C(=O)Oc1c(-c2cnn(CC3(O)COC3)c2)cc(C(F)(F)F)cc1C(F)(F)F)c1ccc(F)cc1 ; [2H]C([2H])([2H])NCC(O)Cn1cc(-c2cc(C(F)(F)F)cc(C(F)(F)F)c2OC(=O)N(c2ccc(F)cc2)C([2H])([2H])[2H])cn1]

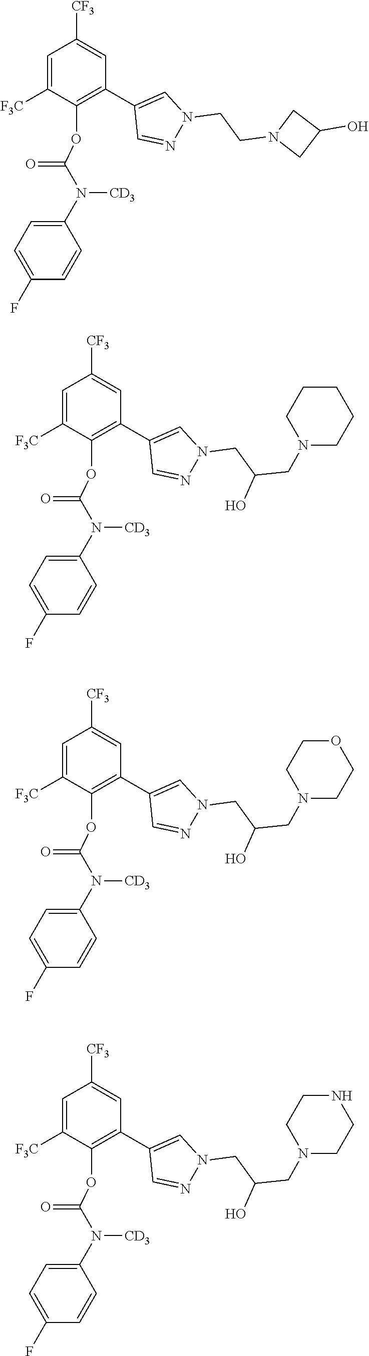 [SMILES: [2H]C([2H])([2H])N(C(=O)Oc1c(-c2cnn(CCN3CC(O)C3)c2)cc(C(F)(F)F)cc1C(F)(F)F)c1ccc(F)cc1 ; [2H]C([2H])([2H])N(C(=O)Oc1c(-c2cnn(CC(O)CN3CCCCC3)c2)cc(C(F)(F)F)cc1C(F)(F)F)c1ccc(F)cc1 ; [2H]C([2H])([2H])N(C(=O)Oc1c(-c2cnn(CC(O)CN3CCOCC3)c2)cc(C(F)(F)F)cc1C(F)(F)F)c1ccc(F)cc1 ; [2H]C([2H])([2H])N(C(=O)Oc1c(-c2cnn(CC(O)CN3CCNCC3)c2)cc(C(F)(F)F)cc1C(F)(F)F)c1ccc(F)cc1]

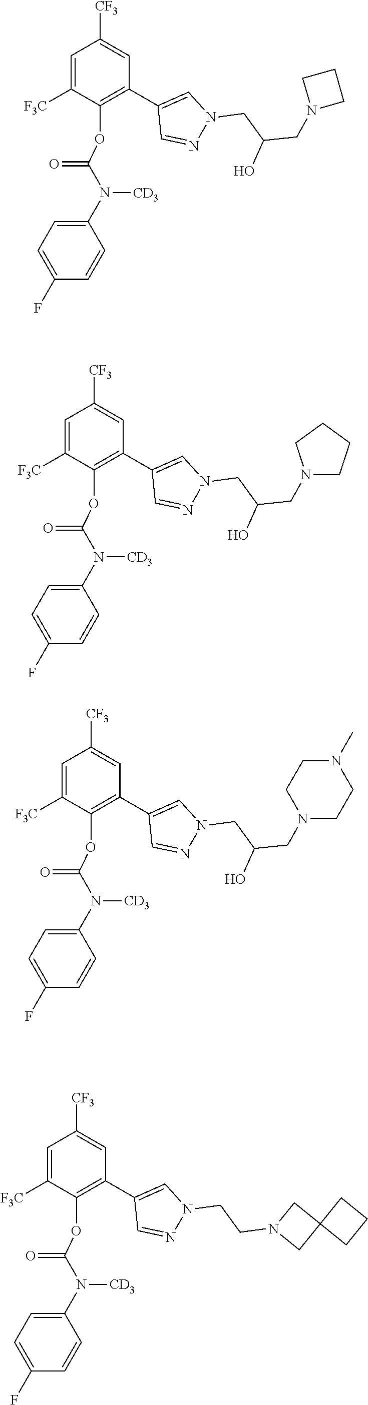 [SMILES: [2H]C([2H])([2H])N(C(=O)Oc1c(-c2cnn(CC(O)CN3CCC3)c2)cc(C(F)(F)F)cc1C(F)(F)F)c1ccc(F)cc1 ; [2H]C([2H])([2H])N(C(=O)Oc1c(-c2cnn(CC(O)CN3CCCC3)c2)cc(C(F)(F)F)cc1C(F)(F)F)c1ccc(F)cc1 ; [2H]C([2H])([2H])N(C(=O)Oc1c(-c2cnn(CC(O)CN3CCN(C)CC3)c2)cc(C(F)(F)F)cc1C(F)(F)F)c1ccc(F)cc1 ; [2H]C([2H])([2H])N(C(=O)Oc1c(-c2cnn(CCN3CC4(CCC4)C3)c2)cc(C(F)(F)F)cc1C(F)(F)F)c1ccc(F)cc1]

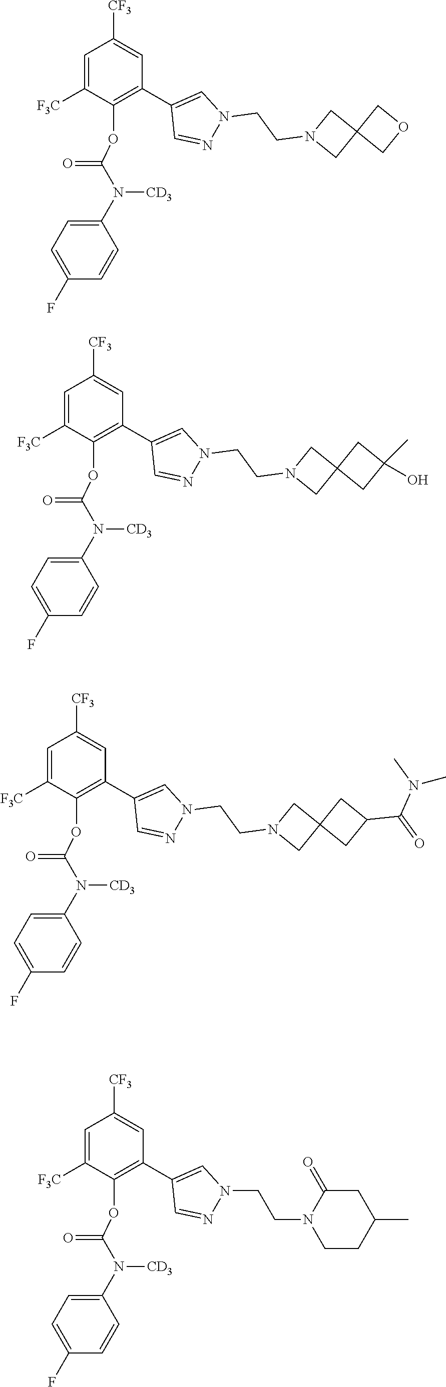 [SMILES: [2H]C([2H])([2H])N(C(=O)Oc1c(-c2cnn(CCN3CC4(COC4)C3)c2)cc(C(F)(F)F)cc1C(F)(F)F)c1ccc(F)cc1 ; [2H]C([2H])([2H])N(C(=O)Oc1c(-c2cnn(CCN3CC4(C3)CC(C)(O)C4)c2)cc(C(F)(F)F)cc1C(F)(F)F)c1ccc(F)cc1 ; [2H]C([2H])([2H])N(C(=O)Oc1c(-c2cnn(CCN3CC4(CC(C(=O)N(C)C)C4)C3)c2)cc(C(F)(F)F)cc1C(F)(F)F)c1ccc(F)cc1 ; [2H]C([2H])([2H])N(C(=O)Oc1c(-c2cnn(CCN3CCC(C)CC3=O)c2)cc(C(F)(F)F)cc1C(F)(F)F)c1ccc(F)cc1]

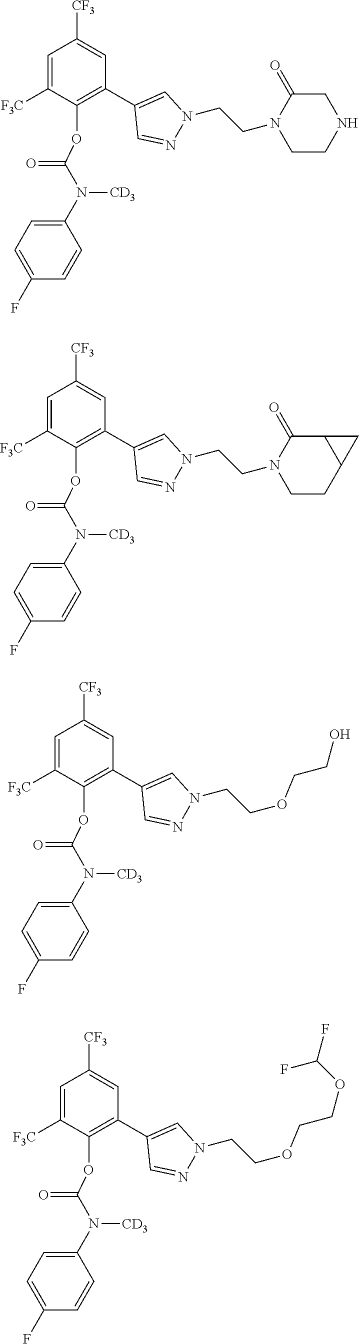 [SMILES: [2H]C([2H])([2H])N(C(=O)Oc1c(-c2cnn(CCN3CCNCC3=O)c2)cc(C(F)(F)F)cc1C(F)(F)F)c1ccc(F)cc1 ; [2H]C([2H])([2H])N(C(=O)Oc1c(-c2cnn(CCN3CCC4CC4C3=O)c2)cc(C(F)(F)F)cc1C(F)(F)F)c1ccc(F)cc1 ; [2H]C([2H])([2H])N(C(=O)Oc1c(-c2cnn(CCOCCO)c2)cc(C(F)(F)F)cc1C(F)(F)F)c1ccc(F)cc1 ; [2H]C([2H])([2H])N(C(=O)Oc1c(-c2cnn(CCOCCOC(F)F)c2)cc(C(F)(F)F)cc1C(F)(F)F)c1ccc(F)cc1]

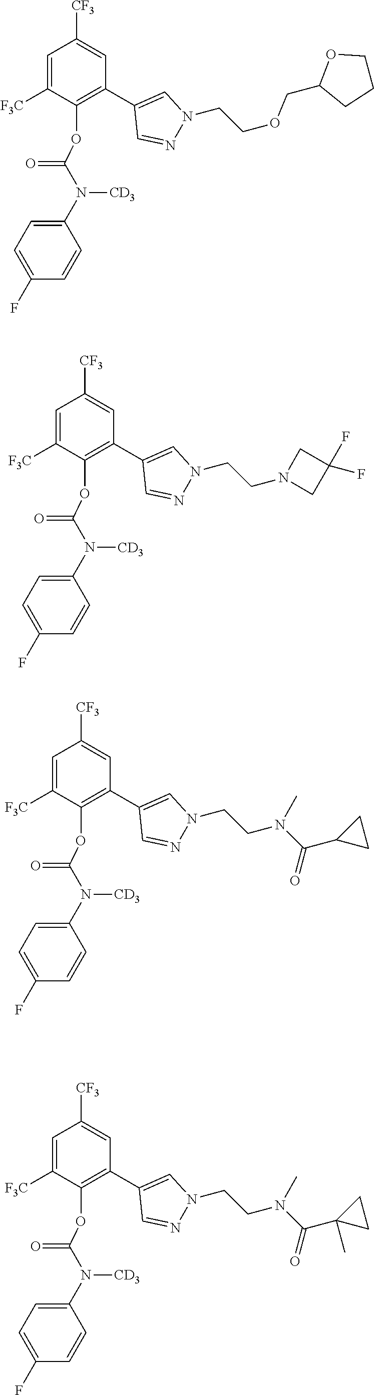 [SMILES: [2H]C([2H])([2H])N(C(=O)Oc1c(-c2cnn(CCOCC3CCCO3)c2)cc(C(F)(F)F)cc1C(F)(F)F)c1ccc(F)cc1 ; [2H]C([2H])([2H])N(C(=O)Oc1c(-c2cnn(CCN3CC(F)(F)C3)c2)cc(C(F)(F)F)cc1C(F)(F)F)c1ccc(F)cc1 ; [2H]C([2H])([2H])N(C(=O)Oc1c(-c2cnn(CCN(C)C(=O)C3CC3)c2)cc(C(F)(F)F)cc1C(F)(F)F)c1ccc(F)cc1 ; [2H]C([2H])([2H])N(C(=O)Oc1c(-c2cnn(CCN(C)C(=O)C3(C)CC3)c2)cc(C(F)(F)F)cc1C(F)(F)F)c1ccc(F)cc1]

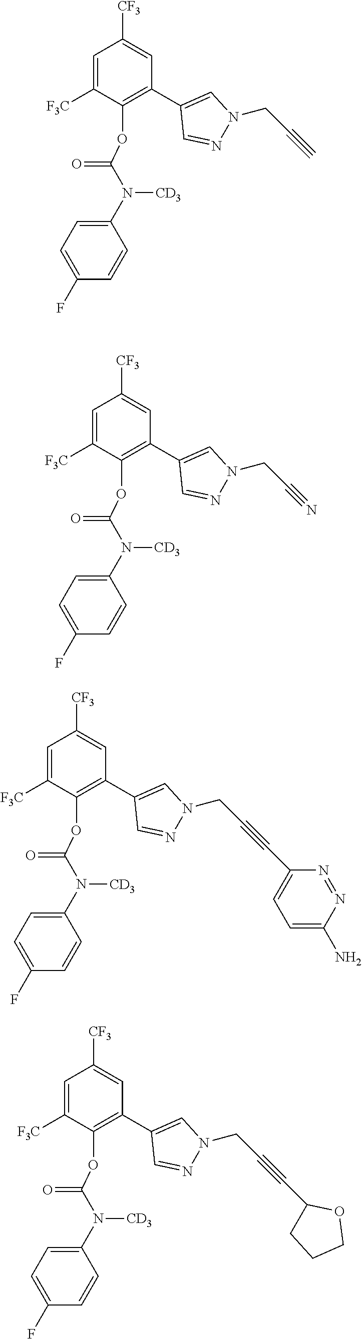

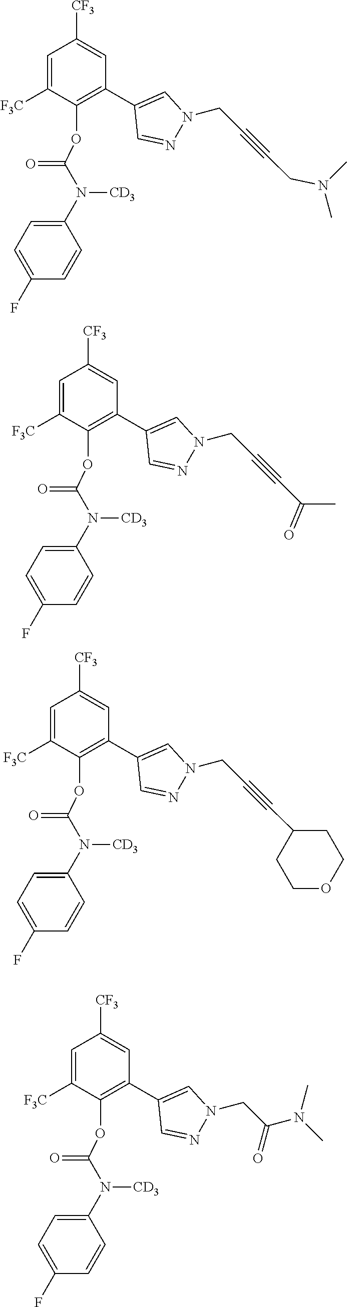 [SMILES: [2H]C([2H])([2H])N(C(=O)Oc1c(-c2cnn(CC#CCN(C)C)c2)cc(C(F)(F)F)cc1C(F)(F)F)c1ccc(F)cc1 ; [2H]C([2H])([2H])N(C(=O)Oc1c(-c2cnn(CC#CC(C)=O)c2)cc(C(F)(F)F)cc1C(F)(F)F)c1ccc(F)cc1 ; [2H]C([2H])([2H])N(C(=O)Oc1c(-c2cnn(CC#CC3CCOCC3)c2)cc(C(F)(F)F)cc1C(F)(F)F)c1ccc(F)cc1 ; [2H]C([2H])([2H])N(C(=O)Oc1c(-c2cnn(CC(=O)N(C)C)c2)cc(C(F)(F)F)cc1C(F)(F)F)c1ccc(F)cc1]

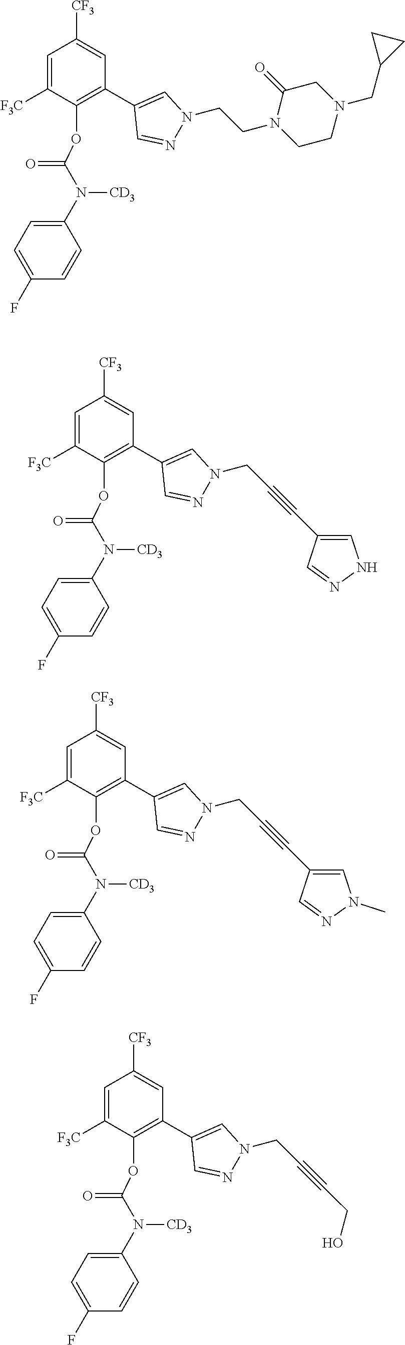 [SMILES: [2H]C([2H])([2H])N(C(=O)Oc1c(-c2cnn(CCN3CCN(CC4CC4)CC3=O)c2)cc(C(F)(F)F)cc1C(F)(F)F)c1ccc(F)cc1 ; [2H]C([2H])([2H])N(C(=O)Oc1c(-c2cnn(CC#Cc3cn[nH]c3)c2)cc(C(F)(F)F)cc1C(F)(F)F)c1ccc(F)cc1 ; [2H]C([2H])([2H])N(C(=O)Oc1c(-c2cnn(CC#Cc3cnn(C)c3)c2)cc(C(F)(F)F)cc1C(F)(F)F)c1ccc(F)cc1 ; [2H]C([2H])([2H])N(C(=O)Oc1c(-c2cnn(CC#CCO)c2)cc(C(F)(F)F)cc1C(F)(F)F)c1ccc(F)cc1]

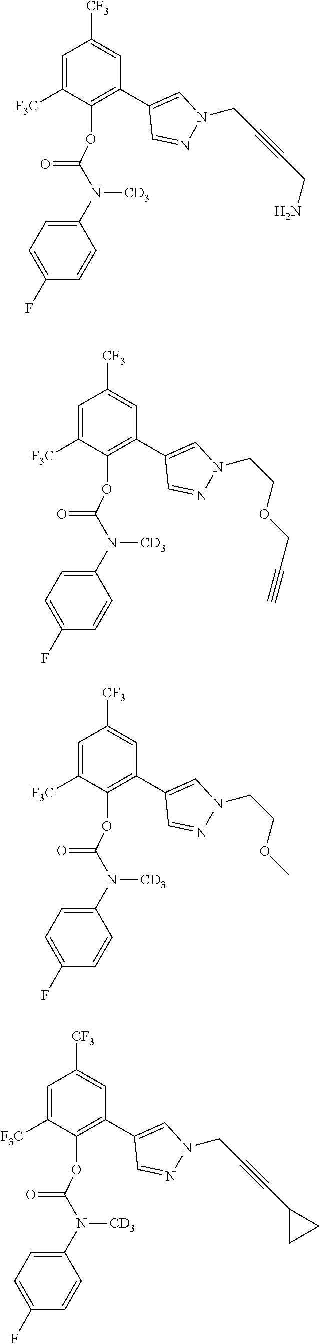 [SMILES: [2H]C([2H])([2H])N(C(=O)Oc1c(-c2cnn(CC#CCN)c2)cc(C(F)(F)F)cc1C(F)(F)F)c1ccc(F)cc1 ; [2H]C([2H])([2H])N(C(=O)Oc1c(-c2cnn(CCOCC#C)c2)cc(C(F)(F)F)cc1C(F)(F)F)c1ccc(F)cc1 ; [2H]C([2H])([2H])N(C(=O)Oc1c(-c2cnn(CCOC)c2)cc(C(F)(F)F)cc1C(F)(F)F)c1ccc(F)cc1 ; [2H]C([2H])([2H])N(C(=O)Oc1c(-c2cnn(CC#CC3CC3)c2)cc(C(F)(F)F)cc1C(F)(F)F)c1ccc(F)cc1]

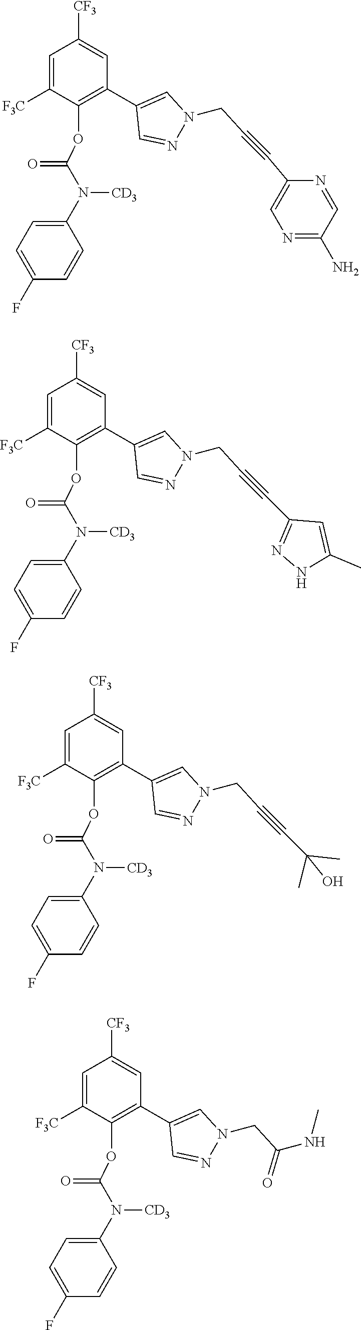 [SMILES: [2H]C([2H])([2H])N(C(=O)Oc1c(-c2cnn(CC#Cc3cnc(N)cn3)c2)cc(C(F)(F)F)cc1C(F)(F)F)c1ccc(F)cc1 ; [2H]C([2H])([2H])N(C(=O)Oc1c(-c2cnn(CC#Cc3cc(C)[nH]n3)c2)cc(C(F)(F)F)cc1C(F)(F)F)c1ccc(F)cc1 ; [2H]C([2H])([2H])N(C(=O)Oc1c(-c2cnn(CC#CC(C)(C)O)c2)cc(C(F)(F)F)cc1C(F)(F)F)c1ccc(F)cc1 ; [2H]C([2H])([2H])N(C(=O)Oc1c(-c2cnn(CC(=O)NC)c2)cc(C(F)(F)F)cc1C(F)(F)F)c1ccc(F)cc1]

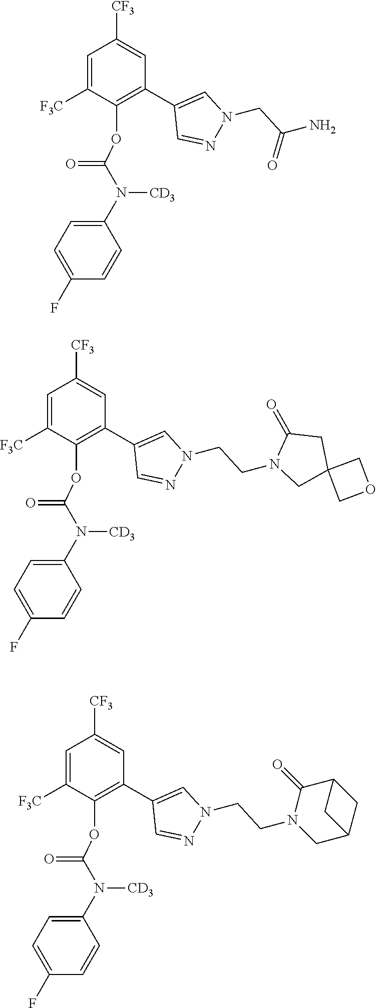

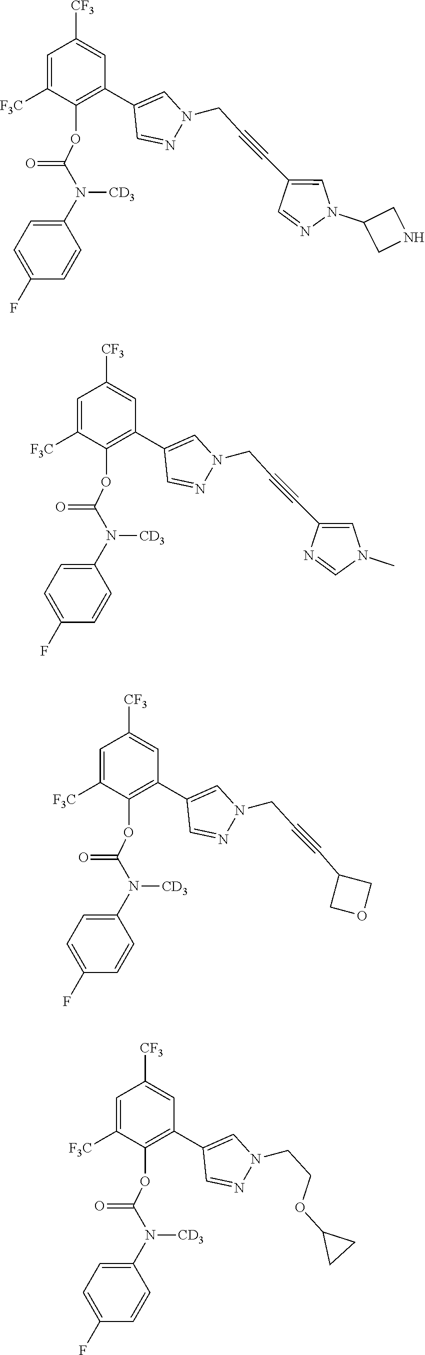 [SMILES: [2H]C([2H])([2H])N(C(=O)Oc1c(-c2cnn(CC#Cc3cnn(C4CNC4)c3)c2)cc(C(F)(F)F)cc1C(F)(F)F)c1ccc(F)cc1 ; [2H]C([2H])([2H])N(C(=O)Oc1c(-c2cnn(CC#Cc3cn(C)cn3)c2)cc(C(F)(F)F)cc1C(F)(F)F)c1ccc(F)cc1 ; [2H]C([2H])([2H])N(C(=O)Oc1c(-c2cnn(CC#CC3COC3)c2)cc(C(F)(F)F)cc1C(F)(F)F)c1ccc(F)cc1 ; [2H]C([2H])([2H])N(C(=O)Oc1c(-c2cnn(CCOC3CC3)c2)cc(C(F)(F)F)cc1C(F)(F)F)c1ccc(F)cc1]

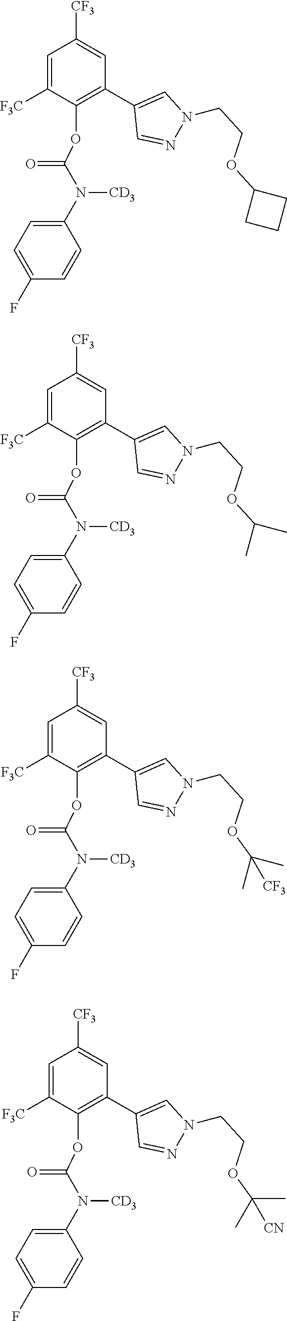 [SMILES: [2H]C([2H])([2H])N(C(=O)Oc1c(-c2cnn(CCOC3CCC3)c2)cc(C(F)(F)F)cc1C(F)(F)F)c1ccc(F)cc1 ; [2H]C([2H])([2H])N(C(=O)Oc1c(-c2cnn(CCOC(C)C)c2)cc(C(F)(F)F)cc1C(F)(F)F)c1ccc(F)cc1 ; [2H]C([2H])([2H])N(C(=O)Oc1c(-c2cnn(CCOC(C)(C)C(F)(F)F)c2)cc(C(F)(F)F)cc1C(F)(F)F)c1ccc(F)cc1 ; [2H]C([2H])([2H])N(C(=O)Oc1c(-c2cnn(CCOC(C)(C)C#N)c2)cc(C(F)(F)F)cc1C(F)(F)F)c1ccc(F)cc1]

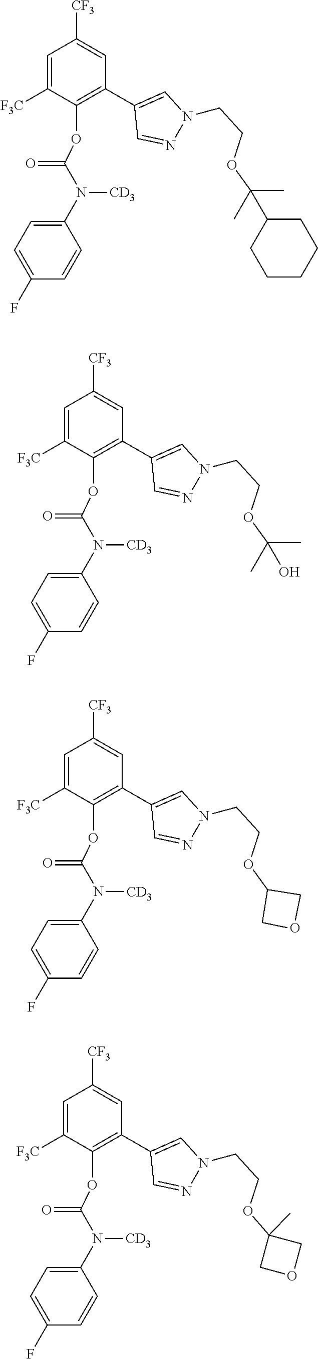 [SMILES: [2H]C([2H])([2H])N(C(=O)Oc1c(-c2cnn(CCOC(C)(C)C3CCCCC3)c2)cc(C(F)(F)F)cc1C(F)(F)F)c1ccc(F)cc1 ; [2H]C([2H])([2H])N(C(=O)Oc1c(-c2cnn(CCOC(C)(C)O)c2)cc(C(F)(F)F)cc1C(F)(F)F)c1ccc(F)cc1 ; [2H]C([2H])([2H])N(C(=O)Oc1c(-c2cnn(CCOC3COC3)c2)cc(C(F)(F)F)cc1C(F)(F)F)c1ccc(F)cc1 ; [2H]C([2H])([2H])N(C(=O)Oc1c(-c2cnn(CCOC3(C)COC3)c2)cc(C(F)(F)F)cc1C(F)(F)F)c1ccc(F)cc1]

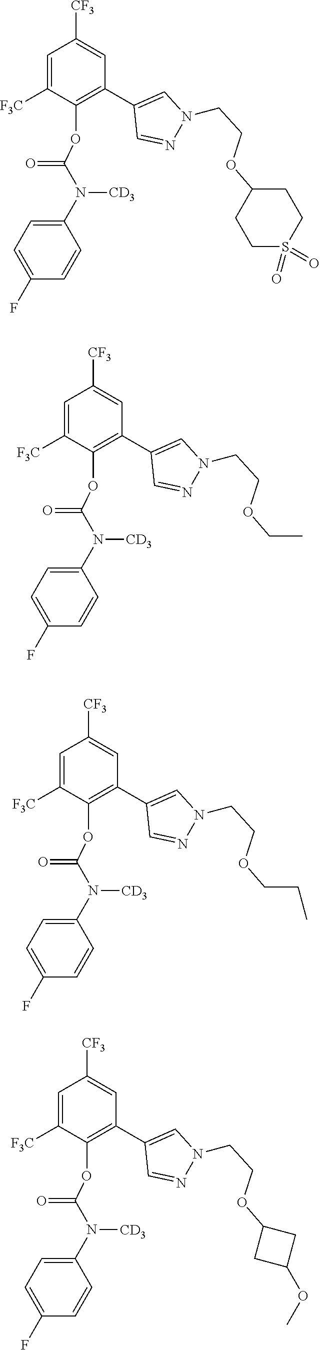 [SMILES: [2H]C([2H])([2H])N(C(=O)Oc1c(-c2cnn(CCOC3CCS(=O)(=O)CC3)c2)cc(C(F)(F)F)cc1C(F)(F)F)c1ccc(F)cc1 ; [2H]C([2H])([2H])N(C(=O)Oc1c(-c2cnn(CCOCC)c2)cc(C(F)(F)F)cc1C(F)(F)F)c1ccc(F)cc1 ; [2H]C([2H])([2H])N(C(=O)Oc1c(-c2cnn(CCOCCC)c2)cc(C(F)(F)F)cc1C(F)(F)F)c1ccc(F)cc1 ; [2H]C([2H])([2H])N(C(=O)Oc1c(-c2cnn(CCOC3CC(OC)C3)c2)cc(C(F)(F)F)cc1C(F)(F)F)c1ccc(F)cc1]

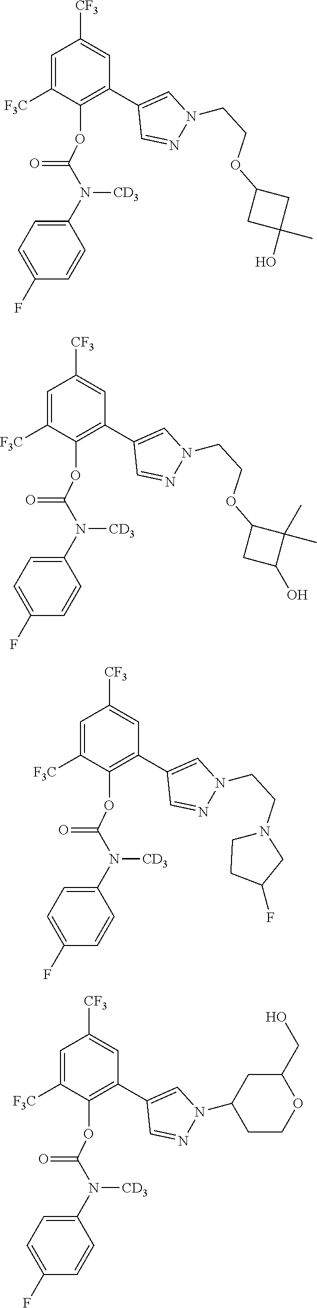 [SMILES: [2H]C([2H])([2H])N(C(=O)Oc1c(-c2cnn(CCOC3CC(C)(O)C3)c2)cc(C(F)(F)F)cc1C(F)(F)F)c1ccc(F)cc1 ; [2H]C([2H])([2H])N(C(=O)Oc1c(-c2cnn(CCOC3CC(O)C3(C)C)c2)cc(C(F)(F)F)cc1C(F)(F)F)c1ccc(F)cc1 ; [2H]C([2H])([2H])N(C(=O)Oc1c(-c2cnn(CCN3CCC(F)C3)c2)cc(C(F)(F)F)cc1C(F)(F)F)c1ccc(F)cc1 ; [2H]C([2H])([2H])N(C(=O)Oc1c(-c2cnn(C3CCOC(CO)C3)c2)cc(C(F)(F)F)cc1C(F)(F)F)c1ccc(F)cc1]

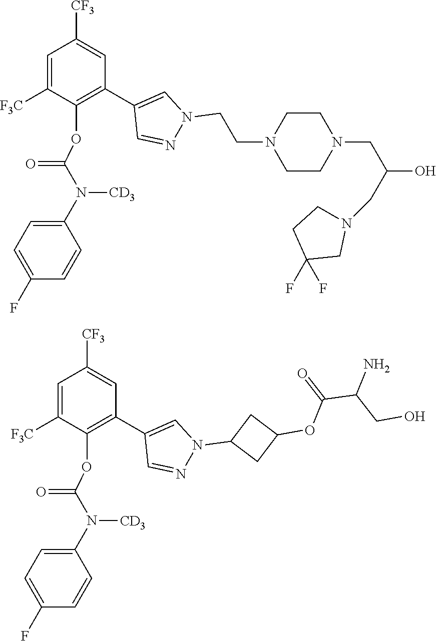 [SMILES: [2H]C([2H])([2H])N(C(=O)Oc1c(-c2cnn(CCN3CCN(CC(O)CN4CCC(F)(F)C4)CC3)c2)cc(C(F)(F)F)cc1C(F)(F)F)c1ccc(F)cc1 ; [2H]C([2H])([2H])N(C(=O)Oc1c(-c2cnn(C3CC(OC(=O)C(N)CO)C3)c2)cc(C(F)(F)F)cc1C(F)(F)F)c1ccc(F)cc1]

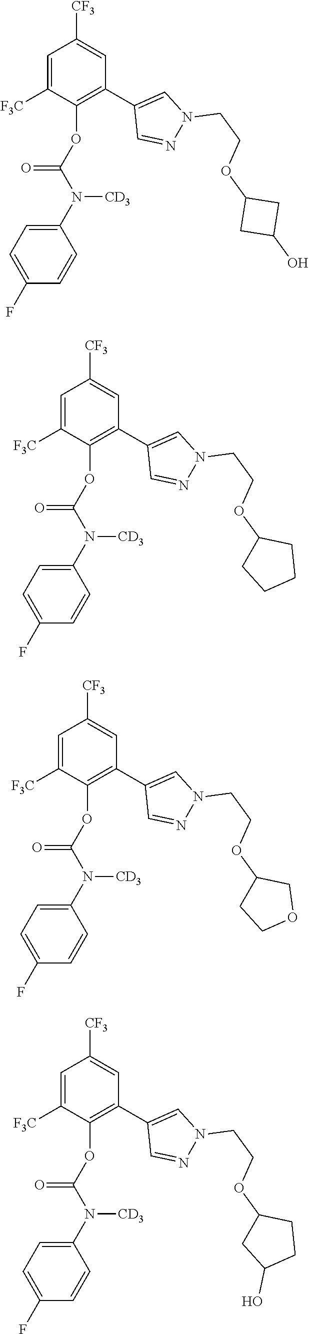 [SMILES: [2H]C([2H])([2H])N(C(=O)Oc1c(-c2cnn(CCOC3CC(O)C3)c2)cc(C(F)(F)F)cc1C(F)(F)F)c1ccc(F)cc1 ; [2H]C([2H])([2H])N(C(=O)Oc1c(-c2cnn(CCOC3CCCC3)c2)cc(C(F)(F)F)cc1C(F)(F)F)c1ccc(F)cc1 ; [2H]C([2H])([2H])N(C(=O)Oc1c(-c2cnn(CCOC3CCOC3)c2)cc(C(F)(F)F)cc1C(F)(F)F)c1ccc(F)cc1 ; [2H]C([2H])([2H])N(C(=O)Oc1c(-c2cnn(CCOC3CCC(O)C3)c2)cc(C(F)(F)F)cc1C(F)(F)F)c1ccc(F)cc1]

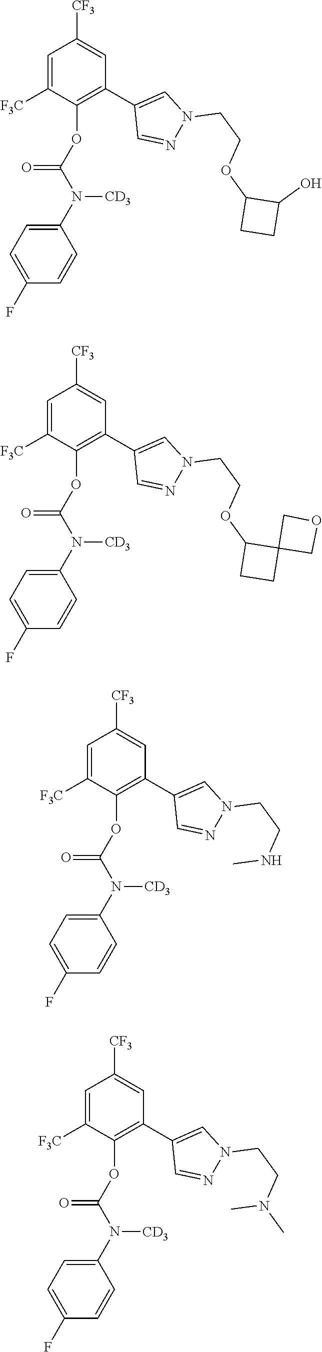 [SMILES: [2H]C([2H])([2H])N(C(=O)Oc1c(-c2cnn(CCOC3CCC3O)c2)cc(C(F)(F)F)cc1C(F)(F)F)c1ccc(F)cc1 ; [2H]C([2H])([2H])N(C(=O)Oc1c(-c2cnn(CCOC3CCC34COC4)c2)cc(C(F)(F)F)cc1C(F)(F)F)c1ccc(F)cc1 ; [2H]C([2H])([2H])N(C(=O)Oc1c(-c2cnn(CCNC)c2)cc(C(F)(F)F)cc1C(F)(F)F)c1ccc(F)cc1 ; [2H]C([2H])([2H])N(C(=O)Oc1c(-c2cnn(CCN(C)C)c2)cc(C(F)(F)F)cc1C(F)(F)F)c1ccc(F)cc1]

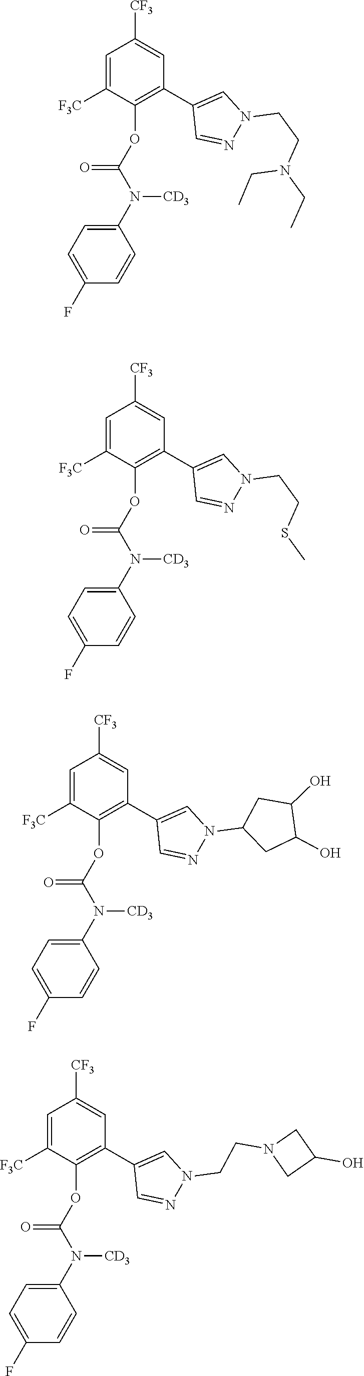 [SMILES: [2H]C([2H])([2H])N(C(=O)Oc1c(-c2cnn(CCN(CC)CC)c2)cc(C(F)(F)F)cc1C(F)(F)F)c1ccc(F)cc1 ; [2H]C([2H])([2H])N(C(=O)Oc1c(-c2cnn(CCSC)c2)cc(C(F)(F)F)cc1C(F)(F)F)c1ccc(F)cc1 ; [2H]C([2H])([2H])N(C(=O)Oc1c(-c2cnn(C3CC(O)C(O)C3)c2)cc(C(F)(F)F)cc1C(F)(F)F)c1ccc(F)cc1 ; [2H]C([2H])([2H])N(C(=O)Oc1c(-c2cnn(CCN3CC(O)C3)c2)cc(C(F)(F)F)cc1C(F)(F)F)c1ccc(F)cc1]

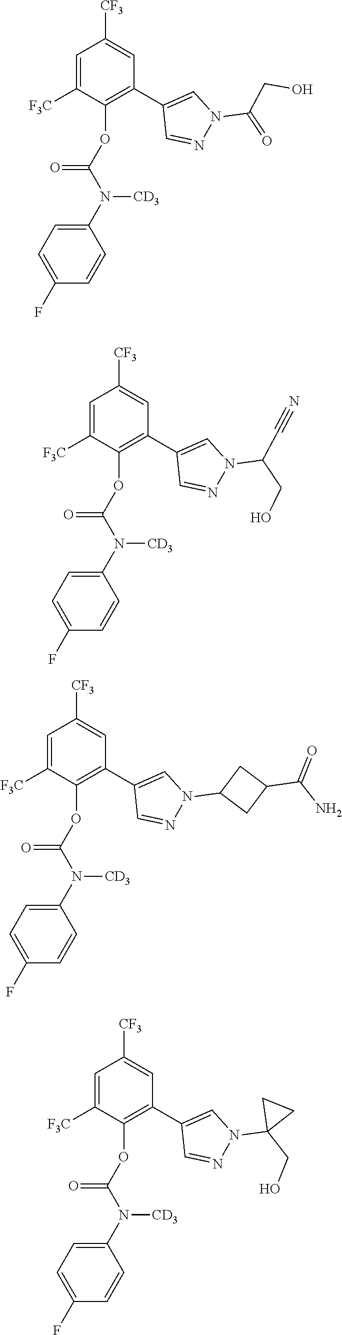

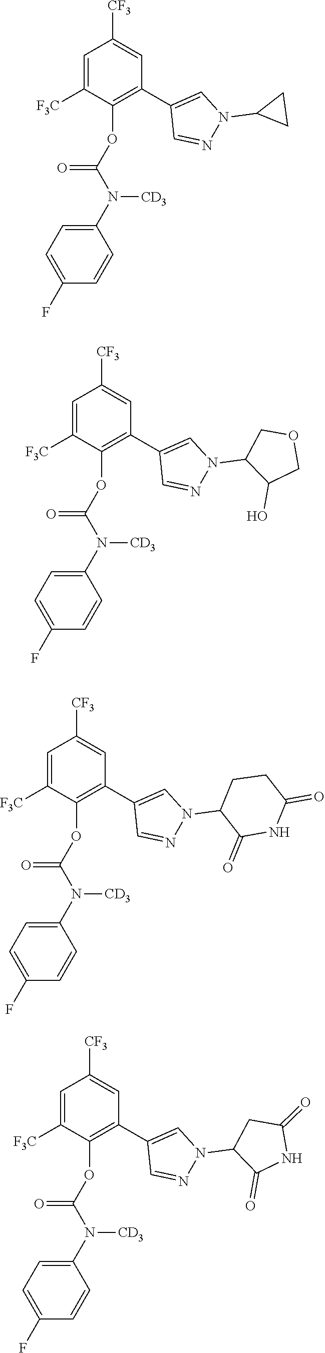 [SMILES: [2H]C([2H])([2H])N(C(=O)Oc1c(-c2cnn(C3CC3)c2)cc(C(F)(F)F)cc1C(F)(F)F)c1ccc(F)cc1 ; [2H]C([2H])([2H])N(C(=O)Oc1c(-c2cnn(C3COCC3O)c2)cc(C(F)(F)F)cc1C(F)(F)F)c1ccc(F)cc1 ; [2H]C([2H])([2H])N(C(=O)Oc1c(-c2cnn(C3CCC(=O)NC3=O)c2)cc(C(F)(F)F)cc1C(F)(F)F)c1ccc(F)cc1 ; [2H]C([2H])([2H])N(C(=O)Oc1c(-c2cnn(C3CC(=O)NC3=O)c2)cc(C(F)(F)F)cc1C(F)(F)F)c1ccc(F)cc1]

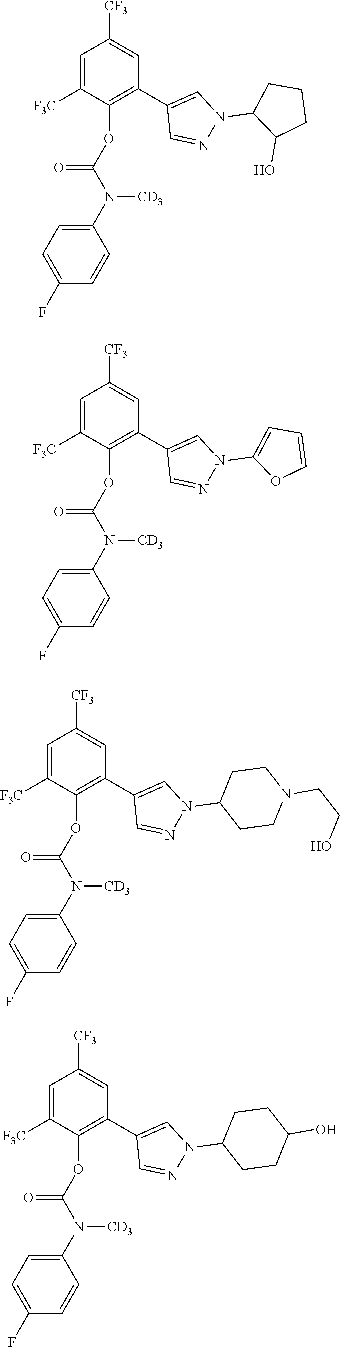 [SMILES: [2H]C([2H])([2H])N(C(=O)Oc1c(-c2cnn(C3CCCC3O)c2)cc(C(F)(F)F)cc1C(F)(F)F)c1ccc(F)cc1 ; [2H]C([2H])([2H])N(C(=O)Oc1c(-c2cnn(-c3ccco3)c2)cc(C(F)(F)F)cc1C(F)(F)F)c1ccc(F)cc1 ; [2H]C([2H])([2H])N(C(=O)Oc1c(-c2cnn(C3CCN(CCO)CC3)c2)cc(C(F)(F)F)cc1C(F)(F)F)c1ccc(F)cc1 ; [2H]C([2H])([2H])N(C(=O)Oc1c(-c2cnn(C3CCC(O)CC3)c2)cc(C(F)(F)F)cc1C(F)(F)F)c1ccc(F)cc1]

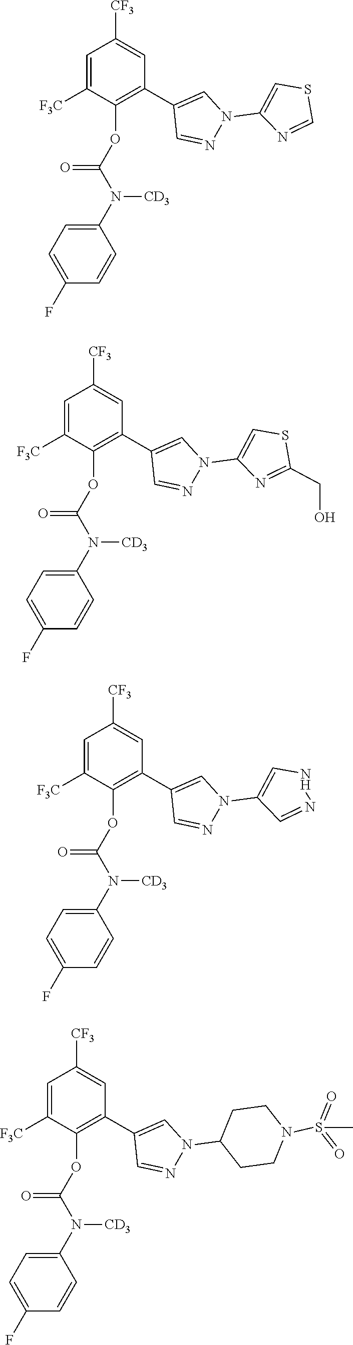 [SMILES: [2H]C([2H])([2H])N(C(=O)Oc1c(-c2cnn(-c3cscn3)c2)cc(C(F)(F)F)cc1C(F)(F)F)c1ccc(F)cc1 ; [2H]C([2H])([2H])N(C(=O)Oc1c(-c2cnn(-c3csc(CO)n3)c2)cc(C(F)(F)F)cc1C(F)(F)F)c1ccc(F)cc1 ; [2H]C([2H])([2H])N(C(=O)Oc1c(-c2cnn(-c3cn[nH]c3)c2)cc(C(F)(F)F)cc1C(F)(F)F)c1ccc(F)cc1 ; [2H]C([2H])([2H])N(C(=O)Oc1c(-c2cnn(C3CCN(S(C)(=O)=O)CC3)c2)cc(C(F)(F)F)cc1C(F)(F)F)c1ccc(F)cc1]

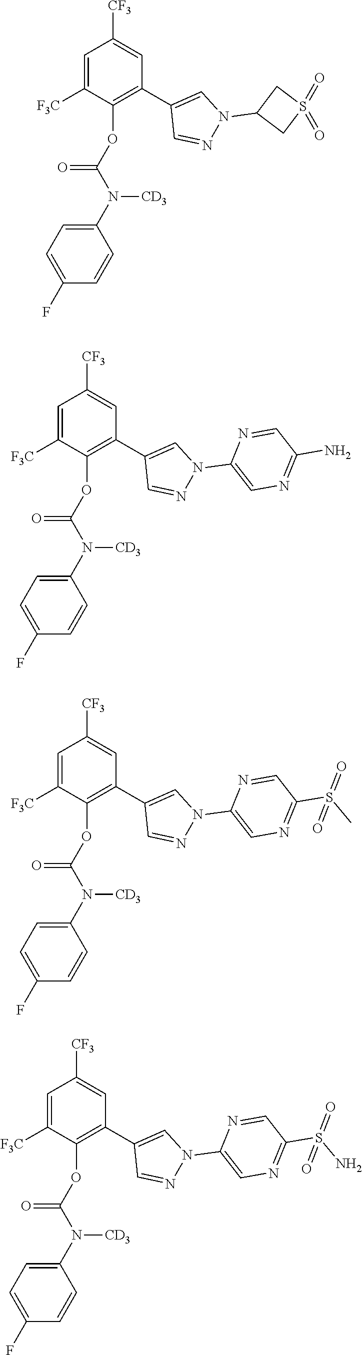 [SMILES: [2H]C([2H])([2H])N(C(=O)Oc1c(-c2cnn(C3CS(=O)(=O)C3)c2)cc(C(F)(F)F)cc1C(F)(F)F)c1ccc(F)cc1 ; [2H]C([2H])([2H])N(C(=O)Oc1c(-c2cnn(-c3cnc(N)cn3)c2)cc(C(F)(F)F)cc1C(F)(F)F)c1ccc(F)cc1 ; [2H]C([2H])([2H])N(C(=O)Oc1c(-c2cnn(-c3cnc(S(C)(=O)=O)cn3)c2)cc(C(F)(F)F)cc1C(F)(F)F)c1ccc(F)cc1 ; [2H]C([2H])([2H])N(C(=O)Oc1c(-c2cnn(-c3cnc(S(N)(=O)=O)cn3)c2)cc(C(F)(F)F)cc1C(F)(F)F)c1ccc(F)cc1]

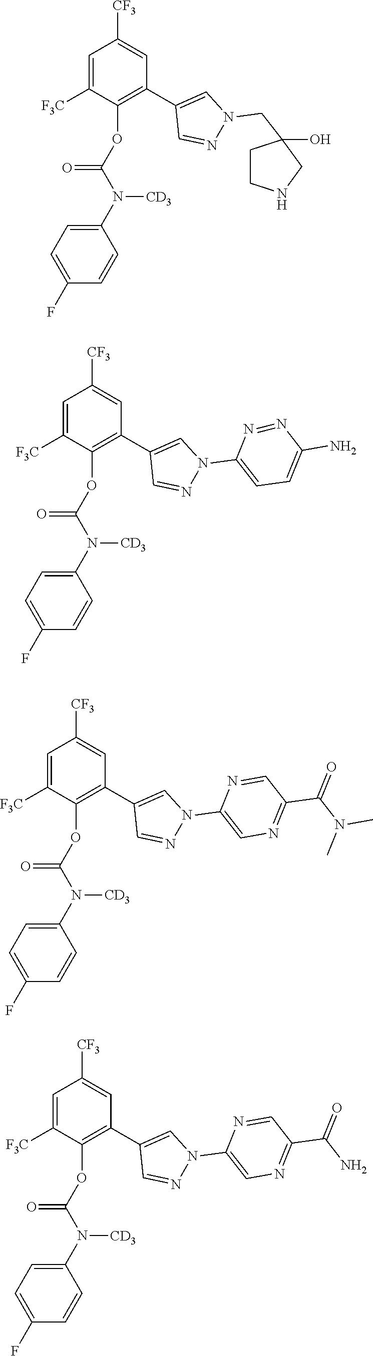 [SMILES: [2H]C([2H])([2H])N(C(=O)Oc1c(-c2cnn(CC3(O)CCNC3)c2)cc(C(F)(F)F)cc1C(F)(F)F)c1ccc(F)cc1 ; [2H]C([2H])([2H])N(C(=O)Oc1c(-c2cnn(-c3ccc(N)nn3)c2)cc(C(F)(F)F)cc1C(F)(F)F)c1ccc(F)cc1 ; [2H]C([2H])([2H])N(C(=O)Oc1c(-c2cnn(-c3cnc(C(=O)N(C)C)cn3)c2)cc(C(F)(F)F)cc1C(F)(F)F)c1ccc(F)cc1 ; [2H]C([2H])([2H])N(C(=O)Oc1c(-c2cnn(-c3cnc(C(N)=O)cn3)c2)cc(C(F)(F)F)cc1C(F)(F)F)c1ccc(F)cc1]

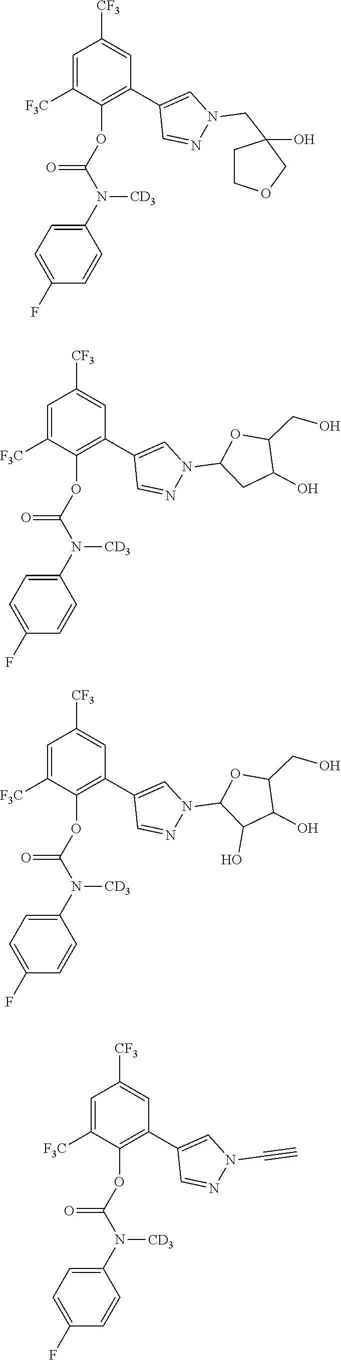 [SMILES: [2H]C([2H])([2H])N(C(=O)Oc1c(-c2cnn(CC3(O)CCOC3)c2)cc(C(F)(F)F)cc1C(F)(F)F)c1ccc(F)cc1 ; [2H]C([2H])([2H])N(C(=O)Oc1c(-c2cnn(C3CC(O)C(CO)O3)c2)cc(C(F)(F)F)cc1C(F)(F)F)c1ccc(F)cc1 ; [2H]C([2H])([2H])N(C(=O)Oc1c(-c2cnn(C3OC(CO)C(O)C3O)c2)cc(C(F)(F)F)cc1C(F)(F)F)c1ccc(F)cc1 ; [2H]C([2H])([2H])N(C(=O)Oc1c(-c2cnn(C#C)c2)cc(C(F)(F)F)cc1C(F)(F)F)c1ccc(F)cc1]

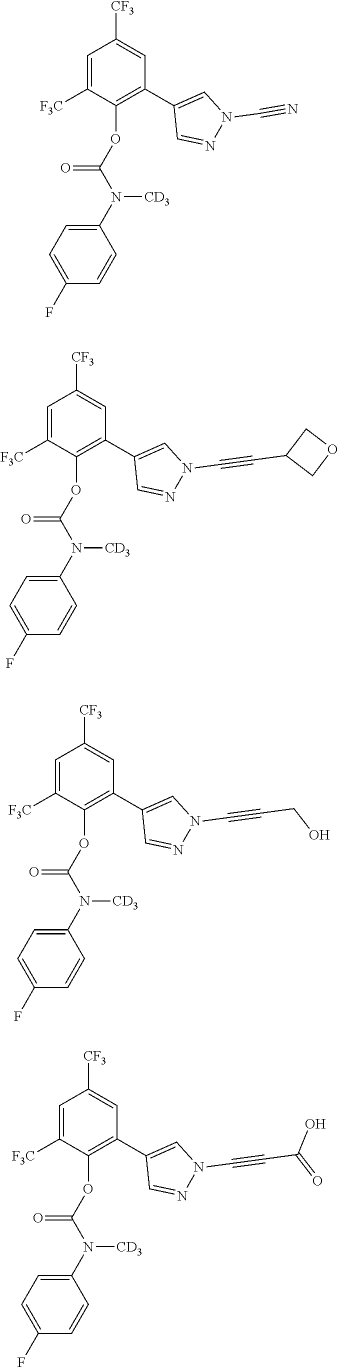 [SMILES: [2H]C([2H])([2H])N(C(=O)Oc1c(-c2cnn(C#N)c2)cc(C(F)(F)F)cc1C(F)(F)F)c1ccc(F)cc1 ; [2H]C([2H])([2H])N(C(=O)Oc1c(-c2cnn(C#CC3COC3)c2)cc(C(F)(F)F)cc1C(F)(F)F)c1ccc(F)cc1 ; [2H]C([2H])([2H])N(C(=O)Oc1c(-c2cnn(C#CCO)c2)cc(C(F)(F)F)cc1C(F)(F)F)c1ccc(F)cc1 ; [2H]C([2H])([2H])N(C(=O)Oc1c(-c2cnn(C#CC(=O)O)c2)cc(C(F)(F)F)cc1C(F)(F)F)c1ccc(F)cc1]

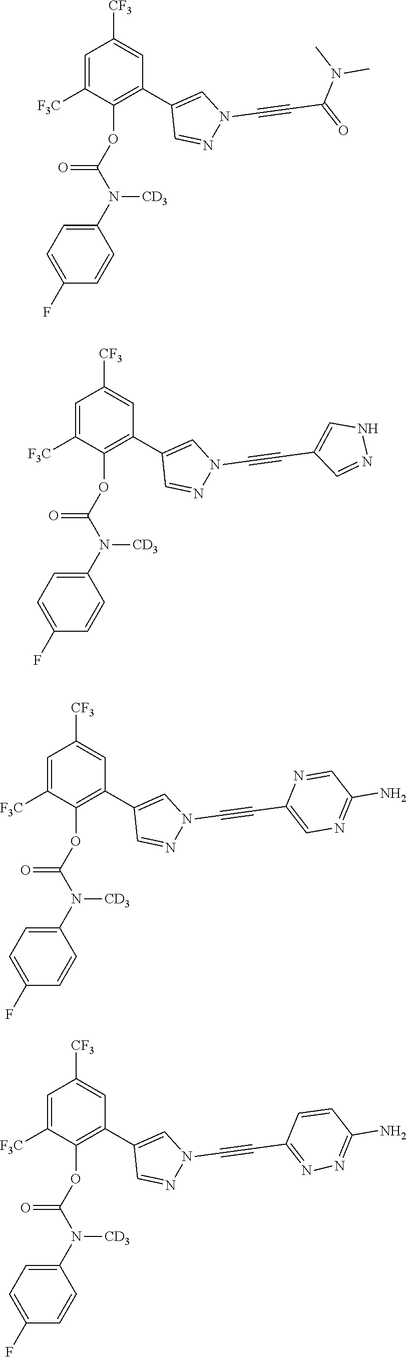 [SMILES: [2H]C([2H])([2H])N(C(=O)Oc1c(-c2cnn(C#CC(=O)N(C)C)c2)cc(C(F)(F)F)cc1C(F)(F)F)c1ccc(F)cc1 ; [2H]C([2H])([2H])N(C(=O)Oc1c(-c2cnn(C#Cc3cn[nH]c3)c2)cc(C(F)(F)F)cc1C(F)(F)F)c1ccc(F)cc1 ; [2H]C([2H])([2H])N(C(=O)Oc1c(-c2cnn(C#Cc3cnc(N)cn3)c2)cc(C(F)(F)F)cc1C(F)(F)F)c1ccc(F)cc1 ; [2H]C([2H])([2H])N(C(=O)Oc1c(-c2cnn(C#Cc3ccc(N)nn3)c2)cc(C(F)(F)F)cc1C(F)(F)F)c1ccc(F)cc1]

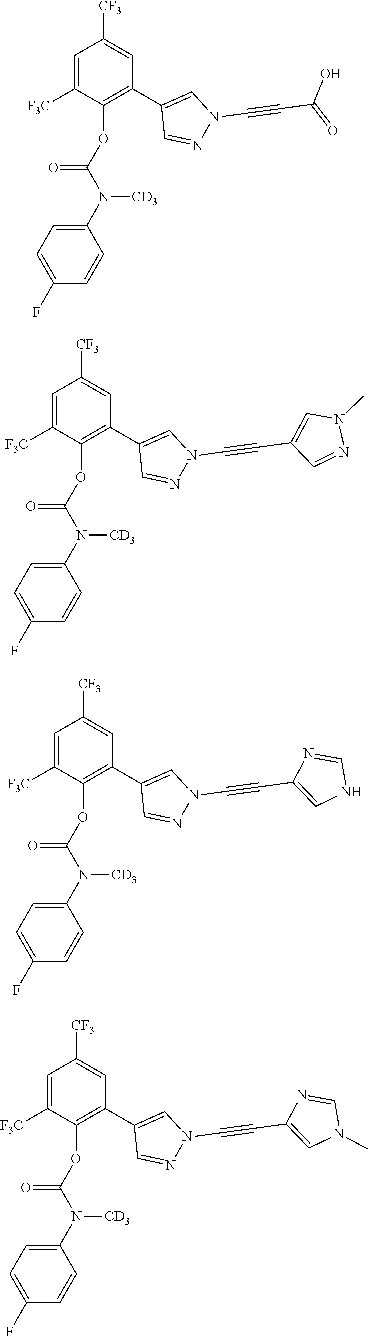 [SMILES: [2H]C([2H])([2H])N(C(=O)Oc1c(-c2cnn(C#CC(=O)O)c2)cc(C(F)(F)F)cc1C(F)(F)F)c1ccc(F)cc1 ; [2H]C([2H])([2H])N(C(=O)Oc1c(-c2cnn(C#Cc3cnn(C)c3)c2)cc(C(F)(F)F)cc1C(F)(F)F)c1ccc(F)cc1 ; [2H]C([2H])([2H])N(C(=O)Oc1c(-c2cnn(C#Cc3c[nH]cn3)c2)cc(C(F)(F)F)cc1C(F)(F)F)c1ccc(F)cc1 ; [2H]C([2H])([2H])N(C(=O)Oc1c(-c2cnn(C#Cc3cn(C)cn3)c2)cc(C(F)(F)F)cc1C(F)(F)F)c1ccc(F)cc1]

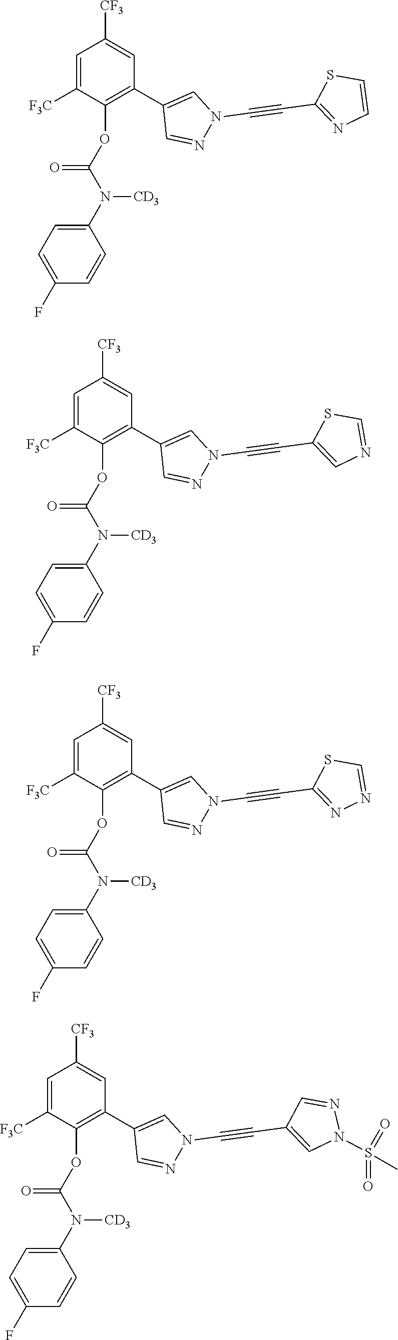 [SMILES: [2H]C([2H])([2H])N(C(=O)Oc1c(-c2cnn(C#Cc3nccs3)c2)cc(C(F)(F)F)cc1C(F)(F)F)c1ccc(F)cc1 ; [2H]C([2H])([2H])N(C(=O)Oc1c(-c2cnn(C#Cc3cncs3)c2)cc(C(F)(F)F)cc1C(F)(F)F)c1ccc(F)cc1 ; [2H]C([2H])([2H])N(C(=O)Oc1c(-c2cnn(C#Cc3nncs3)c2)cc(C(F)(F)F)cc1C(F)(F)F)c1ccc(F)cc1 ; [2H]C([2H])([2H])N(C(=O)Oc1c(-c2cnn(C#Cc3cnn(S(C)(=O)=O)c3)c2)cc(C(F)(F)F)cc1C(F)(F)F)c1ccc(F)cc1]

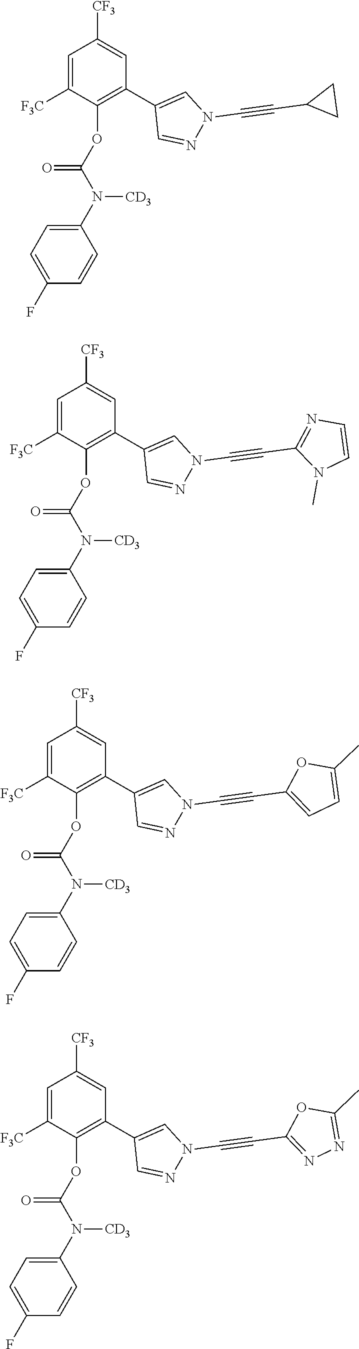 [SMILES: [2H]C([2H])([2H])N(C(=O)Oc1c(-c2cnn(C#CC3CC3)c2)cc(C(F)(F)F)cc1C(F)(F)F)c1ccc(F)cc1 ; [2H]C([2H])([2H])N(C(=O)Oc1c(-c2cnn(C#Cc3nccn3C)c2)cc(C(F)(F)F)cc1C(F)(F)F)c1ccc(F)cc1 ; [2H]C([2H])([2H])N(C(=O)Oc1c(-c2cnn(C#Cc3ccc(C)o3)c2)cc(C(F)(F)F)cc1C(F)(F)F)c1ccc(F)cc1 ; [2H]C([2H])([2H])N(C(=O)Oc1c(-c2cnn(C#Cc3nnc(C)o3)c2)cc(C(F)(F)F)cc1C(F)(F)F)c1ccc(F)cc1]

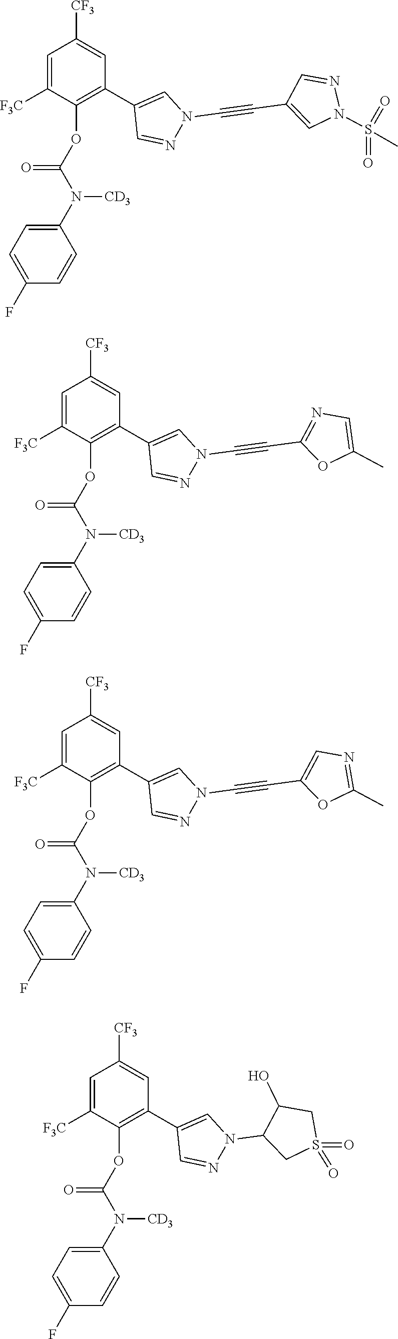 [SMILES: [2H]C([2H])([2H])N(C(=O)Oc1c(-c2cnn(C#Cc3cnn(S(C)(=O)=O)c3)c2)cc(C(F)(F)F)cc1C(F)(F)F)c1ccc(F)cc1 ; [2H]C([2H])([2H])N(C(=O)Oc1c(-c2cnn(C#Cc3ncc(C)o3)c2)cc(C(F)(F)F)cc1C(F)(F)F)c1ccc(F)cc1 ; [2H]C([2H])([2H])N(C(=O)Oc1c(-c2cnn(C#Cc3cnc(C)o3)c2)cc(C(F)(F)F)cc1C(F)(F)F)c1ccc(F)cc1 ; [2H]C([2H])([2H])N(C(=O)Oc1c(-c2cnn(C3CS(=O)(=O)CC3O)c2)cc(C(F)(F)F)cc1C(F)(F)F)c1ccc(F)cc1]

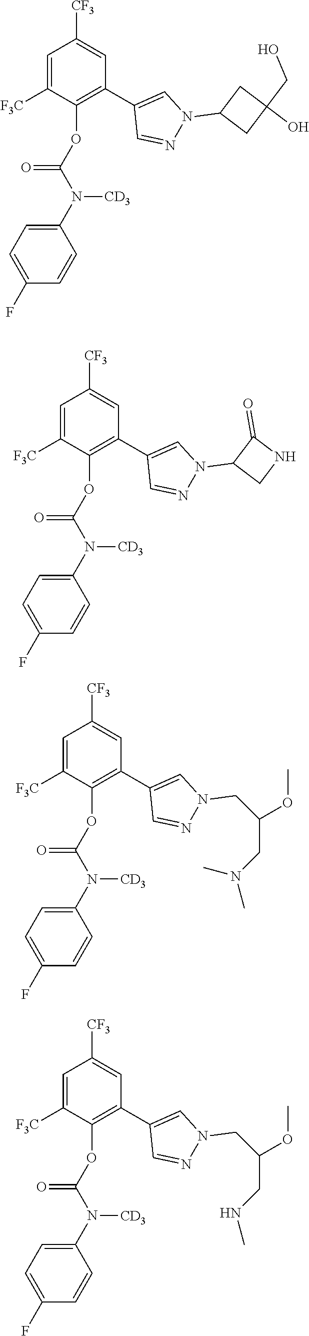 [SMILES: [2H]C([2H])([2H])N(C(=O)Oc1c(-c2cnn(C3CC(O)(CO)C3)c2)cc(C(F)(F)F)cc1C(F)(F)F)c1ccc(F)cc1 ; [2H]C([2H])([2H])N(C(=O)Oc1c(-c2cnn(C3CNC3=O)c2)cc(C(F)(F)F)cc1C(F)(F)F)c1ccc(F)cc1 ; [2H]C([2H])([2H])N(C(=O)Oc1c(-c2cnn(CC(CN(C)C)OC)c2)cc(C(F)(F)F)cc1C(F)(F)F)c1ccc(F)cc1 ; [2H]C([2H])([2H])N(C(=O)Oc1c(-c2cnn(CC(CNC)OC)c2)cc(C(F)(F)F)cc1C(F)(F)F)c1ccc(F)cc1]

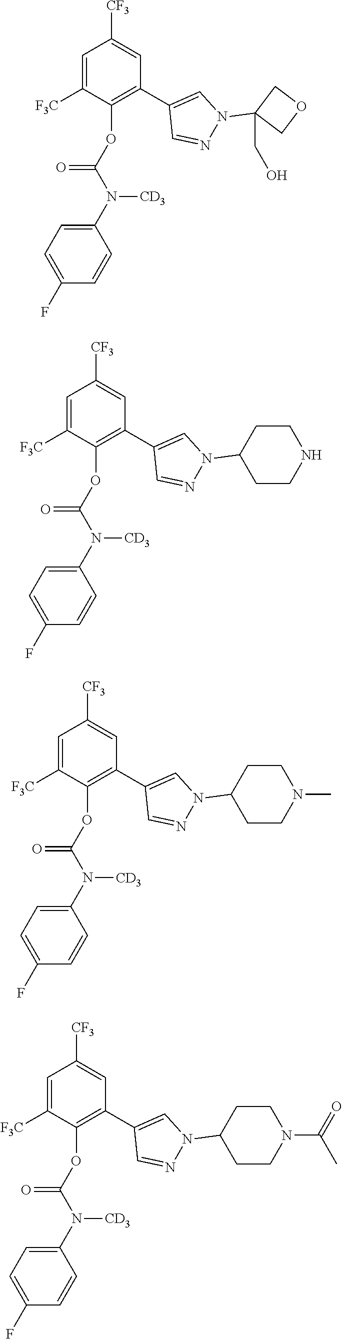 [SMILES: [2H]C([2H])([2H])N(C(=O)Oc1c(-c2cnn(C3(CO)COC3)c2)cc(C(F)(F)F)cc1C(F)(F)F)c1ccc(F)cc1 ; [2H]C([2H])([2H])N(C(=O)Oc1c(-c2cnn(C3CCNCC3)c2)cc(C(F)(F)F)cc1C(F)(F)F)c1ccc(F)cc1 ; [2H]C([2H])([2H])N(C(=O)Oc1c(-c2cnn(C3CCN(C)CC3)c2)cc(C(F)(F)F)cc1C(F)(F)F)c1ccc(F)cc1 ; [2H]C([2H])([2H])N(C(=O)Oc1c(-c2cnn(C3CCN(C(C)=O)CC3)c2)cc(C(F)(F)F)cc1C(F)(F)F)c1ccc(F)cc1]

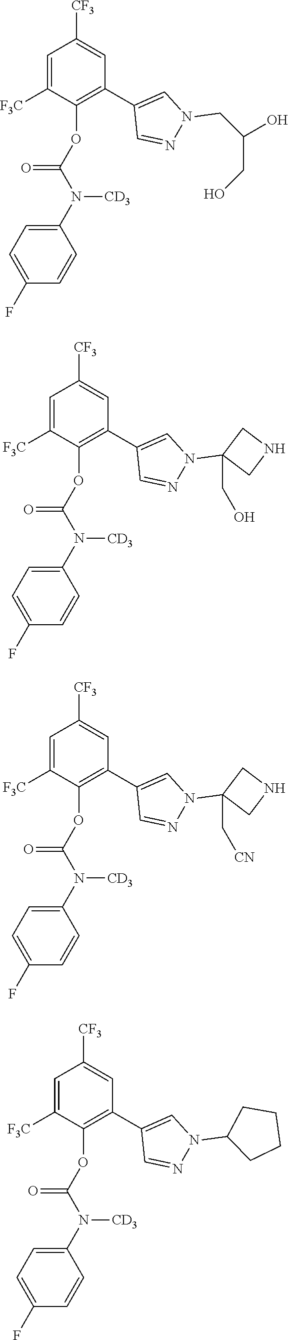 [SMILES: [2H]C([2H])([2H])N(C(=O)Oc1c(-c2cnn(CC(O)CO)c2)cc(C(F)(F)F)cc1C(F)(F)F)c1ccc(F)cc1 ; [2H]C([2H])([2H])N(C(=O)Oc1c(-c2cnn(C3(CO)CNC3)c2)cc(C(F)(F)F)cc1C(F)(F)F)c1ccc(F)cc1 ; [2H]C([2H])([2H])N(C(=O)Oc1c(-c2cnn(C3(CC#N)CNC3)c2)cc(C(F)(F)F)cc1C(F)(F)F)c1ccc(F)cc1 ; [2H]C([2H])([2H])N(C(=O)Oc1c(-c2cnn(C3CCCC3)c2)cc(C(F)(F)F)cc1C(F)(F)F)c1ccc(F)cc1]

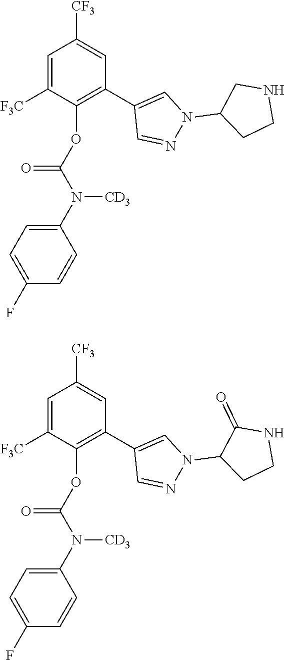 [SMILES: [2H]C([2H])([2H])N(C(=O)Oc1c(-c2cnn(C3CCNC3)c2)cc(C(F)(F)F)cc1C(F)(F)F)c1ccc(F)cc1 ; [2H]C([2H])([2H])N(C(=O)Oc1c(-c2cnn(C3CCNC3=O)c2)cc(C(F)(F)F)cc1C(F)(F)F)c1ccc(F)cc1]

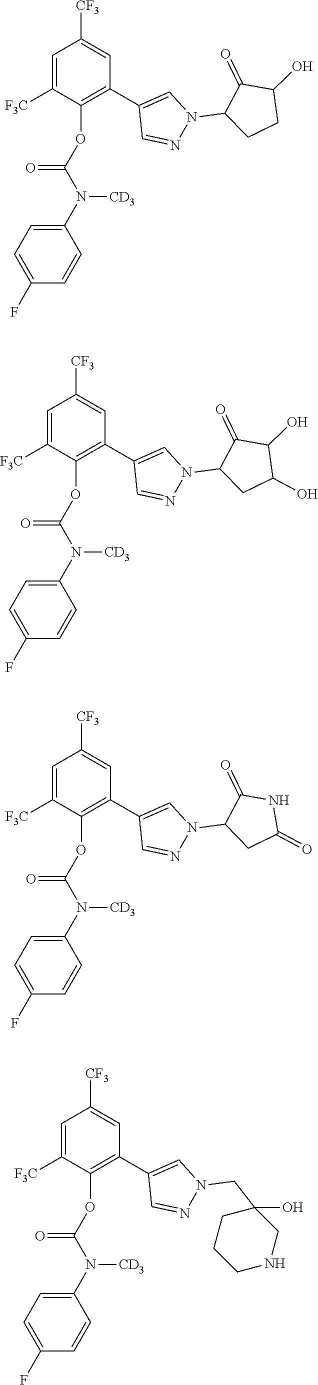 [SMILES: [2H]C([2H])([2H])N(C(=O)Oc1c(-c2cnn(C3CCC(O)C3=O)c2)cc(C(F)(F)F)cc1C(F)(F)F)c1ccc(F)cc1 ; [2H]C([2H])([2H])N(C(=O)Oc1c(-c2cnn(C3CC(O)C(O)C3=O)c2)cc(C(F)(F)F)cc1C(F)(F)F)c1ccc(F)cc1 ; [2H]C([2H])([2H])N(C(=O)Oc1c(-c2cnn(C3CC(=O)NC3=O)c2)cc(C(F)(F)F)cc1C(F)(F)F)c1ccc(F)cc1 ; [2H]C([2H])([2H])N(C(=O)Oc1c(-c2cnn(CC3(O)CCCNC3)c2)cc(C(F)(F)F)cc1C(F)(F)F)c1ccc(F)cc1]

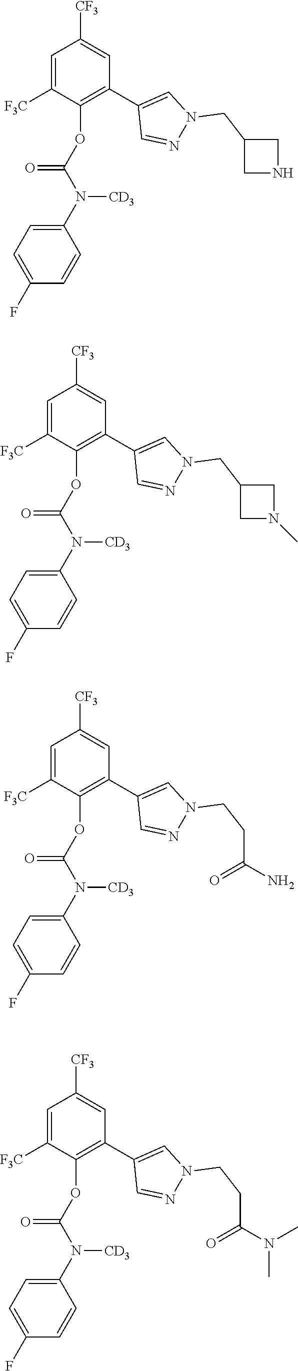 [SMILES: [2H]C([2H])([2H])N(C(=O)Oc1c(-c2cnn(CC3CNC3)c2)cc(C(F)(F)F)cc1C(F)(F)F)c1ccc(F)cc1 ; [2H]C([2H])([2H])N(C(=O)Oc1c(-c2cnn(CC3CN(C)C3)c2)cc(C(F)(F)F)cc1C(F)(F)F)c1ccc(F)cc1 ; [2H]C([2H])([2H])N(C(=O)Oc1c(-c2cnn(CCC(N)=O)c2)cc(C(F)(F)F)cc1C(F)(F)F)c1ccc(F)cc1 ; [2H]C([2H])([2H])N(C(=O)Oc1c(-c2cnn(CCC(=O)N(C)C)c2)cc(C(F)(F)F)cc1C(F)(F)F)c1ccc(F)cc1]

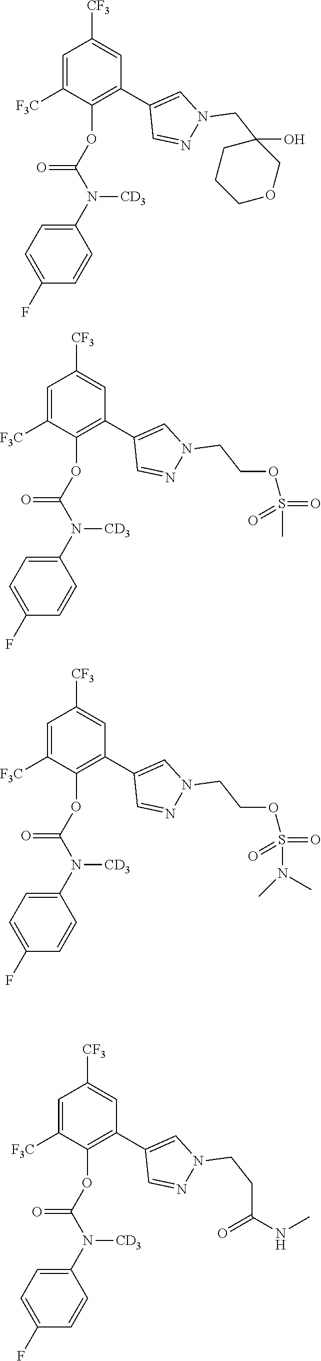 [SMILES: [2H]C([2H])([2H])N(C(=O)Oc1c(-c2cnn(CC3(O)CCCOC3)c2)cc(C(F)(F)F)cc1C(F)(F)F)c1ccc(F)cc1 ; [2H]C([2H])([2H])N(C(=O)Oc1c(-c2cnn(CCOS(C)(=O)=O)c2)cc(C(F)(F)F)cc1C(F)(F)F)c1ccc(F)cc1 ; [2H]C([2H])([2H])N(C(=O)Oc1c(-c2cnn(CCOS(=O)(=O)N(C)C)c2)cc(C(F)(F)F)cc1C(F)(F)F)c1ccc(F)cc1 ; [2H]C([2H])([2H])N(C(=O)Oc1c(-c2cnn(CCC(=O)NC)c2)cc(C(F)(F)F)cc1C(F)(F)F)c1ccc(F)cc1]

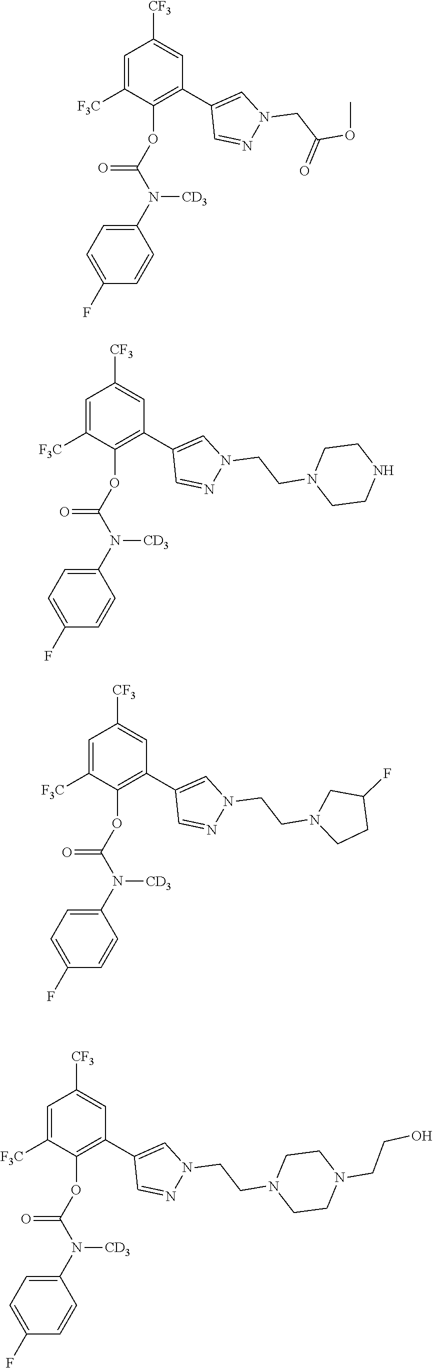 [SMILES: [2H]C([2H])([2H])N(C(=O)Oc1c(-c2cnn(CC(=O)OC)c2)cc(C(F)(F)F)cc1C(F)(F)F)c1ccc(F)cc1 ; [2H]C([2H])([2H])N(C(=O)Oc1c(-c2cnn(CCN3CCNCC3)c2)cc(C(F)(F)F)cc1C(F)(F)F)c1ccc(F)cc1 ; [2H]C([2H])([2H])N(C(=O)Oc1c(-c2cnn(CCN3CCC(F)C3)c2)cc(C(F)(F)F)cc1C(F)(F)F)c1ccc(F)cc1 ; [2H]C([2H])([2H])N(C(=O)Oc1c(-c2cnn(CCN3CCN(CCO)CC3)c2)cc(C(F)(F)F)cc1C(F)(F)F)c1ccc(F)cc1]

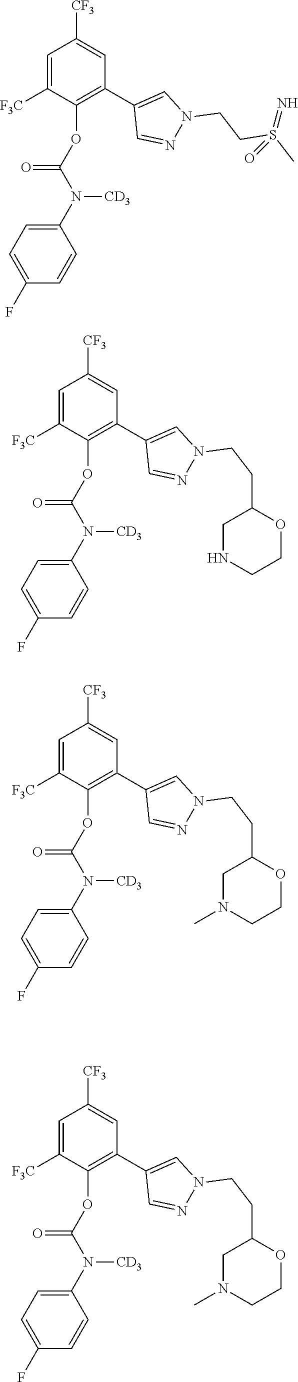 [SMILES: [2H]C([2H])([2H])N(C(=O)Oc1c(-c2cnn(CCS(C)(=N)=O)c2)cc(C(F)(F)F)cc1C(F)(F)F)c1ccc(F)cc1 ; [2H]C([2H])([2H])N(C(=O)Oc1c(-c2cnn(CCC3CNCCO3)c2)cc(C(F)(F)F)cc1C(F)(F)F)c1ccc(F)cc1 ; [2H]C([2H])([2H])N(C(=O)Oc1c(-c2cnn(CCC3CN(C)CCO3)c2)cc(C(F)(F)F)cc1C(F)(F)F)c1ccc(F)cc1 ; [2H]C([2H])([2H])N(C(=O)Oc1c(-c2cnn(CCC3CN(C)CCO3)c2)cc(C(F)(F)F)cc1C(F)(F)F)c1ccc(F)cc1]

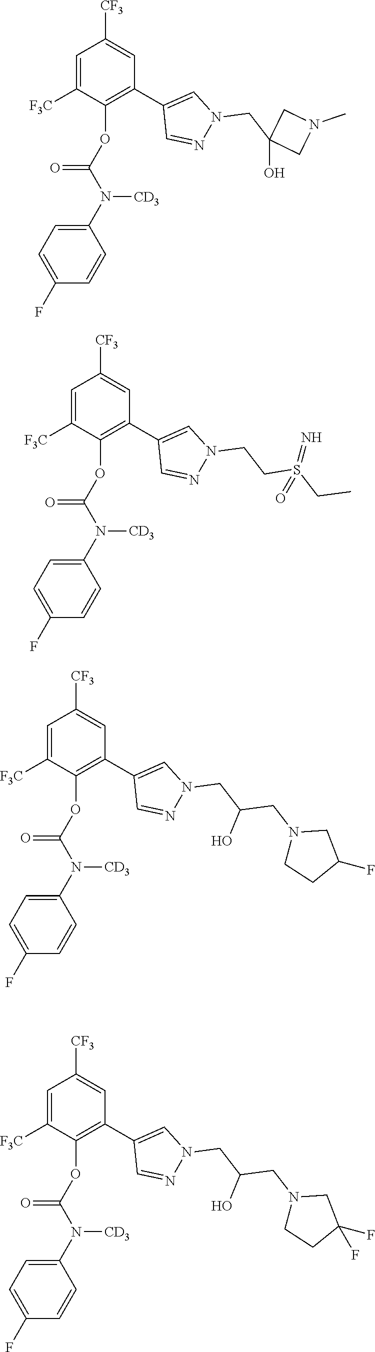 [SMILES: [2H]C([2H])([2H])N(C(=O)Oc1c(-c2cnn(CC3(O)CN(C)C3)c2)cc(C(F)(F)F)cc1C(F)(F)F)c1ccc(F)cc1 ; [2H]C([2H])([2H])N(C(=O)Oc1c(-c2cnn(CCS(=N)(=O)CC)c2)cc(C(F)(F)F)cc1C(F)(F)F)c1ccc(F)cc1 ; [2H]C([2H])([2H])N(C(=O)Oc1c(-c2cnn(CC(O)CN3CCC(F)C3)c2)cc(C(F)(F)F)cc1C(F)(F)F)c1ccc(F)cc1 ; [2H]C([2H])([2H])N(C(=O)Oc1c(-c2cnn(CC(O)CN3CCC(F)(F)C3)c2)cc(C(F)(F)F)cc1C(F)(F)F)c1ccc(F)cc1]

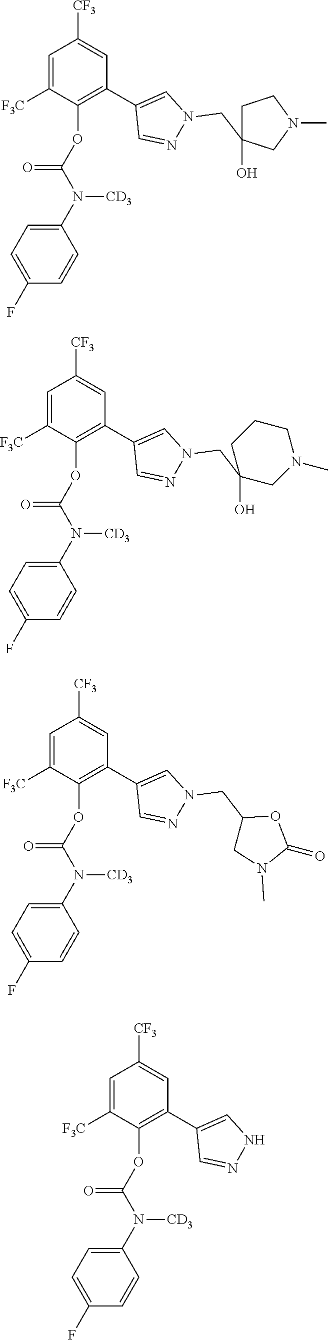 [SMILES: [2H]C([2H])([2H])N(C(=O)Oc1c(-c2cnn(CC3(O)CCN(C)C3)c2)cc(C(F)(F)F)cc1C(F)(F)F)c1ccc(F)cc1 ; [2H]C([2H])([2H])N(C(=O)Oc1c(-c2cnn(CC3(O)CCCN(C)C3)c2)cc(C(F)(F)F)cc1C(F)(F)F)c1ccc(F)cc1 ; [2H]C([2H])([2H])N(C(=O)Oc1c(-c2cnn(CC3CN(C)C(=O)O3)c2)cc(C(F)(F)F)cc1C(F)(F)F)c1ccc(F)cc1 ; [2H]C([2H])([2H])N(C(=O)Oc1c(-c2cn[nH]c2)cc(C(F)(F)F)cc1C(F)(F)F)c1ccc(F)cc1]

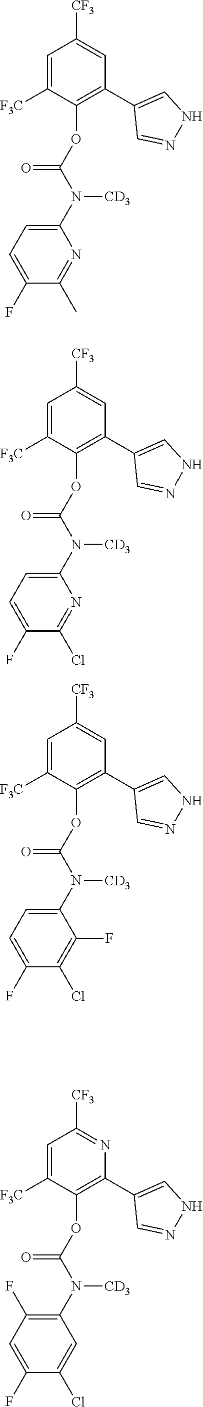

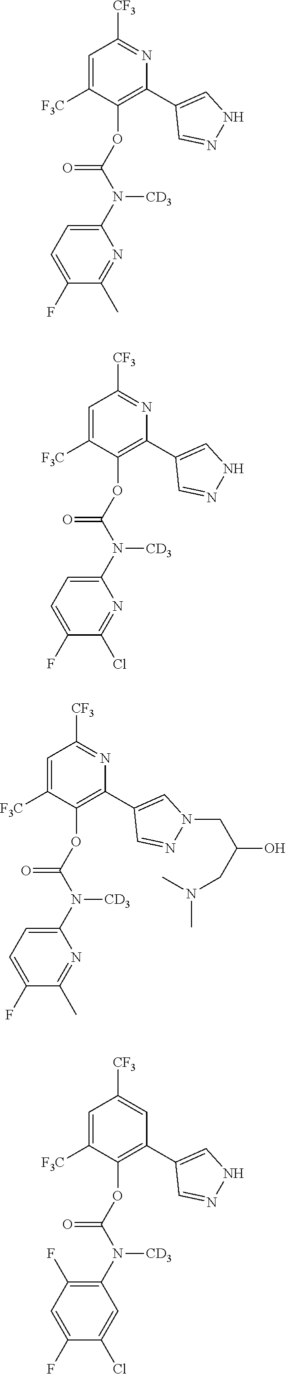

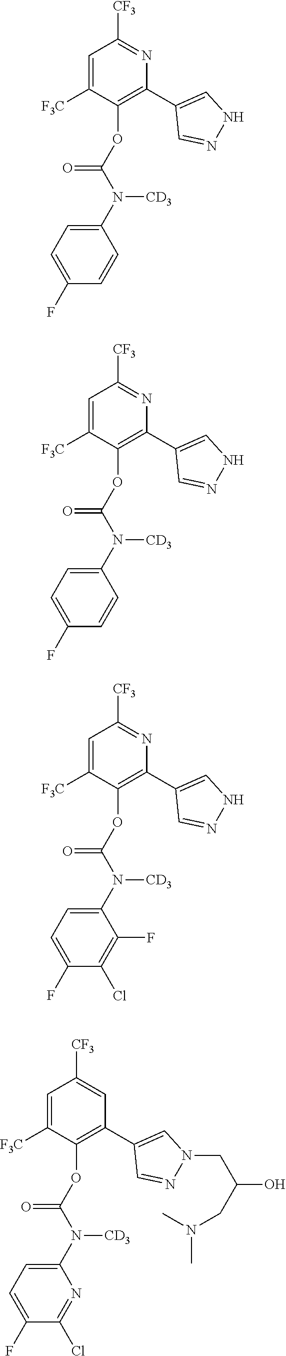 [SMILES: [2H]C([2H])([2H])N(C(=O)Oc1c(C(F)(F)F)cc(C(F)(F)F)nc1-c1cn[nH]c1)c1ccc(F)cc1 ; [2H]C([2H])([2H])N(C(=O)Oc1c(C(F)(F)F)cc(C(F)(F)F)nc1-c1cn[nH]c1)c1ccc(F)cc1 ; [2H]C([2H])([2H])N(C(=O)Oc1c(C(F)(F)F)cc(C(F)(F)F)nc1-c1cn[nH]c1)c1ccc(F)c(Cl)c1F ; [2H]C([2H])([2H])N(C(=O)Oc1c(-c2cnn(CC(O)CN(C)C)c2)cc(C(F)(F)F)cc1C(F)(F)F)c1ccc(F)c(Cl)n1]

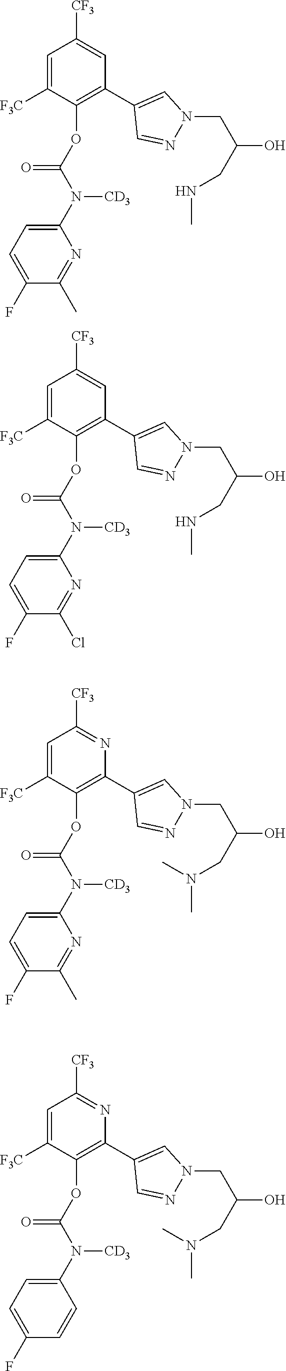

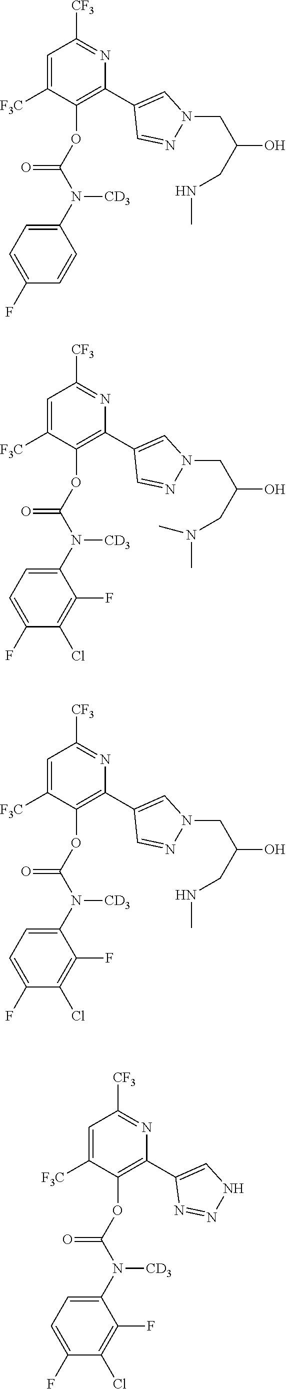 [SMILES: [2H]C([2H])([2H])N(C(=O)Oc1c(C(F)(F)F)cc(C(F)(F)F)nc1-c1cnn(CC(O)CNC)c1)c1ccc(F)cc1 ; [2H]C([2H])([2H])N(C(=O)Oc1c(C(F)(F)F)cc(C(F)(F)F)nc1-c1cnn(CC(O)CN(C)C)c1)c1ccc(F)c(Cl)c1F ; [2H]C([2H])([2H])N(C(=O)Oc1c(C(F)(F)F)cc(C(F)(F)F)nc1-c1cnn(CC(O)CNC)c1)c1ccc(F)c(Cl)c1F ; [2H]C([2H])([2H])N(C(=O)Oc1c(C(F)(F)F)cc(C(F)(F)F)nc1-c1c[nH]nn1)c1ccc(F)c(Cl)c1F]

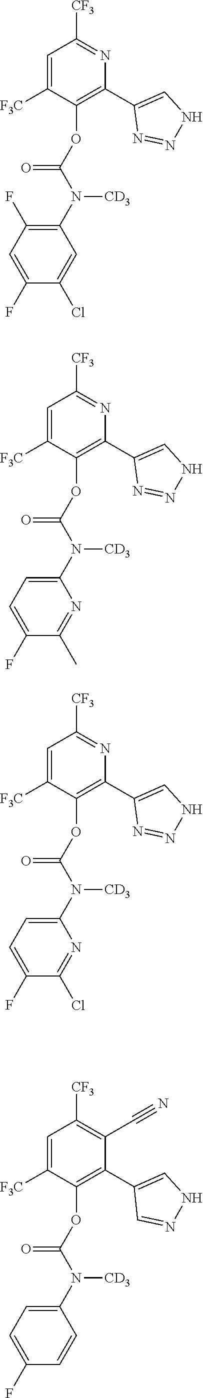

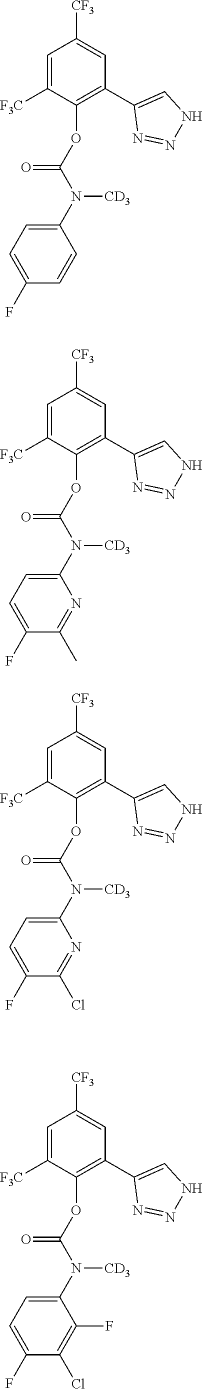

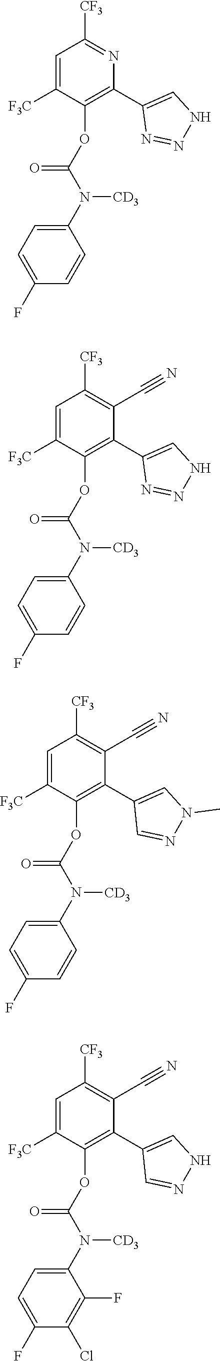 [SMILES: [2H]C([2H])([2H])N(C(=O)Oc1c(C(F)(F)F)cc(C(F)(F)F)nc1-c1c[nH]nn1)c1ccc(F)cc1 ; [2H]C([2H])([2H])N(C(=O)Oc1c(C(F)(F)F)cc(C(F)(F)F)c(C#N)c1-c1c[nH]nn1)c1ccc(F)cc1 ; [2H]C([2H])([2H])N(C(=O)Oc1c(C(F)(F)F)cc(C(F)(F)F)c(C#N)c1-c1cnn(C)c1)c1ccc(F)cc1 ; [2H]C([2H])([2H])N(C(=O)Oc1c(C(F)(F)F)cc(C(F)(F)F)c(C#N)c1-c1cn[nH]c1)c1ccc(F)c(Cl)c1F]

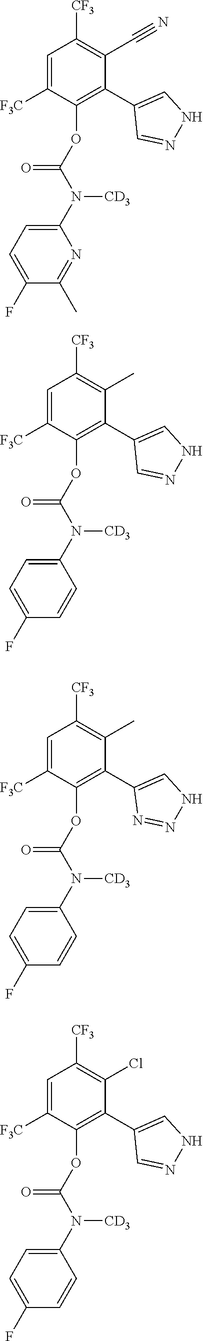

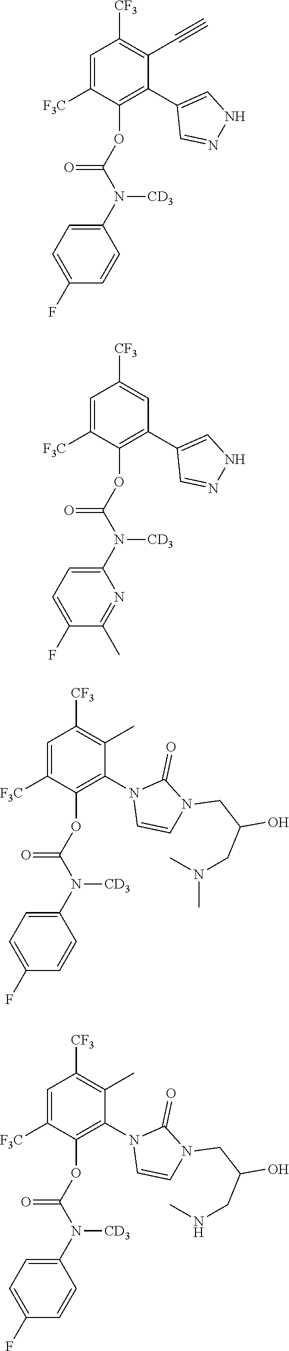

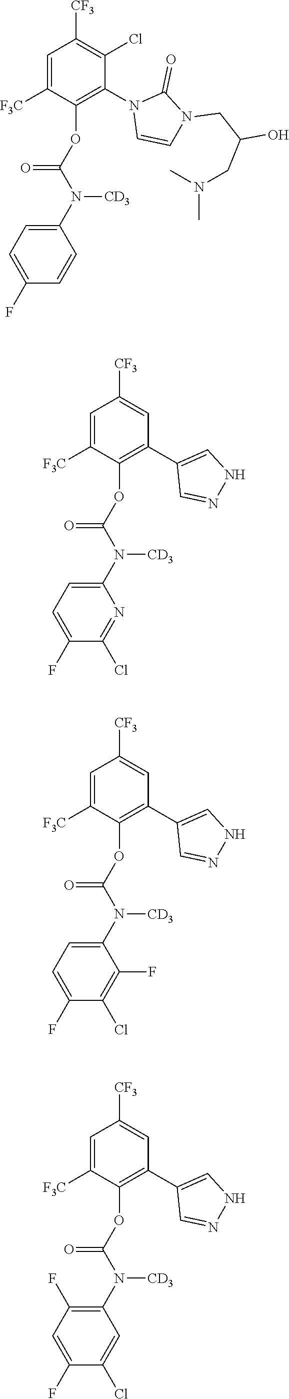 [SMILES: [2H]C([2H])([2H])N(C(=O)Oc1c(C(F)(F)F)cc(C(F)(F)F)c(Cl)c1-n1ccn(CC(O)CN(C)C)c1=O)c1ccc(F)cc1 ; [2H]C([2H])([2H])N(C(=O)Oc1c(-c2cn[nH]c2)cc(C(F)(F)F)cc1C(F)(F)F)c1ccc(F)c(Cl)n1 ; [2H]C([2H])([2H])N(C(=O)Oc1c(-c2cn[nH]c2)cc(C(F)(F)F)cc1C(F)(F)F)c1ccc(F)c(Cl)c1F ; [2H]C([2H])([2H])N(C(=O)Oc1c(-c2cn[nH]c2)cc(C(F)(F)F)cc1C(F)(F)F)c1cc(Cl)c(F)cc1F]

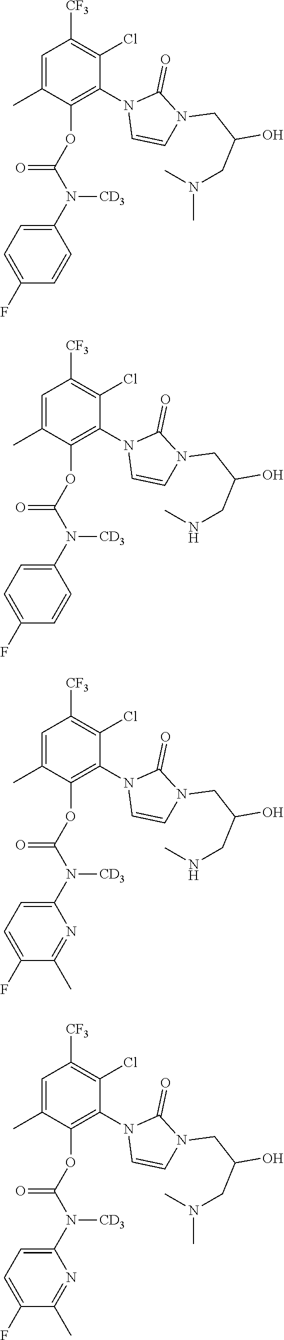

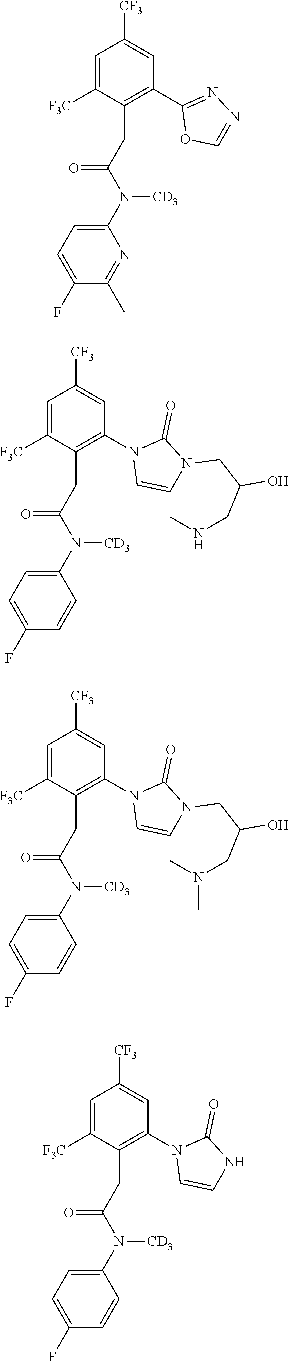

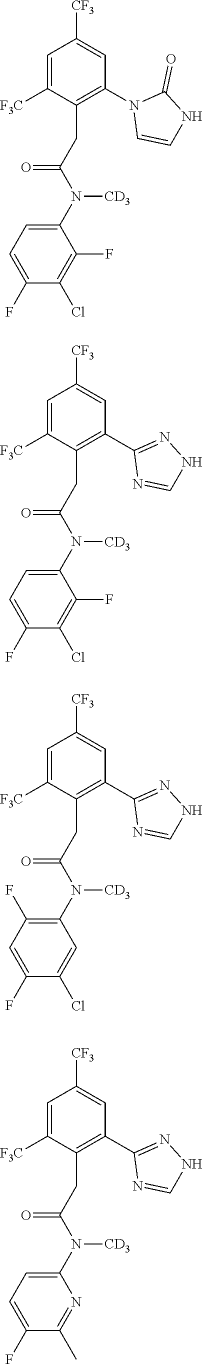

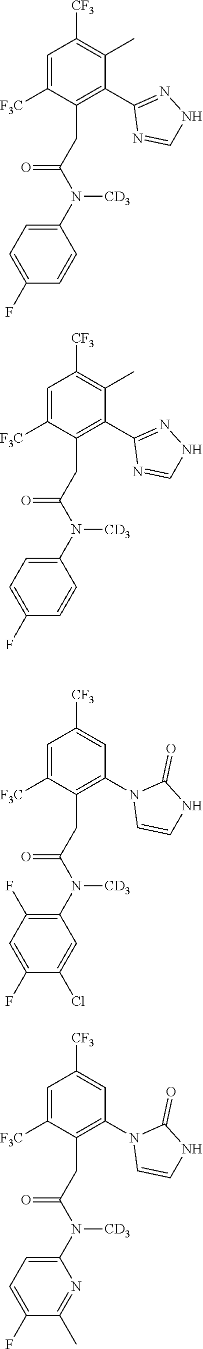

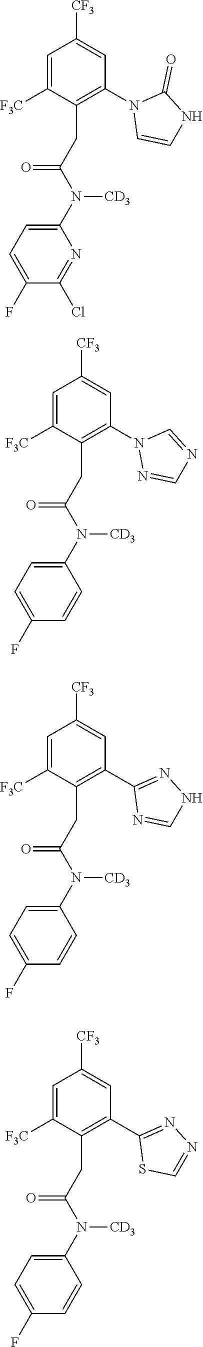

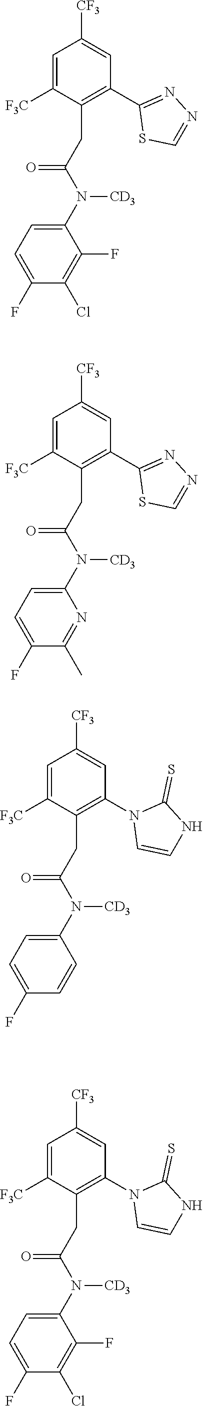

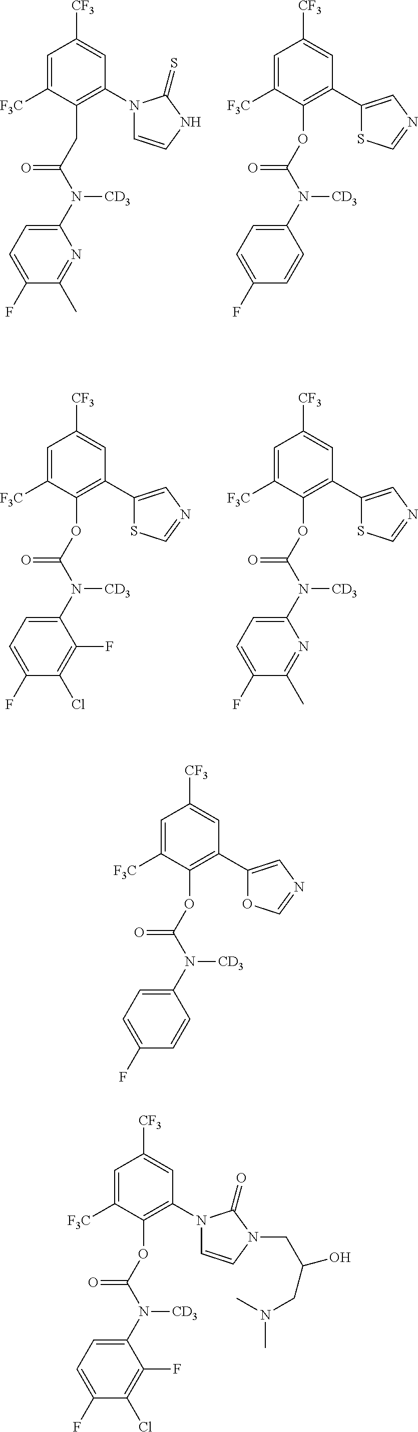 [SMILES: [2H]C([2H])([2H])N(C(=O)Cc1c(-n2cc[nH]c2=S)cc(C(F)(F)F)cc1C(F)(F)F)c1ccc(F)c(C)n1 ; [2H]C([2H])([2H])N(C(=O)Oc1c(-c2cncs2)cc(C(F)(F)F)cc1C(F)(F)F)c1ccc(F)cc1 ; [2H]C([2H])([2H])N(C(=O)Oc1c(-c2cncs2)cc(C(F)(F)F)cc1C(F)(F)F)c1ccc(F)c(Cl)c1F ; [2H]C([2H])([2H])N(C(=O)Oc1c(-c2cncs2)cc(C(F)(F)F)cc1C(F)(F)F)c1ccc(F)c(C)n1 ; [2H]C([2H])([2H])N(C(=O)Oc1c(-c2cnco2)cc(C(F)(F)F)cc1C(F)(F)F)c1ccc(F)cc1 ; [2H]C([2H])([2H])N(C(=O)Oc1c(-n2ccn(CC(O)CN(C)C)c2=O)cc(C(F)(F)F)cc1C(F)(F)F)c1ccc(F)c(Cl)c1F]

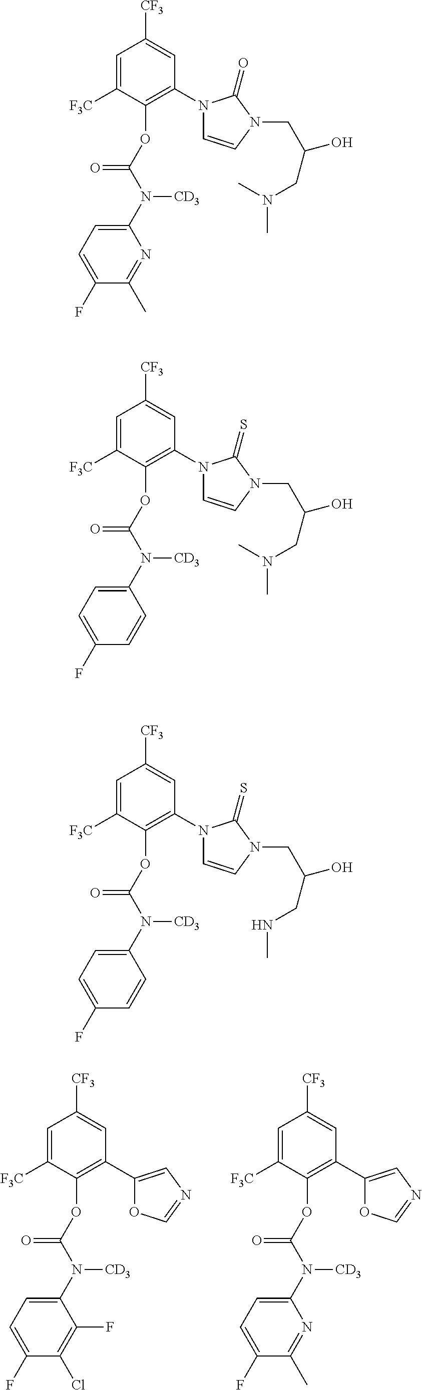

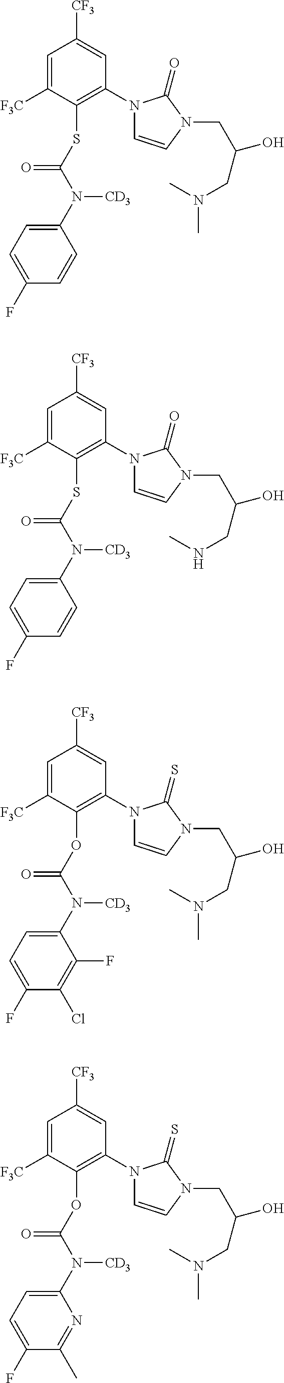 [SMILES: [2H]C([2H])([2H])N(C(=O)Sc1c(-n2ccn(CC(O)CN(C)C)c2=O)cc(C(F)(F)F)cc1C(F)(F)F)c1ccc(F)cc1 ; [2H]C([2H])([2H])N(C(=O)Sc1c(-n2ccn(CC(O)CNC)c2=O)cc(C(F)(F)F)cc1C(F)(F)F)c1ccc(F)cc1 ; [2H]C([2H])([2H])N(C(=O)Oc1c(-n2ccn(CC(O)CN(C)C)c2=S)cc(C(F)(F)F)cc1C(F)(F)F)c1ccc(F)c(Cl)c1F ; [2H]C([2H])([2H])N(C(=O)Oc1c(-n2ccn(CC(O)CN(C)C)c2=S)cc(C(F)(F)F)cc1C(F)(F)F)c1ccc(F)c(C)n1]

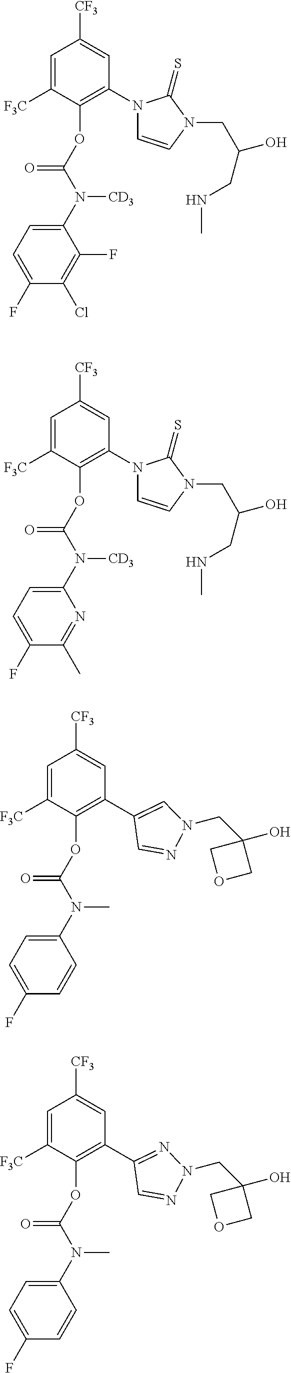

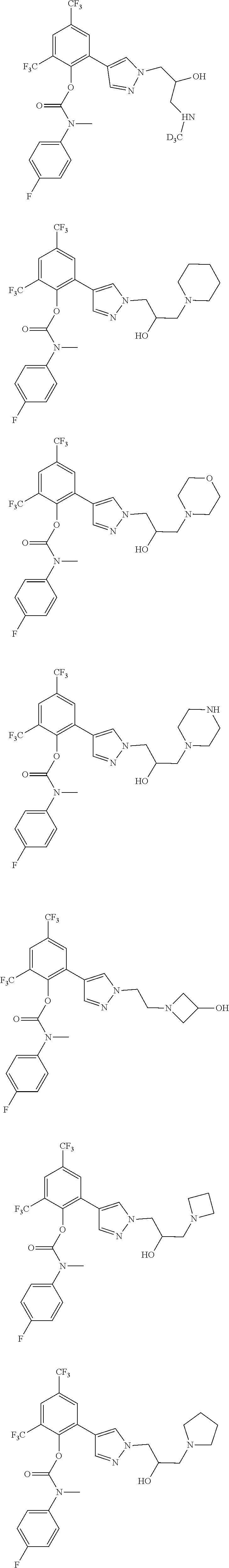

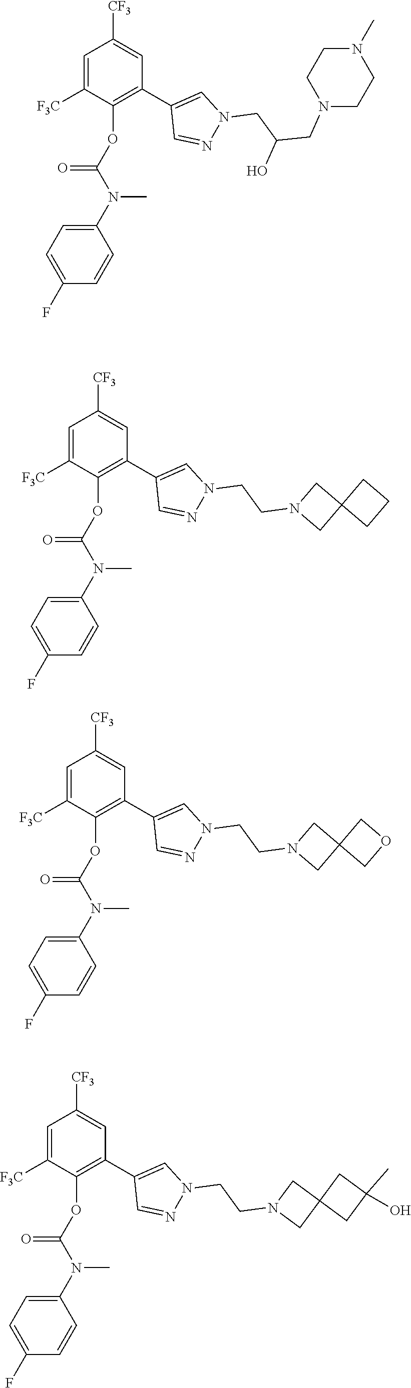 [SMILES: CN1CCN(CC(O)Cn2cc(-c3cc(C(F)(F)F)cc(C(F)(F)F)c3OC(=O)N(C)c3ccc(F)cc3)cn2)CC1 ; CN(C(=O)Oc1c(-c2cnn(CCN3CC4(CCC4)C3)c2)cc(C(F)(F)F)cc1C(F)(F)F)c1ccc(F)cc1 ; CN(C(=O)Oc1c(-c2cnn(CCN3CC4(COC4)C3)c2)cc(C(F)(F)F)cc1C(F)(F)F)c1ccc(F)cc1 ; CN(C(=O)Oc1c(-c2cnn(CCN3CC4(C3)CC(C)(O)C4)c2)cc(C(F)(F)F)cc1C(F)(F)F)c1ccc(F)cc1]

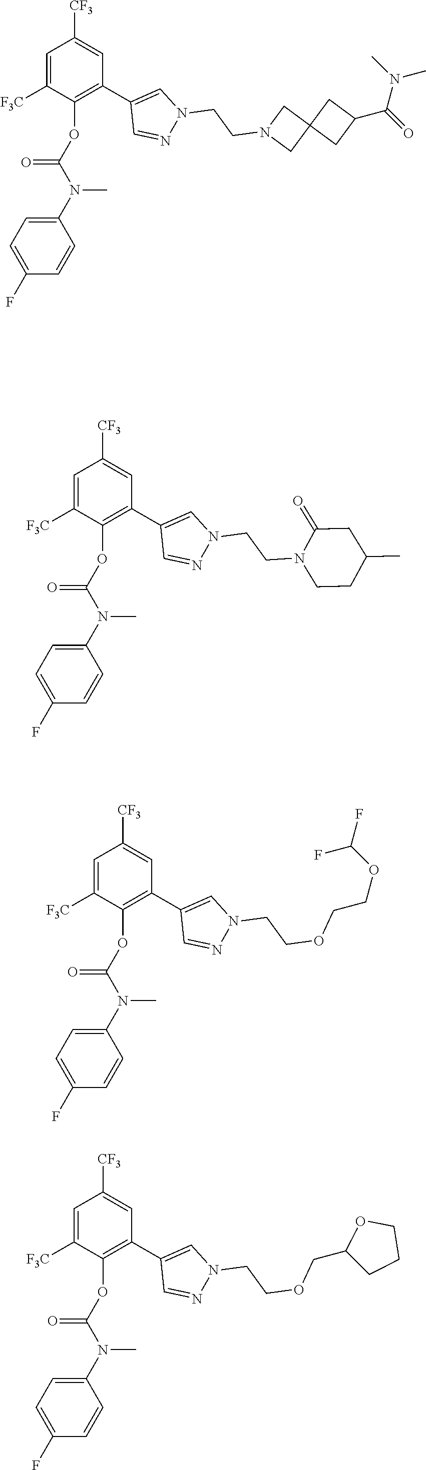 [SMILES: CN(C)C(=O)C1CC2(C1)CN(CCn1cc(-c3cc(C(F)(F)F)cc(C(F)(F)F)c3OC(=O)N(C)c3ccc(F)cc3)cn1)C2 ; CC1CCN(CCn2cc(-c3cc(C(F)(F)F)cc(C(F)(F)F)c3OC(=O)N(C)c3ccc(F)cc3)cn2)C(=O)C1 ; CN(C(=O)Oc1c(-c2cnn(CCOCCOC(F)F)c2)cc(C(F)(F)F)cc1C(F)(F)F)c1ccc(F)cc1 ; CN(C(=O)Oc1c(-c2cnn(CCOCC3CCCO3)c2)cc(C(F)(F)F)cc1C(F)(F)F)c1ccc(F)cc1]

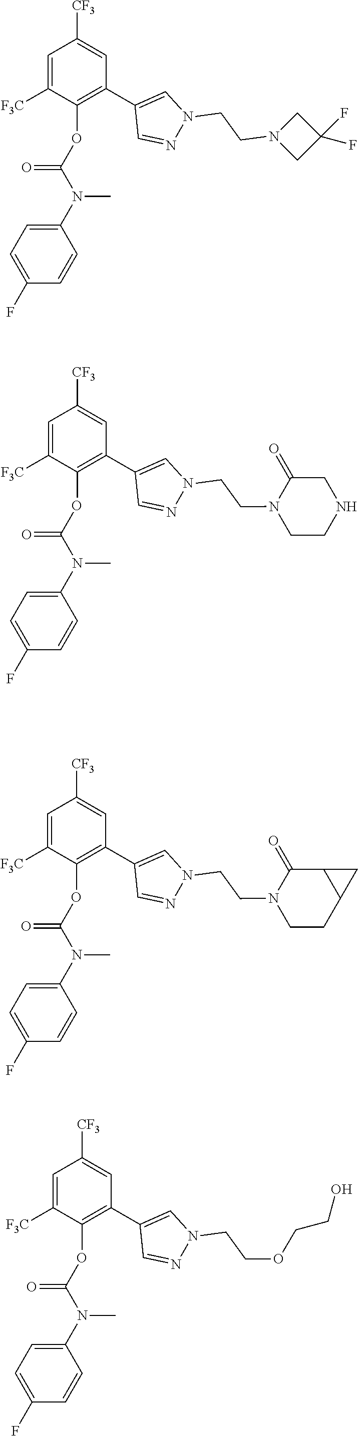 [SMILES: CN(C(=O)Oc1c(-c2cnn(CCN3CC(F)(F)C3)c2)cc(C(F)(F)F)cc1C(F)(F)F)c1ccc(F)cc1 ; CN(C(=O)Oc1c(-c2cnn(CCN3CCNCC3=O)c2)cc(C(F)(F)F)cc1C(F)(F)F)c1ccc(F)cc1 ; CN(C(=O)Oc1c(-c2cnn(CCN3CCC4CC4C3=O)c2)cc(C(F)(F)F)cc1C(F)(F)F)c1ccc(F)cc1 ; CN(C(=O)Oc1c(-c2cnn(CCOCCO)c2)cc(C(F)(F)F)cc1C(F)(F)F)c1ccc(F)cc1]

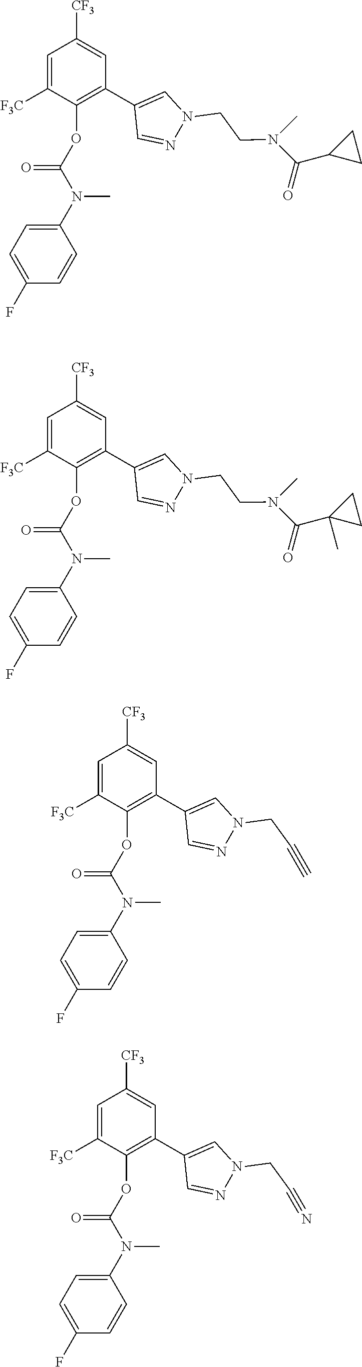 [SMILES: CN(CCn1cc(-c2cc(C(F)(F)F)cc(C(F)(F)F)c2OC(=O)N(C)c2ccc(F)cc2)cn1)C(=O)C1CC1 ; CN(CCn1cc(-c2cc(C(F)(F)F)cc(C(F)(F)F)c2OC(=O)N(C)c2ccc(F)cc2)cn1)C(=O)C1(C)CC1 ; C#CCn1cc(-c2cc(C(F)(F)F)cc(C(F)(F)F)c2OC(=O)N(C)c2ccc(F)cc2)cn1 ; CN(C(=O)Oc1c(-c2cnn(CC#N)c2)cc(C(F)(F)F)cc1C(F)(F)F)c1ccc(F)cc1]

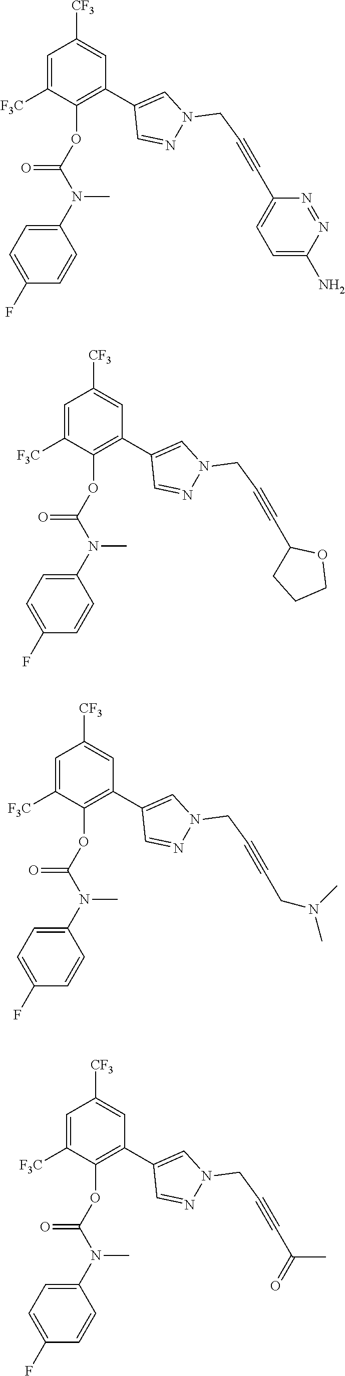 [SMILES: CN(C(=O)Oc1c(-c2cnn(CC#Cc3ccc(N)nn3)c2)cc(C(F)(F)F)cc1C(F)(F)F)c1ccc(F)cc1 ; CN(C(=O)Oc1c(-c2cnn(CC#CC3CCCO3)c2)cc(C(F)(F)F)cc1C(F)(F)F)c1ccc(F)cc1 ; CN(C)CC#CCn1cc(-c2cc(C(F)(F)F)cc(C(F)(F)F)c2OC(=O)N(C)c2ccc(F)cc2)cn1 ; CC(=O)C#CCn1cc(-c2cc(C(F)(F)F)cc(C(F)(F)F)c2OC(=O)N(C)c2ccc(F)cc2)cn1]

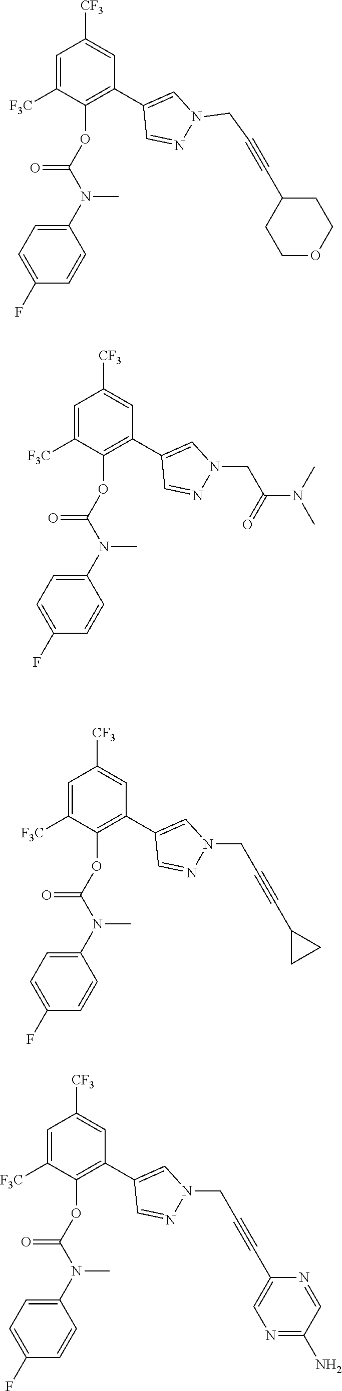 [SMILES: CN(C(=O)Oc1c(-c2cnn(CC#CC3CCOCC3)c2)cc(C(F)(F)F)cc1C(F)(F)F)c1ccc(F)cc1 ; CN(C)C(=O)Cn1cc(-c2cc(C(F)(F)F)cc(C(F)(F)F)c2OC(=O)N(C)c2ccc(F)cc2)cn1 ; CN(C(=O)Oc1c(-c2cnn(CC#CC3CC3)c2)cc(C(F)(F)F)cc1C(F)(F)F)c1ccc(F)cc1 ; CN(C(=O)Oc1c(-c2cnn(CC#Cc3cnc(N)cn3)c2)cc(C(F)(F)F)cc1C(F)(F)F)c1ccc(F)cc1]

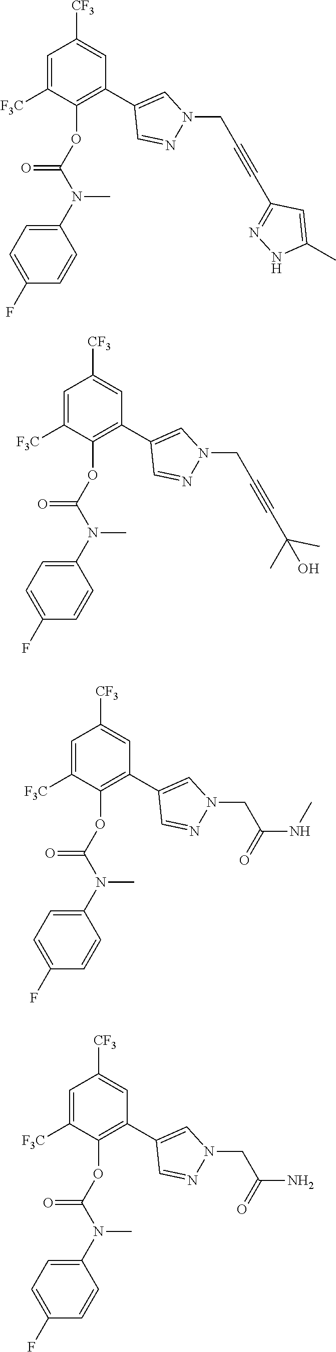 [SMILES: Cc1cc(C#CCn2cc(-c3cc(C(F)(F)F)cc(C(F)(F)F)c3OC(=O)N(C)c3ccc(F)cc3)cn2)n[nH]1 ; CN(C(=O)Oc1c(-c2cnn(CC#CC(C)(C)O)c2)cc(C(F)(F)F)cc1C(F)(F)F)c1ccc(F)cc1 ; CNC(=O)Cn1cc(-c2cc(C(F)(F)F)cc(C(F)(F)F)c2OC(=O)N(C)c2ccc(F)cc2)cn1 ; CN(C(=O)Oc1c(-c2cnn(CC(N)=O)c2)cc(C(F)(F)F)cc1C(F)(F)F)c1ccc(F)cc1]

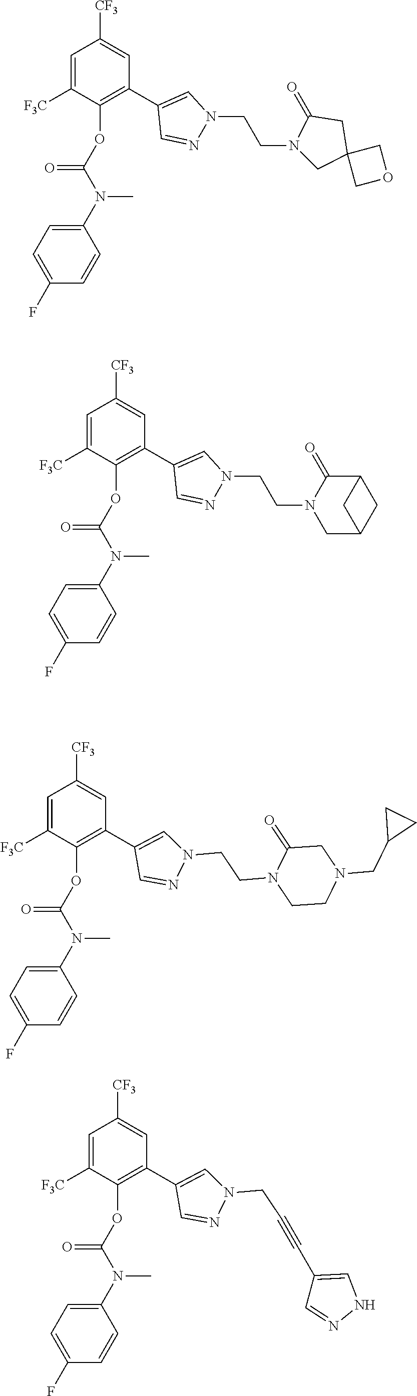 [SMILES: CN(C(=O)Oc1c(-c2cnn(CCN3CC4(COC4)CC3=O)c2)cc(C(F)(F)F)cc1C(F)(F)F)c1ccc(F)cc1 ; CN(C(=O)Oc1c(-c2cnn(CCN3CC4CC(C4)C3=O)c2)cc(C(F)(F)F)cc1C(F)(F)F)c1ccc(F)cc1 ; CN(C(=O)Oc1c(-c2cnn(CCN3CCN(CC4CC4)CC3=O)c2)cc(C(F)(F)F)cc1C(F)(F)F)c1ccc(F)cc1 ; CN(C(=O)Oc1c(-c2cnn(CC#Cc3cn[nH]c3)c2)cc(C(F)(F)F)cc1C(F)(F)F)c1ccc(F)cc1]

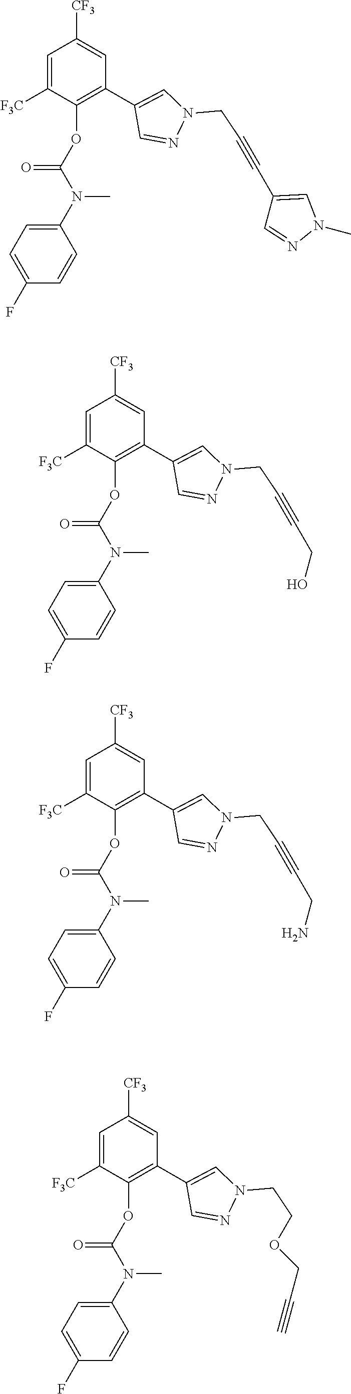 [SMILES: CN(C(=O)Oc1c(-c2cnn(CC#Cc3cnn(C)c3)c2)cc(C(F)(F)F)cc1C(F)(F)F)c1ccc(F)cc1 ; CN(C(=O)Oc1c(-c2cnn(CC#CCO)c2)cc(C(F)(F)F)cc1C(F)(F)F)c1ccc(F)cc1 ; CN(C(=O)Oc1c(-c2cnn(CC#CCN)c2)cc(C(F)(F)F)cc1C(F)(F)F)c1ccc(F)cc1 ; C#CCOCCn1cc(-c2cc(C(F)(F)F)cc(C(F)(F)F)c2OC(=O)N(C)c2ccc(F)cc2)cn1]

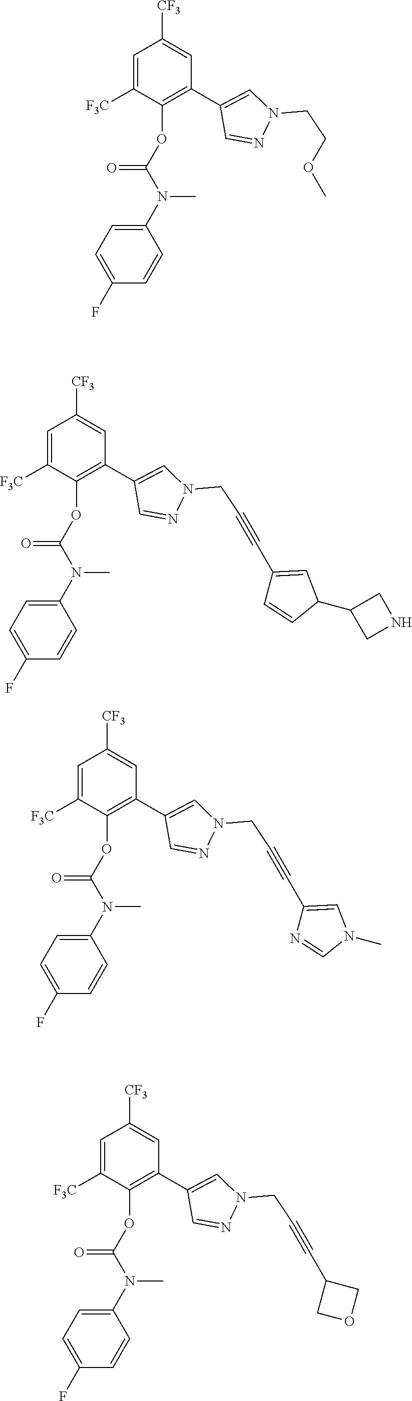 [SMILES: COCCn1cc(-c2cc(C(F)(F)F)cc(C(F)(F)F)c2OC(=O)N(C)c2ccc(F)cc2)cn1 ; CN(C(=O)Oc1c(-c2cnn(CC#CC3=CC(C4CNC4)C=C3)c2)cc(C(F)(F)F)cc1C(F)(F)F)c1ccc(F)cc1 ; CN(C(=O)Oc1c(-c2cnn(CC#Cc3cn(C)cn3)c2)cc(C(F)(F)F)cc1C(F)(F)F)c1ccc(F)cc1 ; CN(C(=O)Oc1c(-c2cnn(CC#CC3COC3)c2)cc(C(F)(F)F)cc1C(F)(F)F)c1ccc(F)cc1]

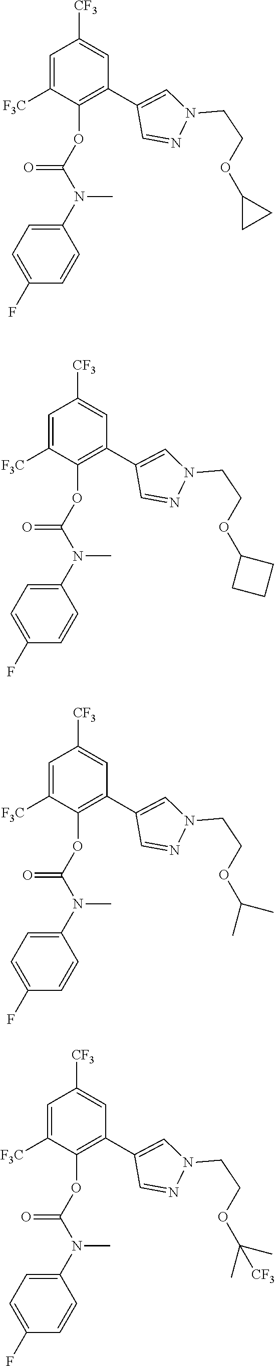 [SMILES: CN(C(=O)Oc1c(-c2cnn(CCOC3CC3)c2)cc(C(F)(F)F)cc1C(F)(F)F)c1ccc(F)cc1 ; CN(C(=O)Oc1c(-c2cnn(CCOC3CCC3)c2)cc(C(F)(F)F)cc1C(F)(F)F)c1ccc(F)cc1 ; CC(C)OCCn1cc(-c2cc(C(F)(F)F)cc(C(F)(F)F)c2OC(=O)N(C)c2ccc(F)cc2)cn1 ; CN(C(=O)Oc1c(-c2cnn(CCOC(C)(C)C(F)(F)F)c2)cc(C(F)(F)F)cc1C(F)(F)F)c1ccc(F)cc1]

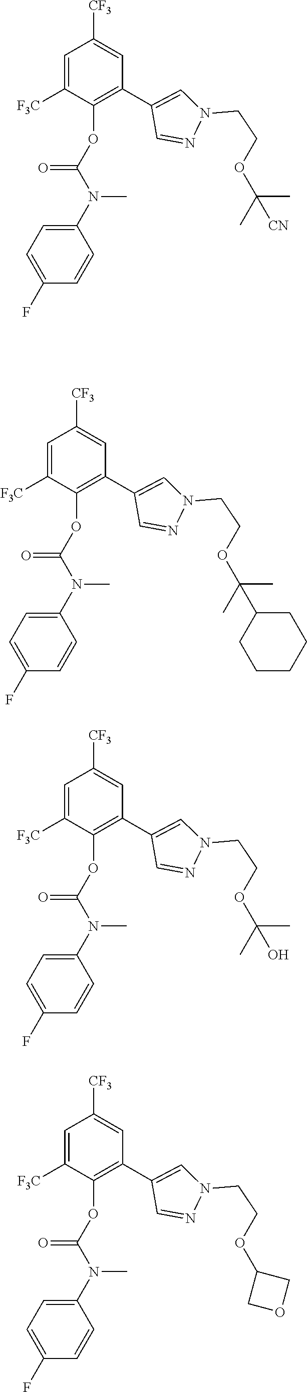 [SMILES: CN(C(=O)Oc1c(-c2cnn(CCOC(C)(C)C#N)c2)cc(C(F)(F)F)cc1C(F)(F)F)c1ccc(F)cc1 ; CN(C(=O)Oc1c(-c2cnn(CCOC(C)(C)C3CCCCC3)c2)cc(C(F)(F)F)cc1C(F)(F)F)c1ccc(F)cc1 ; CN(C(=O)Oc1c(-c2cnn(CCOC(C)(C)O)c2)cc(C(F)(F)F)cc1C(F)(F)F)c1ccc(F)cc1 ; CN(C(=O)Oc1c(-c2cnn(CCOC3COC3)c2)cc(C(F)(F)F)cc1C(F)(F)F)c1ccc(F)cc1]

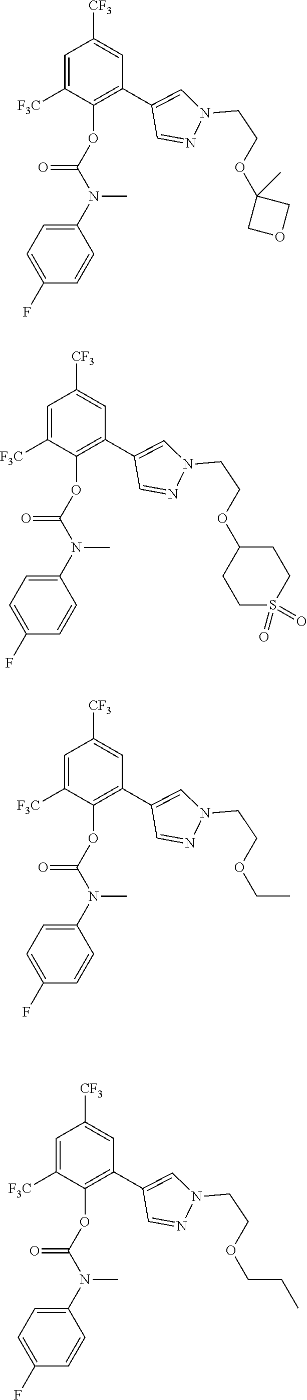 [SMILES: CN(C(=O)Oc1c(-c2cnn(CCOC3(C)COC3)c2)cc(C(F)(F)F)cc1C(F)(F)F)c1ccc(F)cc1 ; CN(C(=O)Oc1c(-c2cnn(CCOC3CCS(=O)(=O)CC3)c2)cc(C(F)(F)F)cc1C(F)(F)F)c1ccc(F)cc1 ; CCOCCn1cc(-c2cc(C(F)(F)F)cc(C(F)(F)F)c2OC(=O)N(C)c2ccc(F)cc2)cn1 ; CCCOCCn1cc(-c2cc(C(F)(F)F)cc(C(F)(F)F)c2OC(=O)N(C)c2ccc(F)cc2)cn1]

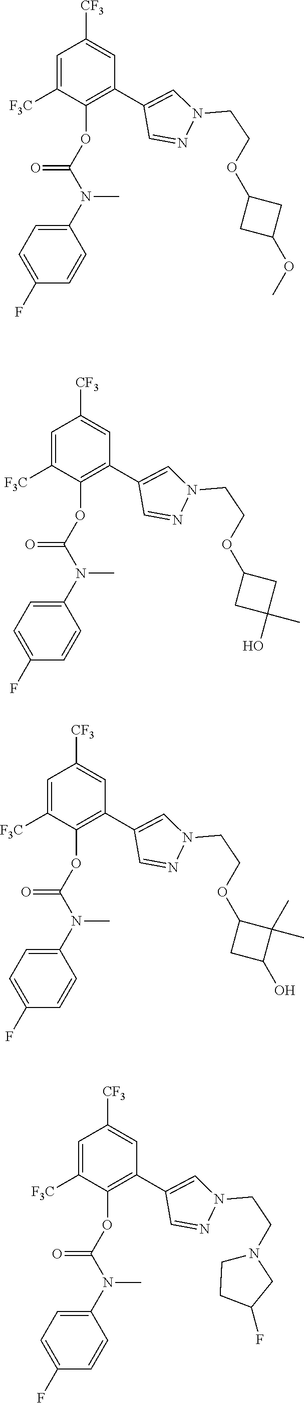 [SMILES: COC1CC(OCCn2cc(-c3cc(C(F)(F)F)cc(C(F)(F)F)c3OC(=O)N(C)c3ccc(F)cc3)cn2)C1 ; CN(C(=O)Oc1c(-c2cnn(CCOC3CC(C)(O)C3)c2)cc(C(F)(F)F)cc1C(F)(F)F)c1ccc(F)cc1 ; CN(C(=O)Oc1c(-c2cnn(CCOC3CC(O)C3(C)C)c2)cc(C(F)(F)F)cc1C(F)(F)F)c1ccc(F)cc1 ; CN(C(=O)Oc1c(-c2cnn(CCN3CCC(F)C3)c2)cc(C(F)(F)F)cc1C(F)(F)F)c1ccc(F)cc1]

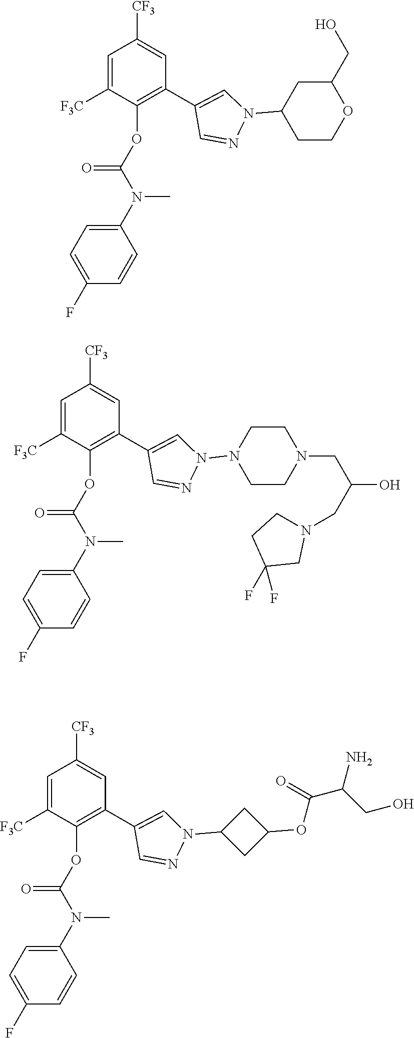

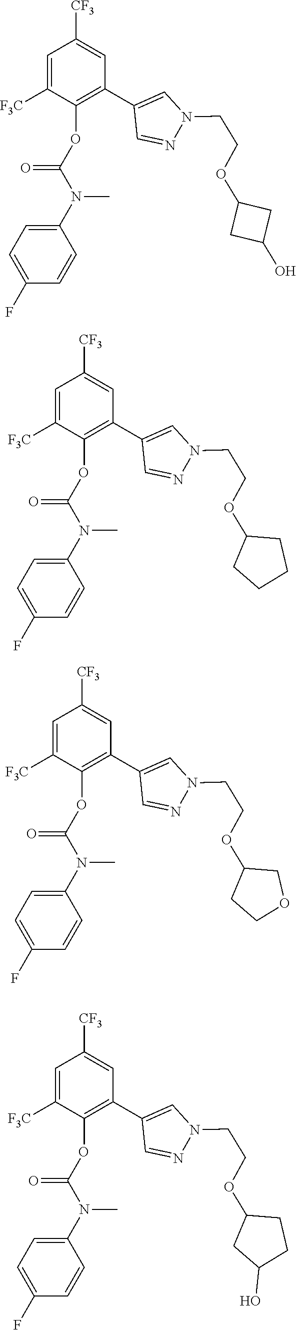 [SMILES: CN(C(=O)Oc1c(-c2cnn(CCOC3CC(O)C3)c2)cc(C(F)(F)F)cc1C(F)(F)F)c1ccc(F)cc1 ; CN(C(=O)Oc1c(-c2cnn(CCOC3CCCC3)c2)cc(C(F)(F)F)cc1C(F)(F)F)c1ccc(F)cc1 ; CN(C(=O)Oc1c(-c2cnn(CCOC3CCOC3)c2)cc(C(F)(F)F)cc1C(F)(F)F)c1ccc(F)cc1 ; CN(C(=O)Oc1c(-c2cnn(CCOC3CCC(O)C3)c2)cc(C(F)(F)F)cc1C(F)(F)F)c1ccc(F)cc1]

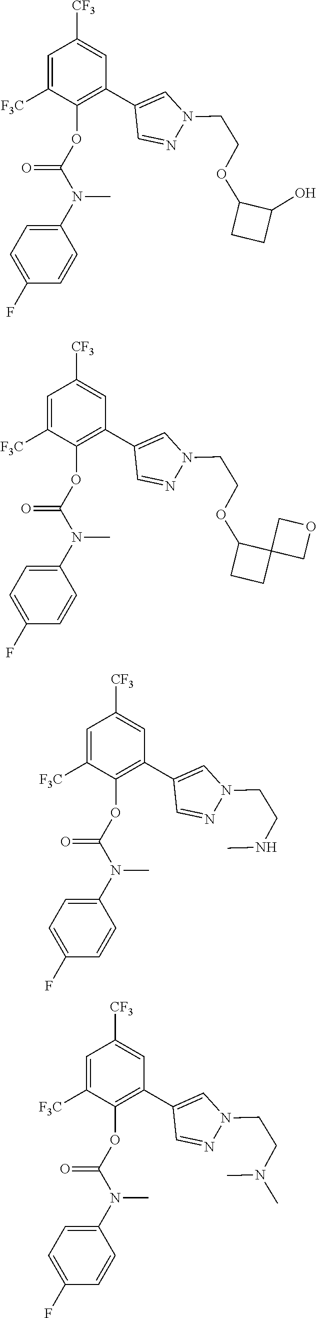 [SMILES: CN(C(=O)Oc1c(-c2cnn(CCOC3CCC3O)c2)cc(C(F)(F)F)cc1C(F)(F)F)c1ccc(F)cc1 ; CN(C(=O)Oc1c(-c2cnn(CCOC3CCC34COC4)c2)cc(C(F)(F)F)cc1C(F)(F)F)c1ccc(F)cc1 ; CNCCn1cc(-c2cc(C(F)(F)F)cc(C(F)(F)F)c2OC(=O)N(C)c2ccc(F)cc2)cn1 ; CN(C)CCn1cc(-c2cc(C(F)(F)F)cc(C(F)(F)F)c2OC(=O)N(C)c2ccc(F)cc2)cn1]

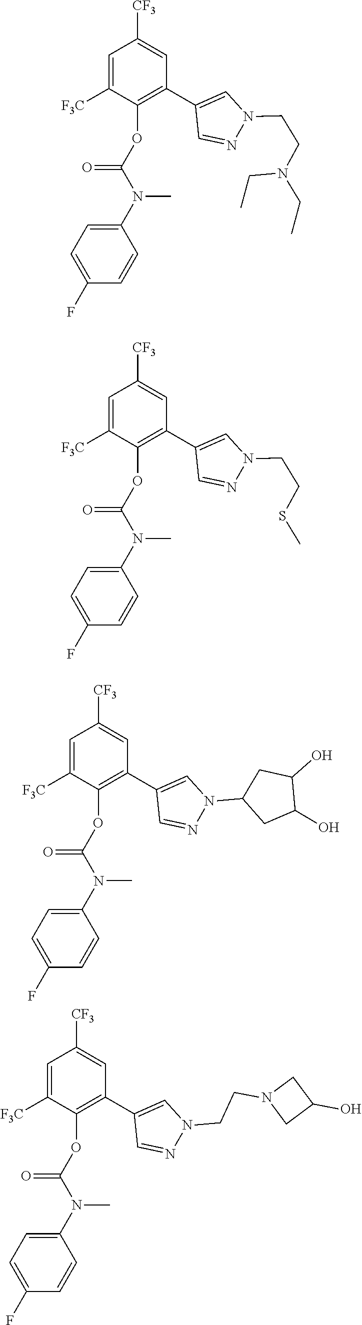

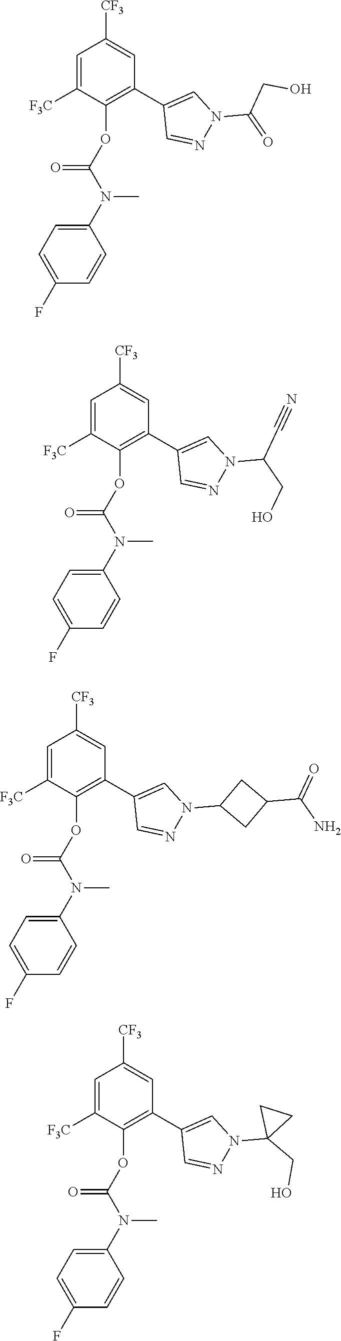

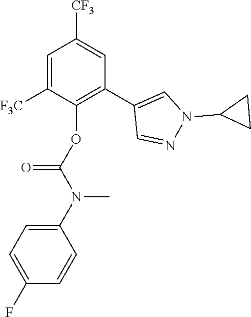 [SMILES: CN(C(=O)Oc1c(-c2cnn(C3CC3)c2)cc(C(F)(F)F)cc1C(F)(F)F)c1ccc(F)cc1]

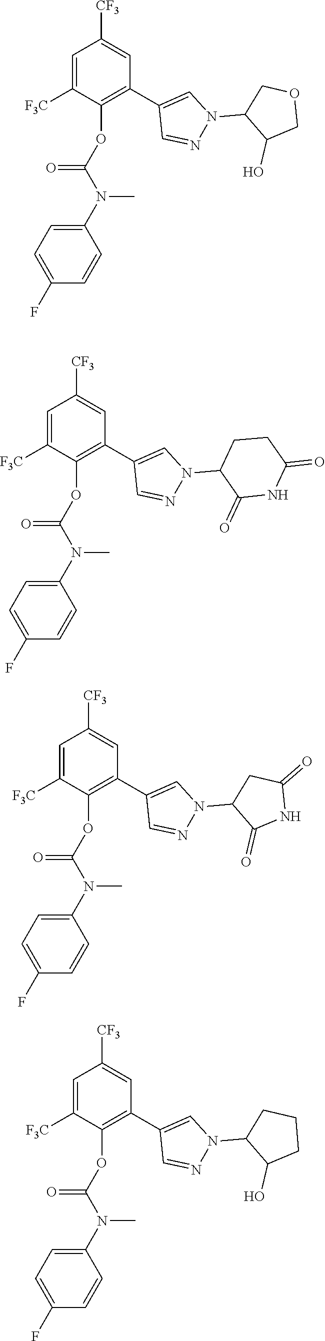 [SMILES: CN(C(=O)Oc1c(-c2cnn(C3COCC3O)c2)cc(C(F)(F)F)cc1C(F)(F)F)c1ccc(F)cc1 ; CN(C(=O)Oc1c(-c2cnn(C3CCC(=O)NC3=O)c2)cc(C(F)(F)F)cc1C(F)(F)F)c1ccc(F)cc1 ; CN(C(=O)Oc1c(-c2cnn(C3CC(=O)NC3=O)c2)cc(C(F)(F)F)cc1C(F)(F)F)c1ccc(F)cc1 ; CN(C(=O)Oc1c(-c2cnn(C3CCCC3O)c2)cc(C(F)(F)F)cc1C(F)(F)F)c1ccc(F)cc1]

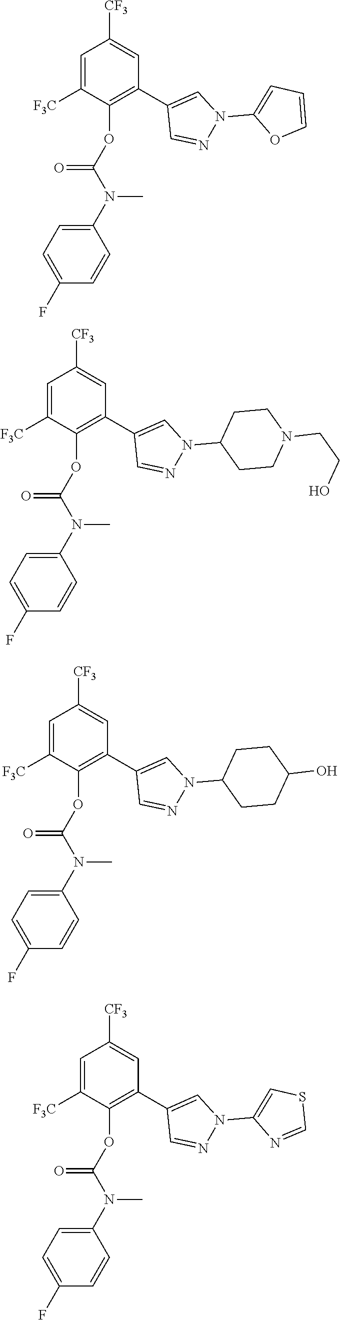

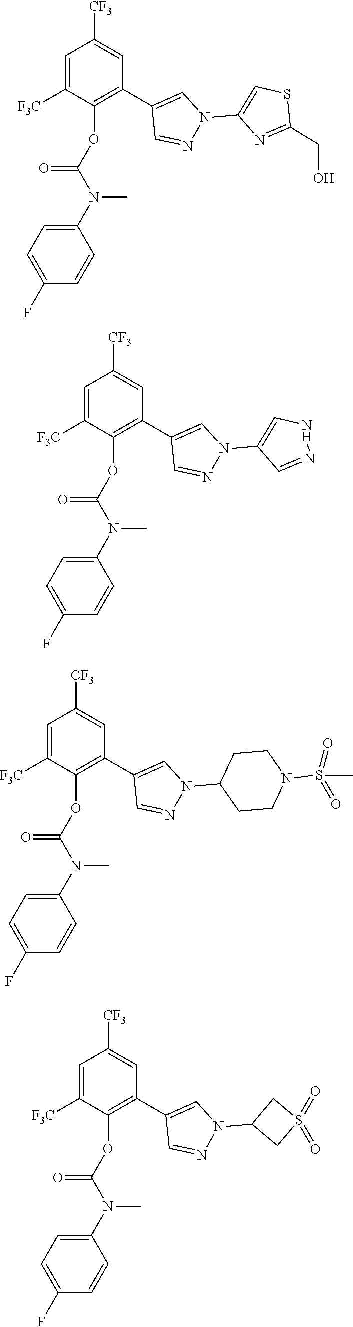

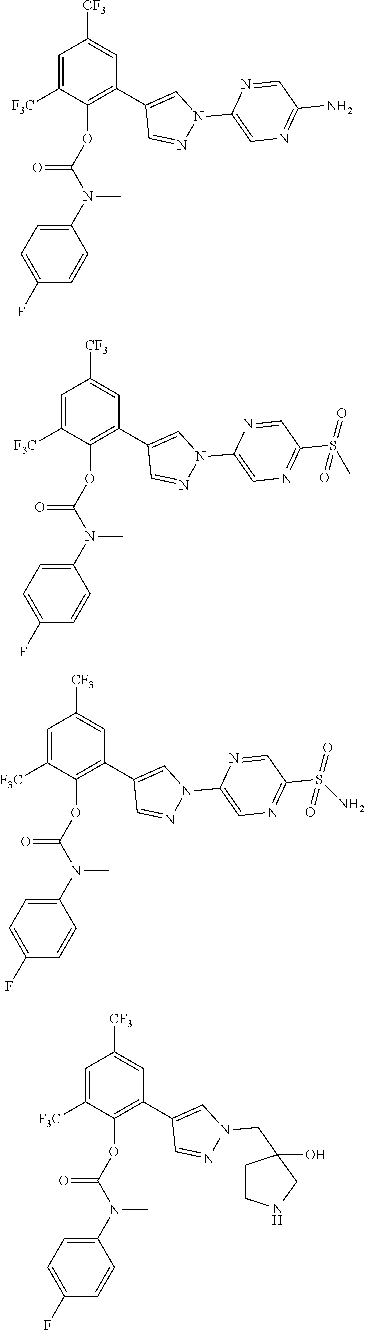 [SMILES: CN(C(=O)Oc1c(-c2cnn(-c3cnc(N)cn3)c2)cc(C(F)(F)F)cc1C(F)(F)F)c1ccc(F)cc1 ; CN(C(=O)Oc1c(-c2cnn(-c3cnc(S(C)(=O)=O)cn3)c2)cc(C(F)(F)F)cc1C(F)(F)F)c1ccc(F)cc1 ; CN(C(=O)Oc1c(-c2cnn(-c3cnc(S(N)(=O)=O)cn3)c2)cc(C(F)(F)F)cc1C(F)(F)F)c1ccc(F)cc1 ; CN(C(=O)Oc1c(-c2cnn(CC3(O)CCNC3)c2)cc(C(F)(F)F)cc1C(F)(F)F)c1ccc(F)cc1]

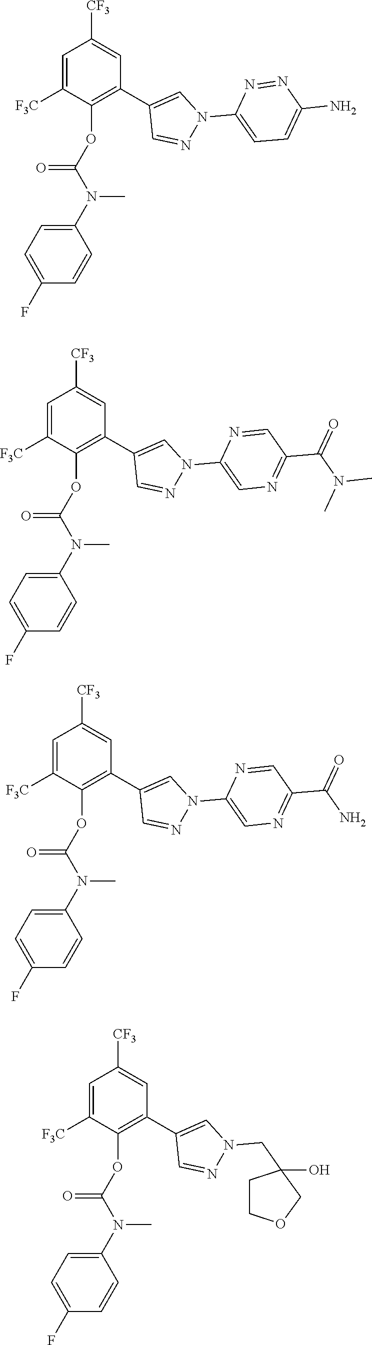 [SMILES: CN(C(=O)Oc1c(-c2cnn(-c3ccc(N)nn3)c2)cc(C(F)(F)F)cc1C(F)(F)F)c1ccc(F)cc1 ; CN(C)C(=O)c1cnc(-n2cc(-c3cc(C(F)(F)F)cc(C(F)(F)F)c3OC(=O)N(C)c3ccc(F)cc3)cn2)cn1 ; CN(C(=O)Oc1c(-c2cnn(-c3cnc(C(N)=O)cn3)c2)cc(C(F)(F)F)cc1C(F)(F)F)c1ccc(F)cc1 ; CN(C(=O)Oc1c(-c2cnn(CC3(O)CCOC3)c2)cc(C(F)(F)F)cc1C(F)(F)F)c1ccc(F)cc1]

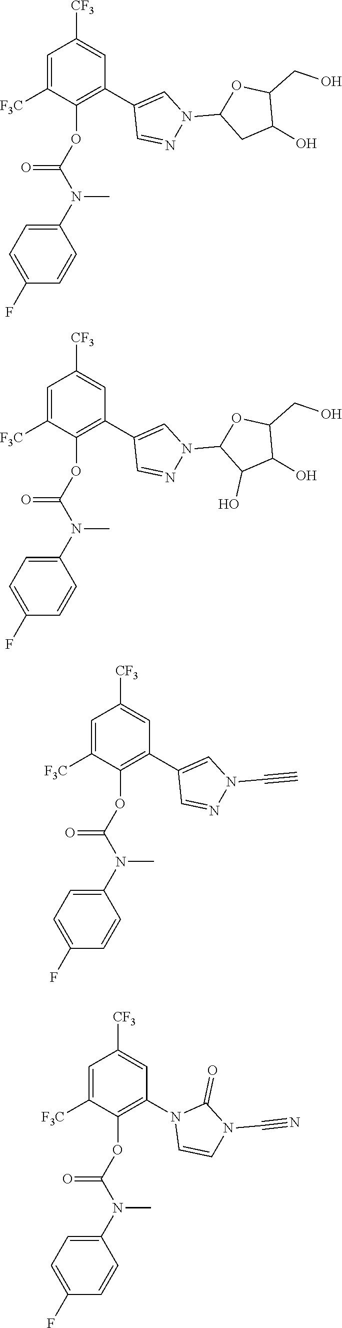 [SMILES: CN(C(=O)Oc1c(-c2cnn(C3CC(O)C(CO)O3)c2)cc(C(F)(F)F)cc1C(F)(F)F)c1ccc(F)cc1 ; CN(C(=O)Oc1c(-c2cnn(C3OC(CO)C(O)C3O)c2)cc(C(F)(F)F)cc1C(F)(F)F)c1ccc(F)cc1 ; C#Cn1cc(-c2cc(C(F)(F)F)cc(C(F)(F)F)c2OC(=O)N(C)c2ccc(F)cc2)cn1 ; CN(C(=O)Oc1c(-n2ccn(C#N)c2=O)cc(C(F)(F)F)cc1C(F)(F)F)c1ccc(F)cc1]

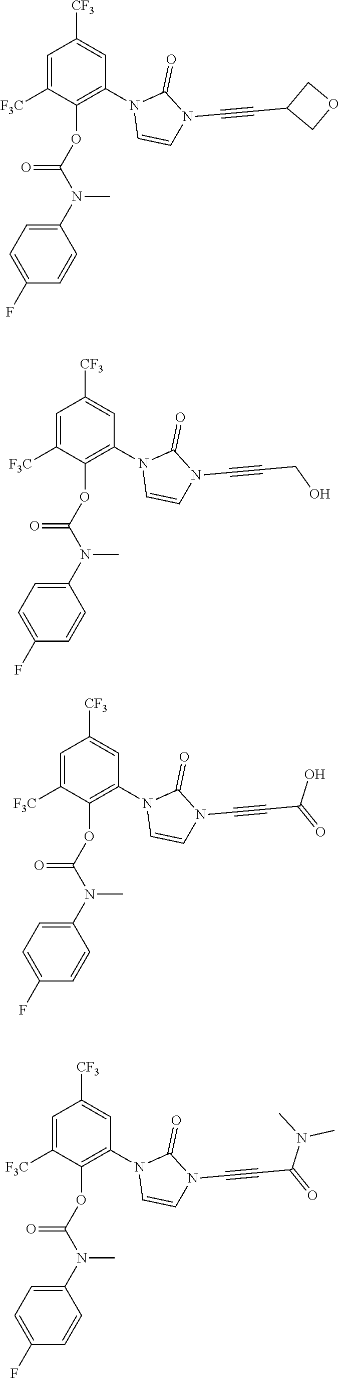 [SMILES: CN(C(=O)Oc1c(-n2ccn(C#CC3COC3)c2=O)cc(C(F)(F)F)cc1C(F)(F)F)c1ccc(F)cc1 ; CN(C(=O)Oc1c(-n2ccn(C#CCO)c2=O)cc(C(F)(F)F)cc1C(F)(F)F)c1ccc(F)cc1 ; CN(C(=O)Oc1c(-n2ccn(C#CC(=O)O)c2=O)cc(C(F)(F)F)cc1C(F)(F)F)c1ccc(F)cc1 ; CN(C)C(=O)C#Cn1ccn(-c2cc(C(F)(F)F)cc(C(F)(F)F)c2OC(=O)N(C)c2ccc(F)cc2)c1=O]

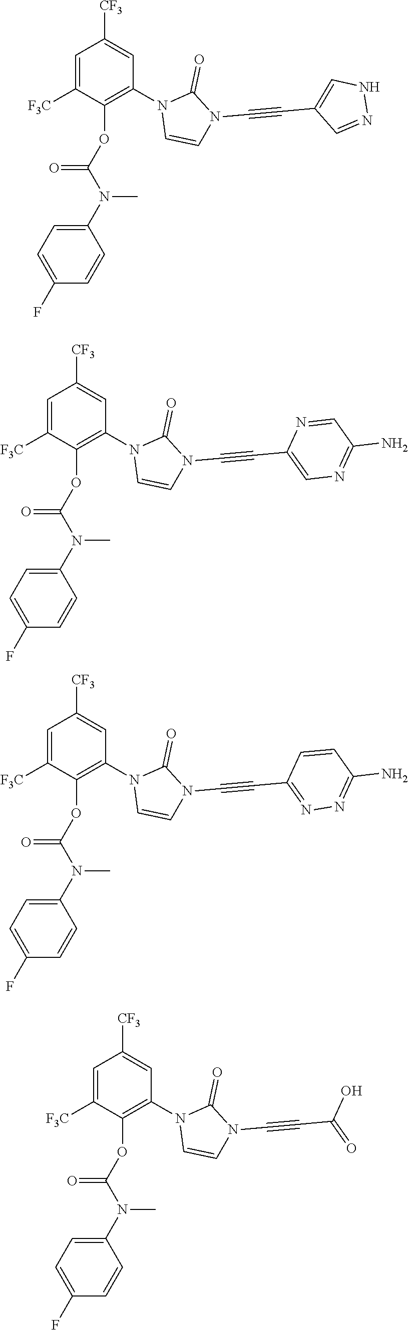 [SMILES: CN(C(=O)Oc1c(-n2ccn(C#Cc3cn[nH]c3)c2=O)cc(C(F)(F)F)cc1C(F)(F)F)c1ccc(F)cc1 ; CN(C(=O)Oc1c(-n2ccn(C#Cc3cnc(N)cn3)c2=O)cc(C(F)(F)F)cc1C(F)(F)F)c1ccc(F)cc1 ; CN(C(=O)Oc1c(-n2ccn(C#Cc3ccc(N)nn3)c2=O)cc(C(F)(F)F)cc1C(F)(F)F)c1ccc(F)cc1 ; CN(C(=O)Oc1c(-n2ccn(C#CC(=O)O)c2=O)cc(C(F)(F)F)cc1C(F)(F)F)c1ccc(F)cc1]

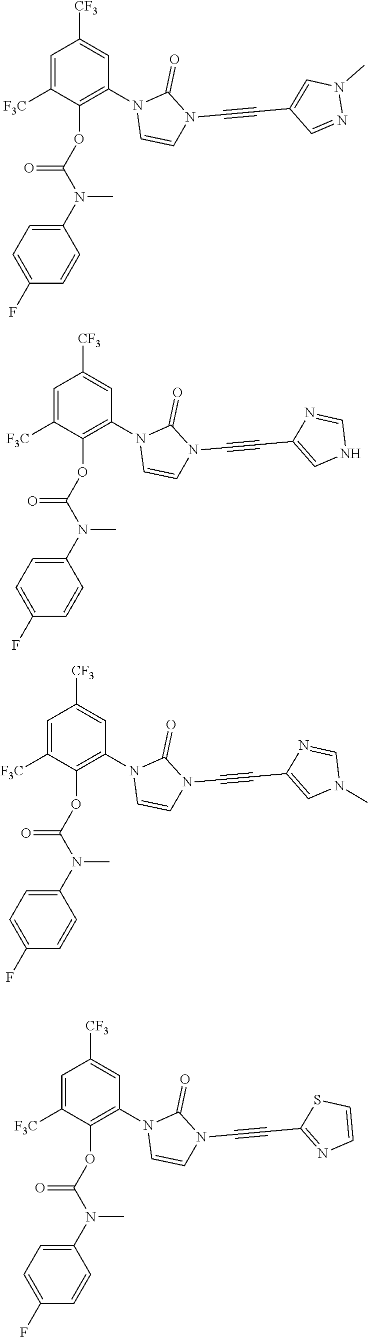 [SMILES: CN(C(=O)Oc1c(-n2ccn(C#Cc3cnn(C)c3)c2=O)cc(C(F)(F)F)cc1C(F)(F)F)c1ccc(F)cc1 ; CN(C(=O)Oc1c(-n2ccn(C#Cc3c[nH]cn3)c2=O)cc(C(F)(F)F)cc1C(F)(F)F)c1ccc(F)cc1 ; CN(C(=O)Oc1c(-n2ccn(C#Cc3cn(C)cn3)c2=O)cc(C(F)(F)F)cc1C(F)(F)F)c1ccc(F)cc1 ; CN(C(=O)Oc1c(-n2ccn(C#Cc3nccs3)c2=O)cc(C(F)(F)F)cc1C(F)(F)F)c1ccc(F)cc1]

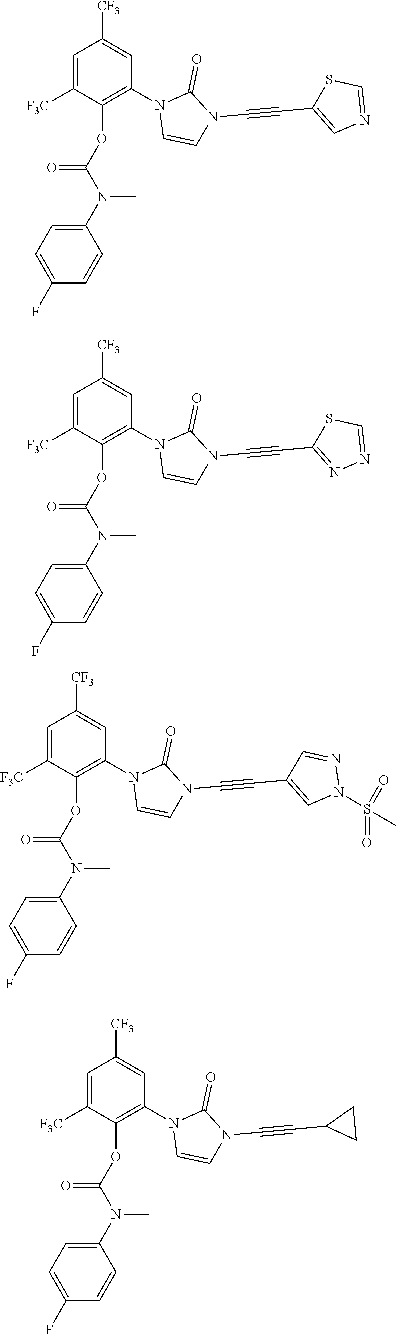 [SMILES: CN(C(=O)Oc1c(-n2ccn(C#Cc3cncs3)c2=O)cc(C(F)(F)F)cc1C(F)(F)F)c1ccc(F)cc1 ; CN(C(=O)Oc1c(-n2ccn(C#Cc3nncs3)c2=O)cc(C(F)(F)F)cc1C(F)(F)F)c1ccc(F)cc1 ; CN(C(=O)Oc1c(-n2ccn(C#Cc3cnn(S(C)(=O)=O)c3)c2=O)cc(C(F)(F)F)cc1C(F)(F)F)c1ccc(F)cc1 ; CN(C(=O)Oc1c(-n2ccn(C#CC3CC3)c2=O)cc(C(F)(F)F)cc1C(F)(F)F)c1ccc(F)cc1]

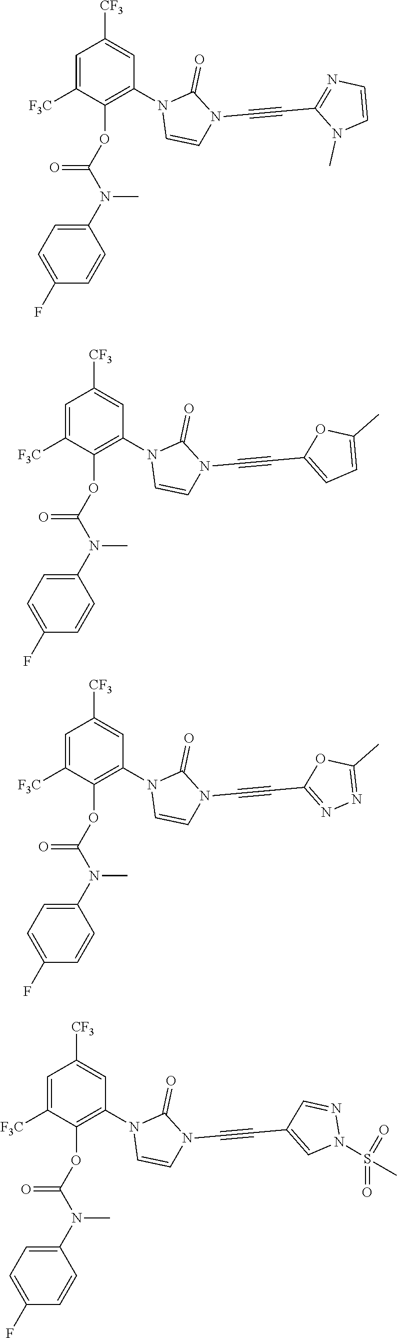 [SMILES: CN(C(=O)Oc1c(-n2ccn(C#Cc3nccn3C)c2=O)cc(C(F)(F)F)cc1C(F)(F)F)c1ccc(F)cc1 ; Cc1ccc(C#Cn2ccn(-c3cc(C(F)(F)F)cc(C(F)(F)F)c3OC(=O)N(C)c3ccc(F)cc3)c2=O)o1 ; Cc1nnc(C#Cn2ccn(-c3cc(C(F)(F)F)cc(C(F)(F)F)c3OC(=O)N(C)c3ccc(F)cc3)c2=O)o1 ; CN(C(=O)Oc1c(-n2ccn(C#Cc3cnn(S(C)(=O)=O)c3)c2=O)cc(C(F)(F)F)cc1C(F)(F)F)c1ccc(F)cc1]

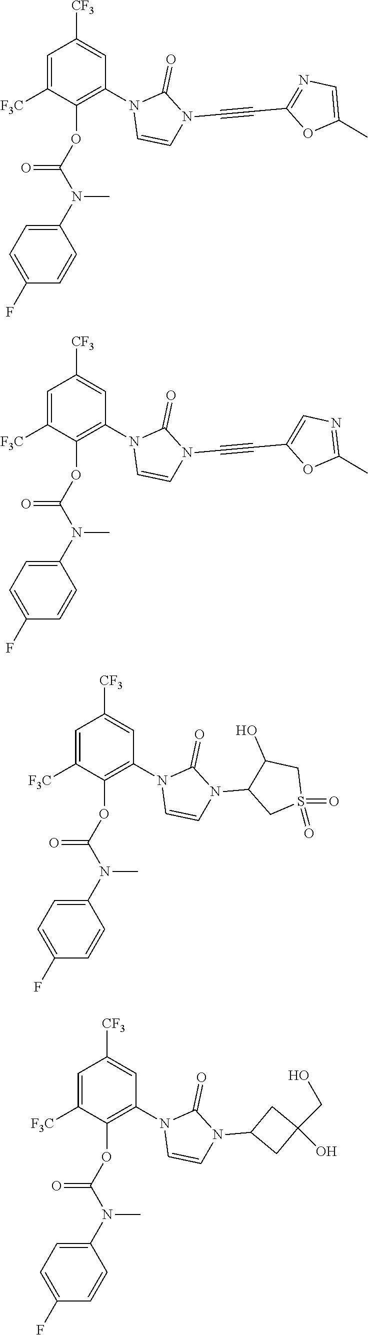 [SMILES: Cc1cnc(C#Cn2ccn(-c3cc(C(F)(F)F)cc(C(F)(F)F)c3OC(=O)N(C)c3ccc(F)cc3)c2=O)o1 ; Cc1ncc(C#Cn2ccn(-c3cc(C(F)(F)F)cc(C(F)(F)F)c3OC(=O)N(C)c3ccc(F)cc3)c2=O)o1 ; CN(C(=O)Oc1c(-n2ccn(C3CS(=O)(=O)CC3O)c2=O)cc(C(F)(F)F)cc1C(F)(F)F)c1ccc(F)cc1 ; CN(C(=O)Oc1c(-n2ccn(C3CC(O)(CO)C3)c2=O)cc(C(F)(F)F)cc1C(F)(F)F)c1ccc(F)cc1]

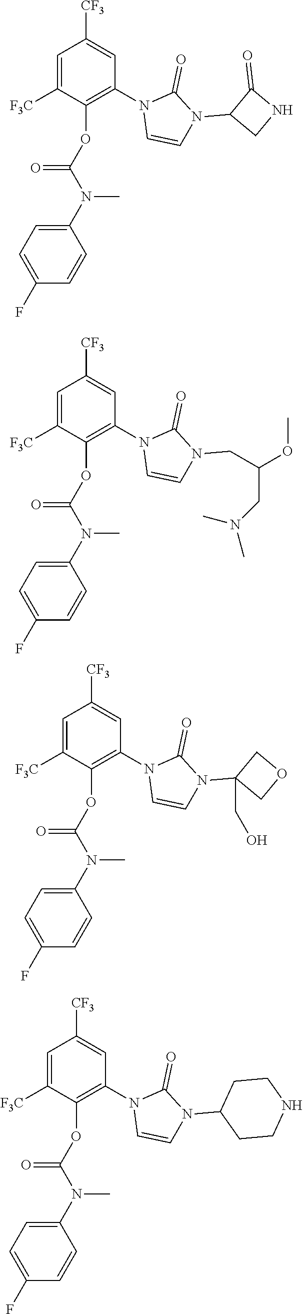 [SMILES: CN(C(=O)Oc1c(-n2ccn(C3CNC3=O)c2=O)cc(C(F)(F)F)cc1C(F)(F)F)c1ccc(F)cc1 ; COC(CN(C)C)Cn1ccn(-c2cc(C(F)(F)F)cc(C(F)(F)F)c2OC(=O)N(C)c2ccc(F)cc2)c1=O ; CN(C(=O)Oc1c(-n2ccn(C3(CO)COC3)c2=O)cc(C(F)(F)F)cc1C(F)(F)F)c1ccc(F)cc1 ; CN(C(=O)Oc1c(-n2ccn(C3CCNCC3)c2=O)cc(C(F)(F)F)cc1C(F)(F)F)c1ccc(F)cc1]

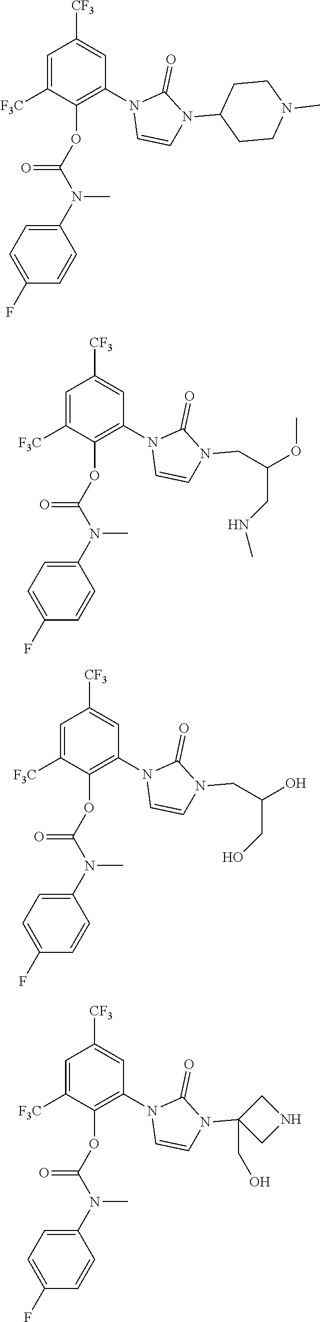 [SMILES: CN1CCC(n2ccn(-c3cc(C(F)(F)F)cc(C(F)(F)F)c3OC(=O)N(C)c3ccc(F)cc3)c2=O)CC1 ; CNCC(Cn1ccn(-c2cc(C(F)(F)F)cc(C(F)(F)F)c2OC(=O)N(C)c2ccc(F)cc2)c1=O)OC ; CN(C(=O)Oc1c(-n2ccn(CC(O)CO)c2=O)cc(C(F)(F)F)cc1C(F)(F)F)c1ccc(F)cc1 ; CN(C(=O)Oc1c(-n2ccn(C3(CO)CNC3)c2=O)cc(C(F)(F)F)cc1C(F)(F)F)c1ccc(F)cc1]

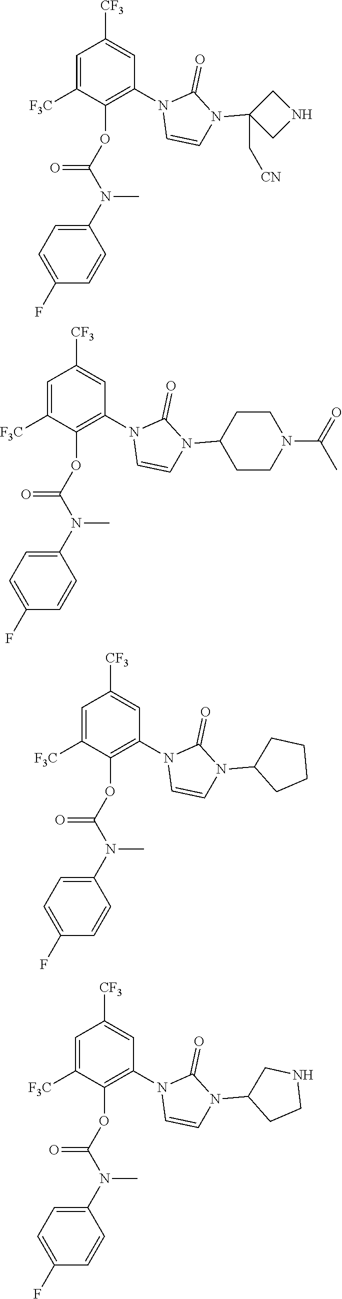

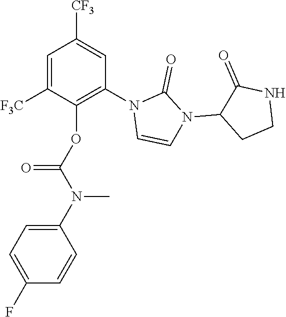 [SMILES: CN(C(=O)Oc1c(-n2ccn(C3CCNC3=O)c2=O)cc(C(F)(F)F)cc1C(F)(F)F)c1ccc(F)cc1]

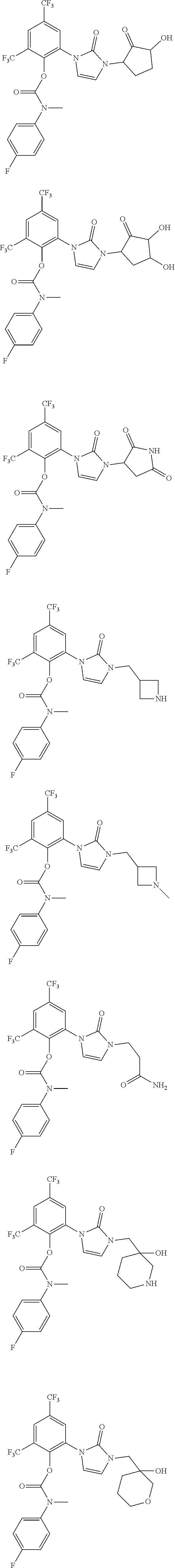

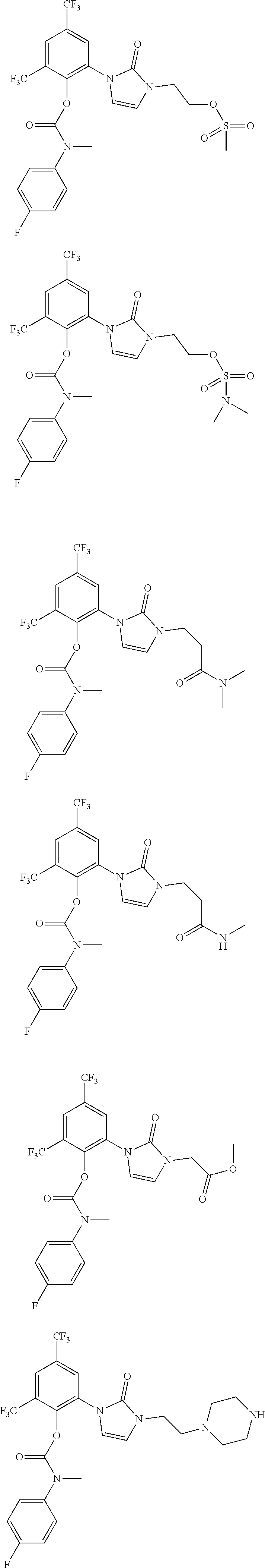 [SMILES: CN(C(=O)Oc1c(-n2ccn(CCOS(C)(=O)=O)c2=O)cc(C(F)(F)F)cc1C(F)(F)F)c1ccc(F)cc1 ; CN(C(=O)Oc1c(-n2ccn(CCOS(=O)(=O)N(C)C)c2=O)cc(C(F)(F)F)cc1C(F)(F)F)c1ccc(F)cc1 ; CN(C)C(=O)CCn1ccn(-c2cc(C(F)(F)F)cc(C(F)(F)F)c2OC(=O)N(C)c2ccc(F)cc2)c1=O ; CNC(=O)CCn1ccn(-c2cc(C(F)(F)F)cc(C(F)(F)F)c2OC(=O)N(C)c2ccc(F)cc2)c1=O ; COC(=O)Cn1ccn(-c2cc(C(F)(F)F)cc(C(F)(F)F)c2OC(=O)N(C)c2ccc(F)cc2)c1=O ; CN(C(=O)Oc1c(-n2ccn(CCN3CCNCC3)c2=O)cc(C(F)(F)F)cc1C(F)(F)F)c1ccc(F)cc1]

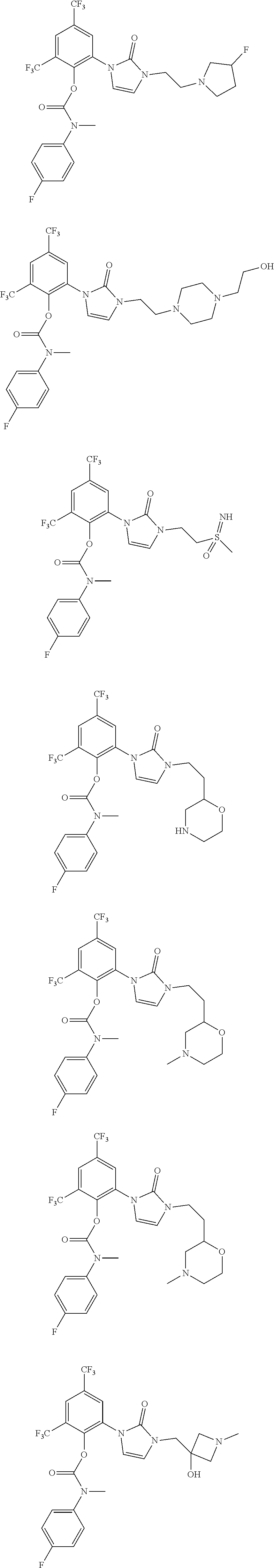

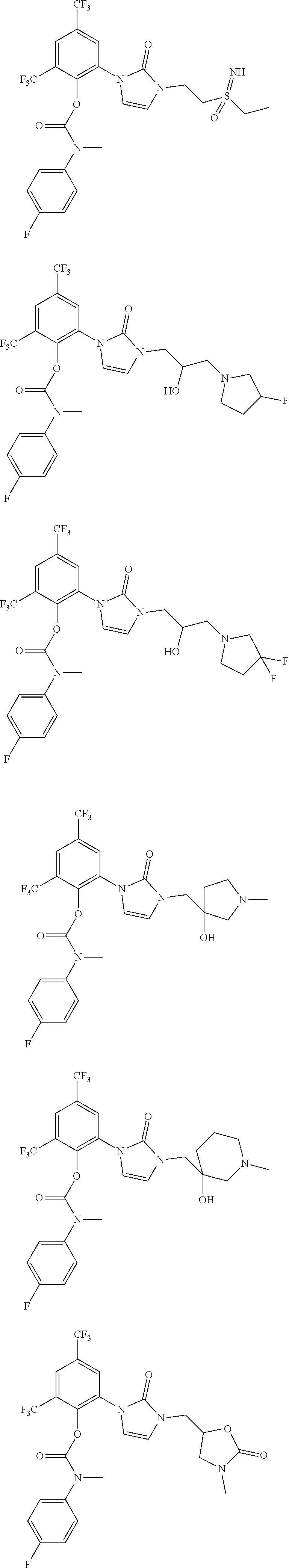 [SMILES: CCS(=N)(=O)CCn1ccn(-c2cc(C(F)(F)F)cc(C(F)(F)F)c2OC(=O)N(C)c2ccc(F)cc2)c1=O ; CN(C(=O)Oc1c(-n2ccn(CC(O)CN3CCC(F)C3)c2=O)cc(C(F)(F)F)cc1C(F)(F)F)c1ccc(F)cc1 ; CN(C(=O)Oc1c(-n2ccn(CC(O)CN3CCC(F)(F)C3)c2=O)cc(C(F)(F)F)cc1C(F)(F)F)c1ccc(F)cc1 ; CN1CCC(O)(Cn2ccn(-c3cc(C(F)(F)F)cc(C(F)(F)F)c3OC(=O)N(C)c3ccc(F)cc3)c2=O)C1 ; CN1CCCC(O)(Cn2ccn(-c3cc(C(F)(F)F)cc(C(F)(F)F)c3OC(=O)N(C)c3ccc(F)cc3)c2=O)C1 ; CN1CC(Cn2ccn(-c3cc(C(F)(F)F)cc(C(F)(F)F)c3OC(=O)N(C)c3ccc(F)cc3)c2=O)OC1=O]

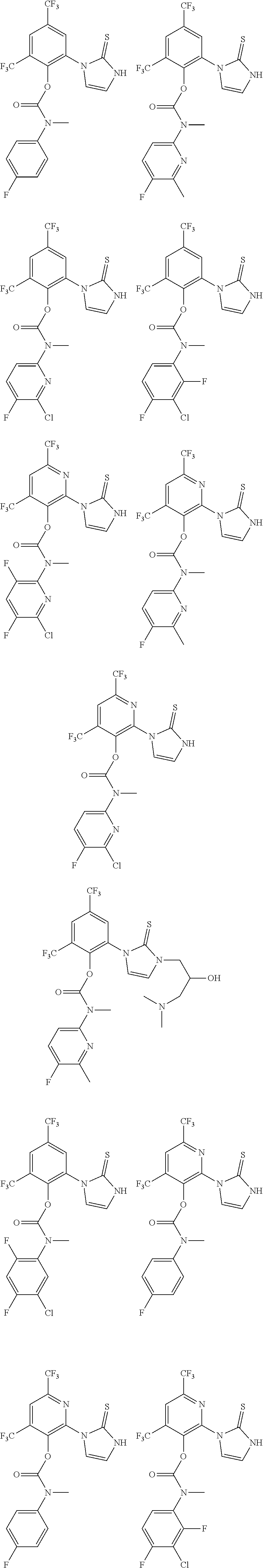

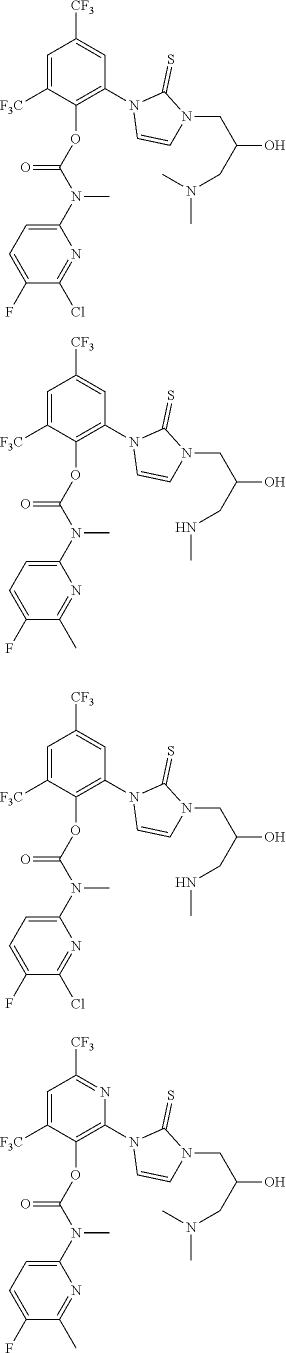

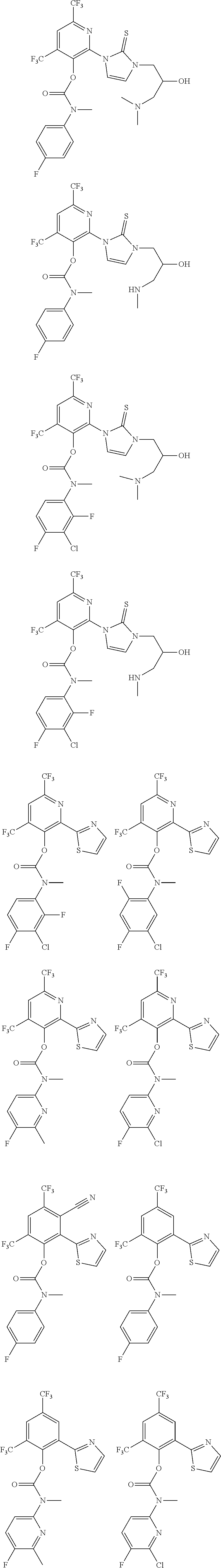

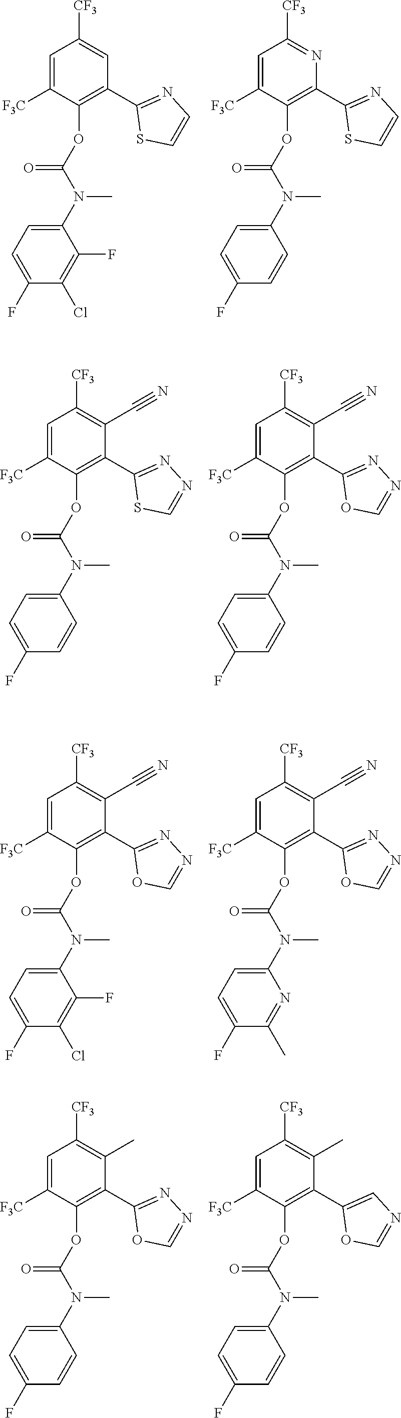

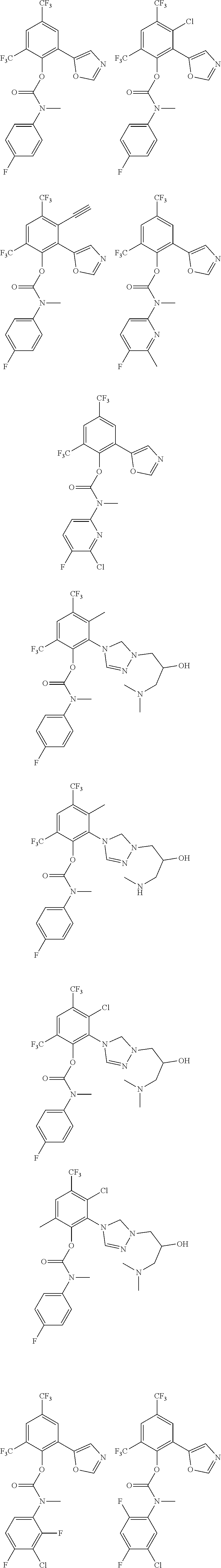

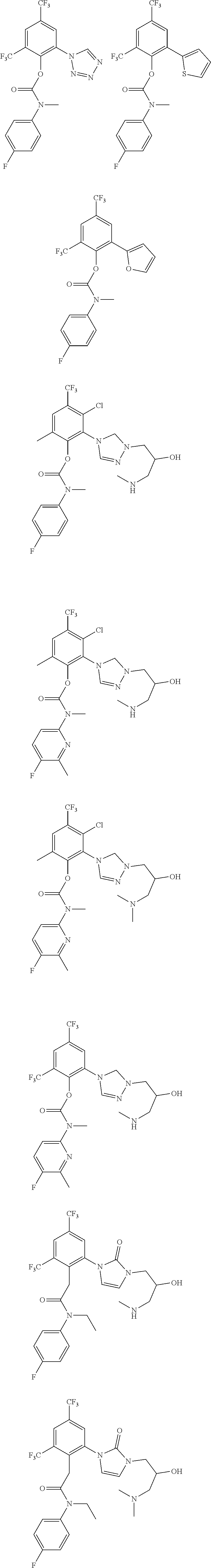

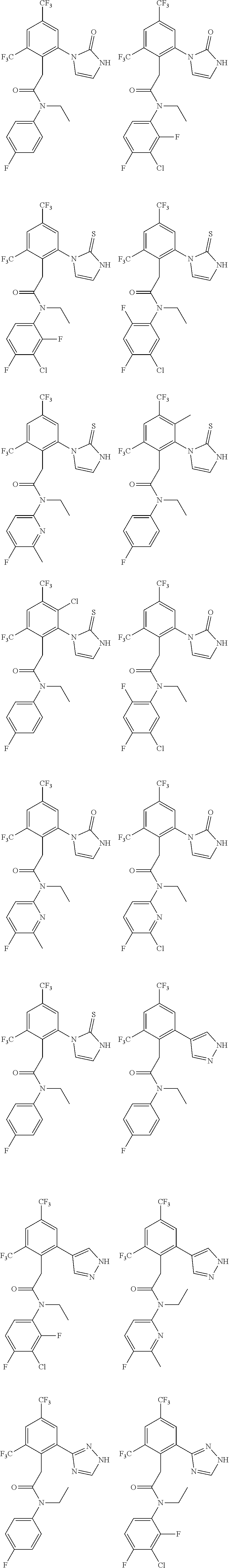

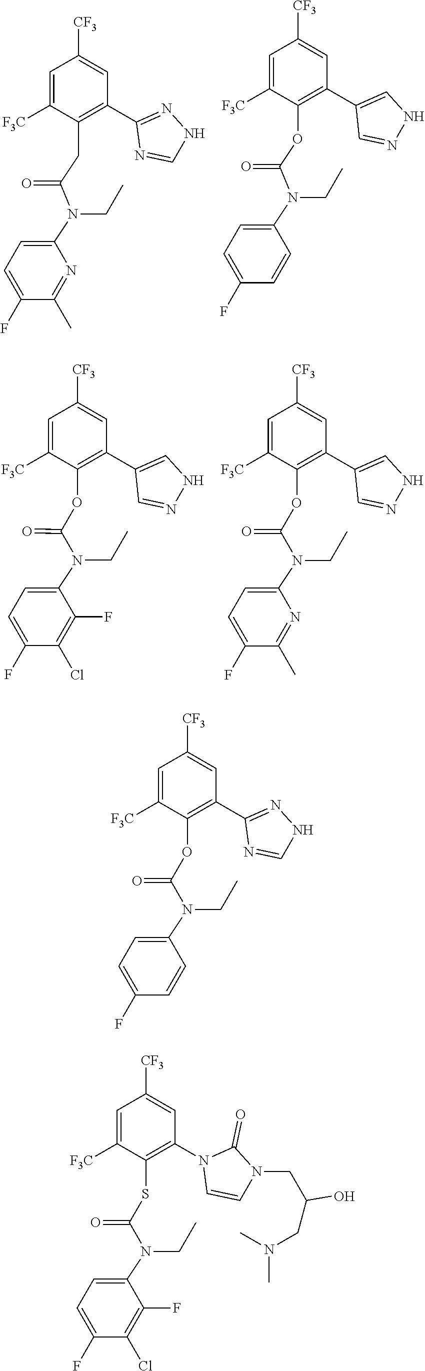

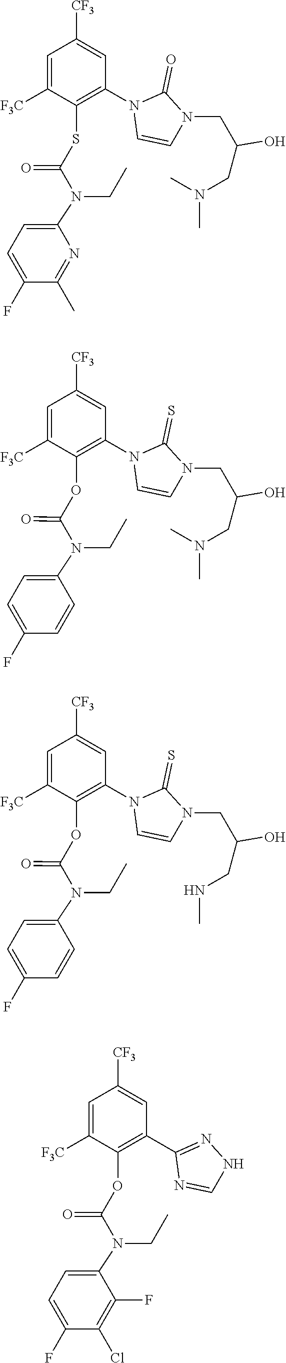

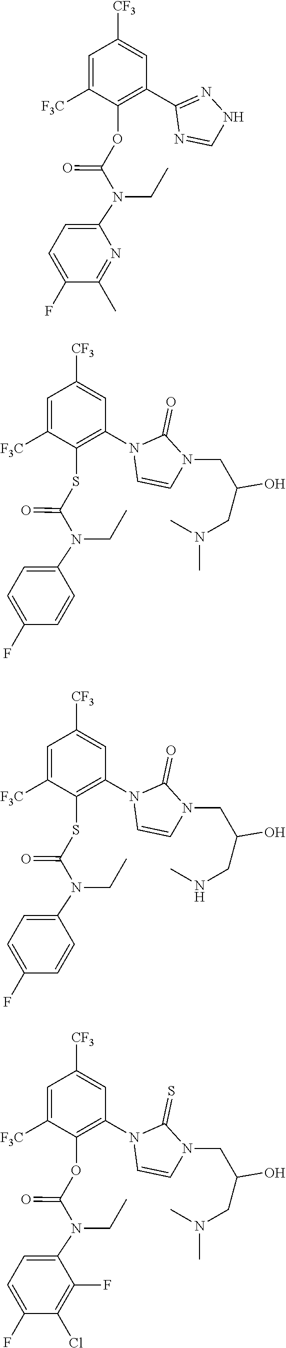

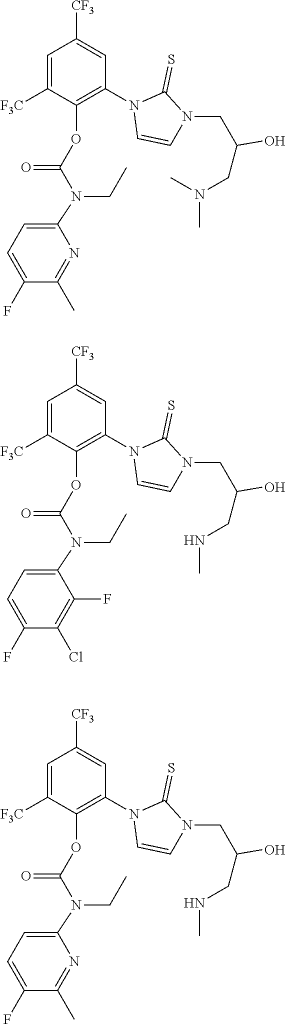

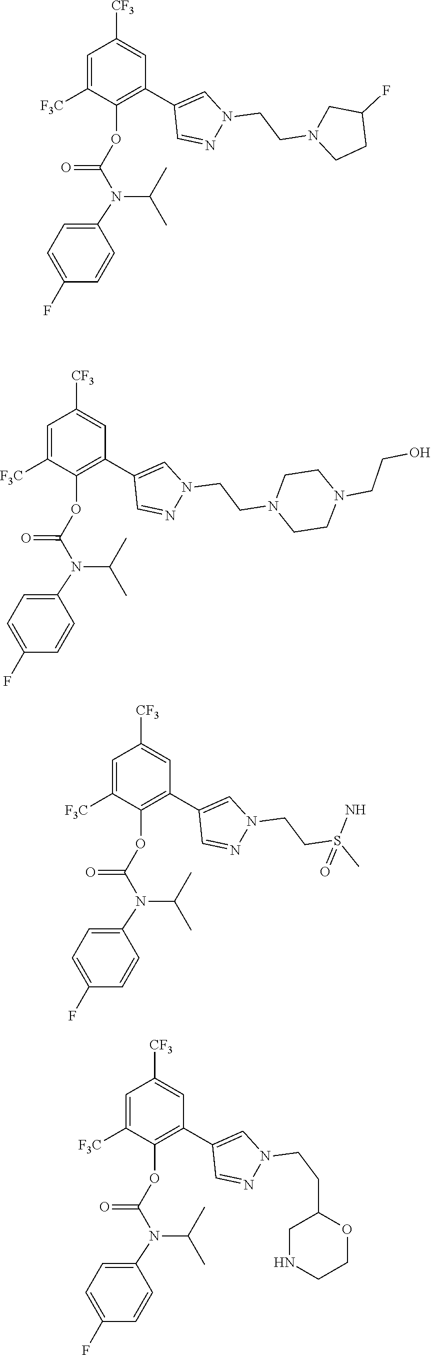

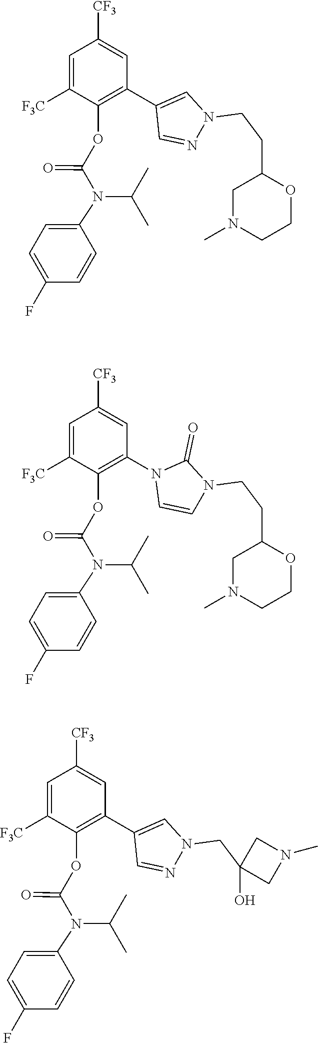 [SMILES: CC(C)N(C(=O)Oc1c(-c2cnn(CCC3CN(C)CCO3)c2)cc(C(F)(F)F)cc1C(F)(F)F)c1ccc(F)cc1 ; CC(C)N(C(=O)Oc1c(-n2ccn(CCC3CN(C)CCO3)c2=O)cc(C(F)(F)F)cc1C(F)(F)F)c1ccc(F)cc1 ; CC(C)N(C(=O)Oc1c(-c2cnn(CC3(O)CN(C)C3)c2)cc(C(F)(F)F)cc1C(F)(F)F)c1ccc(F)cc1]

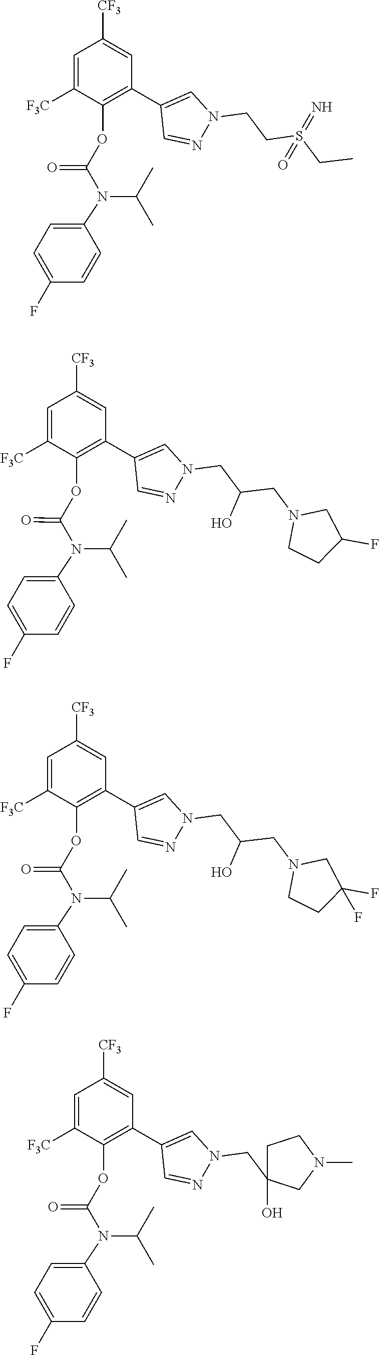 [SMILES: CCS(=N)(=O)CCn1cc(-c2cc(C(F)(F)F)cc(C(F)(F)F)c2OC(=O)N(c2ccc(F)cc2)C(C)C)cn1 ; CC(C)N(C(=O)Oc1c(-c2cnn(CC(O)CN3CCC(F)C3)c2)cc(C(F)(F)F)cc1C(F)(F)F)c1ccc(F)cc1 ; CC(C)N(C(=O)Oc1c(-c2cnn(CC(O)CN3CCC(F)(F)C3)c2)cc(C(F)(F)F)cc1C(F)(F)F)c1ccc(F)cc1 ; CC(C)N(C(=O)Oc1c(-c2cnn(CC3(O)CCN(C)C3)c2)cc(C(F)(F)F)cc1C(F)(F)F)c1ccc(F)cc1]

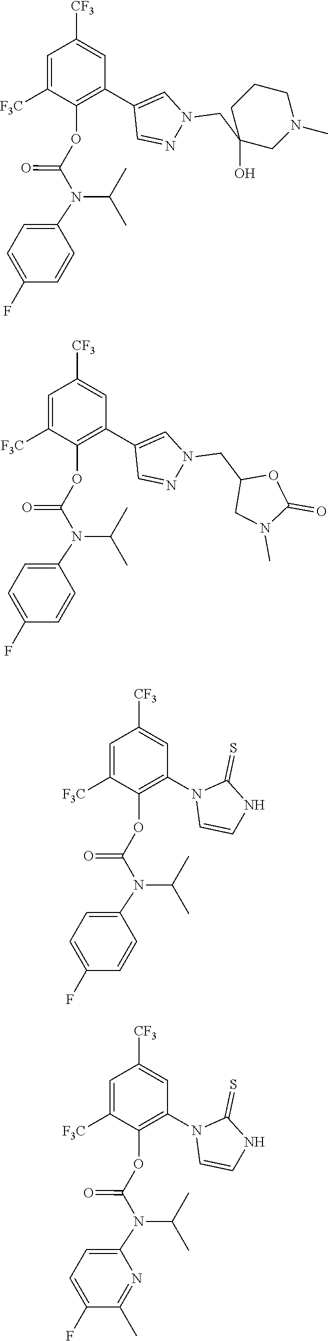 [SMILES: CC(C)N(C(=O)Oc1c(-c2cnn(CC3(O)CCCN(C)C3)c2)cc(C(F)(F)F)cc1C(F)(F)F)c1ccc(F)cc1 ; CC(C)N(C(=O)Oc1c(-c2cnn(CC3CN(C)C(=O)O3)c2)cc(C(F)(F)F)cc1C(F)(F)F)c1ccc(F)cc1 ; CC(C)N(C(=O)Oc1c(-n2cc[nH]c2=S)cc(C(F)(F)F)cc1C(F)(F)F)c1ccc(F)cc1 ; Cc1nc(N(C(=O)Oc2c(-n3cc[nH]c3=S)cc(C(F)(F)F)cc2C(F)(F)F)C(C)C)ccc1F]

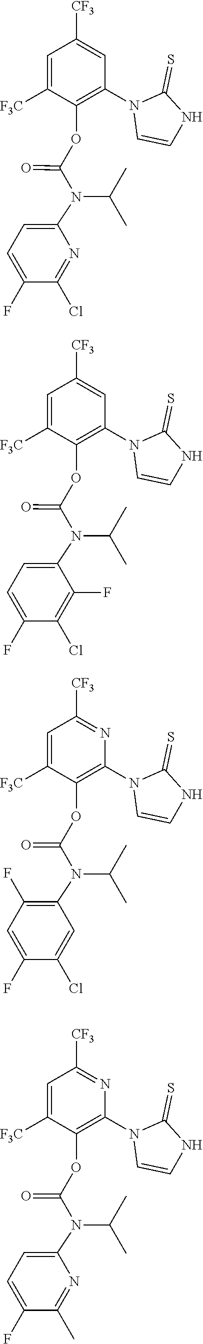

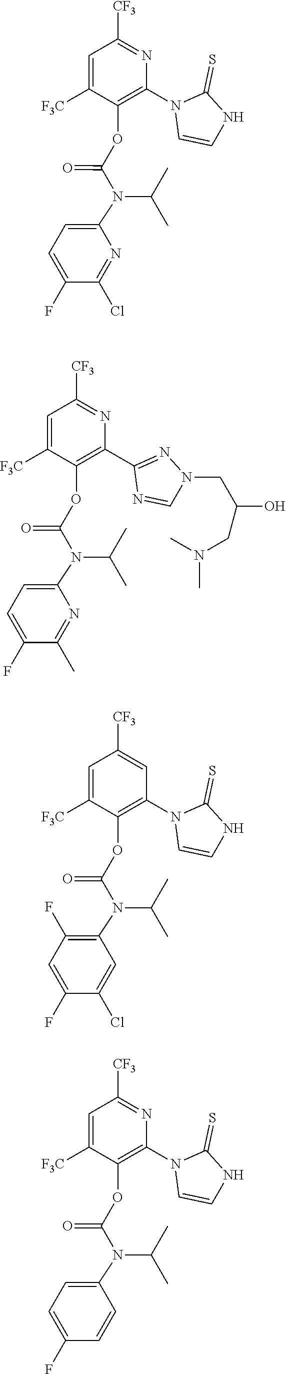

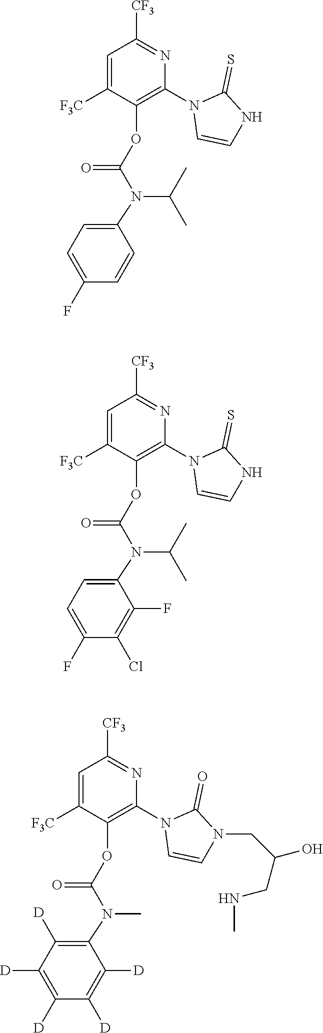 [SMILES: CC(C)N(C(=O)Oc1c(C(F)(F)F)cc(C(F)(F)F)nc1-n1cc[nH]c1=S)c1ccc(F)cc1 ; CC(C)N(C(=O)Oc1c(C(F)(F)F)cc(C(F)(F)F)nc1-n1cc[nH]c1=S)c1ccc(F)c(Cl)c1F ; [2H]c1c([2H])c([2H])c(N(C)C(=O)Oc2c(C(F)(F)F)cc(C(F)(F)F)nc2-n2ccn(CC(O)CNC)c2=O)c([2H])c1[2H]]

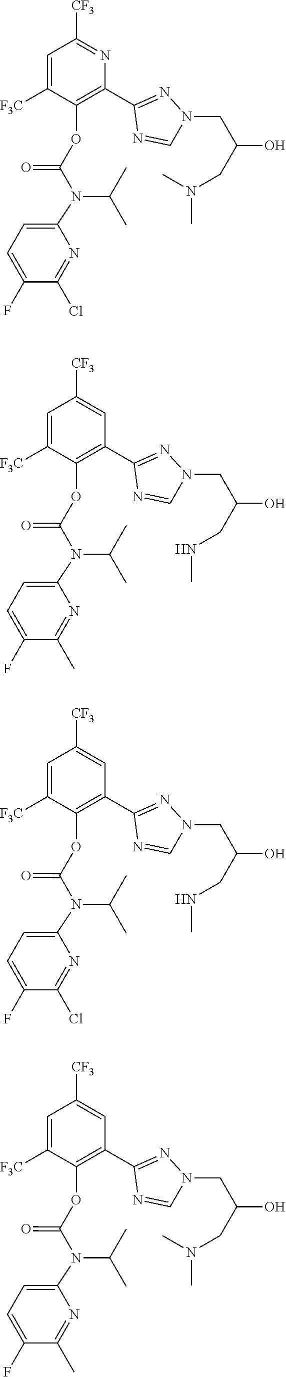

### Salts

Certain compounds of the Formula (I) can exist in the form of salts, for example acid addition salts or, in certain cases salts of organic and inorganic bases such as carboxylate, sulfonate and phosphate salts. The term “salts” embraces addition salts of free acids or free bases which are compounds of the invention. The term “pharmaceutically acceptable salt” refers to salts which possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compounds of this invention.

All such salts are within the scope of this invention, and references to compounds of the Formula (I) include the salt forms of the compounds. The salts of the present invention can be synthesized from the parent compound that contains a basic or acidic moiety by conventional chemical methods such as methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free acid or base forms of these compounds with the appropriate base or acid in water or in an organic solvent, or in a mixture of the two; generally, non-aqueous media such as ether, ethyl acetate, ethanol, isopropanol, or acetonitrile are used.

Acid addition salts (mono- or di-salts) may be formed with a wide variety of acids, both inorganic and organic. Examples of acid addition salts include mono- or di-salts formed with an acid selected from the group consisting of acetic, 2,2-dichloroacetic, adipic, alginic, ascorbic (e.g. L-ascorbic), L-aspartic, benzenesulfonic, benzoic, 4-acetamidobenzoic, butanoic, (+) camphoric, camphor-sulfonic, (+)-(1S)-camphor-10-sulfonic, capric, caproic, caprylic, cinnamic, citric, cyclamic, dodecylsulfuric, ethane-1,2-disulfonic, ethanesulfonic, 2-hydroxyethanesulfonic, formic, fumaric, galactaric, gentisic, glucoheptonic, D-gluconic, glucuronic (e.g. D-glucuronic), glutamic (e.g. L-glutamic), a-oxoglutaric, glycolic, hippuric, hydrohalic acids (e.g. hydrobromic, hydrochloric, hydriodic), isethionic, lactic (e.g. (+)-Llactic, (±)-DL-lactic), lactobionic, maleic, malic, (−)-L-malic, malonic, (±)-DL-mandelic, methanesulfonic, naphthalene-2-sulfonic, naphthalene-1,5-disulfonic, 1-hydroxy-2-naphthoic, nicotinic, nitric, oleic, orotic, oxalic, palmitic, pamoic, phosphoric, propionic, pyruvic, Lpyroglutamic, salicylic, 4-amino-salicylic, sebacic, stearic, succinic, sulfuric, tannic, (+)-Ltartaric, thiocyanic, p-toluenesulfonic, undecylenic and valeric acids, as well as acylated amino acids and cation exchange resins. One particular group of salts consists of salts formed from acetic, hydrochloric, hydriodic, phosphoric, nitric, sulfuric, citric, lactic, succinic, maleic, malic, isethionic, fumaric, benzenesulfonic, toluenesulfonic, methanesulfonic (mesylate), ethanesulfonic, naphthalenesulfonic, valeric, acetic, propanoic, butanoic, malonic, glucuronic and lactobionic acids. One particular salt is the hydrochloride salt. Where the compounds of the formula (I) contain an amine function, these may form quaternary ammonium salts, for example by reaction with an alkylating agent according to methods well known to the skilled person. Such quaternary ammonium compounds are within the scope of formula (I). The compounds of the invention may exist as mono- or di-salts depending upon the pKa of the acid from which the salt is formed. It will be appreciated that for use in medicine the salts of the compounds of formula (I) should be pharmaceutically acceptable. Suitable pharmaceutically acceptable salts will be apparent to those skilled in the art.

Pharmaceutically acceptable salts include those described by Berge, Bighley and Monkhouse, J. Pharm. Sci. 1977, 66, pp. 1-19. Suitable pharmaceutically acceptable acid addition salts may be prepared from an inorganic acid or from an organic acid. Examples of inorganic acids include hydrochloric, hydrobromic, hydroiodic, nitric, carbonic, sulfuric and phosphoric acids. Appropriate organic acids may be selected from aliphatic, cycloaliphatic, aromatic, araliphatic, heterocyclic, carboxylic and sulfonic classes of organic acids, examples of which include formic, acetic, pivalic, propionic, furoic, mucic, isethionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric, camphorsulfonic and galacturonic acid. Examples of pharmaceutically unacceptable acid addition salts include, for example, perchlorates and tetrafluoroborates. However, salts that are not pharmaceutically acceptable may also be prepared as intermediate forms which may then be converted into pharmaceutically acceptable salts. Such non-pharmaceutically acceptable salts forms, which may be useful, for example, in the purification or separation of the compounds of the invention, also form part of the invention. Certain of the compounds of formula (I) may form acid addition salts with one or more equivalents of the acid. The present invention includes within its scope all possible stoichiometric and non-stoichiometric forms.

Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylenediamine, chloroprocaine, choline, diethanolamine, ethylenediamine, tromethamine, meglumine (N-methylglucamine) and procaine. Examples of pharmaceutically unacceptable base addition salts include lithium salts and cyanate salts.

All of these salts may be prepared by conventional means from the corresponding compound according to Formula I by reacting, for example, the appropriate acid or base with the compound according to Formula I. Preferably the salts are in crystalline form, and preferably prepared by crystallization of the salt from a suitable solvent. The person skilled in the art will know how to prepare and select suitable salt forms for example, as described in Handbook of Pharmaceutical Salts: Properties, Selectin and Use by P. H. Stahl and C. G. Wermuth (Wiley-VCH 2002).

### Solvates

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Pharmaceutically acceptable solvates of the compound of the invention are within the scope of the invention. In one embodiment, the pharmaceutically acceptable solvates of the compounds of the invention include the hydrate thereof. In one embodiment, said crystalline form of the compounds of formula (I) is a cocrystal or coformer. Such a cocrystal or coformer may be prepared using water-soluble molecules such as saccharin, caffeine, nicotinamide or carboxylic acids. Coformers may be prepared as described in Emami S et al (2018) BioImpacts 8(4), 305-320, the techniques of which are herein incorporated by reference. It will be understood that the invention includes pharmaceutically acceptable derivatives of compounds of formula (I) and that these are included within the scope of the invention. As used herein “pharmaceutically acceptable derivative” includes any pharmaceutically acceptable ester or salt of such ester of a compound of formula (I) which, upon administration to the recipient is capable of providing (directly or indirectly) a compound of formula (I) or an active metabolite or residue thereof.

### N-Oxides

Compounds of the formula (I) containing an amine function may also form N-oxides. A reference herein to a compound of the formula (I) that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4th Edition, Wiley Interscience. More particularly, N-oxides can be made by the procedure of L. W. Deady (Syn. Commun. 1977, 7, 509-514) in which the amine compound is reacted with m-chloroperoxybenzoic acid (mCPBA), for example, in an inert solvent such as dichloromethane.

### Prodrugs

It will be appreciated by those skilled in the art that certain protected derivatives of compounds of formula (I), which may be made prior to a final deprotection stage, may not possess pharmacological activity as such, but may, in certain instances, be administered orally or parenterally and thereafter metabolised in the body to form compounds of the invention which are pharmacologically active. Such derivatives may therefore be described as “prodrugs”. All such prodrugs of compounds of the invention are included within the scope of the invention. Examples of pro-drug functionality suitable for the compounds of the present invention are described in Drugs of Today, 19, 9, 1983, 499-538 and in Topics in Chemistry, Chapter 31, pp. 306-316 and in “Design of Prodrugs” by H. Bundgaard, Elsevier, 1985, Chapter 1 (the disclosures in which documents are incorporated herein by reference). It will further be appreciated by those skilled in the art, that certain moieties, known to those skilled in the art as “pro-moieties”, for example as described by H. Bundgaard in “Design of Prodrugs” (the disclosure in which document is incorporated herein by reference) may be placed on appropriate functionalities when such functionalities are present within compounds of the invention. Also included within the scope of the compound and various salts of the invention are polymorphs thereof.

### Enantiomers

Where chiral centres are present in compounds of formula (I), the present invention includes within its scope all possible enantiomers and diastereoisomers, including mixtures thereof. The different isomeric forms may be separated or resolved one from the other by conventional methods, or any given isomer may be obtained by conventional synthetic methods or by stereospecific or asymmetric syntheses. The invention also extends to any tautomeric forms or mixtures thereof.

### Isotopes

The subject invention also includes all pharmaceutically acceptable isotopically-labelled compounds which are identical to those recited in formula (I) but for the fact that one or more atoms are replaced by an atom having an atomic mass or mass number different from the atomic mass or mass number most commonly found in nature. Examples of isotopes suitable for inclusion in the compounds of the invention comprise isotopes of hydrogen, such as 2H (D) and H (T), carbon, such as 1 1C, 1 C and 14C, chlorine, such as 6CI, fluorine, such as 18F, iodine, such as 123 1, 125 1 and 1 1 1, nitrogen, such as 1 N and 1 N, oxygen, such as 1 0, 170 and 180, phosphorus, such as 2P, and sulfur, such as S. Certain isotopically-labelled compounds of formula (I), for example, those incorporating a radioactive isotope, are useful in drug and/or substrate tissue distribution studies. The compounds of formula (I) can also have valuable diagnostic properties in that they can be used for detecting or identifying the formation of a complex between a labelled compound and other molecules, peptides, proteins, enzymes or receptors. The detecting or identifying methods can use compounds that are labelled with labelling agents such as radioisotopes, enzymes, fluorescent substances, luminous substances (for example, luminol, luminol derivatives, luciferin, aequorin and luciferase) etc. The radioactive isotopes tritium, i.e. H (T), and carbon-14, i.e. 14C, are particularly useful for this purpose in view of their case of incorporation and ready means of detection. Substitution with heavier isotopes such as deuterium, i.e. 2H (D), may afford certain therapeutic advantages resulting from greater metabolic stability, for example, increased in vivo half-life or reduced dosage requirements, and hence may be preferred in some circumstances. Substitution with positron emitting isotopes, such as 1 1C, 18F, 1 0 and 1 N, can be useful in Positron Emission Topography (PET) studies for examining target occupancy.

Isotopically-labelled compounds of formula (I) can generally be prepared by conventional techniques known to those skilled in the art or by processes analogous to those described in the accompanying Examples and Preparations using appropriate isotopically-labelled reagents in place of the non-labelled reagent previously employed.

### Purity

Since the compounds of formula (I) are intended for use in pharmaceutical compositions it will readily be understood that they are each preferably provided in substantially pure form, for example at least 60% pure, more suitably at least 75% pure and preferably at least 85%, especially at least 98% pure (% are given on a weight for weight basis). Impure preparations of the compounds may be used for preparing the more pure forms used in the pharmaceutical compositions.

### Preparation of the Compounds of the Invention

Compounds of formula (I) may be prepared by the general schemes described herein, using the synthetic method known by those skilled in the art. The following examples illustrate non-limiting embodiments of the invention.

The compounds of the invention may possess one or more stereocenters, and each stereocenter may exist independently in either the R or S configuration. In one embodiment, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In one embodiment, a mixture of one or more isomer is utilized as the therapeutic compound described herein. In another embodiment, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound of the invention, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In one embodiment, the compounds described herein exist in solvated forms with pharmaceutically acceptable solvents such as water, and ethanol. In another embodiment, the compounds described herein exist in unsolvated form.

In one embodiment, the compounds of the invention may exist as tautomers. All tautomers are included within the scope of the compounds presented herein.

In one embodiment, compounds described herein are prepared as prodrugs. A “prodrug” refers to an agent that is converted into the parent drug in vivo. In one embodiment, upon in vivo administration, a prodrug is chemically converted to the biologically, pharmaceutically or therapeutically active form of the compound. In another embodiment, a prodrug is enzymatically metabolized by one or more steps or processes to the biologically, pharmaceutically or therapeutically active form of the compound.

In one embodiment, sites on, for example, the aromatic ring portion of compounds of the invention are susceptible to various metabolic reactions. Incorporation of appropriate substituents on the aromatic ring structures may reduce, minimize or eliminate this metabolic pathway. In one embodiment, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In one embodiment, isotopically-labeled compounds are useful in drug and/or substrate tissue distribution studies. In another embodiment, substitution with heavier isotopes such as deuterium affords greater metabolic stability (for example, increased in vivo half-life or reduced dosage requirements). In yet another embodiment, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In one embodiment, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4th Ed., (Wiley 1992); Carey & Sundberg, Advanced Organic Chemistry 4th Ed., Vols. A and B (Plenum 2000, 2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources or are prepared using procedures described herein.

In one embodiment, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In another embodiment, each protective group is removable by a different means. Protective groups that are cleaved under totally disparate reaction conditions fulfill the requirement of differential removal.

In one embodiment, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In one embodiment, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from:

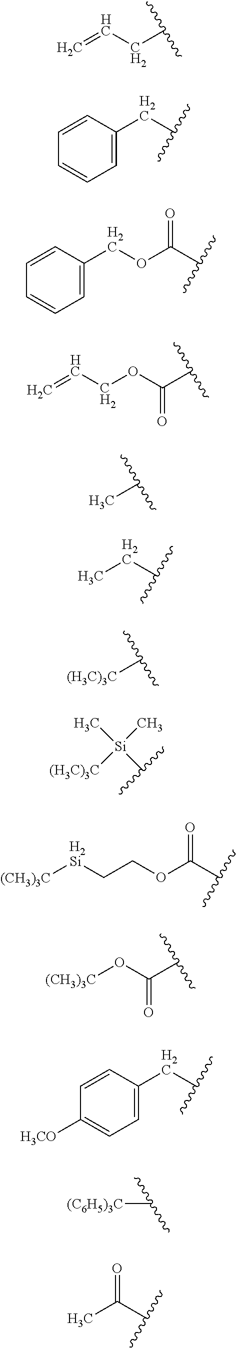

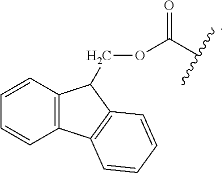 [SMILES: *C(=O)OCC1c2ccccc2-c2ccccc21]

Other protecting groups, plus a detailed description of techniques applicable to the creation of protecting groups and their removal are described in Greene & Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure.

### Methods of the Invention

The invention includes a method of treating or preventing cancer in a subject in need thereof. The method comprises administering to the subject an effective amount of a therapeutic composition comprising a compound of the invention. Cancers that may be treated include tumors that are not vascularized, or not yet substantially vascularized, as well as vascularized tumors. The cancers may comprise non-solid tumors (such as hematological tumors, for example, leukemias and lymphomas) or may comprise solid tumors. Types of cancers to be treated with the compositions of the invention include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

Hematologic cancers are cancers of the blood or bone marrow. Examples of hematological (or hematogenous) cancers that can be treated with the compositions of the invention include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, that can be treated with the compositions of the invention, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases.

In one embodiment, the cancer is selected from the group consisting of lung cancer, colon cancer, colorectal cancer, melanoma, breast cancer, ovarian cancer, prostate cancer, liver cancer, pancreatic cancer, CNS tumors (including brain tumors), neuroblastoma, leukemia, bone cancer, intestinal cancer, lymphoma, and combinations thereof. In one embodiment, the cancer is pancreatic cancer. In one embodiment, the cancer is prostate cancer. In one embodiment, the method further comprises administering to the subject an additional therapeutic agent. In one embodiment, the therapeutic agent is gemcitabine.

The invention also includes a method of treating or preventing pain or inflammation in a subject in need thereof. The method comprises administering to the subject an effective amount of a therapeutic composition comprising a compound of the invention. In one embodiment, the inflammation is selected from the group consisting of arthritic disorders, psoriasis, allergies, opioid tolerance, Crohn's Disease, migraine headaches, periarteritis nodosa, thyroiditis, aplastic anemia, Hodgkin's disease, scleredoma, rheumatic fever, type I diabetes, neuromuscular junction disease including myasthenia gravis, white matter disease including multiple sclerosis, sarcoidosis, nephrotic syndrome, Behcet's syndrome, polymyositis, gingivitis, nephritis, hypersensitivity, swelling occurring after injury including brain edema, and myocardial ischemia. In one embodiment, the arthritic disorder is selected from the group consisting of rheumatoid arthritis, spondyloarthropathies, gouty arthritis, osteoarthritis, systemic lupus erythematosus and juvenile arthritis. In one embodiment, the method further comprises administering to the subject an additional therapeutic agent.

In one embodiment, the pain is selected from the group consisting of pain resulting from cancer, fever and inflammation in a variety of conditions including rheumatic fever, influenza and other viral infections including common cold, low back and neck pain, dysmenorrhea, headache, toothache, sprains and strains, myositis, neuralgia, synovitis, arthritis, including rheumatoid arthritis, degenerative joint diseases (osteoarthritis), gout and ankylosing spondylitis, bursitis, burns, and trauma following surgical and dental procedures. In one embodiment, the method further comprises administering to the subject an additional therapeutic agent.

The invention also includes a method of treating or preventing a disease or disorder associated with the NF-κB pathway in a subject in need thereof. The method comprises administering to the subject an effective amount of a therapeutic composition comprising a compound of the invention. Non-limiting examples of diseases or disorder associated with reactive oxygen species include ischemic diseases, inflammatory diseases, autoimmune diseases, cancer metastasis and invasion, and cachexia.

The invention also includes a method of treating or preventing a disease or disorder associated with reactive oxygen species (ROS) in a subject in need thereof. The method comprises administering to the subject an effective amount of a therapeutic composition comprising a compound of the invention. Non-limiting examples of diseases or disorder associated with reactive oxygen species include arteriosclerosis, myocardial infarction, diabetes, and cancer.

In one embodiment, administering the compound of the invention to the subject allows for administering a lower dose of the therapeutic agent compared to the dose of the therapeutic agent alone that is required to achieve similar results in treating or preventing cancer in the subject. For example, in one embodiment, the compound of the invention enhances the anti-cancer activity of the additional therapeutic compound, thereby allowing for a lower dose of the therapeutic compound to provide the same effect. In another embodiment, administering the compound of the invention to the subject allows for administering a lower dose of the therapeutic agent compared to the dose of the therapeutic agent alone that is required to achieve similar results in treating or preventing pain or inflammation in the subject.

In one embodiment, the compound of the invention and the therapeutic agent are co-administered to the subject. In another embodiment, the compound of the invention and the therapeutic agent are co-formulated and co-administered to the subject.

In one embodiment, the subject is a mammal. In another embodiment, the mammal is a human.

### Combination Therapies

The compounds of the present invention are intended to be useful in combination with one or more additional compounds. In certain embodiments, these additional compounds may comprise compounds of the present invention or therapeutic agents known to treat or reduce the symptoms or effects of cancer. Such compounds include, but are not limited to, chemotherapeutics and the like. In other embodiments, these additional compounds may comprise therapeutic agents known to treat or reduce the symptoms or effects of pain or inflammation.

In one embodiment, the compound of Formula (I) is used in combination with any known FDA-approved cancer drug. In one embodiment, the invention provides a method to treat cancer comprising treating the subject prior to, concurrently with, or subsequently to the administration of a compound disclosed herein, with a complementary therapy for the cancer, such as surgery, chemotherapy, chemotherapeutic agent, radiation therapy, or hormonal therapy or a combination thereof.

Chemotherapeutic agents include cytotoxic agents (e.g., 5-fluorouracil, cisplatin, carboplatin, methotrexate, daunorubicin, doxorubicin, vincristine, vinblastine, oxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci), cytotoxic alkylating agents (e.g., busulfan, chlorambucil, cyclophosphamide, melphalan, or ethylesulfonic acid), alkylating agents (e.g., asaley, AZQ, BCNU, busulfan, bisulphan, carboxyphthalatoplatinum, CBDCA, CCNU, CHIP, chlorambucil, chlorozotocin, cis-platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864), antimitotic agents (e.g., allocolchicine, Halichondrin M, colchicine, colchicine derivatives, dolastatin 10, maytansine, rhizoxin, paclitaxel derivatives, paclitaxel, thiocolchicine, trityl cysteine, vinblastine sulfate, and vincristine sulfate), plant alkaloids (e.g., actinomycin D, bleomycin, L-asparaginase, idarubicin, vinblastine sulfate, vincristine sulfate, mitramycin, mitomycin, daunorubicin, VP-16-213, VM-26, navelbine and taxotere), biologicals (e.g., alpha interferon, BCG, G-CSF, GM-CSF, and interleukin-2), topoisomerase I inhibitors (e.g., camptothecin, camptothecin derivatives, and morpholinodoxorubicin), topoisomerase II inhibitors (e.g., mitoxantron, amonafide, m-AMSA, anthrapyrazole derivatives, pyrazoloacridine, bisantrene HCL, daunorubicin, deoxydoxorubicin, menogaril, N,N-dibenzyl daunomycin, oxanthrazole, rubidazone, VM-26 and VP-16), and synthetics (e.g., hydroxyurea, procarbazine, o,p′-DDD, dacarbazine, CCNU, BCNU, cis-diamminedichloroplatimun, mitoxantrone, CBDCA, levamisole, hexamethylmelamine, all-trans retinoic acid, gliadel and porfimer sodium).

Antiproliferative agents are compounds that decrease the proliferation of cells. Antiproliferative agents include alkylating agents, antimetabolites, enzymes, biological response modifiers, miscellaneous agents, hormones and antagonists, androgen inhibitors (e.g., flutamide and leuprolide acetate), antiestrogens (e.g., tamoxifen citrate and analogs thereof, toremifene, droloxifene and roloxifene), Additional examples of specific antiproliferative agents include, but are not limited to levamisole, gallium nitrate, granisetron, sargramostim strontium-89 chloride, filgrastim, pilocarpine, dexrazoxane, and ondansetron.

The compound of the invention can be administered alone or in combination with other anti-tumor agents, including cytotoxic/antineoplastic agents and anti-angiogenic agents. Cytotoxic/anti-neoplastic agents are defined as agents which attack and kill cancer cells. Some cytotoxic/anti-neoplastic agents are alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents are antimetabolites for tumor cells, e.g., cytosine arabinoside, fluorouracil, methotrexate, mercaptopuirine, azathioprime, and procarbazine. Other cytotoxic/anti-neoplastic agents are antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents are mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents are well known to those of skill in the art. Suitable anti-angiogenic agents for use in the methods and compositions of the present disclosure include anti-VEGF antibodies, including humanized and chimeric antibodies, anti-VEGF aptamers and antisense oligonucleotides. Other known inhibitors of angiogenesis include angiostatin, endostatin, interferons, interleukin 1 (including alpha and beta) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

Other anti-cancer agents that can be used in combination with the disclosed compounds include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; albumin-bound paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; imilimumab; mirtazapine; BrUOG 278; BrUOG 292; RAD0001; CT-011; folfirinox; tipifarnib; R115777; LDE225; calcitriol; AZD6244; AMG 655; AMG 479; BKM120; mFOLFOX6; NC-6004; cetuximab; IM-C225; LGX818; MEK162; BBI608; MEDI4736; vemurafenib; ipilimumab; ivolumab; nivolumab; panobinostat; leflunomide; CEP-32496; alemtuzumab; bevacizumab; ofatumumab; panitumumab; pembrolizumab; rituximab; trastuzumab; STAT3 inhibitors (e.g., STA-21, LLL-3, LLL12, XZH-5, S31-201, SF-1066, SF-1087, STX-0119, cryptotanshinone, curcumin, diferuloylmethane, FLLL11, FLLL12, FLLL32, FLLL62, C3, C30, C188, C188-9, LY5, OPB-31121, pyrimethamine, OPB-51602, AZD9150, etc.); hypoxia inducing factor 1 (HIF-1) inhibitors (e.g., LW6, digoxin, laurenditerpenol, PX-478, RX-0047, vitexin, KC7F2, YC-1, etc.) zinostatin stimalamer, Lynparza (olaparib), talazoparib, niraparib, and rucaparib.

In non-limiting examples, the compounds of the invention may be used in combination with one or more therapeutic agents (or a salt, solvate or prodrug thereof).

In certain embodiments, the compound of the invention may be administered to a subject in conjunction with (e.g. before, simultaneously, or following) any number of relevant treatment modalities including chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-815, 1991; Henderson et al., Immun. 73:316-321, 1991; Bierer et al., Curr. Opin. Immun. 5:763-773, 1993). In a further embodiment, the compounds of the present invention are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the compounds of the present invention are administered following B-cell ablative therapy such as agents that react with CD20, e.g., Rituxan. In another embodiment, the compounds of the present invention are administered in conjunction with Ospemifene, Tamoxifen, Raloxifene, or other drugs such as ICI 182,780 and RU 58668. Tamoxifen and Raloxifene may act as partial antiestrogens, and the drugs such as ICI 182,780 and RU 58668 may act as full antiestrogens. In another embodiment, the compounds of the invention are administered in conjunction with aromatase inhibitors. Non-limiting examples of aromatase inhibitors include Exemestane, Letrozole, and Anastrozole. In one embodiment, the therapeutic agent is gemcitabine.

In certain embodiments, the compounds of the invention may be administered to a subject in conjunction with (e.g. before, simultaneously, or following) an anti-inflammatory agent selected from the group consisting of nonsteroidal agents (“NSAIDS”) such as salicylates (e.g., salsalate, mesalamine, diflunisal, choline magnesium trisalicylate), diclofenac, diflunisal, etodolac, fenoprofen, flurbiprofen, ibuprofen, indomethacin, mefenamic acid, nabumetone, naproxen, piroxicam, phenyl butazone, ketoprofen, S-ketoprofen, ketorolac tromethamine, sulindac, tolmetin). Other anti-inflammatory drugs include steroidal agents such as beclomethasone, betamethasone, cortisone, dexamethasone, fluocinolone, flunisolide, fluticasone proprionate, fluorinated-corticoids, triamcinolone-diacetate, hydrocortisone, prednisolone, methylprednisolone, and prednisone. Immunosuppressive agents (e.g., adrenocorticosteroids, cyclosporin), antihistamines and decongestants (e.g., astemizole (histamine I II-receptor antagonist), azatadine, brompheniramine, clemastine, chlorpheniramine, cromolyn, cyproheptadine, diphenylimidazole, diphenhydramine hydrochloride, hydroxyzine, glycyrrhetic acid, homochlorocyclizine hydrochloride, ketotifen, loratadine, naphazoline, phenindamine, pheniramine, promethazine, terfenadine, trimeprazine, tripelennamine, tranilast, and the decongestants phenylpropanolamine and pseudoephedrine. In one embodiment, the therapeutic agent is a nonsteroidal anti-inflammatory drug (NSAID), as would be understood by one of ordinary skill in the art.

A synergistic effect may be calculated, for example, using suitable methods such as, for example, the Sigmoid-Eₘₐₓ equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

In certain embodiments, the compounds of compounds of Formula (I) may be administered to a subject in conjunction with cancer drugs that target DNA repair factors (i.e. PARP1, PARG, ATM, ATR, DNApk, RAD51, CHK1, WEE1, topoisomerase I, topoisomerase II) and/or act as genotoxic agents (i.e. chemotherapies and radiation/radiotherapy, proton therapy) and induce DNA damage. Compounds of Formula I may be especially useful as radiosensitizers or chemosensitizers, and act synergistically with PARP inhibitors (i.e. olaparib, niraparib, talazoparib, rucaparib) and topoisomerase inhibitors (etoposide, camptothecin, toptecan, doxorubicin, daunorubicin) and ATR inhibitors and DNApk inhibitors.

### Administration/Dosage/Formulations

The regimen of administration may affect what constitutes an effective amount. The therapeutic formulations may be administered to the subject either before or after the onset of cancer. Further, several divided dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, such as a mammal, (e.g., human), may be carried out using known procedures, at dosages and for periods of time effective to treat cancer in the patient. An effective amount of the therapeutic compound necessary to achieve a therapeutic effect may vary according to factors such as the state of the disease or disorder in the patient; the age, sex, and weight of the patient; and the ability of the therapeutic compound to treat a cancer in the patient. Dosage regimens may be adjusted to provide the optimum therapeutic response. For example, several divided doses may be administered daily. In another example, the dose may be proportionally reduced as indicated by the exigencies of the therapeutic situation. A non-limiting example of an effective dose range for a therapeutic compound of the invention is from about 1 mg/kg to about 5,000 mg/kg of body weight/per day. One of ordinary skill in the art would be able to assess the relevant factors and make the determination regarding the effective amount of the therapeutic compound without undue experimentation.

Actual dosage levels of the active ingredients in the pharmaceutical compositions of this invention may be varied to obtain an amount of the active ingredient that is effective to achieve the desired therapeutic response for a particular patient, composition, and mode of administration, without generating excessive side effects in the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical professional, e.g., physician or veterinarian, having ordinary skill in the art may readily determine and prescribe the effective amount of the pharmaceutical composition required. For example, the physician or veterinarian could start with a dosage of the compound of the invention in the pharmaceutical composition at a level that is lower than the level required to achieve the desired therapeutic effect, and then increase the dosage over time until the desired effect is achieved.

In particular embodiments, it is advantageous to formulate the compound in dosage unit form for ease of administration and uniformity of dosage. “Dosage unit form” as used herein refers to a physically discrete unit containing a predetermined quantity of therapeutic compound calculated to produce the desired therapeutic effect, in association with the required pharmaceutical vehicle. The dosage unit forms of the invention can be selected based upon (a) the unique characteristics of the therapeutic compound and the particular therapeutic effect to be achieved, and (b) the limitations inherent in the art of compounding/formulating such a therapeutic compound for the treatment of cancer in a patient.

In one embodiment, the compositions of the invention are formulated using one or more pharmaceutically acceptable excipients or carriers. In one embodiment, the pharmaceutical compositions of the invention comprise a therapeutically effective amount of a compound of the invention and a pharmaceutically acceptable carrier.

The carrier may be a solvent or dispersion medium containing, for example, water, ethanol, polyol (for example, glycerol, propylene glycol, and liquid polyethylene glycol, and the like), vegetable oils, and suitable mixtures thereof. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In some embodiments, it is useful to include isotonic agents, for example, sugars, sodium chloride, or polyalcohols such as mannitol and sorbitol, in the composition. Prolonged absorption of the injectable compositions can be achieved by including in the composition an agent which delays absorption, for example, aluminum monostearate or gelatin. In one embodiment, the pharmaceutically acceptable carrier is DMSO, alone or in combination with other carriers.

The therapeutically effective amount or dose of a compound of the present invention depends on the age, sex and weight of the patient, the current medical condition of the patient and the severity of the cancer in the patient being treated. The skilled artisan is able to determine appropriate doses depending on these and other factors.

The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

Doses of the compound of the invention for administration may be in the range of from about 1 μg to about 10,000 mg, from about 20 μg to about 9,500 mg, from about 40 μg to about 9,000 mg, from about 75 μg to about 8,500 mg, from about 150 μg to about 7,500 mg, from about 200 μg to about 7,000 mg, from about 3050 μg to about 6,000 mg, from about 500 μg to about 5,000 mg, from about 750 μg to about 4,000 mg, from about 1 mg to about 3,000 mg, from about 10 mg to about 2,500 mg, from about 20 mg to about 2,000 mg, from about 25 mg to about 1,500 mg, from about 30 mg to about 1,000 mg, from about 40 mg to about 900 mg, from about 50 mg to about 800 mg, from about 60 mg to about 750 mg, from about 70 mg to about 600 mg, from about 80 mg to about 500 mg, and any and all whole or partial increments therebetween.

In some embodiments, the dose of a compound of the invention is from about 1 mg to about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, the dosage of a second compound as described elsewhere herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

The compounds for use in the method of the invention may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

In one embodiment, the compositions of the invention are administered to the patient from about one to about five times per day or more. In various embodiments, the compositions of the invention are administered to the patient, 1-7 times per day, 1-7 times every two days, 1-7 times every 3 days, 1-7 times every week, 1-7 times every two weeks, and 1-7 times per month. It is readily apparent to one skilled in the art that the frequency of administration of the various combination compositions of the invention will vary from individual to individual depending on many factors including, but not limited to, age, the disease or disorder to be treated, the severity of the disease or disorder to be treated, gender, overall health, and other factors. Thus, the invention should not be construed to be limited to any particular dosing regime and the precise dosage and composition to be administered to any patient is determined by the medical professional taking all other factors about the patient into account.

In the case wherein the patient's status does improve, upon the doctor's discretion the administration of the inhibitor of the invention is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's condition has occurred, a maintenance dose is administered if necessary. Subsequently, the dosage or the frequency of administration, or both, may be reduced to a level at which the improved disease is retained. In some embodiments, a patient may require intermittent treatment on a long-term basis, or upon any recurrence of the disease or disorder.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

In one embodiment, the present invention is directed to a packaged pharmaceutical composition comprising a container holding a therapeutically effective amount of a compound of the invention, alone or in combination with a second pharmaceutical agent; and instructions for using the compound to treat or prevent cancer in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions of the invention include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans) buccal, (trans) urethral, vaginal (e.g., trans- and perivaginally), (intra) nasal and (trans) rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral administration, suitable forms include tablets, dragees, liquids, drops, suppositories, or capsules, caplets and gelcaps. The compositions formulated for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds of the invention may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation involves the use of materials that are solid or semi-solid at room temperature (i.e., having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e., drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) melt.

The present invention also includes a multi-layer tablet comprising a layer providing for the delayed release of one or more compounds of the invention, and a further layer providing for the immediate release of a medication for treatment of G-protein receptor-related diseases or disorders. Using a wax/pH-sensitive polymer mix, a gastric insoluble composition may be obtained in which the active ingredient is entrapped, ensuring its delayed release.

### Parenteral Administration

For parenteral administration, the compounds of the invention may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Additional Administration Forms

Additional dosage forms of this invention include dosage forms as described in U.S. Pat. Nos. 6,340,475; 6,488,962; 6,451,808; 5,972,389; 5,582,837; and 5,007,790. Additional dosage forms of this invention also include dosage forms as described in U.S. Patents Applications Nos. 20030147952; 20030104062; 20030104053; 20030044466; 20030039688; and 20020051820. Additional dosage forms of this invention also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757

### Controlled Release Formulations and Drug Delivery Systems

In one embodiment, the formulations of the present invention may be, but are not limited to, short-term, rapid-offset, as well as controlled, for example, sustained release, delayed release and pulsatile release formulations.

The term sustained release refers to a drug formulation that provides for gradual release of a drug over an extended period of time, and that may, although not necessarily, result in substantially constant blood levels of a drug over an extended time period. The period of time may be as long as a day, a week, or a month or more and should be a release which is longer that the same amount of agent administered in bolus form. The term delayed release is used herein in its conventional sense to refer to a drug formulation that provides for an initial release of the drug after some delay following drug administration and that mat, although not necessarily, includes a delay of from about 10 minutes up to about 12 hours.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation.

In one embodiment of the invention, the compounds of the invention are administered to a patient, alone or in combination with another pharmaceutical agent, using a sustained release formulation.

The term pulsatile release refers to a drug formulation that provides release of the drug in such a way as to produce pulsed plasma profiles of the drug after drug administration.

The term immediate release refers to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Therapeutic Utility

The compounds of the invention, subgroups and examples thereof, are inhibitors of Polq polymerase activity, and which may be useful in preventing or treating disease states or conditions described herein. In addition the compounds of the invention, and subgroups thereof, will be useful in preventing or treating diseases or condition mediated by Polq.

References to the preventing or prophylaxis or treatment of a disease state or condition such as cancer include within their scope alleviating or reducing the incidence of cancer. Thus, for example, it is envisaged that the compounds of the invention will be useful in alleviating or reducing the incidence of cancer.

In one embodiment, it is expected that compounds of Formula I (herein referred to as Polq inhibitors (Polqi)) will show preferential killing of BRCA deficient cells, or another words cancer cells that are defective in homology-directed repair (HDR). This is due to the fact that BRCA deficient ovarian cancer cells were shown to be dependent on Polq for their survival in the presence of genotoxic agents. This synthetic lethal relationship between Polq and HDR was further demonstrated in mouse models. Most importantly, the DNA synthesis activity of Polq was shown to promote the survival of BRCA deficient cells which strongly suggests that pharmacological inhibition of the polymerase domain by compounds presented herein will selectively kill BRCA deficient cancer cells which include but are not limited to cancers originating in the prostate, breast, ovary, pancreas.

Certain hematological cancers, including but not limited to acute myeloid leukemia (AML), have been shown to possess defects in HDR as a result of the effects of particular genetic mutations (i.e. BCR-ABL) or due to certain treatment regiments.

In one embodiment, Polqi presented herein may also be particularly effective in AML or other hematological cancers.

Several factors important for HDR may be defective and/or downregulated in cancer cells, including but not limited to Mre11, Rad50, Nbs1, CtIP, Exo1, PALB2, BARD1, RAD51B, RAD51C, RAD51D, XRCC2 and XRCC3. Therefore, in one embodiment, cancer cells defective or downregulated in one or more of these HDR factors will be susceptible to the Polq inhibitors described herein.

In one embodiment, said HDR genes are selected from any of: ATM, ATR, BRCA1, BRCA2, BARD1, RAD51C, RAD50, CHEK1, CHEK2, FANCA, FANCB, FANCC, FANCD2, FANCE, FANCF, FANCG, FANCI, FANCL, FANCM, PALB2 (FANCN), FANCP (BTBD12), ERCC4 (FANCQ), PTEN, CDK12, MRE1 1, NBS1, NBN, CLASPIN, BLM, WRN, SMARCA2, SMARCA4, LIG1, RPA1, RPA2, BRIP1 and PTEN.

Additionally, Polq inactivation in combination with suppression of DDR factors RAD54 or FANCJ also results in synthetic lethality. Thus, in one embodiment it is expected that Polqi described herein will preferentially kill cancer cells with defects or downregulation of RAD54 and/or FANCJ.

Furthermore, recent studies demonstrate that inactivation of Polq in combination with inhibition of the DDR factor ATR also results in a significant reduction in cell proliferation. Semi-synthetic lethality between Polq and ATM was also identified in earlier studies. Thus, in one embodiment it is expected that Polqi described herein will show preferential killing of cancer cells defective in or downregulated in ATR or ATM DDR factors. In one embodiment, it is also expected that Polqi described herein will exhibit synergistic or additive anti-proliferation effects when combined with ATR inhibitors or ATM inhibitors. In another embodiment, it is also expected that Polqi described herein will show effective killing of cancer cells exhibiting replicative stress, especially when combined with other anti-cancer agents that exacerbate replicative stress, including but not limited to gemcitabine, ATR inhibitors, cytarabine, topoisomerase inhibitors (i.e. etoposide), cisplatin, etc.

Polq was also shown to confer resistance to ionizing radiation (IR), bleomycin, cisplatin, mitomycin C, and topoisomerase inhibitors (etoposide, camptothecin). Therefore, in another embodiment it is expected that Polqi described herein will promote cancer cell sensitivity to a variety of anti-cancer agents including but not limited to IR, bleomycin, cisplatin, mitomycin C and topoisomerase inhibitors.

PARP inhibitors (PARPi) and Polq inactivation both reduce cancer cell resistance to IR. Thus, in another embodiment it is expected that combining Polqi described herein with PARPi will sensitize cancer cells to IR and overcome cancer cell resistance to IR. Furthermore, it has been shown that suppression of Polq combined with a DNApk inhibitor causes cancer cell sensitivity to IR. Thus, in another embodiment it is expected that Polqi described here in will show synergistic anti-proliferation effects when combined with DNApk inhibitors and IR or other anti-cancer agents that cause DNA double-strand breaks.

It has also been shown that suppression of Polq expression confers cellular sensitivity to PARP inhibition in HDR defective cancer cells. Therefore, in one embodiment it is expected that Polqi described herein will act synergistically with PARP inhibitors (PARPi), especially in HDR defective cells. In one embodiment, it is expected that Polqi combined with PARPi including but not limited to Lynparza (olaparib), talazoparib, niraparib, and rucaparib will potentiate the effects of PARPi in solid tumors and hematological malignancies. In one embodiment, is expected that Polqi described herein when combined with PARPi will suppress cancer cell resistance to PARPi.

In one embodiment, Polqi described herein are expected to induce synthetic lethality in cancer cells with defects in or suppression of the expression of non-homologous end-joining NHEJ factors such as LIG4 or KU70/80. In one embodiment, said non-homologous end-joining genes are selected from any one or more of: LIG4, NHEJ1, POLL, POLM, PRKDC, XRCC4, XRCC5, XRCC6, and DCLRE1C. According to a further aspect of the invention there is a provided a compound of formula (I) as defined herein for use in the treatment of tumours which have elevated ligase Ilia levels, reduced ligase IV levels and increased dependence upon MMEJ (altEJ) DSB repair.

Suppression of Polq expression enhances and reduces the off-target effects of genome engineering by CRISPR-Cas9 type RNA-guided endonucleases, described in WO 2017/062754. Thus, in one embodiment it is expected that Polqi described herein will benefit CRISPR-Cas9 based genome engineering by reducing off-target effects and thus increase the fidelity and safety of CRISPR-Cas9 type RNA-guided genome engineering for therapeutics and basic research applications. In one embodiment, it is expected that combining Polqi described herein with DNApk inhibitors will have an even greater effect on increasing the fidelity and safety of CRISPR-Cas9 type RNA-guided genome engineering for therapeutic and basic research applications.

In one aspect, the present invention relates to a method of inhibiting the expression or stability of DNA polymerase theta (Polq) in a subject, the method comprising administering to the subject a compound of Formula (I) or a tautomeric or a stereochemically isomeric form, a pharmaceutically acceptable salt or a solvate thereof.

In one aspect, the present invention relates to a method of treating cancer in a subject in need thereof, the method comprising administering to the subject a compound of Formula (I) or a tautomeric or a stereochemically isomeric form, a pharmaceutically acceptable salt or a solvate thereof. In one embodiment, the method further comprises the step of administering to the subject one or more PARP inhibitors, one or more topoisomerase inhibitors, or an anti-cancer radiotherapy.

In one aspect, the present invention relates to a method of inhibiting the activity of DNA polymerase theta (Polq), the method comprising the step of contacting Polq with a compound of Formula (I) or a tautomeric or a stereochemically isomeric form, a pharmaceutically acceptable salt or a solvate thereof.

### EXPERIMENTAL EXAMPLES

Those skilled in the art recognize, or are able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### Example 1: 2-[1-(2-hydroxyethyl)-1H-1,2,3-triazol-4-yl]-4,6-bis(trifluoromethyl)phenyl N-(4-fluorophenyl)-N-methylcarbamate

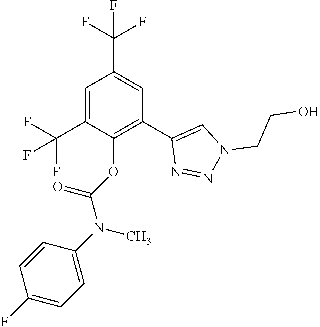 [SMILES: CN(C(=O)Oc1c(-c2cn(CCO)nn2)cc(C(F)(F)F)cc1C(F)(F)F)c1ccc(F)cc1]

### General Synthesis Scheme

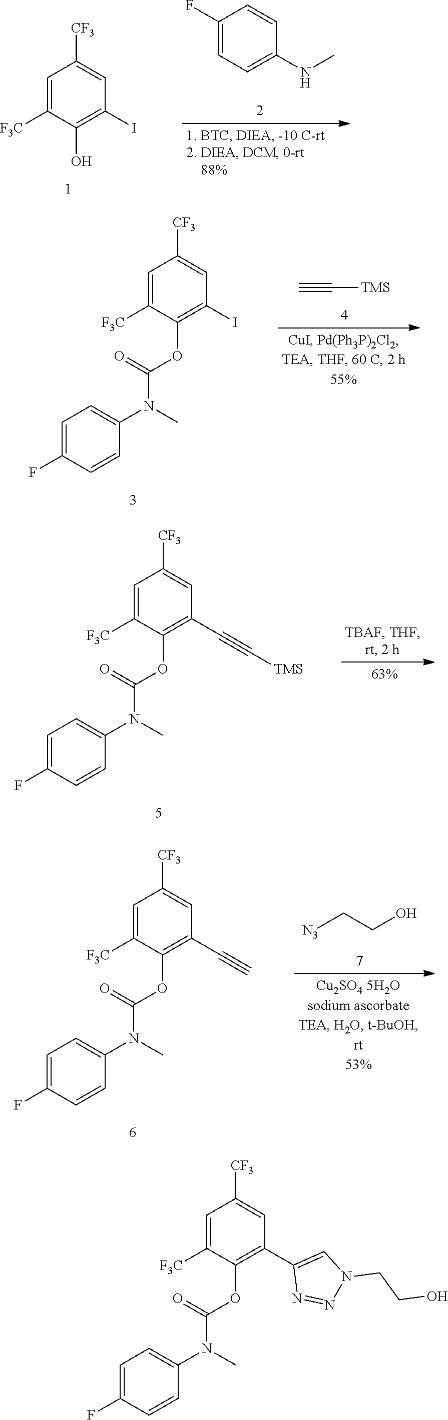

### Step 1: 2-iodo-4,6-bis(trifluoromethyl)phenol

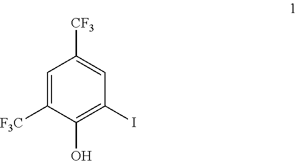 [SMILES: Oc1c(I)cc(C(F)(F)F)cc1C(F)(F)F]

A solution of 2,4-bis(trifluoromethyl)phenol (1.00 g, 4.35 mmol) in THF:H₂O (3:1, 24 mL) was cooled to 0° C. in an ice bath under nitrogen. Iodine (1.18 g, 4.64 mmol) and Na₂CO₃ (491 mg, 4.64 mmol) were added sequentially. The ice bath was removed and the reaction was allowed to warm to RT with stirring overnight. The reaction solution was cooled to 0° C. in an ice bath, quenched with saturated aqueous sodium metabisulfite and stirred at 0° C. until all of the solution turned yellow in color. This mixture was extracted with EtOAc (3×). The combined organic extracts were washed with water and brine, dried over anhydrous sodium sulfate and concentrated. The crude product was purified by column chromatography on silica gel using a gradient solvent system of 0 to 10% of ethyl acetate in hexanes to afford the titled compound as colorless crystalline solid (711 mg, 46%). ¹H NMR (400 MHz, CDCl₃) δ 8.10 (s, 1H), 7.82 (s, 1H), 6.15 (s, 1H).

### Step 2: 2-iodo-4,6-bis(trifluoromethyl)phenyl N-(4-fluorophenyl)-N-methylcarbamate

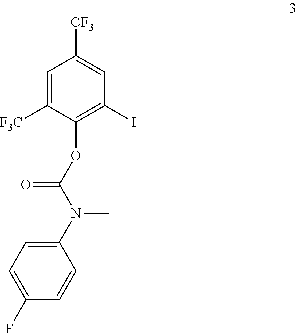 [SMILES: CN(C(=O)Oc1c(I)cc(C(F)(F)F)cc1C(F)(F)F)c1ccc(F)cc1]

To a solution of compound 1 (1.0 g, 2.82 mmol, 1.0 eq) in DCM (10 mL) at −30° C. under N₂ was added DIEA (545 mg, 4.2 mmol, 1.5 eq), and a solution of triphosgene (418 mg, 1.4 mmol, 0.5 eq) in DCM (2 mL). The reaction mixture was stirred at rt for 1 h. The reaction solution was concentrated under reduced pressure, and dissolved in DCM (3 mL). The solution was used for next step without further purification.

To a solution of compound 2 (422 mg, 3.38 mmol, 1.2 eq) in DCM (5 mL), was added the above residue solution at 0 C. The mixture was stirred for 1 h at rt under N2. After completion of the reaction, HCl (1 N, 15 mL) was added. Then the mixture was extracted with DCM (20 mL×2). The combined organic layer was washed with brine, dried with sodium sulfate and filtered. The filtrate was concentrated under reduced pressure to give a residue. The residue was purified by column chromatography to give compound 3 (886 mg, 88%) as off-white solid.

### Step 3

### Procedure for the Preparation of Compound 5

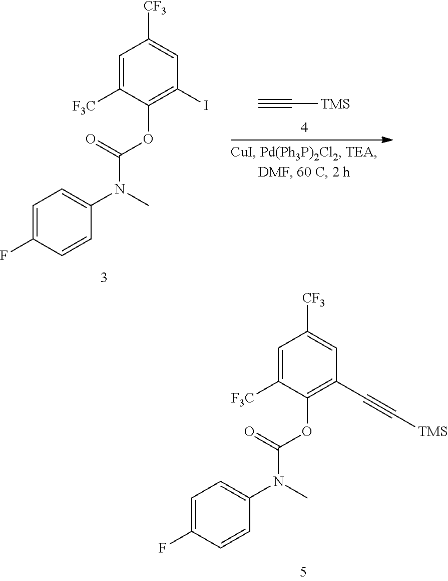

To a solution of compound 3 (100 mg, 0.19 mmol, 1.0 eq) in DMF (3 mL) was added compound 4 (54 mg, 0.295 mmol, 1.5 eq), CuI (8 mg, 0.039 mmol, 0.2 eq), TEA (40 mg, 0.396 mmol, 2 eq), Pd(PPh₃)₂Cl₂ (15 mg, 15%) under inert atmosphere. The reaction was stirred at 60° C. for 2 h. Then the mixture was extracted with DCM (20 mL×2). The combined organic layers were washed with brine, dried over sodium sulfate and filtered. The solution was concentrated under reduced pressure. The residue was purified by column chromatography to give compound 5 (55 mg, 55%) as off-white solid.

### Step 4

### Procedure for the Preparation of Compound 6

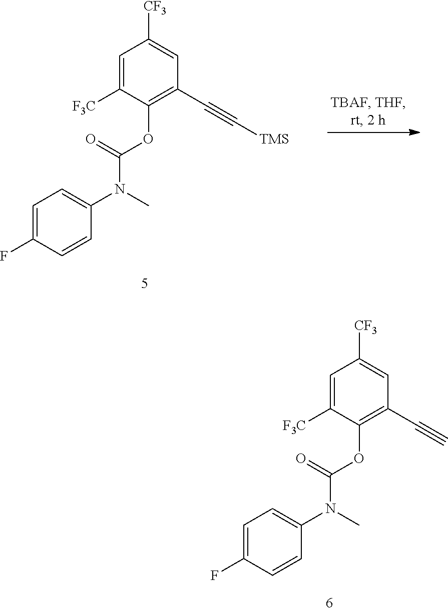 [SMILES: CN(C(=O)Oc1c(C#C[Si](C)(C)C)cc(C(F)(F)F)cc1C(F)(F)F)c1ccc(F)cc1 ; C#Cc1cc(C(F)(F)F)cc(C(F)(F)F)c1OC(=O)N(C)c1ccc(F)cc1]

To a solution of compound 5 (55 mg, 0.19 mmol, 1.0 eq) in THF (5 mL) at rt was added TBAF (1 M, 0.28 mL, 1.5 eq). The reaction mixture was stirred at rt for 2 h. Then the mixture was extracted with DCM (20 mL×2). The combined organic layer was washed with brine, dried over sodium sulfate and filtered. The filtrate was concentrated under reduced pressure. The residue was purified by column chromatography to give compound 6 (35 mg, 63%) as off-white solid.

### Step 5

### Procedure for the Preparation of 2-[1-(2-hydroxyethyl)-1H-1,2,3-triazol-4-yl]-4,6-bis(trifluoromethyl)phenyl N-(4-fluorophenyl)-N-methylcarbamate

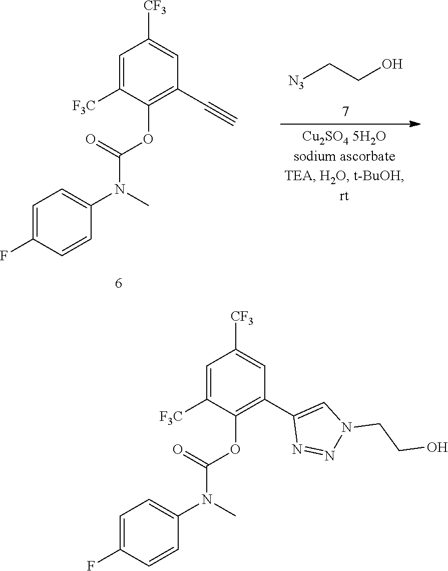 [SMILES: C#Cc1cc(C(F)(F)F)cc(C(F)(F)F)c1OC(=O)N(C)c1ccc(F)cc1 ; [N-]=[N+]=NCCO ; CN(C(=O)Oc1c(-c2cn(CCO)nn2)cc(C(F)(F)F)cc1C(F)(F)F)c1ccc(F)cc1]

To a solution of compound 6 (100 mg, 0.25 mmol, 1.0 eq) in t-BuOH/H₂O (3 mL/3 mL) was added compound 7 (23 mg, 0.25 mmol, 1.0 eq), sodium ascorbate (25 mg, 0.12 mmol, 0.5 eq), TEA (27 mg, 0.27 mmol, 1.1 eq), CuSO₄·5H₂O (19 mg, 0.074 mmol, 0.3 eq) under N₂ at rt. The reaction mixture was stirred for 2 h. Then the mixture was extracted with DCM (20 mL×2). The combined organic layer was washed with brine, dried over Na₂SO₄, filtered, and concentrated under reduced pressure. The residue was purified by column chromatography to give 2-[1-(2-hydroxyethyl)-1H-1,2,3-triazol-4-yl]-4,6-bis(trifluoromethyl)phenyl N-(4-fluorophenyl)-N-methylcarbamate (53 mg, 43%) as off-white solid.

LC-MS: 493 [M+1]⁺

¹H NMR (400 MHz, DMSO-d6): δ 8.67-8.35 (m, 2H), 8.11-8.04 (d, J=8 Hz, 2H), 7.57 (s, 1H), 7.32-7.30 (m, 2H), 7.20 (m, 1H), 5.21-5.15 (m, 2H), 4.53 (s, 2H), 3.83-3.82 (m, 2H), 3.50 (s, 1H), 3.21 (s, 2H).

### Example 2: 2-(2-oxo-2,3-dihydro-1H-imidazol-1-yl)-4,6-bis(trifluoromethyl)phenyl N-(4-fluorophenyl)-N-methylcarbamate

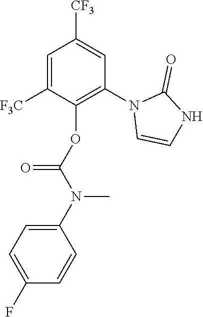 [SMILES: CN(C(=O)Oc1c(-n2cc[nH]c2=O)cc(C(F)(F)F)cc1C(F)(F)F)c1ccc(F)cc1]

### General Synthesis Scheme

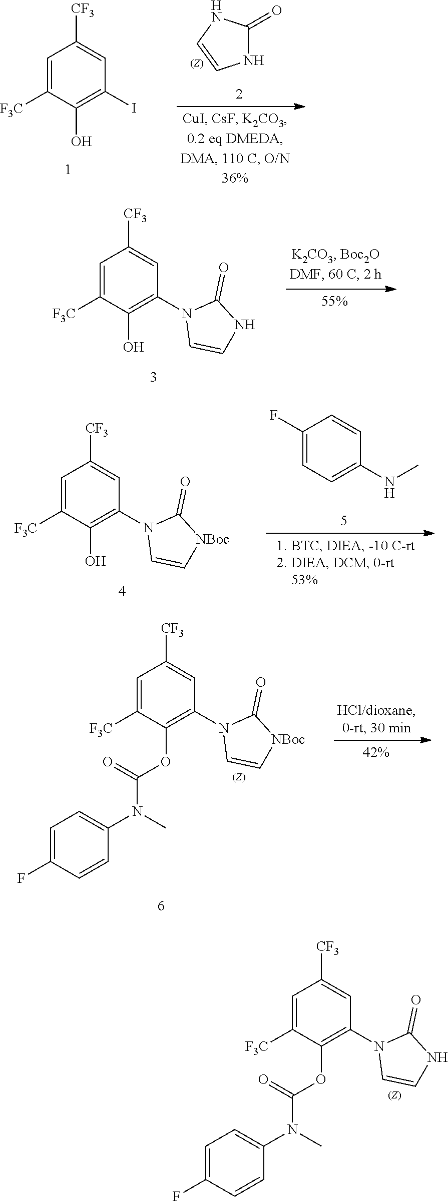

### Step 1

### Procedure for the Preparation of Compound 3 (1-[2-hydroxy-3,5-bis(trifluoromethyl)phenyl]-2,3-dihydro-1H-imidazol-2-one)

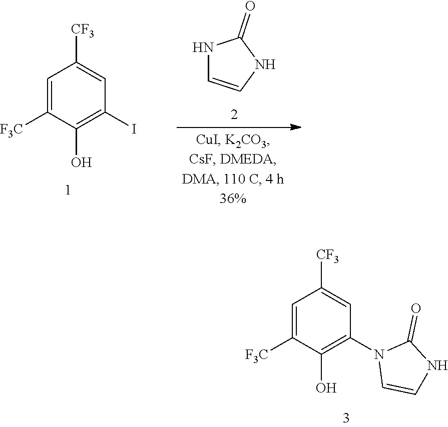 [SMILES: Oc1c(I)cc(C(F)(F)F)cc1C(F)(F)F ; O=c1[nH]cc[nH]1 ; O=c1[nH]ccn1-c1cc(C(F)(F)F)cc(C(F)(F)F)c1O]

Compound 1 was synthesized as described above in Example 1. To a solution of compound 1 (1.0 g, 2.81 mmol, 1.0 eq) and compound 2 (474 mg, 5.63 mmol, 2.0 eq) in DMA (10 mL) at rt was added CuI (270 mg, 1.4 mmol, 0.5 eq), DMEDA (249 mg, 2.81 mmol, 1.0 eq), K2CO₃ (782 mg, 5.62 mmol, 2.0 eq), CsF (855 mg, 5.62 mmol, 2.0 eq). The reaction mixture was heated at 110° C. in an oil-bath for 4 h. After completion of the reaction monitored by TLC (R_{f} 0.3, PE:EA=2:1) and LC-MS, water (20 mL) was added. The mixture was adjusted to pH 6 with HCl (2 N). The mixture was extracted with ethyl acetate (30 mL×2). The combined organic layers were washed with brine, dried over Na₂SO₄ and filtered. The filtrate was concentrated under reduced pressure. The residue was purified by column chromatography (100-200 silica gel, 50% EA in PE) to afford compound 3 (320 mg, 36%) as pale-yellow solid.

### Step 2

### Procedure for the Preparation of Compound 4 (tert-butyl 3-[2-hydroxy-3,5-bis(trifluoromethyl)phenyl]-2-oxo-2,3-dihydro-1H-imidazole-1-carboxylate)

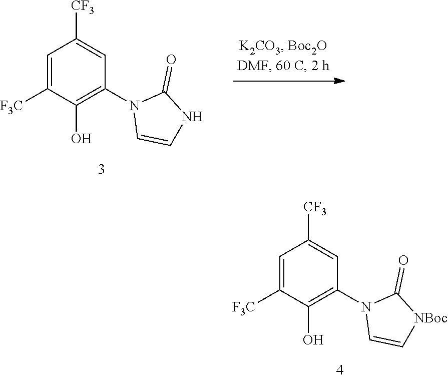 [SMILES: O=c1[nH]ccn1-c1cc(C(F)(F)F)cc(C(F)(F)F)c1O ; CC(C)(C)OC(=O)n1ccn(-c2cc(C(F)(F)F)cc(C(F)(F)F)c2O)c1=O]

To a solution of compound 3 (150 mg, 0.48 mmol, 1.0 eq) and Boc₂O (125 mg, 0.58 mmol, 1.2 eq) in DMF (10 mL) at rt was added K₂CO₃ (167 mg, 1.2 mmol, 2.5 eq). The reaction mixture was heated at 60° C. in an oil-bath for 2 h. The mixture was extracted with ethyl acetate (30 mL×2). The combined organic layer was washed with brine, dried over Na₂SO₄ and filtered. The solution was concentrated under reduced pressure to give a residue. The residue was purified by column chromatography (100-200 silica gel, 20-50% EA in PE) to afford compound 4 (110 mg, 55%) as pale-yellow solid.

### Step 3

### Procedure for the Preparation of Compound 6 (tert-butyl 3-(2-{[(4-fluorophenyl)(methyl)carbamoyl]oxy}-3.5-bis(trifluoromethyl)phenyl)-2-oxo-2,3-dihydro-1H-imidazole-1-carboxylate)

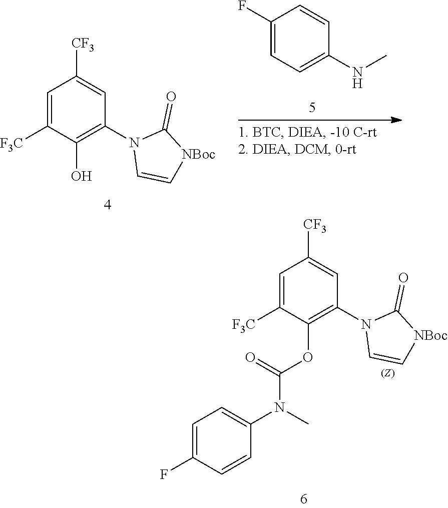

To a solution of compound 4 (110 mg, 0.35 mmol, 1.0 eq) in DCM (10 mL) at −10° C. was added DIEA (60 mg, 0.467 mmol, 1.5 eq) and triphosgene (117 mg, 0.395 mmol, 1.1 eq, in DCM (2 mL)). The reaction mixture was stirred at rt under N₂ for 1 h. The reaction solution was concentrated under reduced pressure to remove the solvent. Then DCM (3 mL) was added to above residue. The solution was used for next step.

To a solution of compound 5 (85 mg, 0.52 mmol, 1.5 eq) and DIEA (68 mg, 0.52 mmol, 1.5 eq) in DCM (5 mL) at 0° C. was added the above solution. The mixture was stirred at rt under N₂ for 1 h. After completion of the reaction checked by TLC (R_{f} 0.5, PE:EA=2:1) and LC-MS, HCl (1 N, 15 mL) was added. Then the mixture was extracted with DCM (20 mL×2). The combined organic layer was washed with brine, dried over Na₂SO₄ and filtered. The filtrate was concentrated under reduced pressure. The residue was purified by column chromatography (100-200 silica gel, 50% EA in PE as eluent) to give compound 6 (80 mg, 53%) as off-white solid.

### Step 4

### Procedure for the Preparation of 2-(2-oxo-2,3-dihydro-1H-imidazol-1-yl)-4,6-bis(trifluoromethyl)phenyl N-(4-fluorophenyl)-N-methylcarbamate

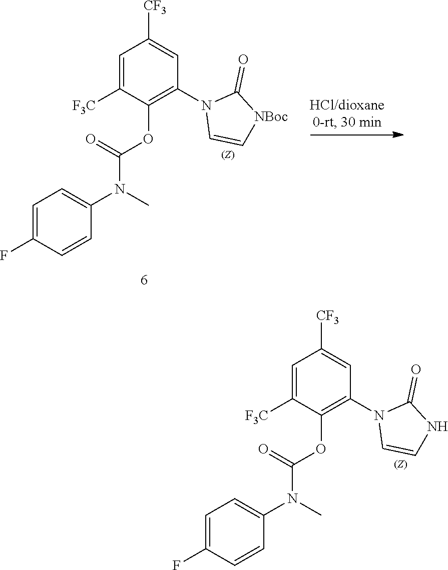 [SMILES: CN(C(=O)Oc1c(-n2ccn(C(=O)OC(C)(C)C)c2=O)cc(C(F)(F)F)cc1C(F)(F)F)c1ccc(F)cc1 ; CN(C(=O)Oc1c(-n2cc[nH]c2=O)cc(C(F)(F)F)cc1C(F)(F)F)c1ccc(F)cc1]

To a solution of HCl in dioxane (3 mL, 4 N) at rt was added compound 6 (80 mg, 0.14 mmol, 1.0 eq). The mixture was stirred for 30 min at rt under N₂. After completion of the reaction by TLC (R_{f} 0.3, PE:EA=1:1), Na₂CO₃ (aq) was added to adjust the solution to pH 8-9. The mixture was extracted with ethyl acetate (30 mL×2). The combined organic layers were washed with brine, dried over Na₂SO₄ and filtered. The filtration was concentrated under reduced pressure. The residue was purified by column chromatography (100-200 silica gel, 50% ethyl acetate in PE as eluent) to afford compound 2-(2-oxo-2,3-dihydro-1H-imidazol-1-yl)-4,6-bis(trifluoromethyl)phenyl N-(4-fluorophenyl)-N-methylcarbamate (28 mg, 42%) as pale-yellow solid.

LC-MS: 464.00 [M+1]⁺

¹H NMR (400 MHz, CD₃OD): δ 8.15-8.18 (m, 1H), 7.99-8.05 (m, 1H), 7.27-7.32 (m, 2H), 7.10-7.16 (m, 2H), 6.69 (s, 0.38H) 6.66 (s, 1H), 6.64 (s, 0.57H), 3.46 (s, 1H), 3.22 (s, 2H).

### Example 3

### 2-(1-(5-((2-(2,6-dioxopiperidin-3-yl)-1,3-dioxoisoindolin-4-yl)amino)-5-oxopentyl)-1H-1,2,3-triazol-4-yl)-4,6-bis(trifluoromethyl)phenyl (4-fluorophenyl) (methyl)carbamate

### Step 1

### (4-Fluorophenyl) (methyl) carbamic Chloride

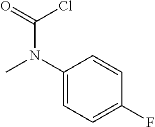

To a solution of 4-fluoro-N-methylaniline (0.58 g, 4.66 mmol) and pyridine (0.73 g, 9.34 mmol) in 12 mL of dichloromethane at 0° C., triphosgene (0.69 g, 2.33 mmol) dissolved in 6 mL dichloromethane was added dropwise under inert atmosphere. The reaction was stirred at ambient temperature (room temperature) for 2 h. The reaction mixture was diluted with 20 mL dichloromethane and extracted with 20 mL IN HCl. The organic layer was separated, dried over with anhydrous sodium sulfate, filtered and concentrated to a solid under reduced pressure. ¹H NMR (400 MHz, CDCl₃) δ 7.15 (m, 2H), 7.04 (m, 2H), 3.3 (s, 3H).; ESIMS: m/z 188.0 [(M+H)⁻].

### Step 2

### 2-iodo-4,6-bis(trifluoromethyl)phenol

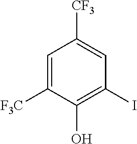 [SMILES: Oc1c(I)cc(C(F)(F)F)cc1C(F)(F)F]

A solution of 2,4-bis(trifluoromethyl)phenol (1.00 g, 4.35 mmol) in THF:H₂O (3:1, 24 mL) was cooled to 0° C. in an ice bath under nitrogen. Iodine (1.18 g, 4.64 mmol) and Na₂CO₃ (491 mg, 4.64 mmol) were added sequentially. The ice bath was removed, and the reaction was allowed to warm to RT with stirring overnight. The reaction solution was cooled to 0° C. in an ice bath, quenched with saturated aqueous sodium metabisulfite and stirred at 0° C. until all the solution turned yellow in color. This mixture was extracted with EtOAc (3×). The combined organic extracts were washed with water and brine, dried over anhydrous sodium sulfate and concentrated. The crude product was purified by column chromatography on silica gel using a gradient solvent system of 0 to 10% of ethyl acetate in hexanes to afford the titled compound as colorless crystalline solid (711 mg, 46%). ¹H NMR (400 MHz, CDCl₃) δ 8.10 (s, 1H), 7.82 (s, 1H), 6.15 (s, 1H).

### Step 3

### 2-iodo-4,6-bis(trifluoromethyl)phenyl (4-fluorophenyl) (methyl)carbamate

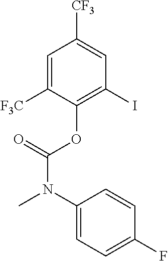 [SMILES: CN(C(=O)Oc1c(I)cc(C(F)(F)F)cc1C(F)(F)F)c1ccc(F)cc1]

2-iodo-4,6-bis(trifluoromethyl)phenol (570 mg, 1.60 mmol) and (4-fluorophenyl) (methyl)carbamic chloride (751 mg, 4.00 mmol) were dissolved into anhydrous pyridine (10 mL). This solution was stirred at 90° C. for 4 hours. The reaction was cooled to RT and concentrated down. The residual solid was partitioned between EtOAc and IN aqueous HCl. The aqueous phase was separated and extracted with EtOAc twice. The combined organic extracts were washed with brine, dried over anhydrous sodium sulfate and concentrated. The crude product was purified by column chromatography on silica gel using a gradient solvent system of 0 to 20% of ethyl acetate in hexanes to afford the titled compound as a yellow oil (683 mg, 84%). ¹H NMR (400 MHz, CDCl₃) δ 8.21-8.27 (1H), 7.84-7.92 (1H), 7.39 (m, 2H), 7.12 (m, 2H), 3.38-3.56 (3H); ESIMS: m/z 508.0 [(M+H)⁺]

### Step 4

### 2,4-bis(trifluoromethyl)-6-((trimethylsilyl)ethynyl)phenyl (4-fluorophenyl) (methyl)carbamate

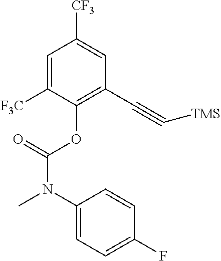 [SMILES: CN(C(=O)Oc1c(C#C[Si](C)(C)C)cc(C(F)(F)F)cc1C(F)(F)F)c1ccc(F)cc1]

To a solution of compound 2-iodo-4,6-bis(trifluoromethyl)phenyl (4-fluorophenyl)(methyl)carbamate (600 mg, 0.147 mmol, 1.0 eq) in DMF (3 mL) was added compound Trimethylsilyl acetylene (324 mg, 0.295 mmol, 1.5 eq), CuI (48 mg, 0.039 mmol, 0.2 eq), TEA (240 mg, 0.396 mmol, 2 eq), Pd(PPh3)2Cl2 (90 mg, 15%) under inert atmosphere. The reaction was stirred at 60° C. for 2 h. Then the mixture was extracted with DCM (20 mL×3). The combined organic layers were washed with brine, dried over sodium sulfate and filtered. The solution was concentrated under reduced pressure. The residue was purified by column chromatography to give compound 7 (330 mg, 46%) as off white solid. 1H NMR (400 MHz, CDCl3) δ 7.88-7.84 (bs, 1H), 7.78-7.71 (bs, 1H), 7.31-7.26 (m, 2H), 7.05-7.00 (t, 2H), 3.48 (s, 1H), 3.31 (s, 2H), 0.36 (s, 8H), 0.24 (s, 1H); ESIMS: m/z 478.4 [(M+H)+]

### Step 5

### 2-ethynyl-4,6-bis(trifluoromethyl)phenyl (4-fluorophenyl) (methyl)carbamate

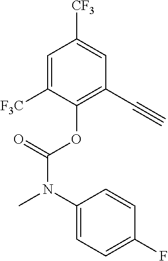 [SMILES: C#Cc1cc(C(F)(F)F)cc(C(F)(F)F)c1OC(=O)N(C)c1ccc(F)cc1]

To a solution of compound 2,4-bis(trifluoromethyl)-6-((trimethylsilyl)ethynyl)phenyl (4-fluorophenyl) (methyl)carbamate 8 (330 mg, 0.689 mmol, 1.0 eq) in THF (5 mL) at r.t was added TBAF (IM, 1.68 mL, 1.5 eq). The reaction mixture was stirred at RT for 2 h. Then the mixture was extracted with DCM (20 mL×2). The combined organic layer was washed with brine, dried over sodium sulfate and filtered. The filtrate was concentrated under reduced pressure. The residue was purified by column chromatography to give compound 9 (190 mg, 68%) as brown solid. 1H NMR (400 MHz, CDCl3) 400 MHz, CDCl3) δ 7.96-7.87 (bs, 1H), 7.83-7.76 (bs, 1H), 7.29-7.24 (m, 2H), 7.05-7.01 (t, 2H), 3.46 (s, 1H), 3.40 (s, 1H), 3.31 (bs, 2H); ESIMS: m/z 406.0 [(M+H)⁺]

### Step 6

### 2-(1-(5-((2-(2,6-dioxopiperidin-3-yl)-1,3-dioxoisoindolin-4-yl)amino)-5-oxopentyl)-1H-1,2,3-triazol-4-yl)-4,6-bis(trifluoromethyl)phenyl (4-fluorophenyl) (methyl)carbamate

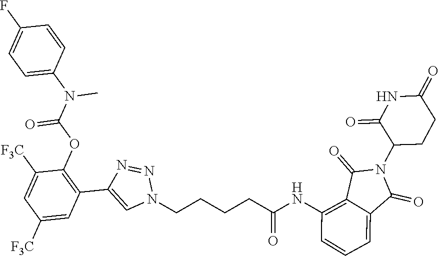 [SMILES: CN(C(=O)Oc1c(-c2cn(CCCCC(=O)Nc3cccc4c3C(=O)N(C3CCC(=O)NC3=O)C4=O)nn2)cc(C(F)(F)F)cc1C(F)(F)F)c1ccc(F)cc1]

### Step 7

### 5-azido-N-(2-(2,6-dioxopiperidin-3-yl)-1,3-dioxoisoindolin-4-yl) pentanamide

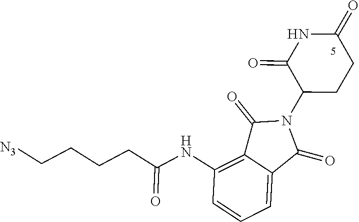 [SMILES: [N-]=[N+]=NCCCCC(=O)Nc1cccc2c1C(=O)N(C1CCC(=O)NC1=O)C2=O]

Anhydrous pyridine (0.96 mmol, 75 μL, 2 eq) was added to a solution of acid (0.48 mmol, 70 μL, 1 eq) and Palmolidamide (0.43 mmol, 120 mg, 0.9 eq) in anhydrous DMA (8 mL). This mixture was cooled to 0° C. and the Propylphosphonic Anhydride (1.92 mmol, 610 μL, 4 eq, in 50% ethyl acetate) was added dropwise. The reaction solution was stirred at 0° C. for 30 min and then allowed to stir at room temperature for overnight. The reaction was quenched with water (20 mL) and extracted with EtOAc (3×). The combined organic extracts were washed with brine, dried over anhydrous sodium sulfate and concentrated. The crude product was purified by column chromatography on silica gel using a gradient solvent system of 0 to 100% EtOAc in hexanes to afford the titled compound as a yellow solid (25 mg, 15%). ¹H NMR (400 MHz, CDCl₃) δ 9.35 (bs, 1H), 8.76-8.74 (d, 1H), 7.90 (bs, 1H), 7.67-7.63 (t, 1H), 7.50-7.45 (d, 1H), 490-4.84 (m, 1H), 3.30-3.26 (t, 2H), 2.88-2.82 (m, 1H), 2.77-2.65 (m, 2H), 2.46-2.42 (t, 2H), 2.13-2.08 (m, 1H), 1.82-1.74 (m, 2H), 1.67-1.62 (m, 2H); ESIMS: m/z 399.2 [(M+H)+].

### Step 8

### 2-(1-(5-((2-(2,6-dioxopiperidin-3-yl)-1,3-dioxoisoindolin-4-yl)amino)-5-oxopentyl)-1H-1,2,3-triazol-4-yl)-4,6-bis(trifluoromethyl)phenyl (4-fluorophenyl) (methyl)carbamate

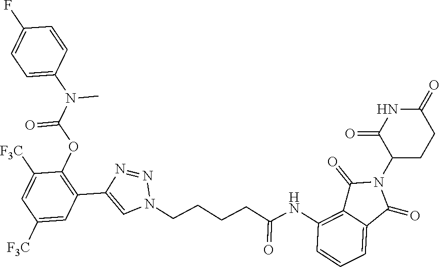 [SMILES: CN(C(=O)Oc1c(-c2cn(CCCCC(=O)Nc3cccc4c3C(=O)N(C3CCC(=O)NC3=O)C4=O)nn2)cc(C(F)(F)F)cc1C(F)(F)F)c1ccc(F)cc1]

To a solution of compound 2-ethynyl-4,6-bis(trifluoromethyl)phenyl (4-fluorophenyl) (methyl)carbamate (20 mg, 0.049 mmol, 1.0 eq) in DCM/MeOH/H₂O (3:1:1) was added compound 5-azido-N-(2-(2,6-dioxopiperidin-3-yl)-1,3-dioxoisoindolin-4-yl) pentanamide (19 mg, 0.049 mmol, 1.0 eq), sodium ascorbate (4 mg, 0.23 mmol, 0.5 eq), TEA (5 μL, 0.52 mmol, 1.1 eq), CuSO₄-5H₂O (4 mg, 0.14 mmol, 0.3 eq) under N₂ at room temperature. The reaction mixture was stirred for 12 h. Then the mixture was extracted with DCM (20 mL×2). The combined organic layer was washed with brine, dried over Na₂SO₄, filtered, and concentrated under reduced pressure. The residue was purified by revers phase (Gilson) to afford the titled compound as a white solid, (10 mg, 25% yield). ¹H NMR (400 MHz, CDCl₃): δ 9.35 (bs, 1H), 8.72-8.70 (d, 1H), 8.36 (bs, 1H), 7.90 (bs, 1H), 7.85-7.78 (m, 1H), 7.66 (s, 1H), 7.64-7.62 (d, 1H), 7.50-7.48 (d, 1H), 7.36-7.35 (m, 1H), 7.11-7.06 (m, 2H), 6.96-6.92 (m, 1H), 485-4.81 (m, 1H), 4.43-4.40 (t, 2H), 3.51 (s, 1H), 3.24 (s, 2H), 2.87-2.80 (m, 1H), 2.78-2.63 (m, 2H), 2.50-2.47 (t, 2H), 2.14-2.07 (m, IH), 2.09-2.01 (m, 2H), 1.78-1.68 (m, 2H); ESIMS:). 805.2 [M+2H]⁺]

### Example 4

### 2-(1-(6-((2-(2,6-dioxopiperidin-3-yl)-1,3-dioxoisoindolin-4-yl)amino)-6-oxohexyl)-1H-1,2,3-triazol-4-yl)-4,6-bis(trifluoromethyl)phenyl (4-fluorophenyl) (methyl)carbamate

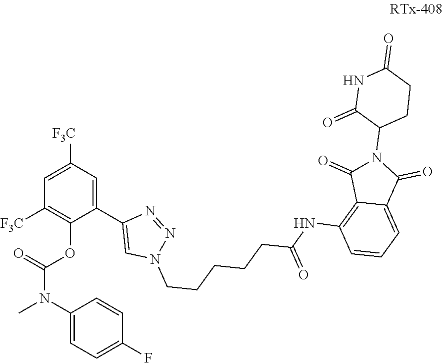 [SMILES: CN(C(=O)Oc1c(-c2cn(CCCCCC(=O)Nc3cccc4c3C(=O)N(C3CCC(=O)NC3=O)C4=O)nn2)cc(C(F)(F)F)cc1C(F)(F)F)c1ccc(F)cc1]

### Step 1

### 6-azido-N-(2-(2,6-dioxopiperidin-3-yl)-1,3-dioxoisoindolin-4-yl) hexanamide

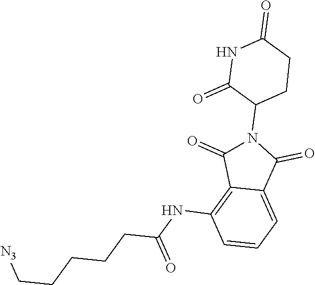 [SMILES: [N-]=[N+]=NCCCCCC(=O)Nc1cccc2c1C(=O)N(C1CCC(=O)NC1=O)C2=O]

Anhydrous pyridine (0.572 mmol, 47 μL, 1.8 eq) was added to a solution of acid (0.318 mmol, 50 mg) and Palmolidamide (0.318 mmol, 86 mg) in anhydrous DMA (5 mL). This mixture was cooled to 0° C. and the Propylphosphonic Anhydride (1.27 mmol, 403 μL4 e.q., in 50% ethyl acetate) was added dropwise. The reaction solution was stirred at 0° C. for 30 min and then allowed to stir at room temperature for overnight. The reaction was quenched with water (20 mL) and extracted with EtOAc (3×). The combined organic extracts were washed with brine, dried over anhydrous sodium sulfate and concentrated. The crude product was purified by column chromatography on silica gel using a gradient solvent system of 0 to 100% EtOAc in hexanes to afford the titled compound as a yellow solid (29 mg, 18%). ¹H NMR (400 MHz, CDCl₃): δ 9.43 (Bs, 1H), 8.76-8.74 (d, 1H), 8.30 (Bs, 1H), 7.67-7.63 (t, 1H), 7.53-7.44 (d, 1H), 4.99-4.86 (m, 1H), 3.24-3.21 (t, 2H), 2.708-2.703 (m, 2H), 2.43-2.39 (t, 2H), 1.72-1.68 (m, 4H), 1.63-1.59 (q, 2H), 1.45-1.37 (q, 2H); ESIMS: m/z 435.1 [(M+Na)⁺].

### Step 2

### 2-(1-(6-((2-(2,6-dioxopiperidin-3-yl)-1,3-dioxoisoindolin-4-yl)amino)-6-oxohexyl)-1H-1,2,3-triazol-4-yl)-4,6-bis(trifluoromethyl)phenyl (4-fluorophenyl) (methyl)carbamate (RTX 408)

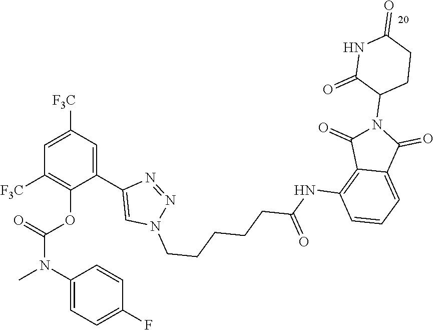 [SMILES: CN(C(=O)Oc1c(-c2cn(CCCCCC(=O)Nc3cccc4c3C(=O)N(C3CCC(=O)NC3=O)C4=O)nn2)cc(C(F)(F)F)cc1C(F)(F)F)c1ccc(F)cc1]

To a solution of compound 2-ethynyl-4,6-bis(trifluoromethyl)phenyl (4-fluorophenyl) (methyl)carbamate (30 mg, 0.072 mmol, 1.0 eq) in DCM/MeOH/H₂O (3:1:1) was added compound 6-azido-N-(2-(2,6-dioxopiperidin-3-yl)-1,3-dioxoisoindolin-4-yl) hexanamide (29 mg, 0.072 mmol, 1.0 eq), sodium ascorbate (7 mg, 0.036 mmol, 0.5 eq), TEA (8 μL, 0.079 mmol, 1.1 eq), CuSO4-5H2O (2 mg, 0.021 mmol, 0.3 eq) under N2 at rt. The reaction mixture was stirred for 12 h. Then the mixture was extracted with DCM (20 mL×2). The combined organic layer was washed with brine, dried over Na₂SO₄, filtered, and concentrated under reduced pressure. The residue was purified by revers phase (Gilson) column chromatography to afford the titled compound (12 mg, 20% Yield) as off white solid. 1H NMR (400 MHz, CDCl₃): δ 9.34 (bs, 1H), 8.72-8.70 (d, 1H), 8.32 (bs, 1H), 8.00-7.93 (bs, 2H), 7.78 (bs, 1H), 7.66-7.63 (d, 1H), 7.58 (bs, 1H), 7.49-7.47 (d, 1H), 7.37-7.34 (m, 1H), 7.09-7.05 (t, 1H), 6.97-6.92 (m, 1H), 4.89-4.85 (m, 1H), 4.38-4.307 (t, 2H), 3.50 (s, 1H), 3.25 (s, 2H), 2.74 (s, 1H), 2.71-2.67 (m, 2H), 2.44-2.41 (m, 2H), 2.15-2.08 (m, 1H), 2.01-2.01.92 (m, 2H), 1.79-1.65 (m, 2H), 1.40-1.34 (m, 2H); ESIMS: 818.2 [M+H]⁺]

### Example 5

### 2-(1-(2-(2-((2-(2,6-dioxopiperidin-3-yl)-1,3-dioxoisoindolin-4-yl)amino)-2-oxoethoxy)ethyl)-1H-1,2,3-triazol-4-yl)-4,6-bis(trifluoromethyl)phenyl (4-fluorophenyl) (methyl)carbamate

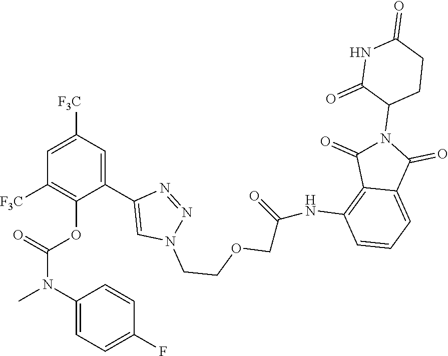 [SMILES: CN(C(=O)Oc1c(-c2cn(CCOCC(=O)Nc3cccc4c3C(=O)N(C3CCC(=O)NC3=O)C4=O)nn2)cc(C(F)(F)F)cc1C(F)(F)F)c1ccc(F)cc1]

### Step 1

### 2-(2-azidoethoxy)-N-(2-(2,6-dioxopiperidin-3-yl)-1,3-dioxoisoindolin-4-yl) acetamide

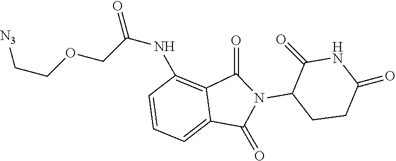 [SMILES: [N-]=[N+]=NCCOCC(=O)Nc1cccc2c1C(=O)N(C1CCC(=O)NC1=O)C2=O]

Anhydrous pyridine (0.72 mmol, 79 μL, 1.8 eq) was added to a solution of acid (0.36 mmol, 53 mg, 1 eq) and Palmolidamide (0.36 mmol, 100 mg) in anhydrous DMA (5 mL). This mixture was cooled to 0° C. and the Propylphosphonic Anhydride (1.44 mmol, 467 μL, 4 e.q., in 50% ethyl acetate) was added dropwise. The reaction solution was stirred at 0° C. for 30 min and then allowed to stir at room temperature for overnight. The reaction was quenched with water (20 mL) and extracted with EtOAc (3×). The combined organic extracts were washed with brine, dried over anhydrous sodium sulfate and concentrated. The crude product was purified by column chromatography on silica gel using a gradient solvent system of 0 to 100% EtOAc in hexanes to afford the titled compound as a yellow semi solid (30 mg, 20%). ¹H NMR (400 MHz, CDCl₃): δ 10.54 (Bs, 1H), 8.91-8.79 (d, 1H), 7.95 (Bs, 1H), 7.72-7.65 (t, 1H), 4.92-4.84 (m, 1H), 4.20 (s, 2H), 3.75-3.70 (t, 2H), 3.57-3.54 (m, 2H), 2.88-2.64 (m, 3H), 2.14-2.10 (m, 1H); ESIMS: m/z 401.3 [(M+H)⁺].

### Step 2

### 2-(1-(2-(2-((2-(2,6-dioxopiperidin-3-yl)-1,3-dioxoisoindolin-4-yl)amino)-2-oxoethoxy)ethyl)-1H-1,2,3-triazol-4-yl)-4,6-bis(trifluoromethyl)phenyl (4-fluorophenyl) (methyl)carbamate

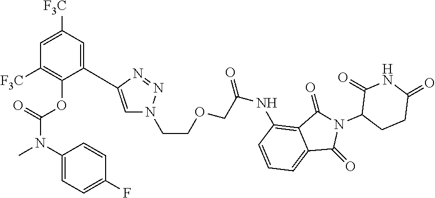 [SMILES: CN(C(=O)Oc1c(-c2cn(CCOCC(=O)Nc3cccc4c3C(=O)N(C3CCC(=O)NC3=O)C4=O)nn2)cc(C(F)(F)F)cc1C(F)(F)F)c1ccc(F)cc1]

To a solution of compound 2-ethynyl-4,6-bis(trifluoromethyl)phenyl (4-fluorophenyl) (methyl)carbamate (25 mg, 0.060 mmol, 1.0 eq) in DCM/MeOH/H₂O (3:1:1) was added compound IK-01-56 (24 mg, 0.060 mmol, 1.0 eq), sodium ascorbate (6 mg, 0.030 mmol, 0.5 eq), TEA (7 μL, 0.066 mmol, 1.1 eq), CuSO4-5H2O (7.4 mg, 0.018 mmol, 0.3 eq) under N₂ at r.t. The reaction mixture was stirred for 12 h. Then the mixture was extracted with DCM (20 mL×2). The combined organic layer was washed with brine, dried over Na₂SO₄, filtered, and concentrated under reduced pressure. The residue was purified by reverse phase (Gilson) column chromatography to give the titled compound (11 mg, 22% Yield) as off white solid. ¹H NMR (400 MHz, CDCl₃): δ 10.50-10.73 (d, 1H), 8.78-8.76 (d, 1H), 8.23-8.18 (m, 1H), 8.09 (bs, 1H), 8.02-7.92 (m, 1H), 7.84-7.76 (d, 1H), 7.66-7.54 (t, 1H), 7.54-7.52 (d, 1H), 7.38-7.35 (t, 1H), 7.05-7.03 (t, 2H), 6.92-6.90 (m, 1H) 4.89-4.76 (m, 1H), 4.69-4.64 (bs, 2H), 4.13 (t, 2H), 4.08-4.05 (m, 2H), 3.42 (s, 1H) 3.20 (s, 2H), 2.81-2.58 (m, 2H), 2.01-1.91 (m, 2H); ESIMS: m/z 807.1 [(M+2H)⁺].

### Example 6

### 2-(1-(2-(2-(2-((2-(2,6-dioxopiperidin-3-yl)-1,3-dioxoisoindolin-4-yl)amino)-2-oxoethoxy)ethoxy)ethyl)-1H-1,2,3-triazol-4-yl)-4,6-bis(trifluoromethyl)phenyl (4-fluorophenyl)(methyl)carbamate

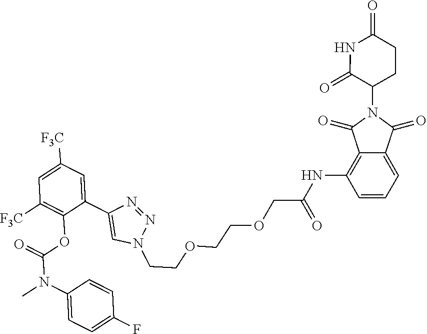 [SMILES: CN(C(=O)Oc1c(-c2cn(CCOCCOCC(=O)Nc3cccc4c3C(=O)N(C3CCC(=O)NC3=O)C4=O)nn2)cc(C(F)(F)F)cc1C(F)(F)F)c1ccc(F)cc1]

### Step 1

### 2-(2-(2-azidoethoxy) ethoxy)-N-(2-(2,6-dioxopiperidin-3-yl)-1,3-dioxoisoindolin-4-yl) acetamide

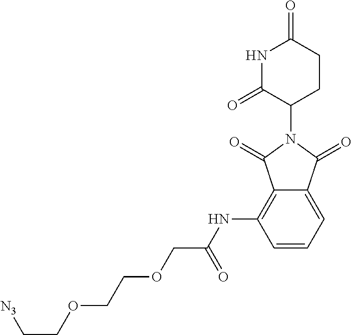 [SMILES: [N-]=[N+]=NCCOCCOCC(=O)Nc1cccc2c1C(=O)N(C1CCC(=O)NC1=O)C2=O]

Anhydrous pyridine (0.72 mmol, 52 μL, 2 eq) was added to a solution of acid (0.36 mmol, 70 mg) and Palmolidamide (0.36 mmol, 100 mg) in anhydrous DMA (4 mL). This mixture was cooled to 0° C. and the Propylphosphonic Anhydride (1.44 mmol, 469 μL, 4 eq., in 50% ethyl acetate) was added dropwise. The reaction solution was stirred at 0° C. for 30 min and then allowed to stir at room temperature for overnight. The reaction was quenched with water (20 mL) and extracted with EtOAc (3×). The combined organic extracts were washed with brine, dried over anhydrous sodium sulfate and concentrated. The crude product was purified by column chromatography on silica gel using a gradient solvent system of 0 to 100% EtOAc in hexanes to afford the titled compound as a yellow solid (22 mg, 13%). ¹H NMR (400 MHz, CDCl₃): δ 10.39 (Bs, 1H), 8.81-8.79 (d, 1H), 8.15 (Bs, 1H), 7.69-7.64 (t, 1H), 7.52-7.50 (d, 1H), 4.94-4.85 (m, 1H), 4.14 (s, 2H), 3.79-3.75 (m, 4H), 3.67-3.64 (t, 2H), 3.33-3.31 (t, 2H), 2.84-2.81 (m, 1H), 2.80-2.67 (m, 2H), 2.12-2.09 (m, 1H); ESIMS: m/z 445.1 [(M+H)⁺].

### Step 2

### 2-(1-(2-(2-(2-((2-(2,6-dioxopiperidin-3-yl)-1,3-dioxoisoindolin-4-yl)amino)-2-oxoethoxy)ethoxy)ethyl)-1H-1,2,3-triazol-4-yl)-4,6-bis(trifluoromethyl)phenyl (4-fluorophenyl)(methyl)carbamate

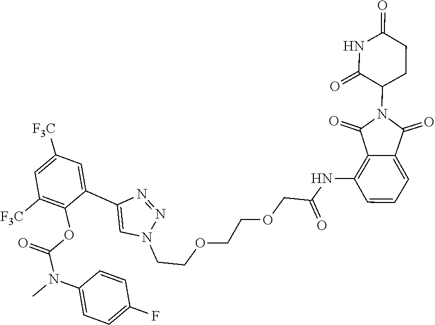 [SMILES: CN(C(=O)Oc1c(-c2cn(CCOCCOCC(=O)Nc3cccc4c3C(=O)N(C3CCC(=O)NC3=O)C4=O)nn2)cc(C(F)(F)F)cc1C(F)(F)F)c1ccc(F)cc1]

To a solution of compound 2-ethynyl-4,6-bis(trifluoromethyl)phenyl (4-fluorophenyl) (methyl)carbamate (20 mg, 0.047 mmol, 1.0 eq) in DCM/MeOH/H₂O (3:1:1) was added compound 2-(2-(2-azidoethoxy) ethoxy)-N-(2-(2,6-dioxopiperidin-3-yl)-1,3-dioxoisoindolin-4-yl) acetamide (19 mg, 0.047 mmol, 1.0 eq), sodium ascorbate (5 mg, 0.023 mmol, 0.5 eq), TEA (8 μL, 0.094 mmol, 1.1 eq), CuSO4-5H₂O (5 mg, 0.014 mmol, 0.3 eq) under N₂ at room temperature. The reaction mixture was stirred for 12 h. Then the mixture was extracted with DCM (20 mL×2). The combined organic layer was washed with brine, dried over Na₂SO₄, filtered, and concentrated under reduced pressure. The residue was purified by reverse phase (Gilson) column chromatography to give the titled compound (10 mg, 25% Yield) as off white solid. ¹H NMR (400 MHz, CDCl₃): ¹H NMR (400 MHz, CDCl₃) δ 10.32-10.29 (d, 1H), 8.74-8.72 (d, 1H), 8.30 (bs, 1H), 7.93-7.87 (t, 1H), 7.79 (s, 1H), 7.74-7.61 (m, 1H), 7.50-7.48 (d, 1H), 7.40-7.37 (m, 2H), 7.09-7.05 (t, 2H), 6.98-6.92 (m, 1H) 4.88-4.78 (m, 1H), 4.54 (bs, 2H), 4.03 (s, 2H), 3.99-3.84 (m, 2H), 3.73-3.69 (m, 4H) 3.50 (s, 1H), 3.23 (s, 2H), 2.83-2.77 (m, 1H), 2.74-2.67 (m, 2H), 2.09 (m, 1H); ESIMS: m/z 851.4 [(M+2H)⁺]

### Example 7

### 2-(1-(9-((2-(2,6-dioxopiperidin-3-yl)-1,3-dioxoisoindolin-4-yl)amino)-9-oxononyl)-1H-1,2,3-triazol-4-yl)-4,6-bis(trifluoromethyl)phenyl (4-fluorophenyl) (methyl)carbamate

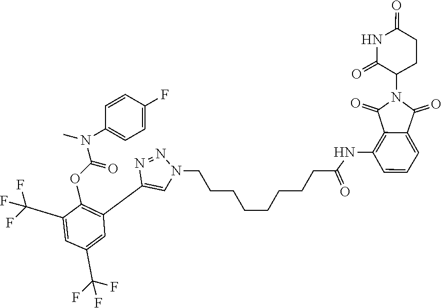 [SMILES: CN(C(=O)Oc1c(-c2cn(CCCCCCCCC(=O)Nc3cccc4c3C(=O)N(C3CCC(=O)NC3=O)C4=O)nn2)cc(C(F)(F)F)cc1C(F)(F)F)c1ccc(F)cc1]

### Step 1

### 9-azido-N-(2-(2,6-dioxopiperidin-3-yl) 1,3-dioxoisoindolin-4-yl) nonanamide

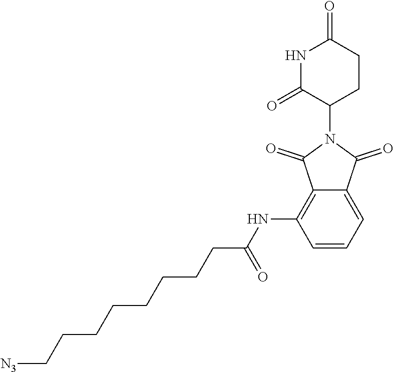 [SMILES: [N-]=[N+]=NCCCCCCCCC(=O)Nc1cccc2c1C(=O)N(C1CCC(=O)NC1=O)C2=O]

Anhydrous pyridine (0.72 mmol, 58 μL, 2 eq) was added to a solution of acid (0.36 mmol, 73 mg) and Palmolidamide (0.36 mmol, 100 mg) in anhydrous DMA (4 mL). This mixture was cooled to 0° C. and the Propylphosphonic Anhydride (1.44 mmol, 467 μL, 4 eq., in 50% ethyl acetate) was added dropwise. The reaction solution was stirred at 0° C. for 30 min and then allowed to stir at room temperature for overnight. The reaction was quenched with water (20 mL) and extracted with EtOAc (3×). The combined organic extracts were washed with brine, dried over anhydrous sodium sulfate and concentrated. The crude product was purified by column chromatography on silica gel using a gradient solvent system of 0 to 100% EtOAc in hexanes to afford the titled compound as white solid (40 mg, 24% Yield). ¹H NMR (400 MHz, CDCl₃): δ 9.34 (bs, 1H), 8.78-8.76 (d, 1H), 8.09 (bs, 1H), 7.66-7.62 (t, 1H), 7.48-7.47 (d, 1H), 4.90-4.82 (m, 1H), 3.28-3.17 (t, 2H), 2.88-2.82 (m, 1H), 2.82-2.64 (m, 2H), 2.54-2.37 (t, 2H), 2.16-2.2.10 (m, 1H), 1.70-1.64 (m, 2H), 1.68-1.49 (m, 2H), 1.28 (bs, 8H); ESIMS: m/z 454.1 [(M+H)⁺]

### Step 2

### 2-(1-(9-((2-(2,6-dioxopiperidin-3-yl)-1,3-dioxoisoindolin-4-yl)amino)-9-oxononyl)-1H-1,2,3-triazol-4-yl)-4,6-bis(trifluoromethyl)phenyl (4-fluorophenyl) (methyl)carbamate

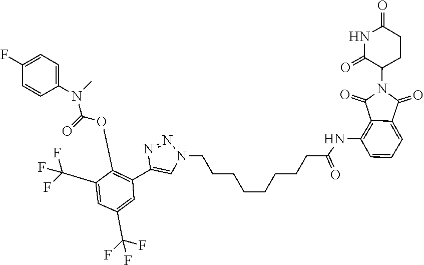 [SMILES: CN(C(=O)Oc1c(-c2cn(CCCCCCCCC(=O)Nc3cccc4c3C(=O)N(C3CCC(=O)NC3=O)C4=O)nn2)cc(C(F)(F)F)cc1C(F)(F)F)c1ccc(F)cc1]

To a solution of compound 2-ethynyl-4,6-bis(trifluoromethyl)phenyl (4-fluorophenyl) (methyl)carbamate (29 mg, 0.070 mmol, 1.0 eq) in DCM/MeOH/H₂O (3:1:1) was added compound 9-azido-N-(2-(2,6-dioxopiperidin-3-yl) 1,3-dioxoisoindolin-4-yl) nonanamide (32 mg, 0.070 mmol, 1.0 eq), sodium ascorbate (7 mg, 0.035 mmol, 0.5 eq), TEA (8 μL, 0.77 mmol, 1.1 eq), CuSO4-5H2O (5 mg, 0.021 mmol, 0.3 eq) under N₂ at rt. The reaction mixture was stirred for 12 h. Then the mixture was extracted with DCM (20 mL×2). The combined organic layer was washed with brine, dried over Na₂SO₄, filtered, and concentrated under reduced pressure. The residue was purified by reverse phase (Gilson) column chromatography to give the titled compound (13 mg, 21% Yield) as off white solid. ¹H NMR (400 MHz, MeOD): δ 8.53-8.51 (d, 1H), 8.38 (bs, 1H), 8.34-8.33 (m, 1H), 8.05 (s, 1H), 7.93-7.87 (d, 1H), 7.68-7.64 (t, 1H), 7.47-7.45 (d, 1H), 7.43-7.41 (m, 1H), 7.21 (m, 1H), 7.12-7.08 (t, 1H), 7.02-6.98 (m, 1H) 5.08-5.00 (m, 1H), 4.43-4.40 (t, 2H), 3.49 (s, 1H), 3.18 (s, 2H), 2.81-2.72 (m, 1H), 2.67-2.60 (m, 2H), 2.40-2.34 (q, 2H), 2.06-2.02 (m, 1H), 1.88-1.86 (m, 2H), 1.63-1.54 (m, 2H), 1.30-1.24 (m, 6H), 1.23-1.19 (t, 2H); ESIMS: m/z 860.2 [(M+H)+]

### Example 8

### 2-(1-(14-((2-(2,6-dioxopiperidin-3-yl)-1,3-dioxoisoindolin-4-yl)amino)-14-oxo-3,6,9,12-tetraoxatetradecyl)-1H-1,2,3-triazol-4-yl)-4,6-bis(trifluoromethyl)phenyl (4-fluorophenyl) (methyl)carbamate

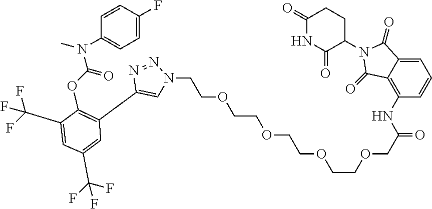 [SMILES: CN(C(=O)Oc1c(-c2cn(CCOCCOCCOCCOCC(=O)Nc3cccc4c3C(=O)N(C3CCC(=O)NC3=O)C4=O)nn2)cc(C(F)(F)F)cc1C(F)(F)F)c1ccc(F)cc1]

### Step 1

### 14-azido-N-(2-(2,6-dioxopiperidin-3-yl)-1,3-dioxoisoindolin-4-yl)-3,6,9,12-tetraoxatetradecanamide

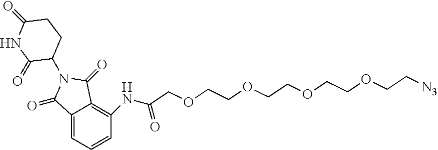 [SMILES: [N-]=[N+]=NCCOCCOCCOCCOCC(=O)Nc1cccc2c1C(=O)N(C1CCC(=O)NC1=O)C2=O]

Anhydrous pyridine (0.58 mmol, 48 μL, 2 eq) was added to a solution of acid (0.29 mmol, 81 mg) and Palmolidamide (0.29 mmol, 80 mg) in anhydrous DMA (4 mL). This mixture was cooled to 0° C. and the Propylphosphonic Anhydride (1.16 mmol, 374 μL, 4 eq., in 50% ethyl acetate) was added drop wise. The reaction solution was stirred at 0° C. for 30 min and then allowed to stir at room temperature for overnight. The reaction was quenched with water (20 mL) and extracted with EtOAc (3×). The combined organic extracts were washed with brine, dried over anhydrous sodium sulfate and concentrated. The crude product was purified by column chromatography on silica gel using a gradient solvent system of 0 to 100% EtOAc in hexanes to afford the titled compound as yellow solid (35 mg, 23% Yield). ¹H NMR (400 MHz, CDCl₃): δ 10.43 (s, 1H), 8.79-8.77 (d, 1H), 8.74 (bs, IH), 7.67-7.63 (d, 1H), 7.52-7.50 (d, 1H), 4.87-4.83 (m, 1H), 4.139-4.134 (d, 2H), 4.07 (s, 2H), 3.75-3.74 (bs, 2H), 3.63-3.62 (m, 4H) 3.61-3.59 (m, 8H), 2.82-2.79 (m, 1H), 2.77-2.67 (m, 2H), 2.12-2.05 (m, 1H); ESIMS: m/z 534.2. [(M+2H)⁺]

### Step 2

### 2-(1-(14-((2-(2,6-dioxopiperidin-3-yl)-1,3-dioxoisoindolin-4-yl)amino)-14-oxo-3,6,9,12-tetraoxatetradecyl)-1H-1,2,3-triazol-4-yl)-4,6-bis(trifluoromethyl)phenyl (4-fluorophenyl) (methyl)carbamate

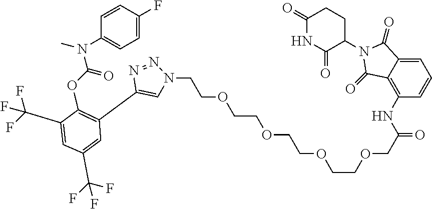 [SMILES: CN(C(=O)Oc1c(-c2cn(CCOCCOCCOCCOCC(=O)Nc3cccc4c3C(=O)N(C3CCC(=O)NC3=O)C4=O)nn2)cc(C(F)(F)F)cc1C(F)(F)F)c1ccc(F)cc1]

To a solution of compound 2-ethynyl-4,6-bis(trifluoromethyl)phenyl (4-fluorophenyl) (methyl)carbamate (35 mg, 0.084 mmol, 1.0 eq) in DCM/MeOH/H₂O (3:1:1) was added compound 14-azido-N-(2-(2,6-dioxopiperidin-3-yl)-1,3-dioxoisoindolin-4-yl)-3,6,9,12-tetraoxatetradecanamide (50 mg, 0.084 mmol, 1.0 eq), sodium ascorbate (8 mg, 0.042 mmol, 0.5 eq), TEA (9 μL, 0.09 mmol, 1.1 eq), CuSO₄-5H2O (5 mg, 0.025 mmol, 0.3 eq) under N₂ at rt. The reaction mixture was stirred for 12 h. Then the mixture was extracted with DCM (20 mL×2). The combined organic layer was washed with brine, dried over Na2SO4, filtered, and concentrated under reduced pressure. The residue was purified by reverse phase (Gilson) column chromatography to give to afford the titled compound as a white off solid (14 mg, 17% Yield). ¹H7 NMR (400 MHz, CDCl₃): δ 10.40 (s, 1H), 8.78-8.75 (d, 1H), 8.35 (bs, 1H), 7.85 (bs, 1H), 7.77-7.66 (bs, 1H), 7.64-7.62 (m, 1H), 7.50-7.48 (d, 1H), 7.40-7.37 (m, 1H), 7.28-7.25 (m, 1H), 7.09-7.05 (t, 2H), 6.98-, 6.92 (m, 1H) 4.85-4.78 (m, 1H), 4.54 (bs, 2H), 4.14-4.05 (m, 2H), 3.89 (bs, 2H), 3.69-3.67 (m, 4H), 3.58-3.53 (m, 6H), 3.52-3.49 (m, 2H), 3.32 (s, 1H), 3.24 (s, 2H), 2.81-2.77 (m, 1H), 2.75-2.66 (m, 2H), 2.06 (m, 1H); ESIMS: m/z 938.2 [(M+H)⁺].

### Example 9

### Step 1

### Tert-butyl 3-chloro-2,4-difluorophenylcarbamate

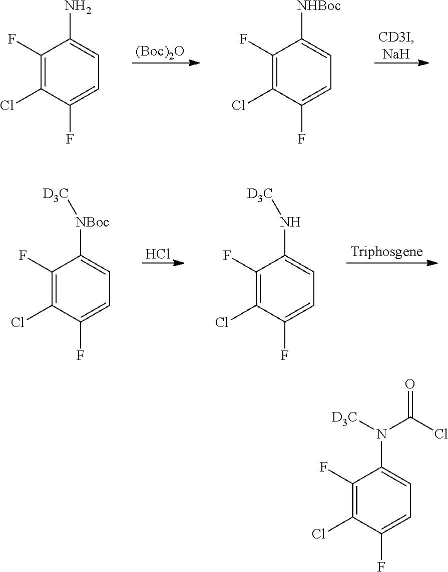

A mixture of 3-chloro-2,4-difluoroaniline (1.5 g, 9.17 mmol) and (Boc)2O (2.5 g, 11.5 mmol) in toluene (30 ml.) was heated at reflux overnight. The reaction was cooled to room temperature and the solvent was removed under reduced pressure. The residue was triturated with hexanes to provide tert-butyl 3-chloro-2,4-difluorophenylcarbamate (2.0 g, 7.59 mmol) as an off-white solid.

### Step 2

### Tert-butyl 3-chloro-2,4-difluorophenyl(methyl-d3)carbamate

To a round-bottom flask under an inert atmosphere of nitrogen was added tert-butyl 3-chloro-2,4-difluorophenylcarbamate (2.0 g, 7.59 mmol), and DMF (20 mL). The mixture was cooled to 0° C. and NaH (360 mg, 9.1 mmol as a 60% dispersion in mineral oil) was added. The solution was stirred for 1 h at 0° C., then CD3I (2.2 g, 15.17 mmol) was added at 0° C. The mixture was stirred overnight at 0° C. and then quenched with sat. aq. NH₄CI at room temperature. The aqueous layer was extracted with EtOAc and the combined organic extracts were concentrated under vacuum. The residue was purified by silica gel column chromatography (eluent: 3% EtOAc in PE) to afford the title compound 1.0 g as an off-white solid.

### Step 3

### 3-chloro-2,4-difluoro-N-(methyl-d3)aniline

To a round-bottom flask under an inert atmosphere of nitrogen was tert-butyl 3-chloro-2,4-difluorophenyl(methyl-d3)carbamate (1.0 g, 3.6 mmol), dioxane (18 mL) and con. HCl (6 mL). The mixture was cooled to RT overnight. Diluted with water 30 mL, adjust the pH To 8, the product was extracted with Et2O, washed with water and brine, dried over Na₂SO₄, removed the solvent to afford the title compound 0.5 g as an light-yellow oil.

### Step 4

### (4-fluorophenyl)(methyl-d3)carbamic Chloride

To a cold solution of triphosgene (0.28 g, 0.9 mmol) in DCM (15 mL) added a solution of N-methyl-4-fluoro aniline (0.3 g, 2.3 mmol) and pyridine (0.38 g, 1.92 mL, 4.7 mmol) in DCM (5 mL) dropwise. After that continued stirring at RT for 16 h. Quenched the reaction mixture with 1N aq.HCl (20 mL) and then extracted with DCM (2×20 mL). The DCM layer separated was dried over anhydrous Na₂SO₄, filtered, and concentrated under reduced pressure to afford the title compound as green oil 0.3 g.

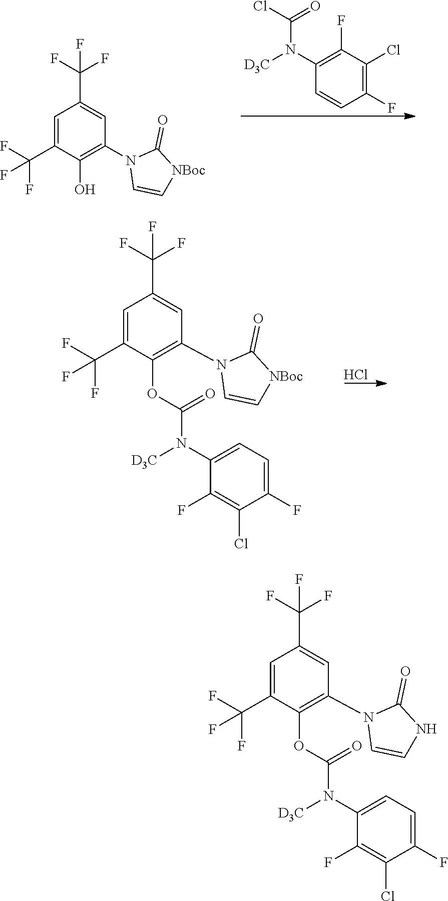

### Step 5

### Tert-butyl 3-(2-((3-chloro-2,4-difluorophenyl)(methyl-d3)carbamoyloxy)-3,5-bis(trifluoromethyl)phenyl)-2-oxo-2,3-dihydro-1H-imidazole-1-carboxylate

To a stirred solution of tert-butyl 3-(2-hydroxy-3,5-bis(trifluoromethyl)phenyl)-2-oxo-2,3-dihydro-1H-imidazole-1-carboxylate (0.2 g, 0.5 mmol) in pyridine (10 mL) at RT and continued stirring at RT for 30 minutes. To the above mixture added (4-fluorophenyl)(methyl-d3)carbamic chloride (0.21 g, 0.97 mmol). The resultant reaction mixture was further stirred at 60° C. overnight. Quenched the reaction mixture with 1N HCl (150 mL) and extracted with EtOAc (2×40 mL). The organic layer separated was combined, dried over anhydrous Na₂SO₄, filtered, and concentrated under reduced pressure and used in next step.

### Step 6

### 2-(2-oxo-2,3-dihydro-1H-imidazol-1-yl)-4,6-bis(trifluoromethyl)phenyl 3-chloro-2,4-difluorophenyl(methyl-d3)carbamate

The crude tert-butyl 3-(2-((3-chloro-2,4-difluorophenyl)(methyl-d3)carbamoyloxy)-3,5-bis(trifluoromethyl)phenyl)-2-oxo-2,3-dihydro-1H-imidazole-1-carboxylate was dissolved in 10 THF (10 mL) at RT and added con. HCl (3 mL). The mixture was stirred at rt overnight, Quenched the reaction mixture with NaHCO₃ aq. and extracted with EtOAc (2×40 mL). The organic layer separated was combined, dried over anhydrous Na₂SO₄, filtered, and concentrated under reduced pressure. The product was purified by prep-HPLC to obtain the titled compound (25 mg yield). ¹H NMR (400 MHz, CDCl3) δ9.91 (s, 1H), 7.94 (d, J=60.0 Hz, 2H), 7.01 (m, 1H), 7.14 (m, 1H), 6.48 (d, J=80.0 Hz, 2H); MS (ESI): m/z 518.93 (M+H)⁺.

### Example 10

### 2-(2-oxo-2,3-dihydro-1H-imidazol-1-yl)-4,6-bis(trifluoromethyl)phenyl 4-fluorophenyl(methyl-d3)carbamate

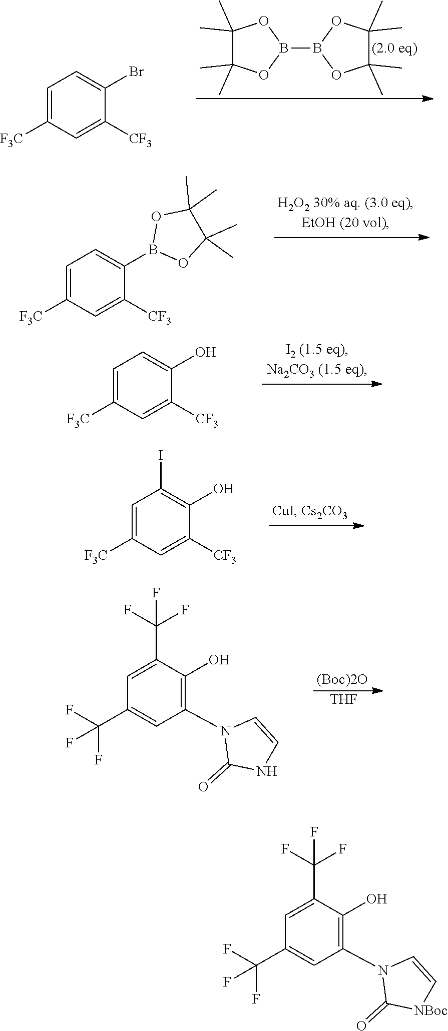

### Step 1

### 2-(2,4-bis(trifluoromethyl)phenyl)-4,4,5,5-tetramethyl-1,3,2-dioxaborolane

A solution of 1-bromo-2,4-bis(trifluoromethyl)benzene 5 g in 1,4 dioxane (50 mL) Was added bispinacolato diboron 5.2 g, potassium acetate 3.3 g, and Pd(dppf)Cl₂·DCM 0.66 g. the mixture was stirred at 100° C. under N₂, overnight. After completion of the reaction, the reaction mixture was concentrated under reduced pressure to afford the crude. Crude was purified by SG (PE/EA=10/1) to get product 3.2 g as a colorless oil.

### Step 2

### 2, 4-bis(trifluoromethyl)phenol

To a cold solution of 2-(2,4-bis(trifluoromethyl)phenyl)-4,4,5,5-tetramethyl-1,3,2-dioxaborolane crude 3.2 g in EtOH (64 mL), added hydrogen peroxide 30% aq. solution (3.2 mL) under inert atmosphere with stirring. The reaction mixture was stirred at rt overnight. The reaction mixture Was diluted with water and the product was extracted with MTBE, then washed with water and Brine, concentrated under reduced pressure. The product 1.25 g was taken as such to next step without further purification.

### Step 3

### 2-iodo-4,6-bis(trifluoromethyl)phenol

A solution of 2, 4-bis(trifluoromethyl)phenol 1.25 g in THF:H₂O (3:1, 31 mL) was cooled 0° C. in an ice bath with stirring. After 15 minutes, was added I₂ 1.5 g, followed by Na₂CO₃ 0.56 g, Allowed the reaction mixture to stir at rt overnight. The reaction mixture was cooled to 0° C. and then quenched with aq. sodium metabisulfite solution followed by extraction with EtOAc. The organic layer collected was washed with water followed by brine, dried over anhydrous Na₂SO₄, filtered and concentrated under reduced pressure. The product was purified by SG (PE/EA=10/1) to get product 1.4 g as a colorless oil.

### Step 4

### 1-(2-hydroxy-3,5-bis(trifluoromethyl)phenyl)-1H-imidazol-2 (3H)-one

To a solution of 2-iodo-4,6-bis(trifluoromethyl)phenol (1.4 g, 3.14 mmol) in DMA (30 mL) added 1H-imidazol-2(3H)-one (0.53 g, 6.28 mmol) K₂CO₃ (0.87 g, 6.28 mmol), CuI (0.3 g, 1.57 mmol) and N1,N2-dimethylethane-1,2-diamine (0.23 g, 3.14 mmol), CsF (0.96 g, 6.28 mmol). After that continued stirring at 110%° for 5 hour under N2, water (3 mL) was added. The mixture was adjusted to pH 6 with HCl (2 N). The mixture was extracted with ethyl acetate (30 mL×2). The combined organic layers were washed with brine, dried over Na₂SO₄ and filtered. The filtrate was concentrated under reduced pressure. The residue was purified by column chromatography (PE/EA=3/1) to afford compound 350 mg as pale-yellow solid.

### Step 5

### Tert-butyl 3-(2-hydroxy-3,5-bis(trifluoromethyl)phenyl)-2-oxo-2,3-dihydro-1H-imidazole-1-carboxylate

To a solution of 1-(2-hydroxy-3,5-bis(trifluoromethyl)phenyl)-1H-imidazol-2 (3H)-one (0.35 g, 1.12 mmol) in THF (30 mL) added (Boc)2O (0.3 g, 1.35 mmol), little DMAP. After that continued stirring at reflux overnight, The mixture was concentrated under reduced pressure. The residue was purified by column chromatography (PE/EA=3/1) to afford compound 330 mg as pink solid.

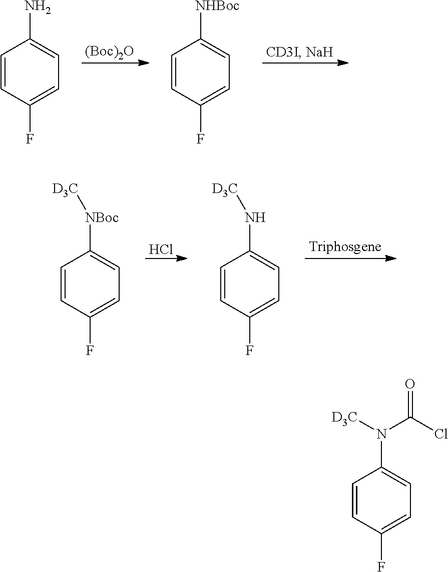

### Step 6

### (4-fluoro-phenyl)-carbamic Acid Tert-Butyl Ester

A mixture of 4-fluoroaniline (1.5 g, 13.5 mmol) and (Boc) 20 (3.54 g, 16.2 mmol) in toluene (30 ml.) was heated at reflux overnight. The reaction was cooled to room temperature and the solvent was removed under reduced pressure. The residue was triturated with hexanes to provide (4-fluoro-phenyl)-carbamic acid tert-butyl ester (2.0 g, 9.47 mmol) as an off-white solid.

### Step 7

### Tert-butyl (4-fluorophenyl)(methyl-d3)carbamate

To a round-bottom flask under an inert atmosphere of nitrogen was added (4-fluoro-phenyl)-carbamic acid tert-butyl ester (2.0 g, 9.47 mmol), and DMF (20 mL). The mixture was cooled to 0° C. and NaH (454 mg, 11.4 mmol as a 60% dispersion in mineral oil) was added. The solution was stirred for 1 h at 0° C., then CD3I (2.74 g, 18.94 mmol) was added at 0° C. The mixture was stirred overnight at 0° C. and then quenched with sat. aq. NH4Cl at room temperature. The aqueous layer was extracted with EtOAc and the combined organic extracts were concentrated under vacuum. The residue was purified by silica gel column chromatography (eluent: 3% EtOAc in PE) to afford the title compound 1.5 g as an off-white solid.

### Step 8

### 4-fluoro-N-(methyl-d3)aniline

To a round-bottom flask under an inert atmosphere of nitrogen was added tert-butyl (4-fluorophenyl)(methyl-d3)carbamate (1.5 g, 6.66 mmol), dioxane (18 mL) and con. HCl (6 mL). The mixture was cooled to RT overnight. Diluted with water 30 mL, adjust the pH To 8, the product was extracted with Et2O, washed with water and brine, dried over Na₂SO₄, removed the solvent to afford the title compound 0.8 g as an light-yellow oil.

### Step 9

### Tert-butyl 4-fluorophenyl(methyl-d3)carbamic Chloride

To a solution of compound 4-fluoro-N-(methyl-d3)aniline (110 mg, 0.86 mmol, 1.0 eq) in DCM (10 mL) at −10° C. was added DIEA (166 mg, 1.3 mmol, 1.5 eq) and triphosgene (115 mg, 0.395 mmol, 0.45 eq, in DCM (2 mL)). The reaction mixture was stirred at rt under N₂ for 8 h. Quenched the reaction mixture with 1N HCl (20 mL) and extracted with DCM (2×10 mL). The organic layer separated was combined, dried over anhydrous Na₂SO₄, filtered, and concentrated under reduced pressure and used in next step.

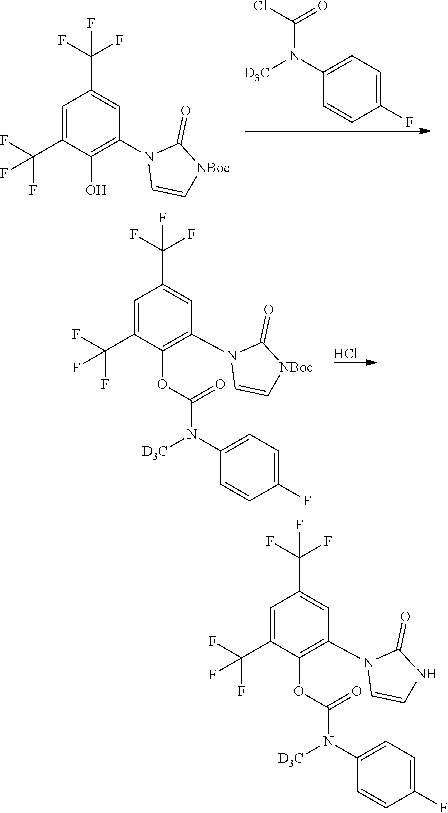

### Step 10

### Tert-butyl 3-(2-((4-fluorophenyl)(methyl-d3)carbamoyloxy)-3,5-bis(trifluoromethyl)phenyl)-2-oxo-2,3-dihydro-1H-imidazole-1-carboxylate

To a stirred solution of tert-butyl 3-(2-hydroxy-3,5-bis(trifluoromethyl)phenyl)-2-oxo-2,3-dihydro-1H-imidazole-1-carboxylate (0.2 g, 0.5 mmol) in pyridine (10 mL) at RT and continued stirring at RT for 30 minutes. To the above mixture added (4-fluorophenyl)(methyl-d3)carbamic chloride (0.19 g, 0.97 mmol). The resultant reaction mixture was further stirred at 60° C. overnight. Quenched the reaction mixture with IN HCl (150 mL) and extracted with EtOAc (2×40 mL). The organic layer separated was combined, dried over anhydrous Na₂SO₄, filtered, and concentrated under reduced pressure and used in next step.

### Step 11

### 2-(2-oxo-2,3-dihydro-1H-imidazol-1-yl)-4,6-bis(trifluoromethyl)phenyl 4-fluorophenyl(methyl-d3)carbamate

The crude tert-butyl 3-(2-((4-fluorophenyl)(methyl-d3)carbamoyloxy)-3,5-bis(trifluoromethyl)phenyl)-2-oxo-2,3-dihydro-1H-imidazole-1-carboxylate was dissolved in 10 THF (10 mL) at RT and added con. HCl (3 mL). The mixture was stirred at rt overnight, Quenched the reaction mixture with NaHCO₃ aq. and extracted with EtOAc (2×40 mL). The organic layer separated was combined, dried over anhydrous Na₂SO₄, filtered, and concentrated under reduced pressure. The product was purified by prep-HPLC to obtain the titled compound (25 mg). ¹H NMR (400 MHz, CDCl3) δ10.41 (s, 1H), 8.0 (d, J=40.0 Hz, 2H), 7.21 (m, 2H), 6.53 (m, 2H), 6.36 (d, J=40.0 Hz, 2H); MS (ESI): m/z 467.55 (M+H)⁺.

### Example 11

### 2-[1-(3-hydroxycyclobutyl)-1H-1,2,3-triazol-4-yl]-4,6-bis(trifluoromethyl)phenyl N-(4-fluorophenyl)-N-methylcarbamate

### The Preparation of Intermediate 9 (2-ethynyl-4,6-bis(trifluoromethyl)phenyl N-(4-fluorophenyl)-N-methylcarbamate) was Described for the Synthesis of Example 3

### Step 1

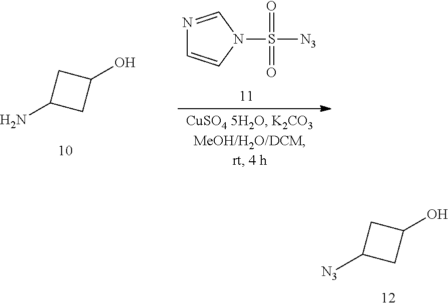 [SMILES: NC1CC(O)C1 ; [N-]=[N+]=NS(=O)(=O)n1ccnc1 ; [N-]=[N+]=NC1CC(O)C1]

A solution of compound 10 (50 mg, 0.57 mmol, 1.0 eq) in MeOH/H₂O/DCM (2 mL/1 mL/1.5 mL) was added compound 11 (169 mg, 0.81 mmol, 3.0 eq) and CuSO₄·5H₂O (2 mg, 0.004 mmol, 0.007 eq). The solution was adjusted to pH 9 with saturated aqueous K₂CO₃ at rt. The mixture was stirred under inert atmosphere at rt for 4 h. Then the mixture was extracted with DCM (20 mL×2). The combined organic layer was washed with brine, dried over Na₂SO₄ and filtered. The filtrate was concentrated under reduced pressure to give compound 12 (35 mg, 53%) as yellow oil.

### Step 2

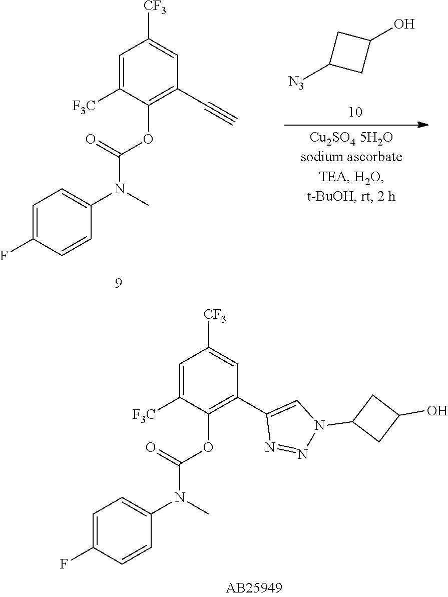 [SMILES: C#Cc1cc(C(F)(F)F)cc(C(F)(F)F)c1OC(=O)N(C)c1ccc(F)cc1 ; [N-]=[N+]=NC1CC(O)C1 ; CN(C(=O)Oc1c(-c2cn(C3CC(O)C3)nn2)cc(C(F)(F)F)cc1C(F)(F)F)c1ccc(F)cc1]

To a solution of compound 9 (115 mg, 0.28 mmol, 1.0 eq) in t-BuOH/H₂O (2 mL/2 mL) under N₂ at rt was added compound 12 (32 mg, 0.28 mmol, 1.0 eq), sodium ascorbate (28 mg, 0.142 mmol, 0.5 eq), TEA (32 mg, 0.31 mmol, 1.1 eq), CuSO₄·5H₂O (21 mg, 0.085 mmol, 0.3 eq). The reaction mixture was stirred for 2 h. Then the mixture was extracted with DCM (20 mL×2). The combined organic layer was washed with brine, dried over Na₂SO₄, filtered and concentrated. The residue was purified by column chromatography to give the titled compound (AB25949; 70.2 mg, 54%) as off-white solid. LC-MS: 519.15 [M+H]⁺

¹H NMR (400 MHz, CDCl₃) δ 8.41-8.52 (t, 1H), 7.79-7.93 (m, 2H), 7.39-7.49 (m, 2H), 7.04-7.18 (m, 2H), 5.19 (s, 0.17H), 4.73-4.77 (s, 1H), 4.35-4.39 (s, 0.84H), 3.31-3.58 (t, 3H), 2.13-2.95 (d, 2H), 2.53-2.69 (m, 2H).

### Examples 12 and 13

### 2-{1-[(3R,4S)-3,4-dihydroxycyclopentyl]-1H-1,2,3-triazol-4-yl}-4,6-bis(trifluoromethyl)phenyl N-(4-fluorophenyl)-(methyl-d3)carbamate (Racemate)

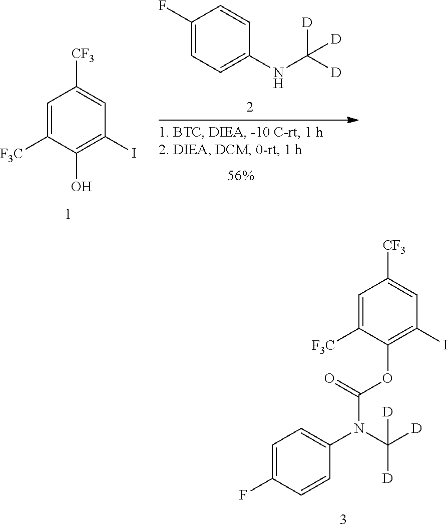 [SMILES: Oc1c(I)cc(C(F)(F)F)cc1C(F)(F)F ; [2H]C([2H])([2H])Nc1ccc(F)cc1 ; [2H]C([2H])([2H])N(C(=O)Oc1c(I)cc(C(F)(F)F)cc1C(F)(F)F)c1ccc(F)cc1]

### Step 1

To a solution of compound 1 (800 mg, 2.25 mmol, 1.0 eq) in DCM (10 mL) at −10° C. was added DIEA (436 mg, 3.37 mmol, 1.5 eq), and triphosgene (300 mg, 1.01 mmol, 0.5 eq) in DCM (2 mL). The reaction mixture was stirred at rt under N₂ for 1 h. The reaction solution was concentrated under reduced pressure to remove the solvent. Then DCM (3 mL) was added to the residue above. The solution was used for the next step.

### Step 2

To a solution of compound 2 (776 mg, 2.92 mmol, 1.5 eq) in DCM (5 mL) at 0° C. was added the solution prepared above. The mixture was stirred at rt under N₂ for 1 h. After completion of the reaction monitored by TLC (R_{f} 0.5, PE:EA=10:1), HCl (1 N, 15 mL) was added. Then the mixture was extracted with DCM (30 mL×2). The combined organic layer was washed with brine, dried with Na₂SO₄ and filtered. The filtrate was concentrated under reduced pressure. The residue was purified by column chromatography to give compound 3 (650 mg, 56%) as yellow oil.

### Step 3

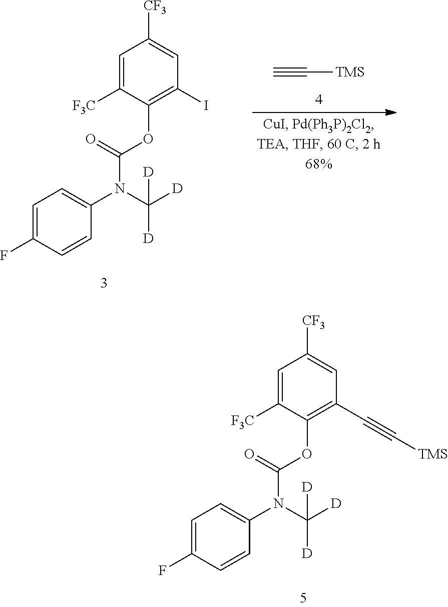

To a solution of compound 3 (650 mg, 1.27 mmol, 1.0 eq) in DMF (9 mL) under inert atmosphere at rt was added compound 4 (348 mg, 1.91 mmol, 1.5 eq), CuI (48 mg, 0.25 mmol, 0.2 eq), TEA (257 mg, 2.54 mmol, 2 eq) and Pd(PPh₃)₂Cl₂ (98 mg, 15% wt). The reaction was stirred at 60° C. for 2 h. Then the mixture was diluted with DCM (40 mL), washed with brine, dried over Na₂SO₄ and filtered. The filtrate was concentrated under reduced pressure. The residue was purified by column chromatography to give compound 6 (420 mg, 68%) as yellow oil.

### Step 4

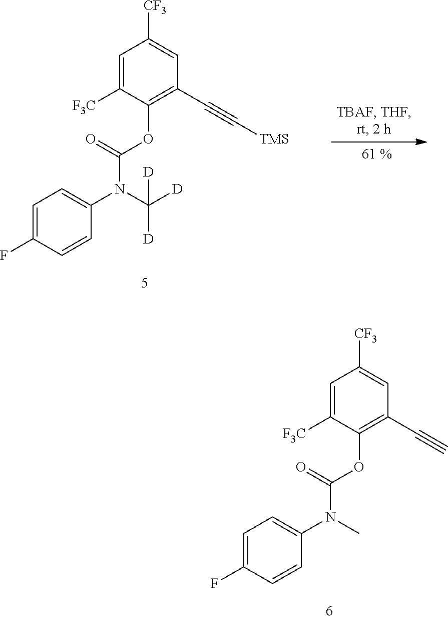 [SMILES: [2H]C([2H])([2H])N(C(=O)Oc1c(C#C[Si](C)(C)C)cc(C(F)(F)F)cc1C(F)(F)F)c1ccc(F)cc1 ; C#Cc1cc(C(F)(F)F)cc(C(F)(F)F)c1OC(=O)N(C)c1ccc(F)cc1]

To a solution of compound 5 (420 mg, 0.88 mmol, 1.0 eq) in THF (5 mL) at rt was added TBAF (1M, 1.3 mL, 1.3 mmol, 1.5 eq). The reaction mixture was stirred at rt for 2 h. The mixture was diluted with DCM (40 mL), washed with water and brine, dried over Na₂SO₄ and filtered. The solution was concentrated under reduced pressure. The residue was purified by column chromatography to give compound 6 (220 mg, 61%) as yellow oil.

### Step 5

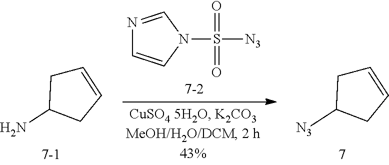 [SMILES: NC1CC=CC1 ; [N-]=[N+]=NS(=O)(=O)n1ccnc1 ; [N-]=[N+]=NC1CC=CC1]

To a solution of compound 7-1 (400 mg, 3.33 mmol, 1.0 eq) in MeOH/H₂O/DCM (2 mL/1.0 mL/1.5 mL) at rt was added compound 7-2 (1.4 g, 6.66 mmol, 2.0 eq), CuSO₄·5H₂O (6 mg, 0.023 mmol, 0.007 eq). The solution was adjusted with saturated aqueous K₂CO₃ to pH 9. The reaction was stirred at rt for 2 h. Then the mixture was extracted with ethyl acetate (20 mL×2). The combined organic layer was washed with brine, dried over Na₂SO₄ and filtered. The filtrate was concentrated under reduced pressure to give crude compound 7 (230 mg, 43%) as yellow oil, which was used for next step without further purification.

### Step 6

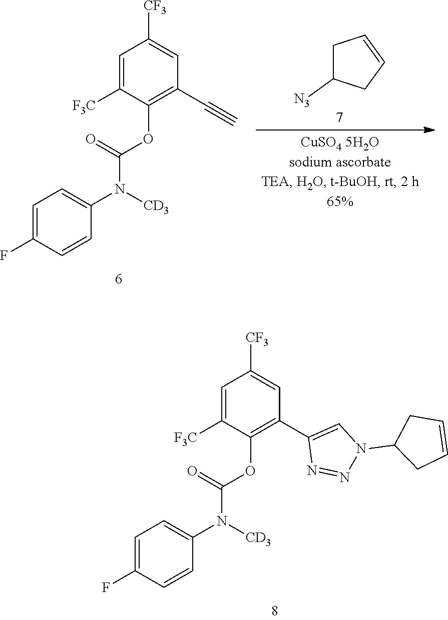 [SMILES: [2H]C([2H])([2H])N(C(=O)Oc1c(C#C)cc(C(F)(F)F)cc1C(F)(F)F)c1ccc(F)cc1 ; [N-]=[N+]=NC1CC=CC1 ; [2H]C([2H])([2H])N(C(=O)Oc1c(-c2cn(C3CC=CC3)nn2)cc(C(F)(F)F)cc1C(F)(F)F)c1ccc(F)cc1]

To a solution of compound 6 (230 mg, 0.56 mmol, 1.0 eq) in t-BuOH/H₂O (2 mL/2 mL) under N₂ at rt was added compound 7 (68 mg, 0.62 mmol, 1.1 eq), sodium ascorbate (55 mg, 0.28 mmol, 0.5 eq), TEA (63 mg, 0.62 mmol, 1.1 eq) and CuSO₄·5H₂O (43 mg, 0.17 mmol, 0.3 eq). The reaction mixture was stirred at rt for 2 h. The reaction was monitored by LC-MS and TLC (R_{f} 0.6, PE:EA=2:1). Then the mixture was diluted with DCM (50 mL), washed with water and brine, dried over Na₂SO₄ and filtered. The solution was concentrated under reduced pressure. The residue was purified by column chromatography to give compound 8 (190 mg, 65%) as white solid.

### Step 7

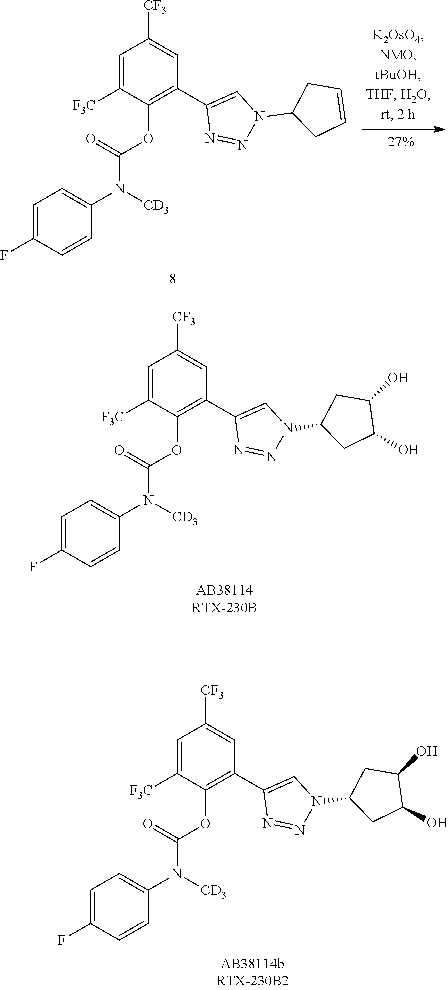 [SMILES: [2H]C([2H])([2H])N(C(=O)Oc1c(-c2cn(C3CC=CC3)nn2)cc(C(F)(F)F)cc1C(F)(F)F)c1ccc(F)cc1 ; [2H]C([2H])([2H])N(C(=O)Oc1c(-c2cn([C@H]3C[C@@H](O)[C@@H](O)C3)nn2)cc(C(F)(F)F)cc1C(F)(F)F)c1ccc(F)cc1 ; [2H]C([2H])([2H])N(C(=O)Oc1c(-c2cn([C@@H]3C[C@@H](O)[C@@H](O)C3)nn2)cc(C(F)(F)F)cc1C(F)(F)F)c1ccc(F)cc1]

To a solution of compound 8 (200 mg, 0.38 mmol, 1.0 eq) in t-BuOH/THF/H₂O (2 mL/2 mL/1 mL) under N₂ at rt was added K₂OsO₄ (6 mg, 0.007 mmol, 0.02 eq), and NMO (68 mg, 0.58 mmol, 1.5 eq). The reaction mixture was stirred for 2 h at rt. The reaction was monitored with LC-MS and TLC (R_{f} 0.3, PE:EA=1:1). The mixture was diluted with water (10 mL) and extracted with DCM (30 mL×2). The combined organic layer was washed with water and brine, dried over Na₂SO₄ and filtered. The solution was concentrated under reduced pressure. The residue was purified by prep-HPLC to give the titled compounds (RTX-230B (cis-isomer, AB38114A, 57.2 mg, 27%)) as white solid and RTX-230B2 (a mixture of cis and trans isomers, AB38114B, 10.7 mg, 5%) as white solid.

RTX-230B

LC-MS: 552.20 [M+H]⁺

¹H NMR (400 MHz, CD₃OD): δ 7.95-8.48 (m, 3H), 7.50-7.53 (m, 1H), 7.10-7.28 (m, 3H), 5.33 (m, 1H), 4.37 (s, 1H), 2.28-2.43 (m, 4H)

### Example 14

### 2-{1-[(3-hydroxyazetidin-3-yl)methyl]-1H-1,2,3-triazol-4-yl}-4,6-bis(trifluoromethyl)phenyl N-(4-fluorophenyl)-N-methylcarbamate

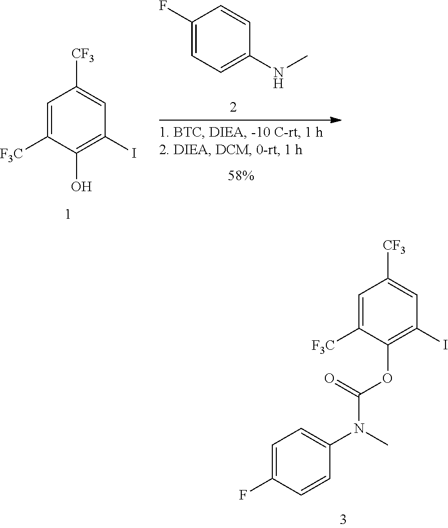

### Step 1

To a solution of compound 1 (1.0 g, 2.82 mmol, 1.0 eq) in DCM (10 mL) at −10° C. was added DIEA (545 mg, 4.2 mmol, 1.5 eq) and triphosgene (418 mg, 1.4 mmol, 0.5 eq) in DCM (2 mL). The reaction mixture was stirred at rt under N₂ for 1 h. The reaction solution was concentrated under reduced pressure to remove the solvent. DCM (3 mL) added to the residue above. The solution was used for next step.

### Step 2

To a solution of compound 2 (422 mg, 3.38 mmol, 1.2 eq) in DCM (5 mL) at 0° C. was added the solution prepared above. The mixture was stirred at rt under N₂ for 1 h. After completion of the reaction checked by TLC (R_{f} 0.5, PE:EA=10:1), HCl (1 N, 15 mL) was added. Then the mixture was extracted with DCM (20 mL×2). The combined organic layer was washed with brine, dried over Na₂SO₄ and filtered. The solution was concentrated under reduced pressure. The residue was purified by column chromatography to give compound 3 (830 mg, 58%) as yellow solid.

### Step 3

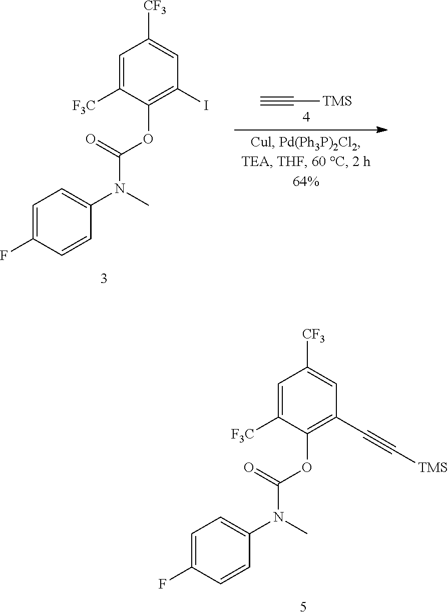

To a solution of compound 3 (830 mg, 1.63 mmol, 1.0 eq) in DMF (3 mL), was added compound 4 (446 mg, 2.45 mmol, 1.5 eq), CuI (62 mg, 0.326 mmol, 0.2 eq), TEA (329 mg, 3.26 mmol, 2.0 eq), and Pd(PPh₃)₂Cl₂ (125 mg, 15% wt) under inert atmosphere at rt. The reaction was stirred at 60° C. for 2 h. Then the mixture was extracted with DCM (30 mL×2). The combined organic layer was washed with brine, dried over Na₂SO₄, filtered, and concentrated under reduced pressure. The residue was purified by column chromatography to give compound 5 (500 mg, 64%) as yellow oil.

### Step 4

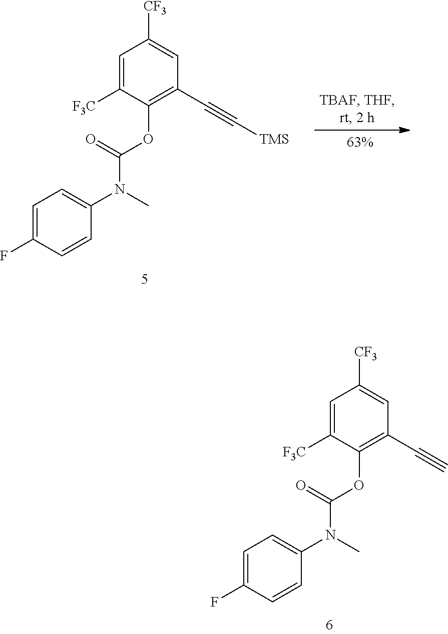 [SMILES: CN(C(=O)Oc1c(C#C[Si](C)(C)C)cc(C(F)(F)F)cc1C(F)(F)F)c1ccc(F)cc1 ; C#Cc1cc(C(F)(F)F)cc(C(F)(F)F)c1OC(=O)N(C)c1ccc(F)cc1]

To a solution of compound 5 (500 mg, 1.04 mmol, 1.0 eq) in THF (5 mL) at rt was added TBAF (1M in THE, 1.6 mL, 1.5 eq). The reaction mixture was stirred at rt for 2 h. Then the mixture was diluted with EA (100 mL), washed with water and brine, dried over Na₂SO₄ and filtered. The filtrate was concentrated under reduced pressure. The residue was purified by column chromatography to give compound 6 (280 mg, 63%) as off-white solid.

### Step 5

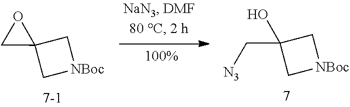 [SMILES: CC(C)(C)OC(=O)N1CC2(CO2)C1 ; CC(C)(C)OC(=O)N1CC(O)(CN=[N+]=[N-])C1]

To a solution of compound 7-1 (100 mg, 0.54 mmol, 1.0 eq) in DMF (3 mL) at rt was added NaN₃ (106 mg, 1.62 mmol, 3.0 eq). The reaction was stirred at 80° C. for 2 h. Then the mixture was diluted with water (10 mL) and extracted with EA (20 mL×2). The organic layers were washed with brine, dried with Na2SO₄, filtered, concentrated under reduced pressure to give crude compound 7 (138 mg, 100%) as yellow oil.

### Step 6

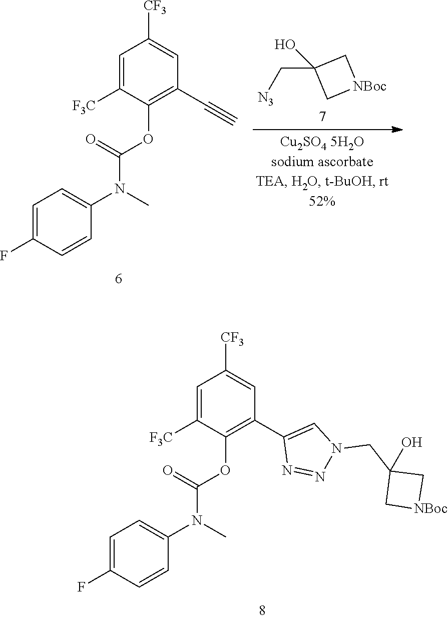

To a solution of compound 6 (160 mg, 0.44 mmol, 1.0 eq) in t-BuOH/H₂O (3 mL/3 mL) under N₂ at rt were added compound 7 (132 mg, 0.57 mmol, 1.3 eq), sodium ascorbate (44 mg, 0.2 mmol, 0.5 eq), TEA (48 mg, 0.48 mmol, 1.1 eq) and CuSO₄-5H₂O (33 mg, 0.132 mmol, 0.3 eq) The reaction mixture was stirred for 2 h. Then the mixture was extracted with DCM (20 mL×2). The combined organic layer was washed with brine, dried over Na₂SO₄ and filtered. The filtrate was concentrated under reduced pressure. The residue was purified by column chromatography to give compound 8 (130 mg, 52%) as off-white solid.

### Step 7

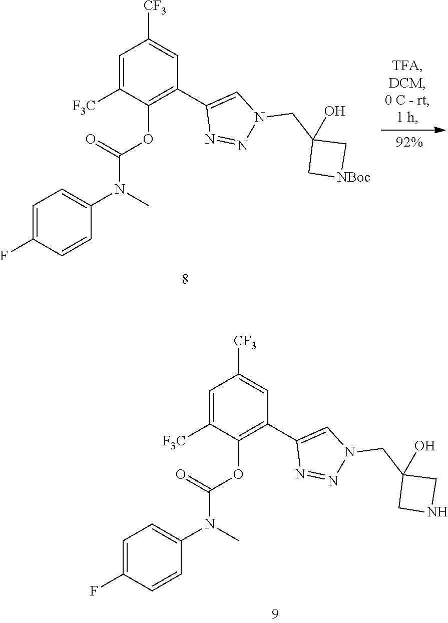 [SMILES: CN(C(=O)Oc1c(-c2cn(CC3(O)CN(C(=O)OC(C)(C)C)C3)nn2)cc(C(F)(F)F)cc1C(F)(F)F)c1ccc(F)cc1 ; CN(C(=O)Oc1c(-c2cn(CC3(O)CNC3)nn2)cc(C(F)(F)F)cc1C(F)(F)F)c1ccc(F)cc1]

To a solution of compound 8 (130 mg, 0.205 mmol, 1.0 eq) in DCM (2 mL) at 0° C. was added TFA (2 mL). The reaction mixture was stirred at rt for 1 h. The mixture was concentrated under reduced pressure. The residue was purified by C18 reverse column (36% ACN in H₂O) and freeze-dried to give the titled compound (9: 101.3 mg, 92%) as a white solid.

LC-MS: 534.10 [M+H]⁺

1H NMR (400 MHz, CDCl₃): δ 8.48 (t, 2H), 8.31-8.35 (t, 1H), 7.81-8.07 (m, 2H), 7.36-7.39 (m, 1.43H), 7.18-7.20 (m, 0.62H), 6.91-7.09 (m, 2H), 4.71-4.79 (m, 2H), 4.15 (s, 4H), 3.47 (s, 1H), 3.17 (s, 2H).

### Example 15

### 2-{1-[(3-hydroxyazetidin-3-yl)methyl]-1H-1,2,3-triazol-4-yl}-4,6-bis(trifluoromethyl)phenyl N-(4-fluorophenyl)-N-(methyl-d3)carbamate

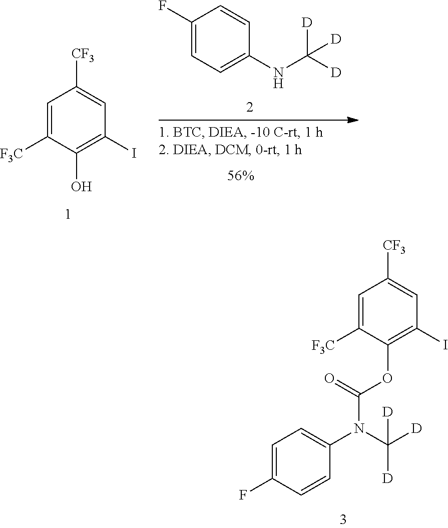 [SMILES: Oc1c(I)cc(C(F)(F)F)cc1C(F)(F)F ; [2H]C([2H])([2H])Nc1ccc(F)cc1 ; [2H]C([2H])([2H])N(C(=O)Oc1c(I)cc(C(F)(F)F)cc1C(F)(F)F)c1ccc(F)cc1]

### Step 1

To a solution of compound 1 (800 mg, 2.25 mmol, 1.0 eq) in DCM (10 mL) at −10° C. was added DIEA (436 mg, 3.37 mmol, 1.5 eq) and a solution of triphosgene (300 mg, 1.01 mmol, 0.5 eq) in DCM (2 mL). The reaction mixture was stirred at rt under N₂ for 1 h. The reaction solution was concentrated under reduced pressure to remove the solvent. Then DCM (3 mL) was added to the residue prepared. The solution was used for next step reaction.

### Step 2

To a solution of compound 2 (776 mg, 2.92 mmol, 1.5 eq) in DCM (5 mL) at 0° C. was added the solution above. The mixture was stirred at rt under N₂ for 1 h. After completion of the reaction, HCl (1 N, 15 mL) was added. Then the mixture was extracted with DCM (30 mL×2). The combined organic layer was washed with brine, dried over Na₂SO₄ and filtered and concentrated under reduced pressure. The residue was purified by column chromatography to give compound 3 (650 mg, 56%) as yellow oil.

### Step 3

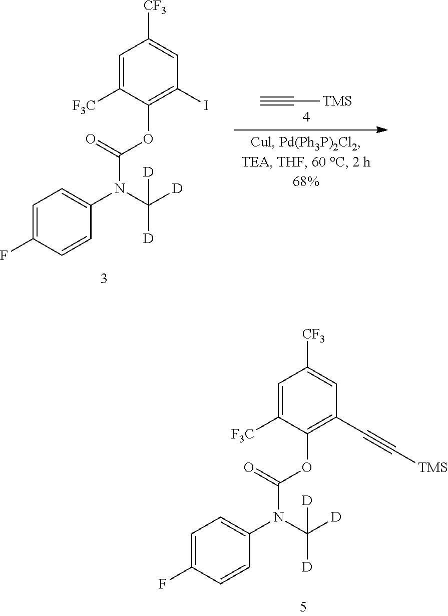

A solution of compound 3 (650 mg, 1.27 mmol, 1.0 eq), compound 4 (348 mg, 1.91 mmol, 1.5 eq), CuI (48 mg, 0.254 mmol, 0.2 eq), TEA (257 mg, 2.54 mmol, 2 eq) and Pd(PPh₃)₂Cl₂ (98 mg, 15% wt) under inert atmosphere in DMF (9 mL) was stirred at 60° C. for 2 h. Then the mixture was cooled to rt, diluted with water (20 mL), extracted with DCM (20 mL×3). The combined organic layer was washed with brine, dried with Na₂SO₄ and filtered. The solution was concentrated under reduced pressure. The residue was purified by column chromatography to give compound 6 (420 mg, 68%) as yellow oil.

### Step 4

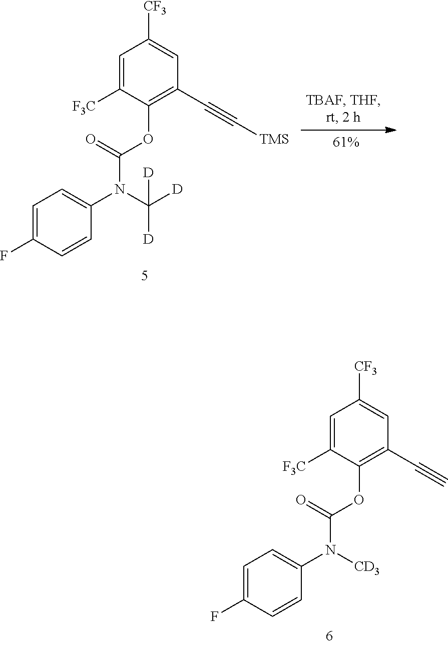 [SMILES: [2H]C([2H])([2H])N(C(=O)Oc1c(C#C[Si](C)(C)C)cc(C(F)(F)F)cc1C(F)(F)F)c1ccc(F)cc1 ; [2H]C([2H])([2H])N(C(=O)Oc1c(C#C)cc(C(F)(F)F)cc1C(F)(F)F)c1ccc(F)cc1]

To a solution of compound 5 (420 mg, 0.875 mmol, 1.0 eq) in THF (5 mL) at rt was added TBAF (1.3 mL, 1.5 eq). The reaction mixture was stirred at rt for 2 h, diluted with DCM (60 mL), washed with brine, dried over Na₂SO₄ and filtered. The solution was concentrated under reduced pressure. The residue was purified by column chromatography to give compound 6 (220 mg, 61%) as yellow oil.

### Step 5

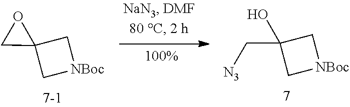 [SMILES: CC(C)(C)OC(=O)N1CC2(CO2)C1 ; CC(C)(C)OC(=O)N1CC(O)(CN=[N+]=[N-])C1]

To a solution of compound 7-1 (100 mg, 0.54 mmol, 1.0 eq) in DMF (3 mL) at rt was added NaN₃ (106 mg, 1.62 mmol, 3.0 eq). The reaction was stirred at 80° C. for 2 h. Then the mixture was diluted with water (10 mL) and extracted with EA (20 mL×2). The organic layers were washed with brine, dried with Na2SO₄, filtered, concentrated under reduced pressure to give crude compound 7 (138 mg, 100%) as yellow oil.

### Step 6

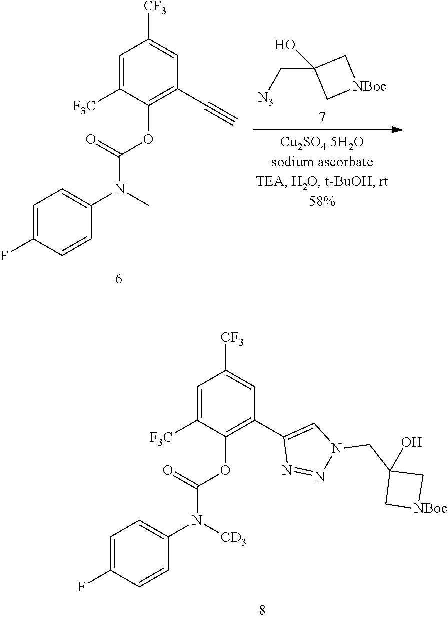

To a solution of compound 6 (220 mg, 0.539 mmol, 1.0 eq) in t-BuOH/H₂O (2 mL/2 m) was added compound 7 (122 mg, 0.539 mmol, 1.0 eq), sodium ascorbate (53 mg, 0.269 mmol, 0.5 eq), TEA (51 mg, 0.50 mmol, 1.1 eq) and CuSO₄·5H₂O (40 mg, 0.16 mmol, 0.3 eq) under N₂ at rt. The reaction mixture was stirred at rt for 2 h. Then the mixture was extracted with DCM (20 mL×2). The combined organic layer was washed with brine, dried with Na₂SO₄ and filtered. The filtrate was concentrated under reduced pressure. The residue was purified by column chromatography to give compound 8 (170 mg, 58%) as white solid.

### Step 7

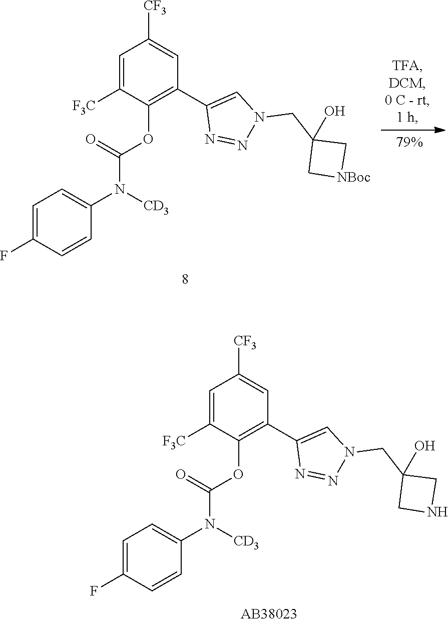 [SMILES: [2H]C([2H])([2H])N(C(=O)Oc1c(-c2cn(CC3(O)CN(C(=O)OC(C)(C)C)C3)nn2)cc(C(F)(F)F)cc1C(F)(F)F)c1ccc(F)cc1 ; [2H]C([2H])([2H])N(C(=O)Oc1c(-c2cn(CC3(O)CNC3)nn2)cc(C(F)(F)F)cc1C(F)(F)F)c1ccc(F)cc1]

To a solution of compound 8 (170 mg, 0.267 mmol, 1.0 eq) in DCM (2 mL) at 0° C. was added TFA (2 mL). The reaction mixture was stirred at rt for 1 h. The mixture was concentrated under reduced pressure. The residue was purified by C18 reverse column (36% MeCN in water) and freeze-dried to give the titled compound (AB38023, 113.7 mg, 79%) as white solid.

LC-MS: 537.00 [M+H]⁺

¹H NMR (400 MHz, CDCl₃): δ 9.35-9.51 (m, 2H), 8.32-8.38 (m, 1H), 7.84-8.07 (t, 2H), 7.37-7.40 (m, 1.6H), 7.20-7.25 (m, 0.45H), 6.94-7.11 (m, 2H), 4.69-4.83 (m, 2H), 4.17 (m, 4H).

### Example 16

### 2-{1-[(3-hydroxyazetidin-3-yl)methyl]-1H-1,2,3-triazol-4-yl}-4,6-bis(trifluoromethyl)phenyl N-(3-chloro-2,4-difluorophenyl)-N-(methyl-d3)carbamate

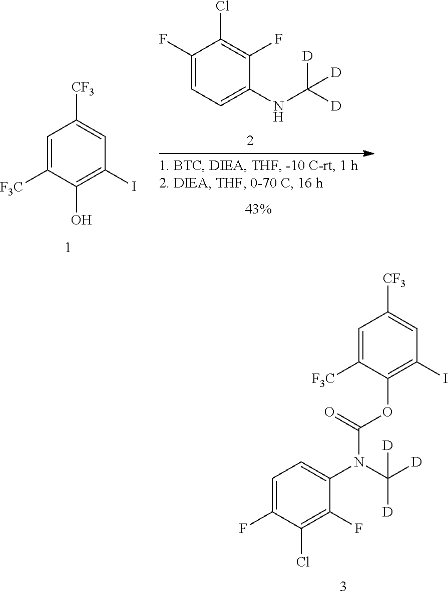 [SMILES: Oc1c(I)cc(C(F)(F)F)cc1C(F)(F)F ; [2H]C([2H])([2H])Nc1ccc(F)c(Cl)c1F ; [2H]C([2H])([2H])N(C(=O)Oc1c(I)cc(C(F)(F)F)cc1C(F)(F)F)c1ccc(F)c(Cl)c1F]

### Step 1

To a solution of compound 1 (500 mg, 2.25 mmol, 1.0 eq) in THF (10 mL) at −10° C. was added DIEA (272 mg, 2.1 mmol, 1.5 eq), and triphosgene (166 mg, 0.63 mmol, 0.4 eq) in THF (2 mL). The reaction mixture was stirred for 1 h at rt under N2.

To a solution of compound 2 (278 mg, 1.54 mmol, 1.1 eq) in THF (5 mL) was added the above solution at 0° C. The mixture was heated for 16 h at 70° C. under N₂. After completion of the reaction monitored by TLC (R_{f} 0.5, PE:EA=10:1), the mixture was diluted with ethyl acetate (60 mL). The organic layer was washed with brine, dried with Na₂SO₄ and filtered. The filtration was concentrated under reduced pressure. The residue was purified by column chromatography to give compound 3 (340 mg, 43%) as yellow oil.

### Step 2

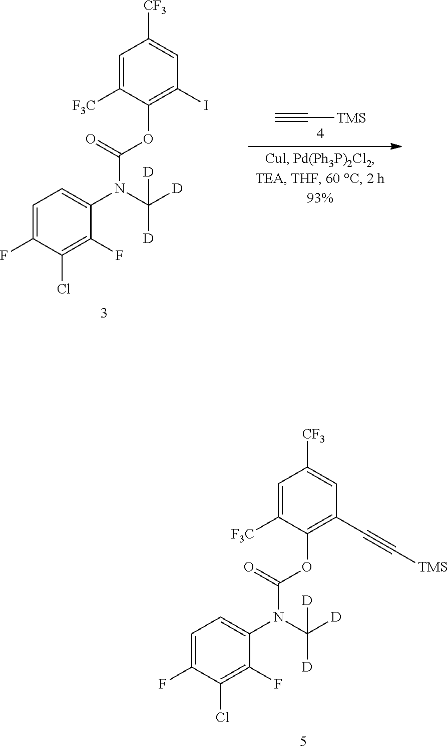

To a solution of compound 3 (340 mg, 1.27 mmol, 1.0 eq) in DMF (9 mL) was added compound 4 (77 mg, 0.79 mmol, 1.3 eq), CuI (23 mg, 0.121 mmol, 0.2 eq), TEA (123 mg, 1.21 mmol, 2 eq) and Pd(PPh₃)₂Cl₂ (51 mg, 15% wt) under inert atmosphere at rt. The reaction was stirred for 2 h at 60° C. The mixture was diluted with DCM (50 mL). The organic layer was washed with brine, dried with Na₂SO₄ and filtered. The filtration was concentrated under reduced pressure. The residue was purified by column chromatography to give compound 5 (300 mg, 93%) as red oil.

### Step 3

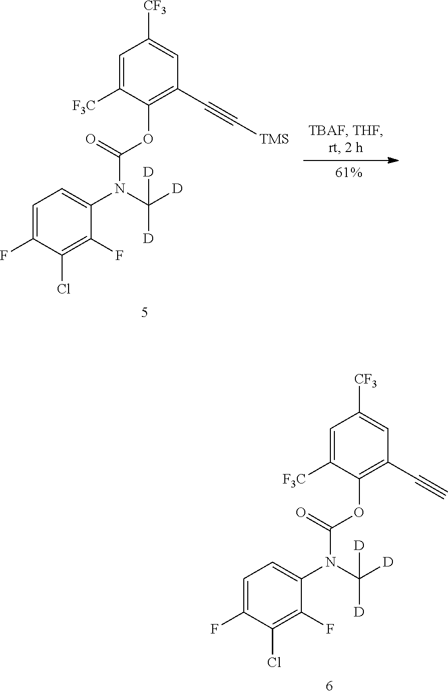 [SMILES: [2H]C([2H])([2H])N(C(=O)Oc1c(C#C[Si](C)(C)C)cc(C(F)(F)F)cc1C(F)(F)F)c1ccc(F)c(Cl)c1F ; [2H]C([2H])([2H])N(C(=O)Oc1c(C#C)cc(C(F)(F)F)cc1C(F)(F)F)c1ccc(F)c(Cl)c1F]

To a solution of compound 5 (300 mg, 0.49 mmol, 1.0 eq) in THF (2 mL), was added TBAF (1.2 mL, 2.5 eq) at rt. The reaction mixture was stirred for 2 h at rt. The reaction was monitored by LC-MS and TLC (R_{f} 0.6, PE:EA=10:1). Then the mixture was diluted with DCM (50 mL), washed with brine, dried with Na2SO4 and filtered. The filtration was concentrated under reduced pressure. The residue was purified by column chromatography to give compound 6 (180 mg, 69%) as yellow solid.

### Step 4

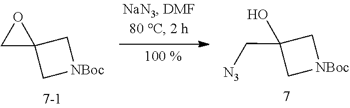 [SMILES: CC(C)(C)OC(=O)N1CC2(CO2)C1 ; CC(C)(C)OC(=O)N1CC(O)(CN=[N+]=[N-])C1]

To a solution of compound 7-1 (100 mg, 0.54 mmol, 1.0 eq) in DMF (3 mL) was added NaN₃ (106 mg, 1.62 mmol, 3.0 eq) at rt. The reaction was stirred at 80° C. for 2 h. Then the mixture was extracted with ethyl acetate (20 mL×2). The combined organic layer was washed with brine, dried with Na₂SO₄ and filtered. The filtration was concentrated under reduced pressure to give compound 7 (138 mg, 100%) as yellow oil.

### Step 5

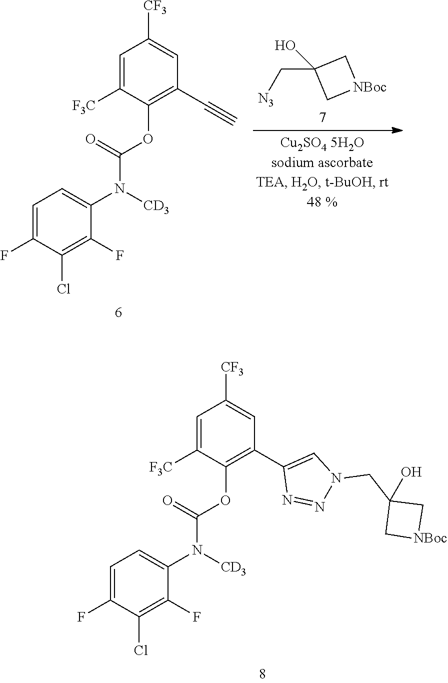

To a solution of compound 6 (180 mg, 0.39 mmol, 1.0 eq) in t-BuOH/H₂O (2 mL/2 mL) was added compound 7 (90 mg, 0.39 mmol, 1.0 eq), sodium ascorbate (39 mg, 0.20 mmol, 0.5 eq), TEA (44 mg, 0.43 mmol, 1.1 eq) and CuSO₄·5H₂O (30 mg, 0.12 mmol, 0.3 eq) under N₂ at rt. The reaction mixture was stirred for 2 h at rt. The reaction was monitored by TLC (R_{f} 0.4, PE:EA=1:1). The mixture was extracted with DCM (20 mL×2). The combined organic layer was washed with brine, dried with Na₂SO₄ and filtered. The filtration was concentrated under reduced pressure. The residue was purified by column chromatography to give compound 8 (130 mg, 48%) as white solid.

### Step 6

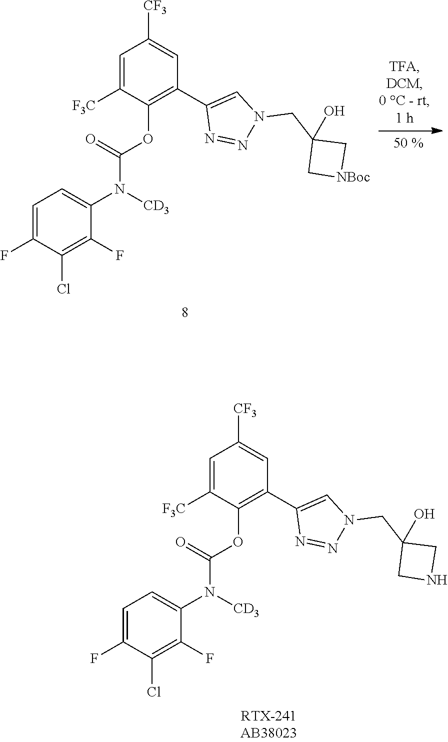 [SMILES: [2H]C([2H])([2H])N(C(=O)Oc1c(-c2cn(CC3(O)CN(C(=O)OC(C)(C)C)C3)nn2)cc(C(F)(F)F)cc1C(F)(F)F)c1ccc(F)c(Cl)c1F ; [2H]C([2H])([2H])N(C(=O)Oc1c(-c2cn(CC3(O)CNC3)nn2)cc(C(F)(F)F)cc1C(F)(F)F)c1ccc(F)c(Cl)c1F]

To a solution of compound 8 (130 mg, 0.188 mmol, 1.0 eq) in DCM (2 mL), was added TFA (2 mL) at 0° C. The reaction mixture was stirred at rt for 1 h. The mixture was concentrated under reduced pressure. The residue was purified by reverse phase C18 column (38% MeCN in H₂O), and freeze-dried to give the titled compound (AB38024, 56.6 mg, 50%) as yellow solid.

LCMS [M+H]⁺: 589.10

¹H NMR (400 MHz, CDCl₃): δ 9.62 (s, 2H), 7.96-8.31 (m, 2H), 7.78-7.85 (m, 1H), 7.34-7.35 (m, 0.66H), 7.16-7.18 (m, 0.34H), 6.88-7.03 (m, 1H), 4.81-4.93 (m, 2H), 4.28 (m, 4H).

### Example 17

### 2-[1-(3-hydroxycyclobutyl)-1H-1,2,3-triazol-4-yl]-4,6-bis(trifluoromethyl)phenyl N-(4-fluorophenyl)-N-methylcarbamate

### Step 1

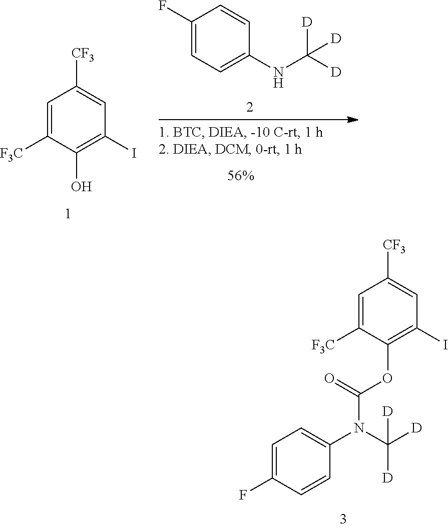 [SMILES: Oc1c(I)cc(C(F)(F)F)cc1C(F)(F)F ; [2H]C([2H])([2H])Nc1ccc(F)cc1 ; [2H]C([2H])([2H])N(C(=O)Oc1c(I)cc(C(F)(F)F)cc1C(F)(F)F)c1ccc(F)cc1]

To a solution of compound 1 (800 mg, 2.25 mmol, 1.0 eq) in DCM (10 mL) at −10° C. was added DIEA (436 mg, 3.37 mmol, 1.5 eq) and a solution of triphosgene (300 mg, 1.01 mmol, 0.5 eq) in DCM (2 mL). The reaction mixture was stirred for 1 h at rt under N₂. The reaction solution was concentrated under reduced pressure to remove the solvent. DCM (3 mL) was added to above residue. The solution was used for next step.

### Step 2

To a solution of compound 2 (776 mg, 2.92 mmol, 1.5 eq) in DCM (5 mL) at 0° C. was added the solution prepared above. The mixture was stirred for 1 h at rt under N₂. After completion of the reaction checked by TLC (R_{f} 0.5, PE:EA=10:1), HCl (1 M, 15 mL) was added. Then the mixture was extracted with DCM (30 mL×2). The combined organic layer was washed with brine, dried with Na₂SO₄ and filtered. The filtration was concentrated under reduced pressure. The residue was purified by column chromatography to give compound 3 (650 mg, 56%) as yellow oil.

### Step 3

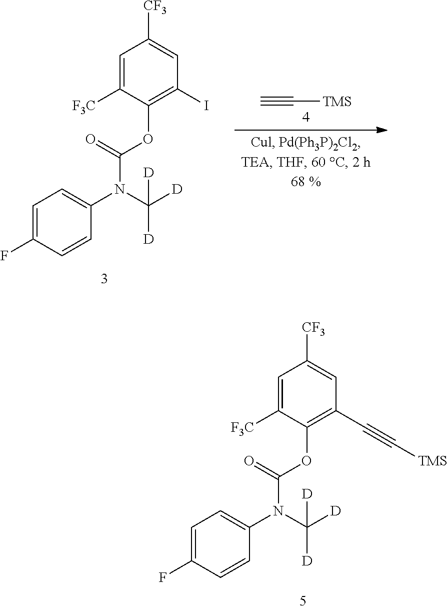

To a solution of compound 3 (650 mg, 1.27 mmol, 1.0 eq) in DMF (9 mL) at rt was added compound 4 (348 mg, 1.91 mmol, 1.5 eq), CuI (48 mg, 0.254 mmol, 0.2 eq), TEA (257 mg, 2.54 mmol, 2 eq) and Pd(PPh₃)₂Cl₂ (98 mg, 15% wt) under inert atmosphere. The reaction was stirred at 60° C. for 2 h. Then the mixture was extracted with DCM (20 mL×2). The combined organic layer was washed with brine, dried with Na2SO4 and filtered. The filtration was concentrated under reduced pressure. The residue was purified by column chromatography to give compound 5 (420 mg, 68%) as yellow oil.

### Step 4

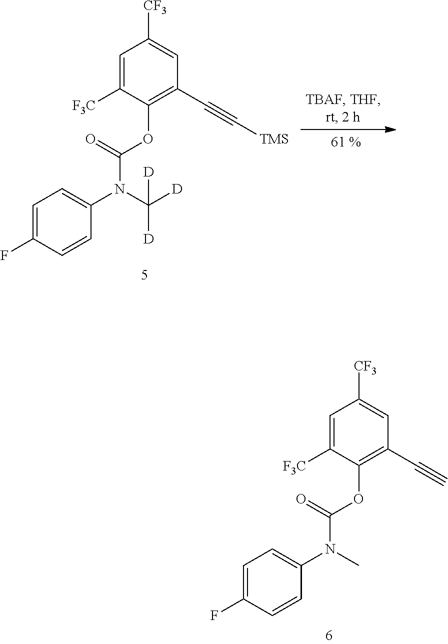 [SMILES: [2H]C([2H])([2H])N(C(=O)Oc1c(C#C[Si](C)(C)C)cc(C(F)(F)F)cc1C(F)(F)F)c1ccc(F)cc1 ; C#Cc1cc(C(F)(F)F)cc(C(F)(F)F)c1OC(=O)N(C)c1ccc(F)cc1]

To a solution of compound 5 (420 mg, 0.875 mmol, 1.0 eq) in THF (5 mL) at rt was added TBAF (1 M in THF, 1.3 mL, 1.5 eq). The reaction mixture was stirred for 2 h at rt. Then the mixture was diluted with DCM (40 mL), washed with brine, dried with Na₂SO₄ and filtered. The filtrate was concentrated under reduced pressure. The residue was purified by column chromatography to give compound 6 (220 mg, 61%) as yellow oil.

### Step 5

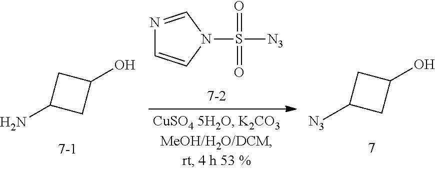

To a solution of compound 7-1 (50 mg, 0.57 mmol, 1.0 eq) in MeOH/H₂O/DCM (2 mL/1 mL/1.5 mL) was added compound 7-2 (169 mg, 0.81 mmol, 1.4 eq), CuSO₄·5H₂O (2 mg, 0.004 mmol, 0.007 eq). The mixture at rt was adjusted with K₂CO₃ (sat. aq) to pH 9. The mixture was stirred under inert atmosphere at rt for 4 h. Then the mixture was extracted with DCM (20 mL×2). The combined organic layer was washed with brine, dried with Na₂SO₄ and filtered. The filtrate was concentrated under reduced pressure to give compound 7 (35 mg, 53%) as yellow oil.

### Step 6

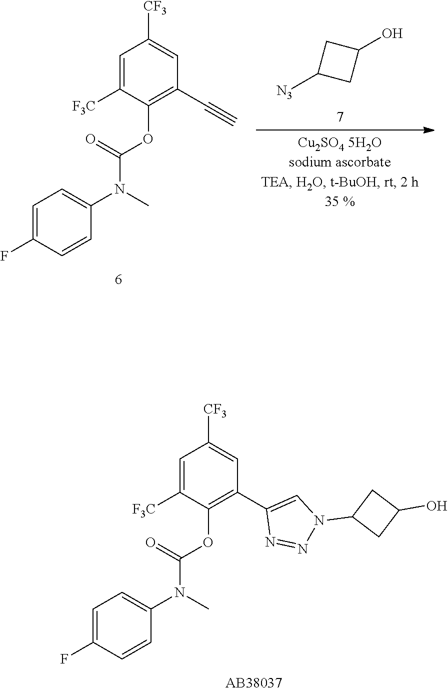 [SMILES: C#Cc1cc(C(F)(F)F)cc(C(F)(F)F)c1OC(=O)N(C)c1ccc(F)cc1 ; [N-]=[N+]=NC1CC(O)C1 ; CN(C(=O)Oc1c(-c2cn(C3CC(O)C3)nn2)cc(C(F)(F)F)cc1C(F)(F)F)c1ccc(F)cc1]

To a solution of compound 6 (110 mg, 0.269 mmol, 1.0 eq) in t-BuOH/H₂O (2 mL/2 mL) was added compound 7 (31 mg, 0.269 mmol, 1.0 eq), sodium ascorbate (27 mg, 0.135 mmol, 0.5 eq), TEA (30 mg, 0.296 mmol, 1.1 eq) and CuSO₄·5H₂O (20 mg, 0.08 mmol, 0.3 eq) under N₂ at rt. The reaction mixture was stirred at rt for 2 h. Then the mixture was extracted with DCM (30 mL×2). The combined organic layer was washed with brine, dried with Na₂SO₄ and filtered, and concentrated under reduced pressure. The residue was purified by column chromatography to give the titled compound (AB38037, 49.7 mg, 35%) as yellow solid.

LCMS: 522.25 [M+H]⁺

¹H NMR (400 MHz, CDCl₃): δ 8.40-8.51 (m, 1H), 7.63-7.96 (m, 2H), 7.24-7.49 (m, 2H), 7.03-7.18 (m, 2H), 5.18 (m, 0.17H), 4.72-4.76 (m, 1H), 4.34-4.38 (m, 0.82H), 2.94-3.12 (m, 2H), 2.50-2.68 (m, 2H), 2.37 (s, 1H).

### Example 18

### 2-[1-(1,1-dioxo-1λ⁶-thioctan-3-yl)-1H-1,2,3-triazol-4-yl]-4,6-bis(trifluoromethyl)phenyl N-(3-chloro-2,4-difluorophenyl)-N-(methy-d3)carbamate

### Step 1

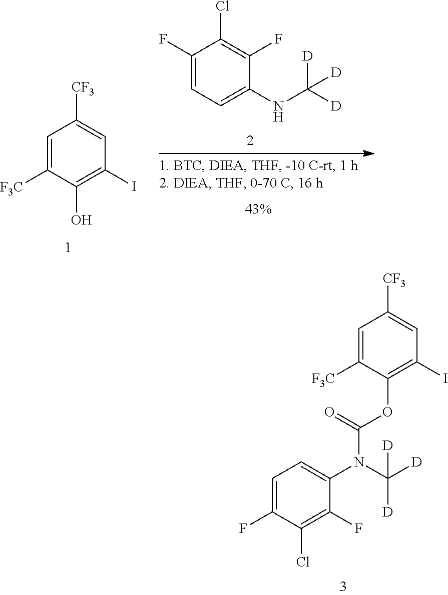 [SMILES: Oc1c(I)cc(C(F)(F)F)cc1C(F)(F)F ; [2H]C([2H])([2H])Nc1ccc(F)c(Cl)c1F ; [2H]C([2H])([2H])N(C(=O)Oc1c(I)cc(C(F)(F)F)cc1C(F)(F)F)c1ccc(F)c(Cl)c1F]

To a solution of compound 1 (500 mg, 2.25 mmol, 1.0 eq) in THF (10 mL) at −10° C. was added DIEA (272 mg, 2.1 mmol, 1.5 eq), and triphosgene (166 mg, 0.63 mmol, 0.4 eq) in THF (2 mL). The reaction mixture was stirred for 1 h at rt under N₂.

To a solution of compound 2 (278 mg, 1.54 mmol, 1.1 eq) in THF (5 mL) was added the above solution at 0° C. The mixture was heated for 16 h at 70° C. under N₂. After completion of the reaction monitored by TLC (R_{f} 0.5, PE:EA=10:1), the mixture was diluted with ethyl acetate (60 mL). The organic layer was washed with brine, dried with Na₂SO₄ and filtered. The filtration was concentrated under reduced pressure. The residue was purified by column chromatography to give compound 3 (340 mg, 43%) as yellow oil.

### Step 2

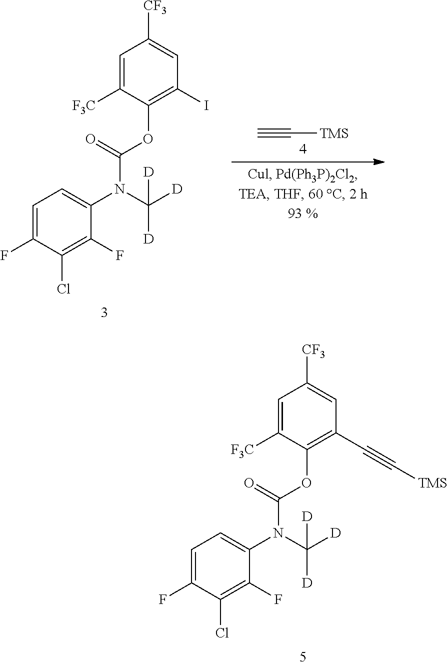

To a solution of compound 3 (340 mg, 1.27 mmol, 1.0 eq) in DMF (9 mL) was added compound 4 (77 mg, 0.79 mmol, 1.3 eq), CuI (23 mg, 0.121 mmol, 0.2 eq), TEA (123 mg, 1.21 mmol, 2 eq) and Pd(PPh₃)₂Cl₂ (51 mg, 15% wt) under inert atmosphere at rt. The reaction was stirred for 2 h at 60° C. The mixture was diluted with DCM (50 mL). The organic layer was washed with brine, dried with Na₂SO₄ and filtered. The filtration was concentrated under reduced pressure. The residue was purified by column chromatography to give compound 5 (300 mg, 93%) as red oil.

### Step 3

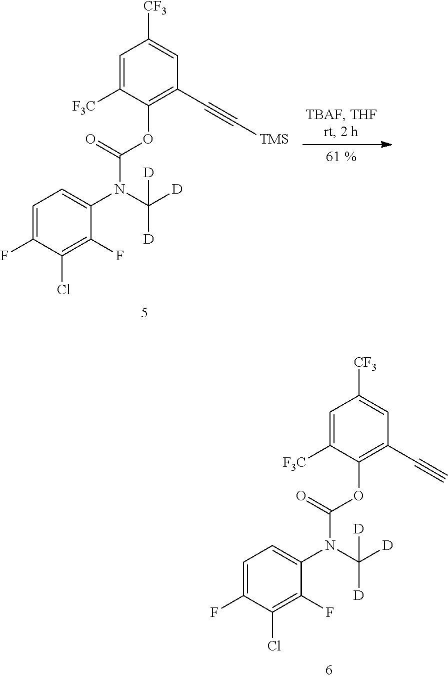 [SMILES: [2H]C([2H])([2H])N(C(=O)Oc1c(C#C[Si](C)(C)C)cc(C(F)(F)F)cc1C(F)(F)F)c1ccc(F)c(Cl)c1F ; [2H]C([2H])([2H])N(C(=O)Oc1c(C#C)cc(C(F)(F)F)cc1C(F)(F)F)c1ccc(F)c(Cl)c1F]

To a solution of compound 5 (300 mg, 0.49 mmol, 1.0 eq) in THF (2 mL), was added TBAF (1.2 mL, 2.5 eq) at rt. The reaction mixture was stirred for 2 h at rt. The reaction was monitored by LC-MS and TLC (R_{f} 0.6, PE:EA=10:1). Then the mixture was diluted with DCM (50 mL), washed with brine, dried with Na₂SO₄ and filtered. The filtration was concentrated under reduced pressure. The residue was purified by column chromatography to give compound 6 (180 mg, 69%) as yellow solid.

### Step 4

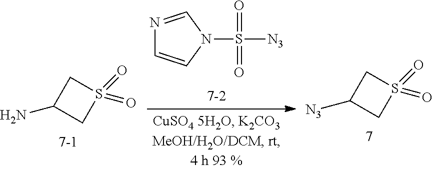

To a solution of compound 7-1 (50 mg, 0.41 mmol, 1.0 eq) in MeOH/H₂O/DCM (2 mL/1 mL/1.5 mL) was added compound 7-2 (260 mg, 1.23 mmol, 3.0 eq), CuSO₄·5H₂O (1 mg, 0.002 mmol, 0.007 eq) at rt. Aqueous K₂CO₃ (saturated) was added to adjust the solution to pH 9. The reaction was stirred for 2 h. Then the mixture was extracted with ethyl acetate (20 mL×2). The combined organic layer was washed with brine, dried with Na₂SO₄ and filtered. The filtrate was concentrated under reduced pressure to give compound 7 (56 mg, 93%) as yellow oil.

### Step 5

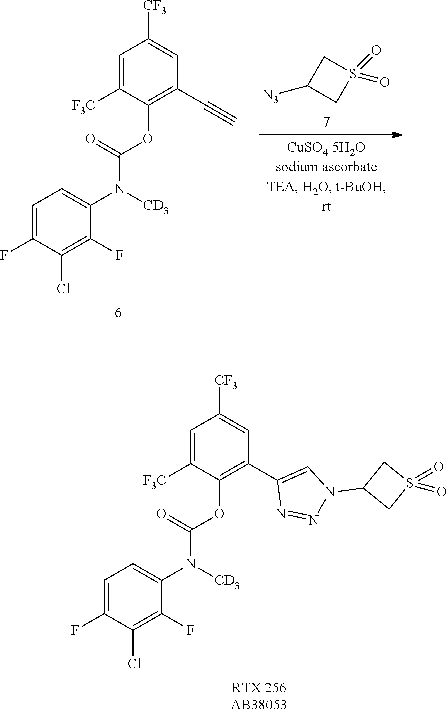 [SMILES: [2H]C([2H])([2H])N(C(=O)Oc1c(C#C)cc(C(F)(F)F)cc1C(F)(F)F)c1ccc(F)c(Cl)c1F ; [N-]=[N+]=NC1CS(=O)(=O)C1 ; [2H]C([2H])([2H])N(C(=O)Oc1c(-c2cn(C3CS(=O)(=O)C3)nn2)cc(C(F)(F)F)cc1C(F)(F)F)c1ccc(F)c(Cl)c1F]

To a solution of compound 6 (145 mg, 0.31 mmol, 1.0 eq) in t-BuOH/H₂O (2 mL/2 mL) were added compound 7 (46 mg, 0.31 mmol, 1.0 eq), sodium ascorbate (32 mg, 0.157 mmol, 0.5 eq), TEA (36 mg, 0.35 mmol, 1.1 eq) and CuSO₄·5H₂O (24 mg, 0.094 mmol, 0.3 eq) under N₂ at rt. The reaction mixture was stirred for 2 h at rt. After completion of the reaction checked by TLC (R_{f} 0.4, PE:EA, 1:1). Then the mixture was extracted with DCM (20 mL×2). The combined organic layer was washed with brine, dried with Na₂SO₄ and filtered. The filtrate was concentrated under reduced pressure. The residue was purified by column chromatography to give the titles compound (AB38053, 38.5 mg, 20%) as white solid.

LCMS: 608.20 [M+H]⁺

¹H NMR (400 MHz, CDCl₃): δ 8.85-8.96 (d, 1H), 8.48-8.57 (d, 1H), 8.06-8.16 (d, 1H), 7.34-7.68 (m, 2H), 5.69-5.72 (m, 1H), 4.96-5.02 (m, 2H), 4.75-4.85 (m, 2H).

### Examples 19

### tert-butyl 3-(2-{[(cyanomethyl)(4-fluorophenyl)carbamoyl]oxy}-3,5-bis(trifluoromethyl)phenyl)-2-oxo-2,3-dihydro-1H-imidazole-1-carboxylate

### Step 1

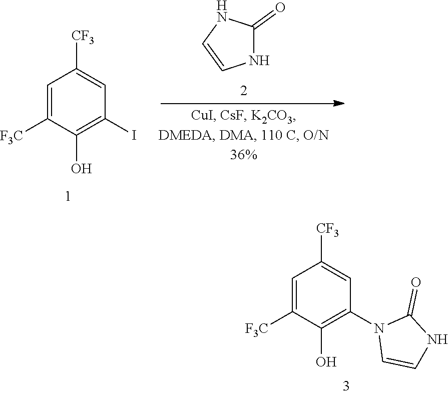 [SMILES: Oc1c(I)cc(C(F)(F)F)cc1C(F)(F)F ; O=c1[nH]cc[nH]1 ; O=c1[nH]ccn1-c1cc(C(F)(F)F)cc(C(F)(F)F)c1O]

To a solution of compound 1 (1.0 g, 2.8 mmol, 1.0 eq) and compound 2 (474 mg, 5.6 mmol, 2.0 eq) in DMA (10 mL) at rt were added CuI (270 mg, 1.4 mmol, 0.5 eq), DMEDA (249 mg, 2.8 mmol, 1.0 eq), K₂CO₃ (782 mg, 5.6 mmol, 2.0 eq), CsF (855 mg, 5.62 mmol, 2.0 eq). The reaction mixture was heated at 110° C. in an oil-bath for 4 h. After completion of the reaction checked by TLC (R_{f} 0.3, PE:EA=2:1), water (20 mL) was added and adjusted to pH 6 with HCl (1 N). The mixture was extracted with ethyl acetate (30 mL×3). The combined organic layer was washed with brine, dried with Na₂SO₄ and filtered. The filtrate was concentrated under reduced pressure. The residue was purified by column chromatography (100-200 silica gel, 50% EA in PE as eluent) to afford compound 3 (320 mg, 36%) as pale-yellow solid.

### Step 2

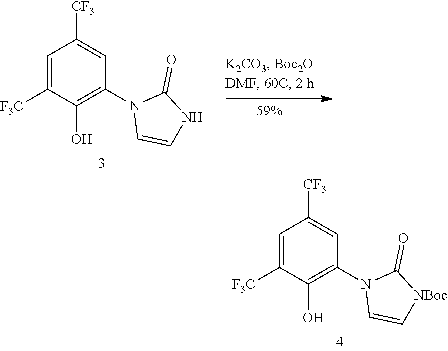 [SMILES: O=c1[nH]ccn1-c1cc(C(F)(F)F)cc(C(F)(F)F)c1O ; CC(C)(C)OC(=O)n1ccn(-c2cc(C(F)(F)F)cc(C(F)(F)F)c2O)c1=O]

To a solution of compound 3 (320 mg, 0.48 mmol, 1.0 eq) and Boc₂O (125 mg, 0.58 mmol, 1.2 eq) in DMF (10 mL) at rt was added K₂CO₃ (167 mg, 1.2 mmol, 2.5 eq). The reaction mixture was heated at 60° C. in an oil-bath for 2 h. After completion of the reaction checked with LC-MS and by TLC (R_{f} 0.4, PE:EA=2:1), the mixture was extracted with EA (30 mL×2). The combined organic layer was washed with brine, dried with Na₂SO₄ and filtered. The filtrate was concentrated under reduced pressure. The residue was purified by column chromatography (100-200 silica gel, 50% EA in PE as eluent) to afford compound 4 (250 mg, 59%) as pale yellow solid.

### Step 3

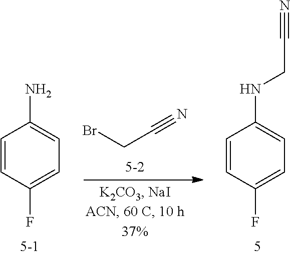

To a solution of compound 5-1 (1.0 g, 9.0 mmol, 1.0 eq) and compound 5-2 (1.19 g, 9.9 mmol, 1.1 eq) in MeCN (10 mL) at rt was added K₂CO₃ (1.88 g, 13.5 mmol, 1.5 eq), NaI (405 mg, 2.7 mmol, 0.3 eq). The reaction mixture was heated at 60° C. in an oil-bath for 10 h. After completion of the reaction checked with TLC (R_{f} 0.4, PE:EA=10:1), the mixture was diluted with ethyl acetate (60 mL), washed with brine, dried with Na₂SO₄ and filtered. The filtrate was concentrated under reduced pressure. The residue was purified by column chromatography (100-200 silica gel, 50% EA in PE as eluent) to afford compound 5 (500 mg, 37%) as pale-yellow solid.

### Step 4

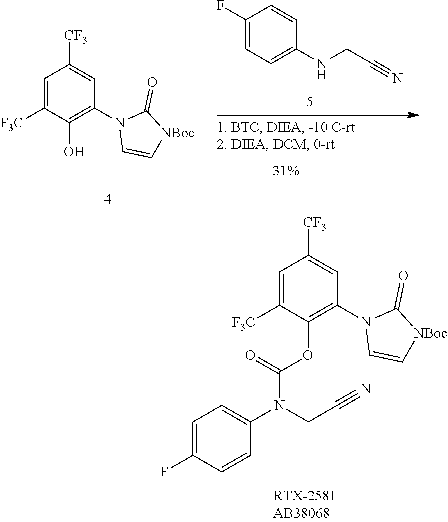 [SMILES: CC(C)(C)OC(=O)n1ccn(-c2cc(C(F)(F)F)cc(C(F)(F)F)c2O)c1=O ; N#CCNc1ccc(F)cc1 ; CC(C)(C)OC(=O)n1ccn(-c2cc(C(F)(F)F)cc(C(F)(F)F)c2OC(=O)N(CC#N)c2ccc(F)cc2)c1=O]

To a solution of compound 4 (100 mg, 0.242 mmol, 1.0 eq) and DIEA (47 mg, 0.363 mmol, 1.5 eq) in DCM (10 mL) at −10° C. was added a solution of triphosgene (36 mg, 0.121 mmol, 0.5 eq) in DCM (2 mL). The reaction mixture was stirred at rt under N₂ for 1 h. The reaction solution was concentrated under reduced pressure to remove the solvent. Then DCM (3 mL) was added to the residue above. The solution was used for next step.

To a solution of compound 5 (71 mg, 0.477 mmol, 1.5 eq) and DIEA (47 mg, 0.363 mmol, 1.5 eq) in DCM (5 mL) at 0° C. was added the solution above. The mixture was stirred for 1 h at rt under N₂. After completion of the reaction by TLC (R_{f} 0.5, PE:EA=2:1), HCl (1 M, 15 mL) was added. Then the mixture was extracted with DCM (20 mL×2). The combined organic layer was washed with brine, dried with Na₂SO₄ and filtered. The filtration was concentrated under reduced pressure. The residue was purified by column chromatography (100-200 silica gel, 50% EA in PE as eluent) to give the titled compound (AB38068, 30 mg, 21%) as off-white solid.

LCMS: 606.40 [M+H+H₂O]⁺

¹H NMR (400 MHz, CD₃OD): δ 7.86-8.01 (m, 2H), 7.35 (m, 1H), 7.15 (m, 1H), 6.95 (s, 1H), 6.32 (d, 1H), 4.65 (s, 2H).

### Example 20

### 2-(2-oxo-2,3-dihydro-1H-imidazol-1-yl)-4,6-bis(trifluoromethyl)phenyl N-(cyanomethyl)-N-(4-fluorophenyl)carbamate

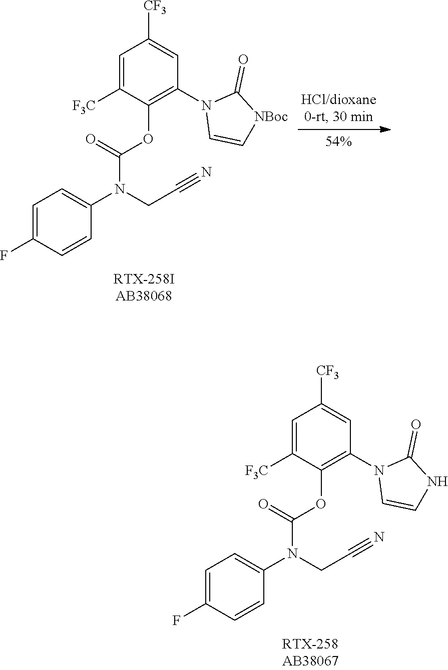 [SMILES: CC(C)(C)OC(=O)n1ccn(-c2cc(C(F)(F)F)cc(C(F)(F)F)c2OC(=O)N(CC#N)c2ccc(F)cc2)c1=O ; N#CCN(C(=O)Oc1c(-n2cc[nH]c2=O)cc(C(F)(F)F)cc1C(F)(F)F)c1ccc(F)cc1]

A mixture of HCl/dioxane (3 mL, 4 N) and tert-butyl 3-(2-{[(cyanomethyl)(4-fluorophenyl)carbamoyl]oxy}-3,5-bis(trifluoromethyl)phenyl)-2-oxo-2,3-dihydro-1H-imidazole-1-carboxylate (AB38068, RTX-258I) (55 mg, 0.09 mmol, 1.0 eq) was stirred for 30 min at rt under N₂. After completion of the reaction checked with TLC (R_{f} 0.3, PE:EA=1:1), the mixture was adjusted to pH 8-9 with Na₂CO₃ (aq). The mixture was extracted with ethyl acetate (30 mL×2). The combined organic layer was washed with brine, dried with Na₂SO₄ and filtered, and concentrated under reduced pressure. The residue was purified by column chromatography (100-200 silica gel, 50% EA in PE as eluent) to afford the titled compound (AB38067, 24.8 mg, 54%) as white solid.

LC-MS: 489.25 [M+H]⁺

¹H NMR (400 MHz, CD₃OD): δ 9.75 (s, 1H), 7.88-8.05 (m, 2H), 7.33-7.35 (m, 1.69H), 7.12-7.17 (m, 0.3H), 6.31-6.53 (d, 2H), 4.69 (s, 2H)

### Example 21

### 2-(1H-1,2,3,4-tetrazol-5-yl)-4,6-bis(trifluoromethyl)phenyl N-(4-fluorophenyl)-N-(methyl-d3)carbamate

### Step 1

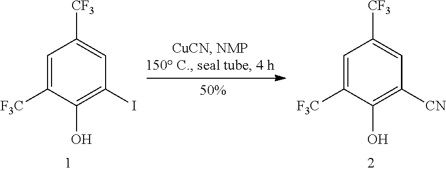 [SMILES: Oc1c(I)cc(C(F)(F)F)cc1C(F)(F)F ; N#Cc1cc(C(F)(F)F)cc(C(F)(F)F)c1O]

To a solution of compound 1 (1.0 g, 2.8 mmol, 1.0 eq) in NMP (10 mL) at rt was added CuCN (503 mg, 5.6 mmol, 2.0 eq). The reaction mixture was heated at 150° C. in an oil-bath for 4 h. After completion of the reaction checked by LC-MS and TLC (R_{f} 0.2, DCM:MeOH=10:1), water (20 mL) was added. The solution was adjusted to pH 6 with 1 N HCl. The mixture was extracted with ethyl acetate (30 mL×2). The combined organic layer was washed with brine, dried with Na₂SO₄ and filtered. The filtrate was concentrated under the reduced pressure. The residue was purified by column chromatography (100-200 silica gel, 12% MeOH in DCM as eluent) to afford compound 2 (360 mg, 50%) as pale green solid.

### Step 2

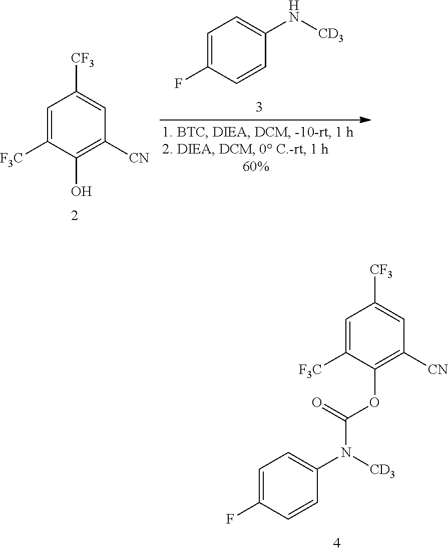 [SMILES: N#Cc1cc(C(F)(F)F)cc(C(F)(F)F)c1O ; [2H]C([2H])([2H])Nc1ccc(F)cc1 ; [2H]C([2H])([2H])N(C(=O)Oc1c(C#N)cc(C(F)(F)F)cc1C(F)(F)F)c1ccc(F)cc1]

To a solution of compound 2 (260 mg, 1.02 mmol, 1.0 eq) and DIEA (200 mg, 1.53 mmol, 1.5 eq) in DCM (10 mL) at −10° C. was added triphosgene (151 mg, 0.51 mmol, 0.5 eq in 2 mL of DCM). The reaction mixture was stirred at rt under N₂ for 1 h. The reaction solution was concentrated under reduced pressure to remove the solvent. Then DCM (3 mL) was added. The solution was used for the next step reaction.

To a solution of compound 3 (251 mg, 1.52 mmol, 1.5 eq) and DIEA (198 mg, 1.53 mmol, 1.5 eq) in DCM (5 mL) at 0° C. was added the chloroformate solution prepared above. The mixture was stirred for 1 h. After completion of the reaction checked with TLC (R_{f} 0.5, PE:EA=10:1), HCl solution (1 N, 15 mL) was added. Then the mixture was extracted with DCM (20 mL×2). The combined organic layer was washed with brine, dried with Na₂SO₄ and filtered. The filtrate was concentrated under the reduced pressure. The residue was purified by column chromatography (100-200 silica gel, 15% EA in PE as eluent) to give compound 4 (250 mg, 60%) as off-white solid.

### Step 3

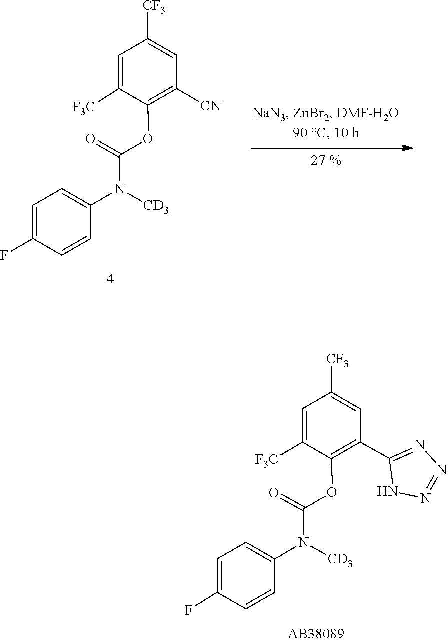 [SMILES: [2H]C([2H])([2H])N(C(=O)Oc1c(C#N)cc(C(F)(F)F)cc1C(F)(F)F)c1ccc(F)cc1 ; [2H]C([2H])([2H])N(C(=O)Oc1c(-c2nnn[nH]2)cc(C(F)(F)F)cc1C(F)(F)F)c1ccc(F)cc1]

To a solution of compound 4 (150 mg, 0.58 mmol, 1.0 eq) in DMF/H₂O (6 mL/2 mL) at rt was added sodium azide (153 mg, 2.35 mmol, 4.0 eq) and ZnBr₂ (66 mg, 0.29 mmol, 0.5 eq). The reaction mixture was heated at 90° C. under N₂ for 10 h. The reaction solution was purified by prep-HPLC to give the titled compound (AB38089, 45.8 mg, 27%) as off-white solid.

LC-MS: 453.15 [M+H+H2O]⁺

¹H NMR (400 MHz, DMSO-d6): δ 8.63-8.67 (d, 1H), 8.258-8.37 (d, 1H), 7.54-7.55 (m, 1.24H), 7.21-7.30 (m, 2.87H).

### Examples 22 and 23

### Tert-butyl 4-(2-{[(4-fluorophenyl)(methyl-d3)carbamoyl]methyl}-3,5-bis(trifluoromethyl)phenyl)-1H-pyrazole-1-carboxylate

### N-(4-fluorophenyl)-N-(methyl-d3)-2-[2-(1H-pyrazol-4-yl)-4,6-bis(trifluoromethyl)phenyl]acetamide

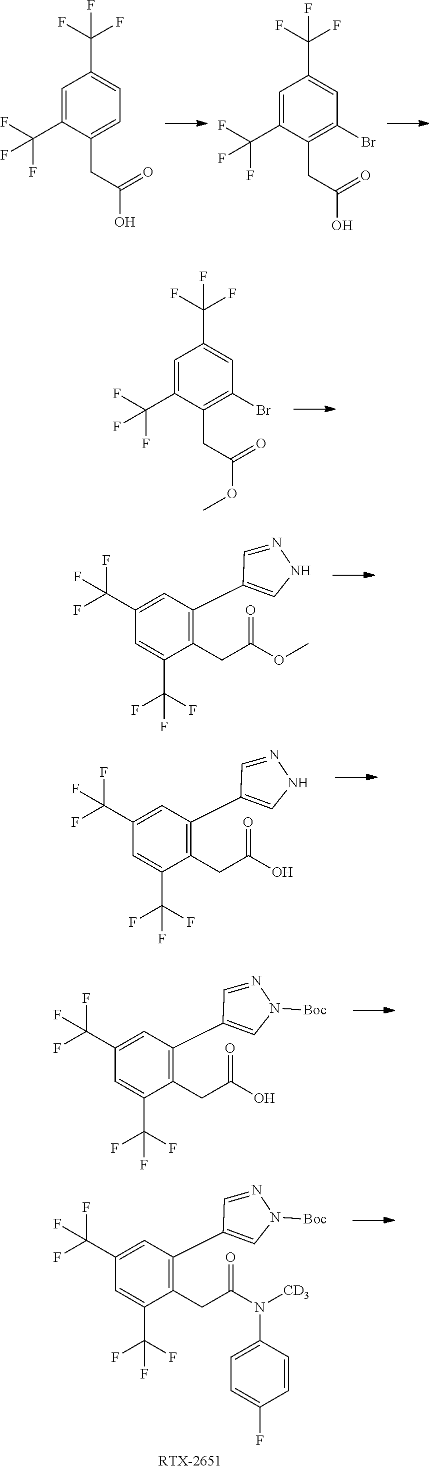

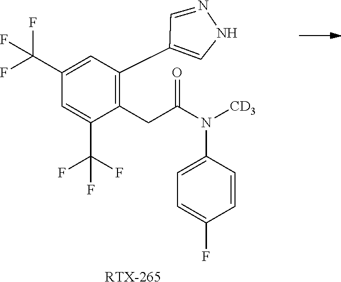

### Step 1

### Synthesis of methyl 2-(2,4-bis(trifluoromethyl)phenyl)acetate

A solution of 2-(2,4-bis(trifluoromethyl)phenyl)acetic acid 2.0 g in MeOH (30 mL) Was added one drop of H2SO4, the mixture was stirred at 68° C. overnight. After completion of the reaction, the reaction mixture was concentrated under reduced pressure to afford the crude. The Crude was dissolved with EA and washed with water and brine. Removed the solvent to get product 2.0 g.

### Step 2

### Synthesis of methyl 2-(2-bromo-4,6-bis(trifluoromethyl)phenyl)acetate

A solution of methyl 2-(2,4-bis(trifluoromethyl)phenyl)acetate 2.0 g in DCE (30 mL) Was added Na2S2O8 (4.76 g), trifluoromethanesulfonic acid 8.3 g in a seal tube was purged with N₂ for 5 min. Pd(OAc)2 0.15 g, NBS 2.37 g was added the mixture was stirred at 80° C. for 2 hour. After completion of the reaction, the reaction mixture was poured into ice-water, the product was extracted with EA, the EA was washed with water and brine. Removed the solvent to get crude product 2.0 g.

### Step 3

### Synthesis of 2-(2-bromo-4,6-bis(trifluoromethyl)phenyl)acetic Acid

A solution of 2-(2,4-bis(trifluoromethyl)phenyl)acetic acid (0.5 g, 1.84 mmol) in TFA 7 mL, and H2SO4 2 mL, NBS (0.49 g, 2.76 mmol) was added, stirred at rt for two days, poured the mixture in to water, the solid was collected and washed with water. Dried at vacuum to get white solid 0.5 g.

### Step 4

### Synthesis of methyl 2-(2-bromo-4,6-bis(trifluoromethyl)phenyl)acetate

A solution of 2-(2-bromo-4,6-bis(trifluoromethyl)phenyl)acetic acid (0.5 g) in MeOH 15 mL, added one drop of H2SO4, stirred at reflux overnight. Removed the solvent, the residue was dissolved with EA and washed with water. Removed the solvent to get light yellow oil 0.3 g.

### Step 5

### Synthesis of tert-butyl 4-(2-(2-methoxy-2-oxoethyl)-3,5-bis(trifluoromethyl)phenyl)-1H-pyrazole-1-carboxylate

A mixture of methyl 2-(2-bromo-4,6-bis(trifluoromethyl)phenyl)acetate (2.5 g, 6.85 mmol) and tert-butyl 4-(4,4,5,5-tetramethyl-1,3,2-dioxaborolan-2-yl)-1H-pyrazole-1-carboxylate (3.02 g, 10.27 mmol), Na2CO3 (1.81 g, 17.12 mmol), Pd(dppf)Cl2·CH2Cl2 (150 mg) in dioxane 40 ml and water 10 mL was heated at 85° C. overnight under N₂, poured into water, the product was extracted with EA, removed the solvent and the residued was purified by SG (DCM/MeOH=20/1) to get the compound as yellow solid 1.2 g.

### Step 6

### Synthesis of 2-(2-(1H-pyrazol-4-yl)-4,6-bis(trifluoromethyl)phenyl)acetic Acid

The methyl 2-(2-(1H-pyrazol-4-yl)-4,6-bis(trifluoromethyl)phenyl)acetate (1.0 g, 2.84 mmol) was dissolved in a mixture of 20 mL THF and 10 mL of MeOH, added 1 N LiOH aq. 10 mL The mixture was stirred at rt overnight and removed the solvent and diluted with water, acidified with Acetic acid, the product was extracted with DCM, removed the solvent to get product 1.0 g

### Step 7

### Synthesis of 2-(2-(1-(tert-butoxycarbonyl)-1H-pyrazol-4-yl)-4,6 bis(trifluoromethyl)phenyl) acetic Acid

The product (1.0 g, 2.84 mmol) was dissolved in 20 mL DCM, added (Boc) 20 (750 mg, 3.41 mmol), DIEA (750 mg, 5.68 mmol) and some DMAP. The mixture was stirred at rt overnight. The reaction mixture was washed with 1 N HCl aq., and dried over Na₂SO_{4′} removed the solvent to get product 1.2 g.

### Step 8

### Synthesis of tert-butyl 4-(2-(2-((4-fluorophenyl)(methyl-d3) amino)-2-oxoethyl)-3,5-bis (trifluoromethyl)phenyl)-1H-pyrazole-1-carboxylate

The mixture of 2-(2-(1-(tert-butoxycarbonyl)-1H-pyrazol-4-yl)-4,6 bis(trifluoromethyl)phenyl)acetic acid (0.25 g, 0.6 mmol), 4-fluoro-N-(methyl-d3)aniline (150 mg, 1.2 mmol), EDCI (140 mg, 0.72 mmol), HOBt (100 mg, 0.72 mmol) and DIEA 0.5 g,

Was stirred at rt overnight and washed with water, removed the solvent and purified by prep-HPLC to get the titled product 80 mg.

¹H NMR (400 MHz, CDCl₃) δ 7.80 (m, 4H), 7.13 (s, 4H) 3.51 (s, 2H); MS (ESI): m/z 448.84 (M+H)⁺.

### Example 24

### 2-[1-(4-hydroxy-1,1-dioxo-1λ⁶-thiolan-3-yl)-1H-1,2,3-triazol-4-yl]-4,6-bis(trifluoromethyl)phenyl N-(3-chloro-2,4-difluorophenyl)-N-(methyl-d3)carbamate

### Step 1

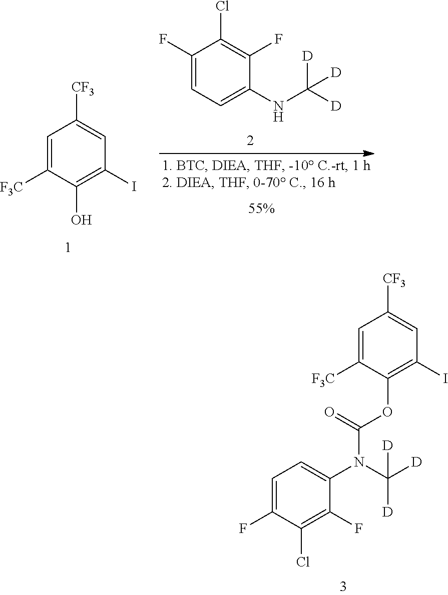 [SMILES: Oc1c(I)cc(C(F)(F)F)cc1C(F)(F)F ; [2H]C([2H])([2H])Nc1ccc(F)c(Cl)c1F ; [2H]C([2H])([2H])N(C(=O)Oc1c(I)cc(C(F)(F)F)cc1C(F)(F)F)c1ccc(F)c(Cl)c1F]

To a solution of compound 1 (500 mg, 2.25 mmol, 1.0 eq) in THF (10 mL) at −10° C. was added DIEA (272 mg, 2.1 mmol, 1.5 eq) and triphosgene (166 mg, 0.63 mmol, 0.4 eq) in THF (2 mL). The reaction mixture was stirred at rt under N₂ for 1 h, and used for the next step reaction.

### Step 2

To a solution of compound 2 (278 mg, 1.54 mmol, 1.1 eq) in THF (5 mL) at 0° C. was added the solution prepared above. The mixture was heated at 70° C. under N₂ for 16 h. After completion of the reaction monitored with LC-MS and TLC (R_{f} 0.5, PE:EA=10:1). Then the mixture was diluted with ethyl acetate (60 mL). The solution was washed with brine, dried over Na₂SO₄ and filtered. The filtrate was concentrated under reduced pressure. The residue was purified by column chromatography to give compound 3 (440 mg, 55%) as yellow oil.

### Step 3

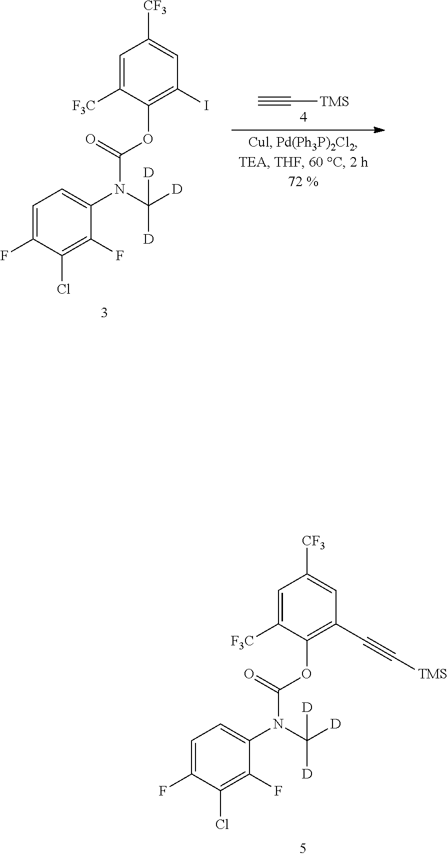

To a solution of compound 3 (440 mg, 0.78 mmol, 1.0 eq) in DMF (9 mL) at rt was added compound 4 (77 mg, 0.94 mmol, 1.2 eq), CuI (30 mg, 0.16 mmol, 0.2 eq), TEA (159 mg, 1.56 mmol, 2.0 eq) and Pd(PPh₃)₂Cl₂ (66 mg, 15% wt) under inert atmosphere. The reaction was stirred at 60° C. for 2 h. After completion of the reaction checked by TLC (R_{f} 0.9, PE:EA=10:1), the mixture was diluted with water (10 mL) and extracted with DCM (20 mL×2). The combined organic layer was washed with brine, dried over Na₂SO4 and filtered. The filtrate was concentrated under reduced pressure. The residue was purified by column chromatography to give compound 5 (300 mg, 72%) as red oil.

### Step 4

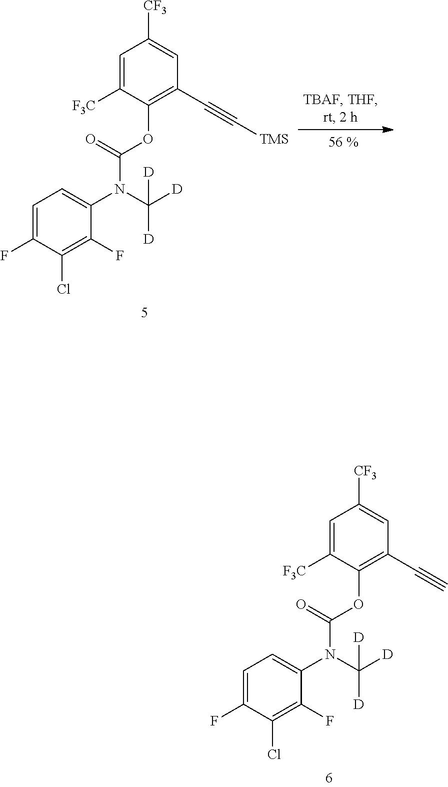 [SMILES: [2H]C([2H])([2H])N(C(=O)Oc1c(C#C[Si](C)(C)C)cc(C(F)(F)F)cc1C(F)(F)F)c1ccc(F)c(Cl)c1F ; [2H]C([2H])([2H])N(C(=O)Oc1c(C#C)cc(C(F)(F)F)cc1C(F)(F)F)c1ccc(F)c(Cl)c1F]

To a solution of compound 5 (300 mg, 0.486 mmol, 1.0 eq) in THF (2 mL) at rt was added TBAF (1 M in THF, 1.2 mL, 2.5 eq). The reaction mixture was stirred for 2 h at rt. The mixture was diluted with DCM (40 mL), washed with brine, dried over Na₂SO₄ and filtered. The filtrate was concentrated under reduced pressure. The residue was purified by column chromatography to give compound 6 (145 mg, 56%) as yellow solid.

### Step 5

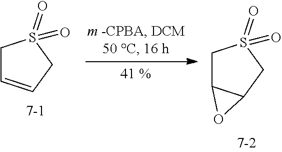 [SMILES: O=S1(=O)CC=CC1 ; O=S1(=O)CC2OC2C1]

To a solution of compound 7-1 (2.0 g, 16.9 mmol, 1.0 eq) in DCM (5 mL) at 0° C. was added m-CPBA (15.4 g, 101.6 mmol, 6.0 eq). The mixture was heated at 50° C. for 16 h. The mixture was diluted with DCM (60 mL), washed with sodium sulfite, water and brine, dried over Na₂SO₄ and filtered. The solution was concentrated under reduced pressure. The residue was purified by column chromatography to give compound 7-2 (950 mg, 41%) as white solid.

### Step 6

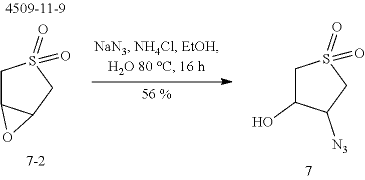 [SMILES: O=S1(=O)CC2OC2C1 ; [N-]=[N+]=NC1CS(=O)(=O)CC1O]

To a solution of compound 7-2 (200 mg, 1.49 mmol, 1.0 eq) in EtOH/H₂O (4/1, 5 mL) at 0° C. was added sodium azide (107 mg, 1.64 mmol, 1.1 eq), NH4Cl (87 mg, 1.64 mmol, 1.1 eq). The mixture was heated at 80° C. for 16 h. The reaction was diluted with water (10 mL), extracted with ethyl acetate (30 mL×2). The combined organic layers were washed with brine, dried over Na₂SO₄ and filtered. The filtrate was concentrated under reduced pressure. The residue was purified by column chromatography to give compound 7 (150 mg, 56%) as white oil.

### Step 7

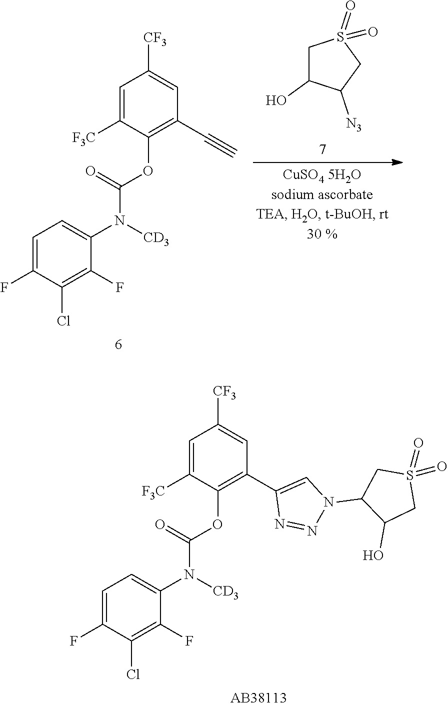 [SMILES: [2H]C([2H])([2H])N(C(=O)Oc1c(C#C)cc(C(F)(F)F)cc1C(F)(F)F)c1ccc(F)c(Cl)c1F ; [N-]=[N+]=NC1CS(=O)(=O)CC1O ; [2H]C([2H])([2H])N(C(=O)Oc1c(-c2cn(C3CS(=O)(=O)CC3O)nn2)cc(C(F)(F)F)cc1C(F)(F)F)c1ccc(F)c(Cl)c1F]

To a solution of compound 6 (100 mg, 0.22 mmol, 1.0 eq) in 1-BuOH/H₂O (2 mL/2 mL) under N₂ at rt was added compound 7 (43 mg, 0.24 mmol, 1.1 eq), sodium ascorbate (21 mg, 0.11 mmol, 0.5 eq), TEA (44 mg, 0.43 mmol, 2.0 eq) and CuSO₄·5H₂O (17 mg, 0.065 mmol, 0.3 eq). The reaction mixture was stirred at rt for 2 h. The reaction was monitored by TLC (R_{f} 0.2, PE:EA=1:1). The mixture was extracted with DCM (20 mL×2). The combined organic layer was washed with brine, dried over Na₂SO₄ and filtered. The solution was concentrated under reduced pressure. The residue was purified by column chromatography to give the titled compound (AB38113, 41.4 mg, 30%) as white solid.

LC-MS: 638.20 [M+H]⁺

¹H NMR (400 MHz, CDCl₃): δ 8.11-8.13 (s, 1.0H), 7.87-7.99 (m, 2H), 7.24-7.41 (m, 1.56H), 7.21-7.24 (m, 0.48H), 7.01-7.13 (m, 1H), 4.92-5.46 (m, 2H), 3.3.3-3.99 (m, 4H).

### Example 25

### 2-{1-[3-(dimethylamino)-2-hydroxypropyl]-1H-pyrazol-4-yl}-4,6-bis(trifluoromethyl)phenyl N-(4-fluorophenyl)-N-(methyl-d3)carbamate

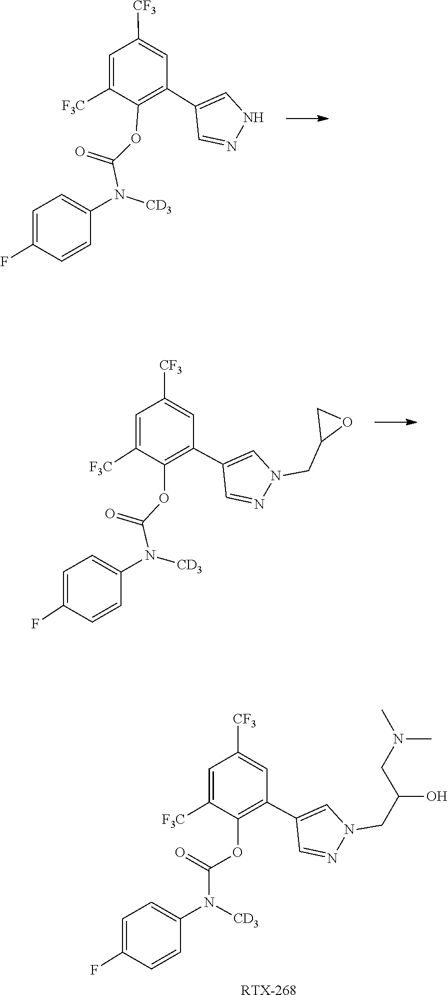

### Step 1

To a solution of 2-(1H-pyrazol-4-yl)-4,6-bis(trifluoromethyl)phenyl 4-fluorophenyl(methyl-d3)carbamate (400 mg, 0.89 mmol) in dry DMF 20 mL was added KI (445 mg, 2.68 mmol), K3PO4 (950 mg, 4.47 mmol), 2-(bromomethyl) oxirane (245 mg, 1.8 mmol). The mixture was stirred at rt under N₂ for 1 days. LCMS indicated about 95% product wanted. Filtrated, The product in DMF was used in next step directly.

### Step 2

2M dimethylamine in THF 5 mL was added to the above DMF solution 10 mL and stirred at rt overnight, LCMS found product. Filtrated and the product was extracted with DCM, removed the solvent and purified by SG to get titled compound (RTX-268; 100 mg).

¹H NMR (400 MHz, CDCl3) δ 7.75 (m, 4H), 7.41 (s, 2H), 7.02 (s, 2H) 4.28 (m, 2H), 4.17 (m, 2H), 2.38 (m, 8H); MS (ESI): m/z 552.06 (M+H)⁺.

### Examples 26 and 27

### 2-(1H-pyrazol-4-yl)-4,6-bis(trifluoromethyl)phenyl 4-fluorophenyl(methyl-d3)carbamate

### 2-(1-(2-amino-2-oxoethyl)-1H-pyrazol-4-yl)-4,6-bis(trifluoromethyl)phenyl 4-fluorophenyl(methyl-d3)carbamate

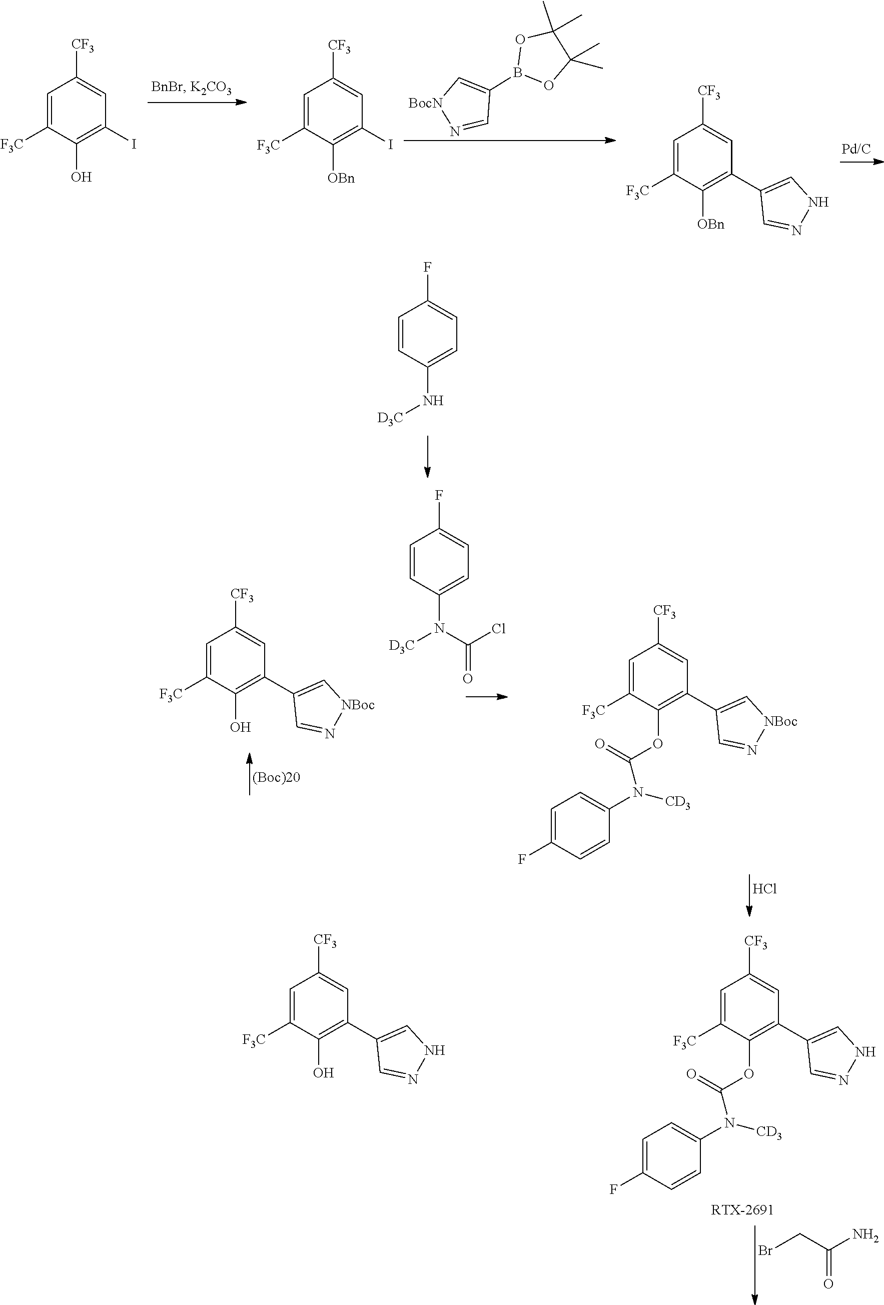

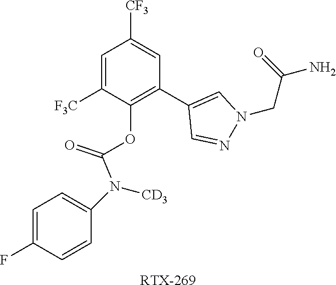

### Step 1

### Synthesis of 2-(benzyloxy)-1-iodo-3,5-bis(trifluoromethyl)benzene

A solution of 2-iodo-4,6-bis(trifluoromethyl)phenol (2.2 g, 6.2 mmol) in ketone (30 mL) Was added K₂CO3 1.0 BnBr (1.4 g, 7.7 mmol), the mixture was stirred at reflux for 8 hour, After completion of the reaction, the reaction mixture was concentrated under reduced pressure to afford the crude.

Crude was purified by SG (PE/EA=50/1) to get product 2.6 g as a colorless oil.

### Step 2

### Synthesis of 4-(2-(benzyloxy)-3,5-bis(trifluoromethyl)phenyl)-1H-pyrazole

A mixture of 2-(benzyloxy)-1-iodo-3,5-bis(trifluoromethyl)benzene (2.6 g, 5.9 mmol) and tert-butyl 4-(4,4,5,5-tetramethyl-1,3,2-dioxaborolan-2-yl)-1H-pyrazole-1-carboxylate (2.6 g, 9.36 mmol), Na2CO3 (1.2 g, 11.7 mmol), Pd(dppf)Cl2·CH2Cl2 (300 mg) in dioxane 40 ml and water 10 mL was heated at 100° C. overnight under N₂, removed the solvent and the product was extracted with EA, and purified by SG (DCM/MeOH=50/1) to get product 1.5 g.

### Step 3

### Synthesis of 2-(1H-pyrazol-4-yl)-4,6-bis(trifluoromethyl)phenol

A solution of 4-(2-(benzyloxy)-3,5-bis(trifluoromethyl)phenyl)-1H-pyrazole (1.5 g, 3.88 mmol) in MeOH (20 mL) and 10% Pd/C 0.2 g was stirred at rt under H₂ (15 psi) overnight and filtrated, removed the solvent to get product 1.2 g.

### Step 3

### Synthesis of tert-butyl 4-(2-hydroxy-3,5-bis(trifluoromethyl)phenyl)-1H-pyrazole-1-carboxylate

The 2-(1H-pyrazol-4-yl)-4,6-bis(trifluoromethyl)phenol (1.2 g, 4.0 mmol) was dissolved in 20 mL DCM, added (Boc) 20 (1.06 g, 4.9 mmol), DIEA (1.3 g, 10.1 mmol) and some DMAP. The mixture was stirred at rt overnight. The reaction mixture was washed with 1 N HCl aq., and dried over Na₂SO₄, removed the solvent to get product 1.0 g.

### Step 4

### Synthesis of tert-butyl 4-(2-((4-fluorophenyl)(methyl-d3)carbamoyloxy)-3,5-bis(trifluoromethyl)phenyl)-1H-pyrazole-1-carboxylate

To a stirred solution of tert-butyl 4-(2-hydroxy-3,5-bis(trifluoromethyl)phenyl)-1H-pyrazole-1-carboxylate (1.0 g, 0.4 mmol) in pyridine (10 mL) at RT and continued stirring at RT for 30 minutes. To the above mixture added (4-fluorophenyl)(methyl-d3)carbamic chloride (0.19 g, 0.97 mmol). The resultant reaction mixture was further stirred at 60° C. overnight. Quenched the reaction mixture with 1N HCl (150 mL) and extracted with EtOAc (2×40 mL). The organic layer separated was combined, dried over anhydrous Na₂SO₄, filtered, and concentrated under reduced pressure, the product was purified by SG (PE/EA=4/1) to get white solid 0.4 g

### Step 5

### Synthesis of 2-(1H-pyrazol-4-yl)-4,6-bis(trifluoromethyl)phenyl 4-fluorophenyl(methyl-d3)carbamate

The product was dissolved in 6 mL THF, added 2 mL con. HCl, the mixture was stirred at rt overnight and removed the solvent the product was purified by prep-HPLC to get the titled compound (Example 26, RTX-269I; 0.2 g).

### Step 6

### Synthesis of 2-(1-(2-amino-2-oxoethyl)-1H-pyrazol-4-yl)-4,6-bis(trifluoromethyl)phenyl 4-fluorophenyl(methyl-d3)carbamate

2-(1H-pyrazol-4-yl)-4,6-bis(trifluoromethyl)phenyl 4-fluorophenyl(methyl-d3)carbamate (0.2 g, 0.44 mmol) was dissolved in 4 mL MeCN, added K₂CO₃ (153 mg, 1.11 mmol) 2-bromoacetamide (92 mg, 0.67 mmol). The mixture was stirred at reflux overnight and cooled to rt, filtrated and washed with MeCN, removed the solvent and purified by prep-HPLC. The product was washed with NaHCO₃ aq., extracted with EA. Removed the solvent to get the titled compound (Example 27, RTX-269; 100 mg).

¹H NMR (400 MHz, DMSO-d6) δ 8.06 (m, 4H), 7.45 (m, 6H); MS (ESI): m/z 507.97 (M+H)⁺.

### Example 28

### 2-[1-(1-methanesulfonylazetidin-3-yl)-1H-1,2,3-triazol-4-yl]-4,6-bis(trifluoromethyl)phenyl N-(3-chloro-2,4-difluorophenyl)-N-(methyl-d3)carbamate

### Step 1

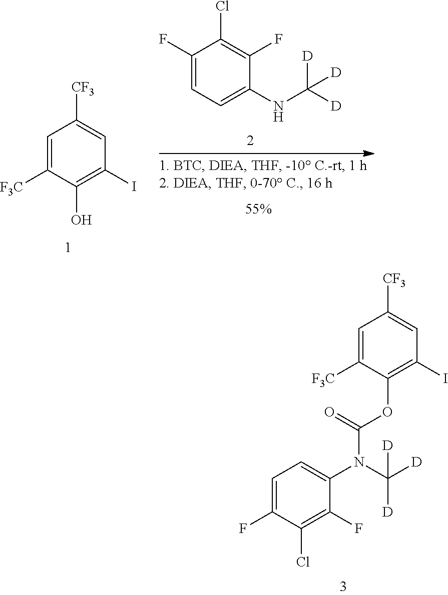 [SMILES: Oc1c(I)cc(C(F)(F)F)cc1C(F)(F)F ; [2H]C([2H])([2H])Nc1ccc(F)c(Cl)c1F ; [2H]C([2H])([2H])N(C(=O)Oc1c(I)cc(C(F)(F)F)cc1C(F)(F)F)c1ccc(F)c(Cl)c1F]

To a solution of compound 1 (500 mg, 2.25 mmol, 1.0 eq) in THF (10 mL) at −10° C. was added DIEA (272 mg, 2.1 mmol, 1.5 eq) and triphosgene (166 mg, 0.63 mmol, 0.4 eq) in THF (2 mL). The reaction mixture was stirred at rt under N₂ for 1 h and used for next step.

### Step 2

To a solution of compound 2 (278 mg, 1.54 mmol, 1.1 eq) in THF (5 mL) at 0° C. was added the chloroformate solution prepared above. The mixture was heated at 70° C. under N₂ for 16 h. The reaction was monitored with TLC (R_{f} 0.5, PE:EA=10:1), and diluted with EA (60 mL), washed with brine, dried over Na₂SO₄ and filtered. The filtrate was concentrated under reduced pressure. The residue was purified by column chromatography to give compound 3 (440 mg, 55%) as yellow oil.

### Step 3

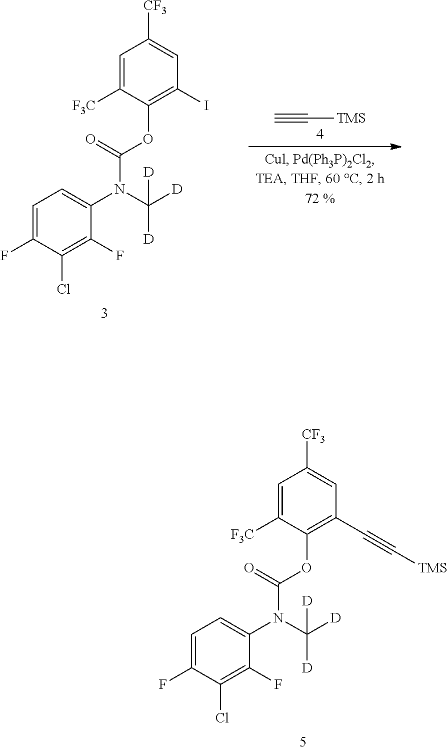

To a solution of compound 3 (440 mg, 0.78 mmol, 1.0 eq) in DMF (9 mL) was added compound 4 (77 mg, 0.939 mmol, 1.2 eq), CuI (30 mg, 0.156 mmol, 0.2 eq), TEA (159 mg, 1.56 mmol, 2 eq) and Pd(PPh₃)₂Cl₂ (66 mg, 15% wt) under inert atmosphere at rt. The reaction was stirred at 60° C. for 2 h. After completion of the reaction monitored by TLC (R_{f} 0.9, PE:EA=10:1), the mixture was diluted with water (20 mL) and extracted with DCM (20 mL×2). The combined organic layer was washed with brine, dried over Na₂SO₄ and filtered. The solution was concentrated under reduced pressure. The residue was purified by column chromatography to give compound 5 (300 mg, 72%) as red oil.

### Step 4

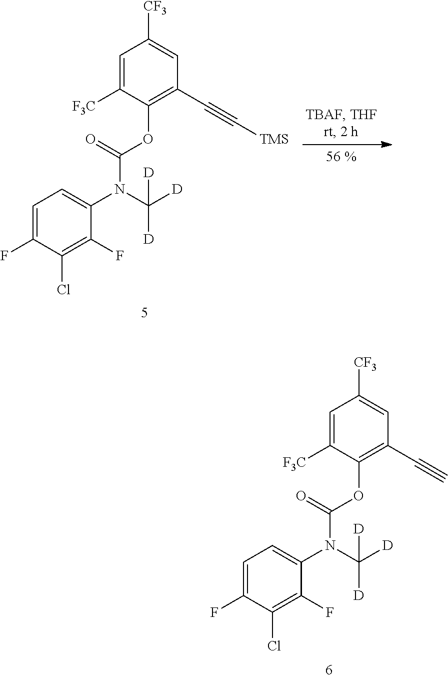 [SMILES: [2H]C([2H])([2H])N(C(=O)Oc1c(C#C[Si](C)(C)C)cc(C(F)(F)F)cc1C(F)(F)F)c1ccc(F)c(Cl)c1F ; [2H]C([2H])([2H])N(C(=O)Oc1c(C#C)cc(C(F)(F)F)cc1C(F)(F)F)c1ccc(F)c(Cl)c1F]

To a solution of compound 5 (300 mg, 0.49 mmol, 1.0 eq) in THF (2 mL) at rt was added TBAF (IM in THF, 1.2 mL, 1.2 mmol, 2.5 eq). The reaction mixture was stirred at rt for 2 h, diluted with DCM (40 mL). The mixture was washed with water and brine, dried over Na₂SO₄ and filtered. The filtrate was concentrated under reduced pressure. The residue was purified by column chromatography to give compound 6 (145 mg, 56%) as yellow solid.

### Step 5

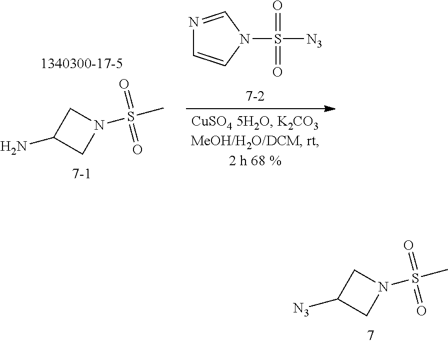

To a solution of compound 7-1 (100 mg, 0.66 mmol, 1.0 eq) in MeOH/H₂O/DCM (2 mL/1 mL/1.5 mL) at rt was added compound 7-2 (280 mg, 1.31 mmol, 2.0 eq), CuSO₄·5H₂O (2 mg, 0.004 mmol, 0.007 eq). The mixture was adjusted to pH 9 with K₂CO₃ (sat. aq). The reaction was stirred at rt for 2 h. The reaction was diluted with water, extracted with ethyl acetate (20 mL×2). The combined organic layer was washed with water and brine, dried over Na₂SO₄ and filtered. The filtrate was concentrated under reduced pressure to give crude compound 7 (80 mg, 68%) as yellow oil.

### Step 6

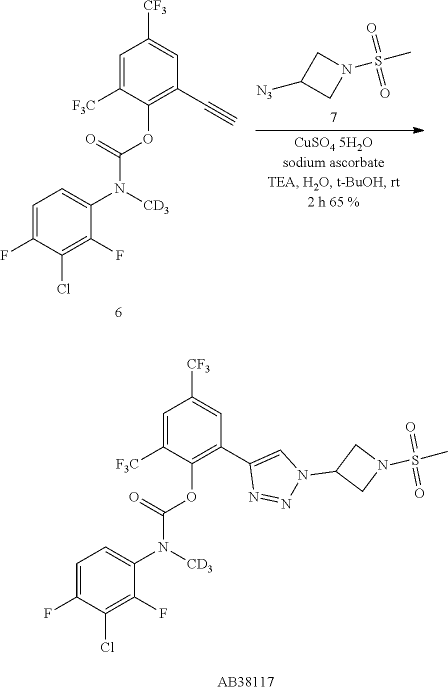

To a solution of compound 6 (100 mg, 0.22 mmol, 1.0 eq) in t-BuOH/H₂O (2 mL/2 mL) under N₂ at rt was added compound 7 (43 mg, 0.24 mmol, 1.1 eq), sodium ascorbate (21 mg, 0.11 mmol, 0.5 eq), TEA (44 mg, 0.43 mmol, 2.0 eq) and CuSO₄·5H₂O (17 mg, 0.065 mmol, 0.3 eq). The reaction mixture was stirred at rt for 2 h. After the reaction was monitored by LC-MS and TLC (R_{f} 0.4, PE:EA=1:1), quenched with water (10 mL), extracted with DCM (20 mL×2). The combined organic layer was washed with water and brine, dried over Na₂SO₄ and filtered. The solution was concentrated under reduced pressure, and purified by silica gel column chromatography to give the titled compound (AB38117: 52.1 mg, 65%) as white solid.

LC-MS: 637.15 [M+H]⁺

¹H NMR (400 MHz, CD₃OD): δ 8.44-8.61 (t, 2H), 7.98-8.06 (d, 1H), 7.42-7.68 (m, 1H), 7.15-7.41 (m, 1H), 5.60-5.66 (m, 1H), 4.44-4.57 (m, 4H), 3.09-3.10 (d, 1H).

### Example 29

### 2-{1-[2-hydroxy-3-(methylamino) propyl]-1H-1,2,3-triazol-4-yl}-4,6-bis(trifluoromethyl)phenyl N-(4-fluorophenyl)-N-(methyl-d3)carbamate

### Step 1

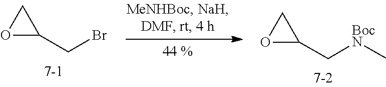

To a solution of compound 7-1 (1.0 g, 7.63 mmol, 1.0 eq) in DMF (10 mL) at rt was added NaH (366 mg, 9.14 mmol, 1.20 eq.). The reaction was stirred at rt for 0.5 h. Then tert-butyl methylcarbamate (1.1 g, 8.00 mmol, 1.05 eq) was added to the reaction mixture. The reaction was stirred at rt for 4 h. The reaction was monitored by TLC (R_{f} 0.6, PE:EA=5:1). The reaction was diluted with water (20 mL), and extracted with ethyl acetate (20 mL×2). The combined organic layer was washed with brine, dried with Na₂SO₄ and filtered. The filtration was concentrated under reduced pressure. The residue was purified by column chromatography to give compound 7-2 (600 mg, 44%) as yellow solid.

### Step 2

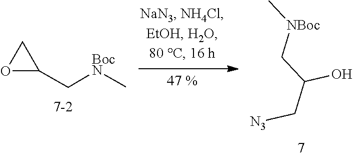 [SMILES: CN(CC1CO1)C(=O)OC(C)(C)C ; CN(CC(O)CN=[N+]=[N-])C(=O)OC(C)(C)C]

To a solution of compound 7-2 (600 mg, 3.2 mmol, 1.0 eq) in EtOH/H₂O (4/1, 5 mL) at 0° C. was added NaN₃ (250 mg, 3.85 mmol, 1.2 eq), NH₄Cl (211 mg, 3.85 mmol, 1.2 eq). The mixture was stirred at 80° C. for 16 h. After completion of the reaction by TLC (R_{f} 0.5, PE:EA=3:1), the mixture was diluted with ethyl acetate (60 mL), washed with brine, dried with Na₂SO₄ and filtered. The filtration was concentrated under reduced pressure. The residue was purified by column chromatography to give compound 7 (350 mg, 47%) as white solid.

### Step 3

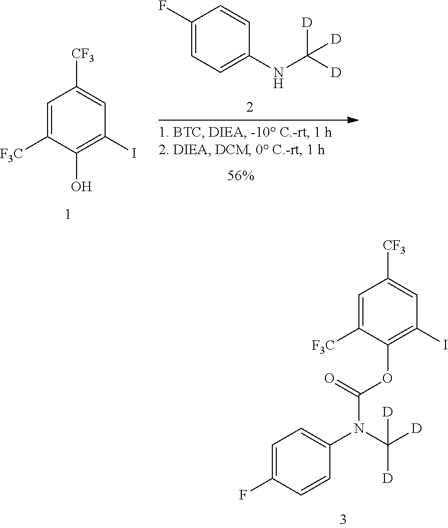 [SMILES: Oc1c(I)cc(C(F)(F)F)cc1C(F)(F)F ; [2H]C([2H])([2H])Nc1ccc(F)cc1 ; [2H]C([2H])([2H])N(C(=O)Oc1c(I)cc(C(F)(F)F)cc1C(F)(F)F)c1ccc(F)cc1]

To a solution of compound 1 (800 mg, 2.25 mmol, 1.0 eq) in DCM (10 mL) at −10° C. was added DIEA (436 mg, 3.37 mmol, 1.5 eq) and a solution of triphosgene (300 mg, 1.01 mmol, 0.5 eq) in DCM (2 mL). The reaction mixture was stirred at rt under N₂ for 1 h. The reaction solution was concentrated under reduced pressure to remove the solvent. Then DCM (3 mL) added to the residue. The solution was used for next step.

### Step 4

To a solution of compound 2 (776 mg, 2.92 mmol, 1.5 eq) in DCM (5 mL) at 0° C. was added the solution above. The mixture was stirred at rt under N₂ for 1 h. After completion of the reaction monitored with TLC (R_{f} 0.5, PE:EA=10:1), HCl (1 N, 15 mL) was added. Then the mixture was extracted with DCM (30 mL×2). The combined organic layer was washed with brine, dried with Na₂SO₄ and filtered. The filtration was concentrated under reduced pressure. The residue was purified by column chromatography to give compound 3 (650 mg, 56%) as yellow oil.

### Step 5

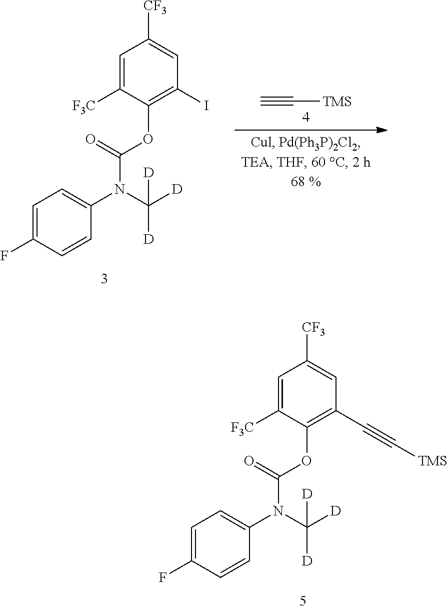

To a solution of compound 3 (650 mg, 1.27 mmol, 1.0 eq) in DMF (9 mL) was added compound 4 (348 mg, 1.91 mmol, 1.5 eq), CuI (48 mg, 0.254 mmol, 0.2 eq), triethylamine (257 mg, 2.54 mmol, 2 eq) and Pd(PPh₃)₂Cl₂ (98 mg, 15% wt) under inert atmosphere at rt. The reaction was stirred at 60° C. for 2 h. Then the mixture was diluted with DCM (40 mL), washed with brine, dried with Na₂SO₄ and filtered. The filtrate was concentrated under reduced pressure. The residue was purified by column chromatography to give compound 5 (420 mg, 68%) as yellow oil.

### Step 6

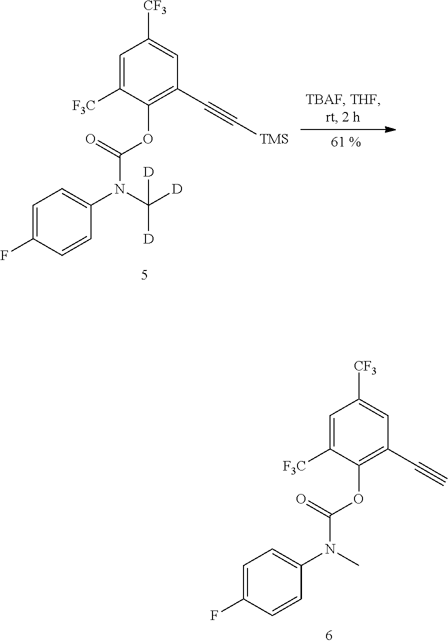 [SMILES: [2H]C([2H])([2H])N(C(=O)Oc1c(C#C[Si](C)(C)C)cc(C(F)(F)F)cc1C(F)(F)F)c1ccc(F)cc1 ; C#Cc1cc(C(F)(F)F)cc(C(F)(F)F)c1OC(=O)N(C)c1ccc(F)cc1]

To a solution of compound 5 (420 mg, 0.875 mmol, 1.0 eq) in THF (5 mL), was added TBAF (1.3 mL, 1.5 eq) at rt. The reaction mixture was stirred for 2 h at rt. Then the mixture was extracted with DCM (20 mL×2). The combined organic layer was washed with brine, dried with Na₂SO₄ and filtered. The filtration was concentrated under reduced pressure. The residue was purified by column chromatography to give compound 6 (220 mg, yield: 61%) as yellow oil.

### Step 7

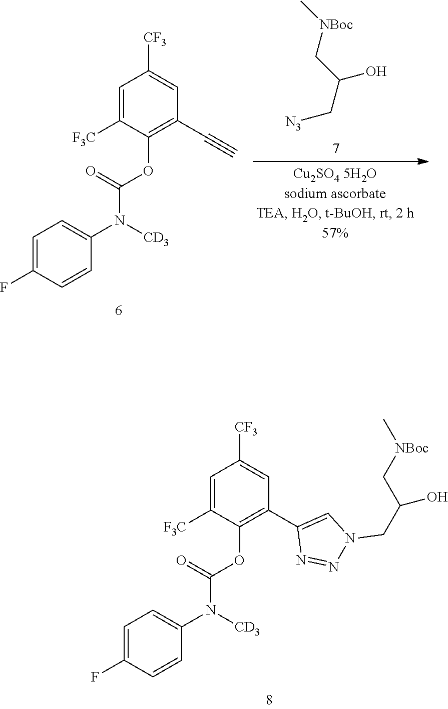 [SMILES: [2H]C([2H])([2H])N(C(=O)Oc1c(C#C)cc(C(F)(F)F)cc1C(F)(F)F)c1ccc(F)cc1 ; CN(CC(O)CN=[N+]=[N-])C(=O)OC(C)(C)C ; [2H]C([2H])([2H])N(C(=O)Oc1c(-c2cn(CC(O)CN(C)C(=O)OC(C)(C)C)nn2)cc(C(F)(F)F)cc1C(F)(F)F)c1ccc(F)cc1]

To a solution of compound 6 (200 mg, 0.49 mmol, 1.0 eq) in t-BuOH/H₂O=2 mL/2 mL, was added compound 7 (170 mg, 0.735 mmol, 1.5 eq), sodium ascorbate (48 mg, 0.245 mmol, 0.5 eq), TEA (100 mg, 0.98 mmol, 2.0 eq) and CuSO₄·5H2O (37 mg, 0.147 mmol, 0.3 eq) under N₂ at rt. The reaction mixture was stirred for 2 h at rt. After completion of the reaction by TLC (R_{f} 0.2, PE:EA=1:1). Then the mixture was extracted with DCM (20 mL×2). The combined organic layer was washed with brine, dried with Na2SO4 and filtered. The filtration was concentrated under reduced pressure. The residue was purified by column chromatography to give 8 (180 mg, yield: 57%) as white solid.

### Step 8

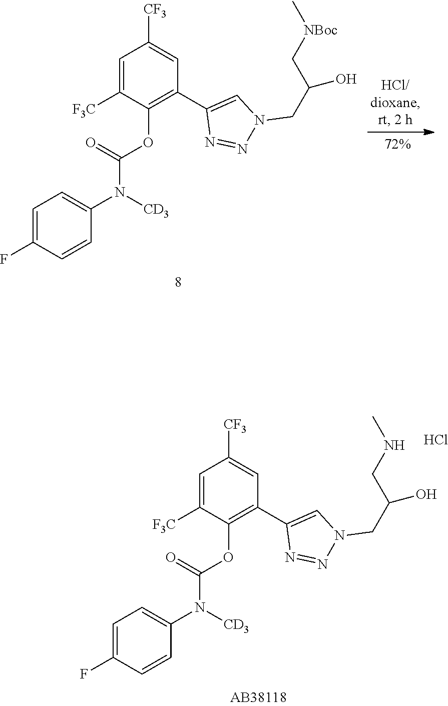 [SMILES: [2H]C([2H])([2H])N(C(=O)Oc1c(-c2cn(CC(O)CN(C)C(=O)OC(C)(C)C)nn2)cc(C(F)(F)F)cc1C(F)(F)F)c1ccc(F)cc1 ; [2H]C([2H])([2H])N(C(=O)Oc1c(-c2cn(CC(O)CNC)nn2)cc(C(F)(F)F)cc1C(F)(F)F)c1ccc(F)cc1 ; Cl]

To a solution of compound 8 (180 mg, 0.28 mmol, 1.0 eq) in dioxane (2 mL), was added HCl/dioxane (4 M, 10 mL) at rt. The reaction mixture was stirred for 2 h at rt. After completion of the reaction by TLC (R_{f} 0.5, DCM:MeOH=10:1). Then the mixture was concentrated under reduced pressure. The residue was purified by C18 column (45% MeCN in H₂O) to afford the titled compound as white solid (AB38118; 110 mg, yield: 72%).

LCMS [M+H]⁺: 539.25

¹H NMR (400 MHz, CD₃OD): δ 8.17-8.54 (m, 2H), 8.01 (d, J=24.0 Hz, 1H), 7.34-7.57 (m, 2H), 7.09-7.23 (m, 2H), 4.69-4.72 (m, 1H), 4.60 (m, 1H), 4.37 (m, 1H), 3.25-3.28 (m, 1H), 2.92-2.99 (m, 1H), 2.74 (s, 2H), 2.70 (s, 1H).

### Example 30

### 2-{1-[3-(dimethylamino)-2-hydroxypropyl]-1H-1,2,3-triazol-4-yl}-4,6-bis(trifluoromethyl)phenyl N-(4-fluorophenyl)-N-(methyl-d3)carbamate

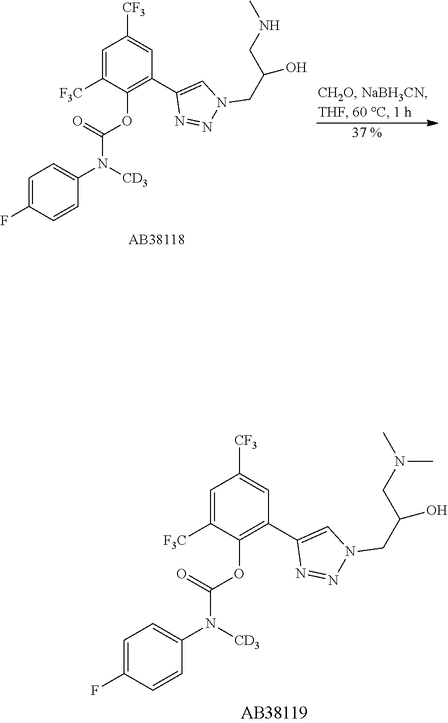 [SMILES: [2H]C([2H])([2H])N(C(=O)Oc1c(-c2cn(CC(O)CNC)nn2)cc(C(F)(F)F)cc1C(F)(F)F)c1ccc(F)cc1 ; [2H]C([2H])([2H])N(C(=O)Oc1c(-c2cn(CC(O)CN(C)C)nn2)cc(C(F)(F)F)cc1C(F)(F)F)c1ccc(F)cc1]

To a solution of 2-{1-[2-hydroxy-3-(methylamino) propyl]-1H-1,2,3-triazol-4-yl}-4,6-bis(trifluoromethyl)phenyl N-(4-fluorophenyl)-N-(methyl-d3)carbamate (AB38118) (82.0 mg, 0.15 mmol, 1.0 eq) in THF (2 mL), was added CH₂O (128 mg, 1.57 mmol, 10.0 eq), NaBH₃CN (15.1 mg, 0.228 mmol, 1.5 eq) under N₂ at rt. The reaction mixture was heated for 1 h at 60° C. After completion of the reaction by TLC (R_{f} 0.4, DCM:MeOH=10:1). Then the mixture was extracted with EA (20 mL×2). The combined organic layer was washed with brine, dried with Na₂SO₄ and filtered. The filtration was concentrated under reduced pressure. The residue was purified by Prep-HPLC (0.1 TFA in MeCN—H2O) to give the titled compound (AB38119; 31.2 mg, yield: 37%) as white solid.

LCMS [M+H]⁺: 553.30

¹H NMR (400 MHz, CD₃OD): δ 8.18-8.53 (m, 2H), 8.02 (d, J=24.0 Hz, 1H), 7.34-7.56 (m, 2H), 7.09-7.23 (m, 2H), 4.66-4.72 (m, 1H), 4.47-4.59 (m, 2H), 3.33-3.36 (m, 1H), 3.09-3.15 (m, 1H), 2.92 (d, J=16.0 Hz, 6H)

### Example 31

### 2-(1,3,4-oxadiazol-2-yl)-4,6-bis(trifluoromethyl)phenyl N-(4-fluorophenyl)-N-(methyl-d3)carbamate

### Step 1

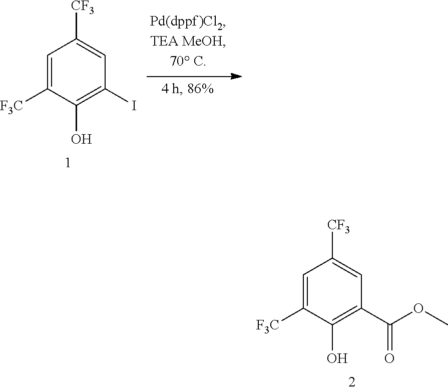 [SMILES: Oc1c(I)cc(C(F)(F)F)cc1C(F)(F)F ; COC(=O)c1cc(C(F)(F)F)cc(C(F)(F)F)c1O]

To a solution of compound 1 (500 mg, 1.40 mmol, 1.0 eq) in MeOH (30 mL) were added TEA (707 mg, 7.00 mmol, 5.0 eq) and Pd(dppf)Cl₂ (100 mg, 0.10 mmol, 0.1 eq.). The reaction mixture was stirred for 4 h at 85° C. LC-MS analysis indicated the desired product was generated and the starting material was gone. Then the mixture was extracted with ethyl acetate (20 mL×2). The combined organic layer was washed with brine, dried over Na₂SO₄, and filtered. The filtration was concentrated under reduced pressure. The residue was purified by column chromatography to give compound 2 (350 mg, 86%) as white solid.

TLC: PE/EA=10/1

R_{f} (Compound 1)=0.2

R_{f} (Compound 2)=0.8

LC-MS: 287.15 [M−1]⁺

### Step 2

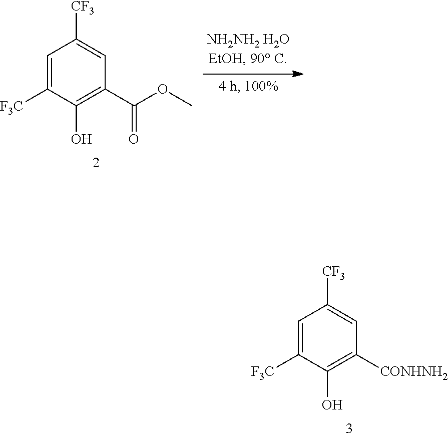

To a solution of compound 2 (250 mg, 0.87 mmol, 1.0 eq) in EtOH (5 mL) was added NH₂NH₂·H₂O (4 mL) at rt. The mixture was stirred for 4 h at 90° C. LC-MS analysis indicated the desired product was formed and the starting material was gone. The mixture was filtered and the filtrate was concentrated under reduced pressure to give compound 3 (250 mg, 100%) as yellow solid.

TLC: DCM/MeOH=10/1

R_{f} (Compound 2)=0.8

R_{f} (Compound 3)=0.1

LC-MS: 289.15 [M+1]⁺

### Step 3

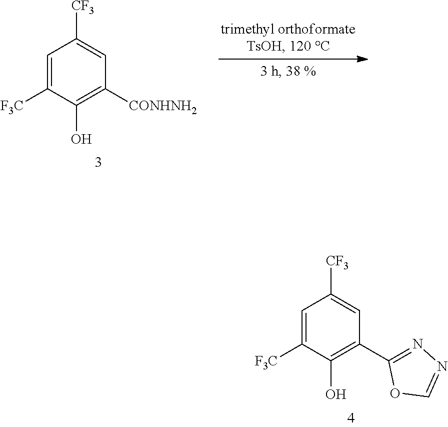

To a solution of compound 3 (250 mg, 0.87 mmol, 1.0 eq) in trimethyl orthoformate (368 mg, 3.47 mmol, 4.0 eq) was added TsOH (74 mg, 0.43 mmol, 0.5 eq) at RT. The reaction mixture was stirred for 3 h at 120° C. LC-MS analysis indicated the desired product was formed and the starting material was gone. Then the mixture was extracted with ethyl acetate (20 mL×2). The combined organic layer was washed with brine, dried over Na₂SO₄, and filtered. The filtrate was concentrated under reduced pressure. The residue was purified by column chromatography to give compound 4 (100 mg, 38%) as yellow solid.

TLC: PE/EA=1/1

R_{f} (Compound 3)=0.1

R_{f} (Compound 4)=0.4

LC-MS: 297.10 [M−1]⁺

### Step 4

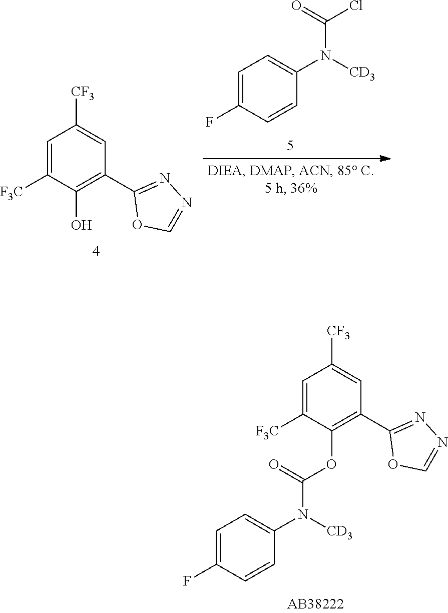 [SMILES: Oc1c(-c2nnco2)cc(C(F)(F)F)cc1C(F)(F)F ; [2H]C([2H])([2H])N(C(=O)Cl)c1ccc(F)cc1 ; [2H]C([2H])([2H])N(C(=O)Oc1c(-c2nnco2)cc(C(F)(F)F)cc1C(F)(F)F)c1ccc(F)cc1]

To a solution of compound 4 (100 mg, 0.33 mmol, 1.0 eq) in ACN (4 mL) were added DIEA (86 mg, 0.67 mmol, 2.0 eq), compound 5 (96 mg, 0.50 mmol, 1.5 eq) and DMAP (20 mg, 0.17 mmol, 0.5 eq). The reaction mixture was stirred for 5 h at 85° C. LC-MS indicated the desired product was formed and the starting material was gone. Then the mixture was extracted with ethyl acetate (20 mL×2). The combined organic layer was washed with brine, dried with Na₂SO₄, and filtered. The filtrate was concentrated under reduced pressure. The residue was purified by column chromatography to give titled compound (AB38222: 54.9 mg, 36%) as white solid.

LC-MS [M+H]⁺: 453.25

¹H NMR (400 MHz, CD₃OD) δ9.20 (s, 1H), 8.67-8.61 (m, 1H), 8.31-8.22 (m, 1H), 7.60-7.57 (m, 1H), 7.40-7.36 (m, 1H), 7.18-7.10 (m, 2H)

### Example 32

### 2-(1H-1,2,4-triazol-1-yl)-4,6-bis(trifluoromethyl)phenyl N-(4-fluorophenyl)-N-(methyl-d3)carbamate

### Step 1

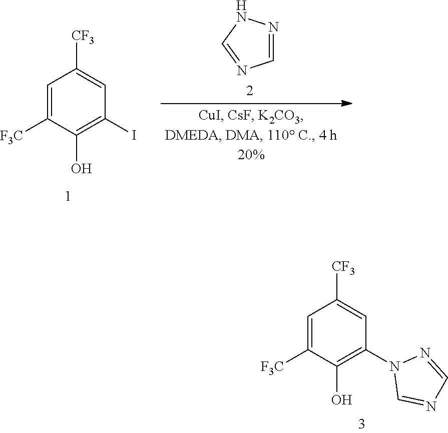 [SMILES: Oc1c(I)cc(C(F)(F)F)cc1C(F)(F)F ; c1nc[nH]n1 ; Oc1c(-n2cncn2)cc(C(F)(F)F)cc1C(F)(F)F]

To a solution of compound 1 (300 mg, 0.845 mmol, 1.0 eq) and compound 2 (117 mg, 1.69 mmol, 2.0 eq) in DMA (10 mL) were added CuI (81 mg, 0.42 mmol, 0.5 eq), DMEDA (75 mg, 0.845 mmol, 1.0 eq), K₂CO₃ (235 mg, 1.69 mmol, 2.0 eq), CsF (257 mg, 1.69 mmol, 2.0 eq) at rt. The reaction mixture was heated at 110° C. in an oil-bath for 4 h. After completion of the reaction monitored by TLC (R_{f} 0.3, PE:EA=2:1), water (20 mL) was added and adjusted to pH 6-7 with 1 N HCl. The mixture was extracted with EA (30 mL×2). The combined organic layer was washed with brine, dried over Na₂SO₄ and filtered. The filtrate was concentrated under reduced pressure. The residue was purified by column chromatography (100-200 silica gel, 50% EA in PE as eluent) to afford compound 3 (50 mg, 20%) as pale-yellow solid.

### Step 2

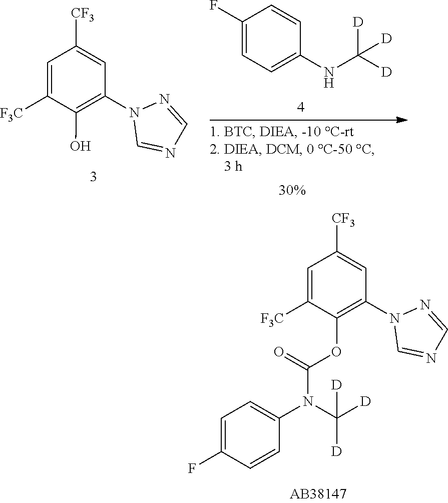 [SMILES: Oc1c(-n2cncn2)cc(C(F)(F)F)cc1C(F)(F)F ; [2H]C([2H])([2H])Nc1ccc(F)cc1 ; [2H]C([2H])([2H])N(C(=O)Oc1c(-n2cncn2)cc(C(F)(F)F)cc1C(F)(F)F)c1ccc(F)cc1]

To a solution of compound 3 (100 mg, 0.336 mmol, 1.0 eq) in DCM (10 mL) at −10° C. was added DIEA (65 mg, 0.505 mmol, 1.5 eq), and a solution of triphosgene (49 mg, 0.168 mmol, 0.5 eq) in DCM (2 mL). The reaction mixture was stirred for 1 h at rt under N₂. The reaction solution was concentrated under reduced pressure to remove the solvent, diluted with DCM (3 mL). The solution was used for the next step.

### Step 3

To a solution of compound 4 (82 mg, 0.505 mmol, 1.5 eq) and DIEA (65 mg, 0.505 mmol, 1.5 eq) and Py (0.5 mL) in DCM (5 mL) at 0° C. was added the solution above. The mixture was stirred at 50° C. for 3 h under N₂. After completion of the reaction monitored by TLC (R_{f} 0.6, DCM:MeOH=10:1), the mixture was diluted with DCM (50 mL), washed with brine, dried over Na₂SO₄ and filtered. The filtrate was concentrated under reduced pressure. The residue was purified by prep-HPLC (C18) to give the titled compound (AB38147: 47 mg, 30%) as white solid.

LC-MS: 452.15 [M+H]⁺

¹H NMR (400 MHz, CD₃OD): δ 8.77 (s, 0.8H), 8.53 (s, 0.2H), 8.16-8.25 (m, 2H), 8.06 (s, 0.2H), 7.98 (s, 0.8H), 7.27-7.24 (m, 1H), 7.15-7.03 (m, 3H).

### Example 33

### 2-{1-[1-(2-aminoethanesulfonyl) azetidin-3-yl]-1H-1,2,3-triazol-4-yl}-4,6-bis(trifluoromethyl)phenyl N-(3-chloro-2,4-difluorophenyl)-N-(methyl-d3)carbamate

### Step 1

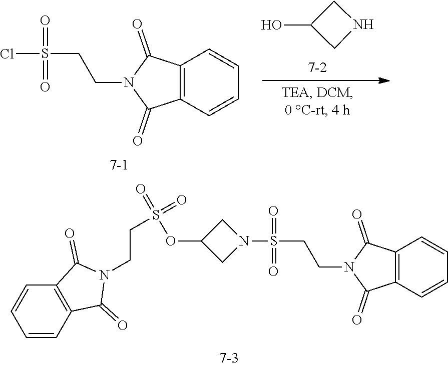 [SMILES: O=C1c2ccccc2C(=O)N1CCS(=O)(=O)Cl ; OC1CNC1 ; O=C1c2ccccc2C(=O)N1CCS(=O)(=O)OC1CN(S(=O)(=O)CCN2C(=O)c3ccccc3C2=O)C1]

To a solution of compound 7-3 (1.0 g, 9.13 mmol, 1.0 eq) in DCM (10 mL) at 0° C. was added DIEA (3.54 g, 27.39 mmol, 3.0 eq) and compound 7-1 (2.75 g, 10.04 mmol, 1.10 eq.). The reaction was stirred at rt for 4 h. After completion of the reaction checked by TLC (R_{f} 0.6, PE:EA=5:1), water (20 mL) was added to quench the reaction. Then the mixture was extracted with DCM (20 mL×2). The combined organic layer was washed with brine, dried over Na₂SO₄ and filtered. The filtrate was concentrated under reduced pressure. The residue was purified by column chromatography to give compound 7-3 (1.2 g, 60%) as yellow oil.

### Step 2

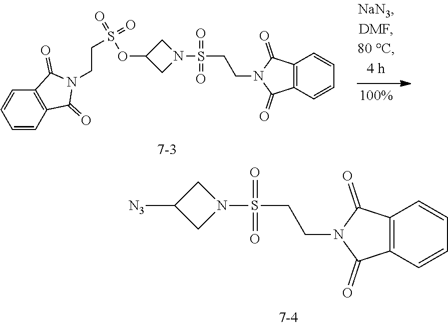 [SMILES: O=C1c2ccccc2C(=O)N1CCS(=O)(=O)OC1CN(S(=O)(=O)CCN2C(=O)c3ccccc3C2=O)C1 ; [N-]=[N+]=NC1CN(S(=O)(=O)CCN2C(=O)c3ccccc3C2=O)C1]

To a solution of compound 7-3 (400 mg, 0.73 mmol, 1.0 eq) in DMF (5 mL) at rt was added NaN₃ (145 mg, 2.19 mmol, 3.0 eq). The mixture was stirred at 80° C. for 4 h. After completion of the reaction monitored by TLC (R_{f} 0.5, PE:EA=3:1), water (20 mL) was added to quench the reaction. Then the mixture was extracted with EA (30 mL×2). The combined organic layer was washed with brine, dried over Na₂SO₄ and filtered. The filtrate was concentrated under reduced pressure to give crude compound 7-4 (300 mg, 100%) as yellow oil.

### Step 3

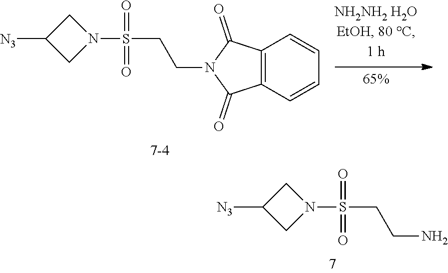 [SMILES: [N-]=[N+]=NC1CN(S(=O)(=O)CCN2C(=O)c3ccccc3C2=O)C1 ; [N-]=[N+]=NC1CN(S(=O)(=O)CCN)C1]

To a solution of compound 7-4 (300 mg, 0.895 mmol, 1.0 eq) in EtOH (10 mL) at rt was added NH₂NH2 H₂O (447 mg, 8.95 mmol, 10 eq). The reaction mixture was stirred at 80° C. under N₂ for 1 h. The reaction was monitored by TLC (R_{f} 0.1, PE:EA=3:1). Then the reaction mixture was concentrated under reduced pressure. The residue was dissolved in water (20 mL). The solution was extracted with DCM (30 mL×2). The combined organic layer was washed with brine, dried over Na₂SO₄ and filtered. The filtrate was concentrated under reduced pressure to give crude compound 7 (120 mg, 65%) as yellow oil.

### Step 4

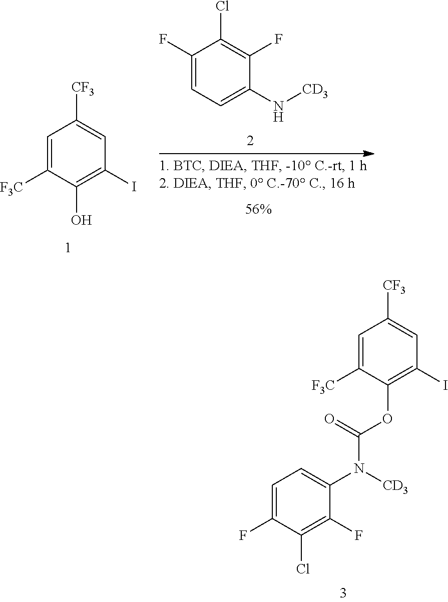 [SMILES: Oc1c(I)cc(C(F)(F)F)cc1C(F)(F)F ; [2H]C([2H])([2H])Nc1ccc(F)c(Cl)c1F ; [2H]C([2H])([2H])N(C(=O)Oc1c(I)cc(C(F)(F)F)cc1C(F)(F)F)c1ccc(F)c(Cl)c1F]

To a solution of compound 1 (800 mg, 2.25 mmol, 1.0 eq) in DCM (10 mL) at −10° C. was added DIEA (436 mg, 3.37 mmol, 1.5 eq), and a solution of triphosgene (300 mg, 1.01 mmol, 0.5 eq) in DCM (2 mL). The reaction mixture was stirred at rt for 1 h under N₂. The reaction solution was concentrated under reduced pressure to remove the solvent. Then DCM (5 mL) was added to the residue. The solution was used for the next step.

To a solution of compound 2 (776 mg, 2.92 mmol, 1.5 eq) in DCM (5 mL) at 0° C. was added the residue solution above. The mixture was heated at 70° C. for 16 h under N₂. After completion of the reaction monitored by TLC (R_{f} 0.5, PE:EA=10:1), HCl (1 M, 15 mL) was added to quench the reaction. Then the mixture was extracted with DCM (30 mL×2). The combined organic layer was washed with brine, dried over Na₂SO₄ and filtered. The filtrate was concentrated under reduced pressure. The residue was purified by column chromatography to give compound 3 (650 mg, 56%) as yellow oil.

### Step 5

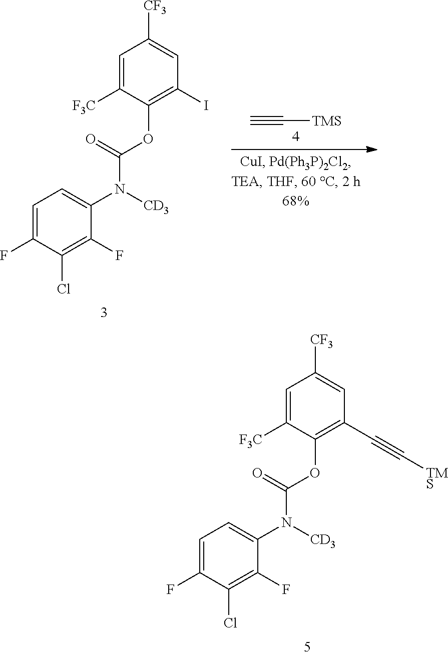

To a solution of compound 3 (650 mg, 1.27 mmol, 1.0 eq) in THF (9 mL) were added compound 4 (348 mg, 1.91 mmol, 1.5 eq), CuI (48 mg, 0.254 mmol, 0.2 eq), TEA (257 mg, 2.54 mmol, 2 eq) and Pd(PPh₃)₂Cl₂ (98 mg, 15% wt) under inert atmosphere at rt. The reaction was stirred at 60° C. for 2 h. Then the mixture was extracted with DCM (20 mL×2). The combined organic layer was washed with brine, dried over Na₂SO₄ and filtered. The filtrate was concentrated under reduced pressure. The residue was purified by column chromatography to give compound 5 (420 mg, 68%) as yellow oil.

### Step 6

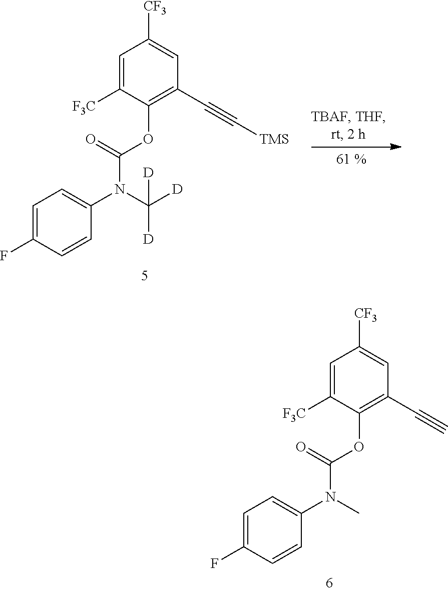 [SMILES: [2H]C([2H])([2H])N(C(=O)Oc1c(C#C[Si](C)(C)C)cc(C(F)(F)F)cc1C(F)(F)F)c1ccc(F)cc1 ; C#Cc1cc(C(F)(F)F)cc(C(F)(F)F)c1OC(=O)N(C)c1ccc(F)cc1]

To a solution of compound 5 (420 mg, 0.875 mmol, 1.0 eq) in THF (5 mL) was added TBAF (1.3 mL, 1.5 eq) at rt. The reaction mixture was stirred for at rt 2 h. Then the mixture was diluted with DCM (40 mL), washed with brine, dried over Na₂SO₄ and filtered. The filtrate was concentrated under reduced pressure. The residue was purified by column chromatography to give compound 6 (220 mg, 61%) as yellow oil.

### Step 7

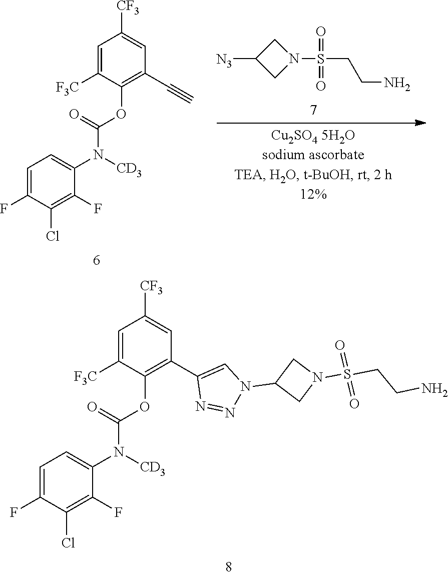 [SMILES: [2H]C([2H])([2H])N(C(=O)Oc1c(C#C)cc(C(F)(F)F)cc1C(F)(F)F)c1ccc(F)c(Cl)c1F ; [N-]=[N+]=NC1CN(S(=O)(=O)CCN)C1 ; [2H]C([2H])([2H])N(C(=O)Oc1c(-c2cn(C3CN(S(=O)(=O)CCN)C3)nn2)cc(C(F)(F)F)cc1C(F)(F)F)c1ccc(F)c(Cl)c1F]

To a solution of compound 6 (30 mg, 0.065 mmol, 1.0 eq) in t-BuOH/H₂O (2 mL/2 mL) were added compound 7 (20 mg, 0.097 mmol, 1.5 eq), sodium ascorbate (7.0 mg, 0.032 mmol, 0.5 eq), TEA (20 mg, 0.195 mmol, 3.0 eq) and CuSO₄·5H₂O (5 mg, 0.02 mmol, 0.3 eq) under N₂ at rt. The reaction mixture was stirred at rt for 2 h. After completion of the reaction monitored by TLC (R_{f} 0.1, PE:EA=1:2), the mixture was extracted with DCM (20 mL×2). The combined organic layer was washed with brine, dried over Na2SO4 and filtered. The filtrate was concentrated under reduced pressure. The residue was purified by column chromatography to give the titled compound (AB38171: 5.16 mg, 12%) as white solid.

LC-MS: 666.25 [M+H]⁺

¹H NMR (400 MHz, CD₃OD): δ 8.57-8.40 (m, 2H), 8.07 (s, 0.5H), 7.99 (s, 0.5H), 7.44-7.25 (m, 1H), 7.23-7.17 (m, 1H), 5.69-5.62 (m, 1H), 4.64-4.59 (m, 4H), 3.58 (t, 2H), 3.46 (t, 2H)

### Example 34

### 2-(5-oxo-4,5-dihydro-1H-1,2,4-triazol-4-yl)-4,6-bis(trifluoromethyl)phenyl N-(4-fluorophenyl)-N-(methyl-d3)carbamate

### Step 1

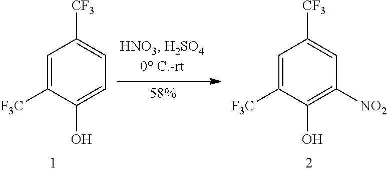 [SMILES: Oc1ccc(C(F)(F)F)cc1C(F)(F)F ; O=[N+]([O-])c1cc(C(F)(F)F)cc(C(F)(F)F)c1O]

To a solution of compound 1 (2.0 g, 8.7 mmol, 1.0 eq) in H2SO4 (20 mL) was added HNO₃ (927 mg, 9.57 mmol, 1.1 eq, 68%) at 0° C. The reaction mixture was stirred at rt for 1 h. After completion, the reaction solution was poured into water (50 mL). The mixture was extracted with ethyl acetate (50 mL×2). The combined organic layer was washed with brine, dried over Na₂SO₄, and filtered. The filtration was concentrated under reduced pressure to afford compound 2 as pale-yellow solid (1.4 g, 58%).

### Step 2

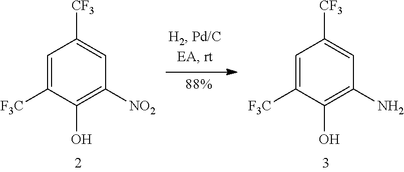 [SMILES: O=[N+]([O-])c1cc(C(F)(F)F)cc(C(F)(F)F)c1O ; Nc1cc(C(F)(F)F)cc(C(F)(F)F)c1O]

To a solution of compound 2 (1.4 g, 5.09 mmol, 1.0 eq) in ethyl acetate (20 mL) was added Pd/C (280 mg) at rt. The reaction mixture was stirred for 6 h at rt. After completion of the reaction (monitored by TLC (R_{f}=0.6, DCM/MeOH=10/1)). The mixture was concentrated under reduced pressure to afford compound 3 (1.1 g, 88%) as brown solid.

### Step 3

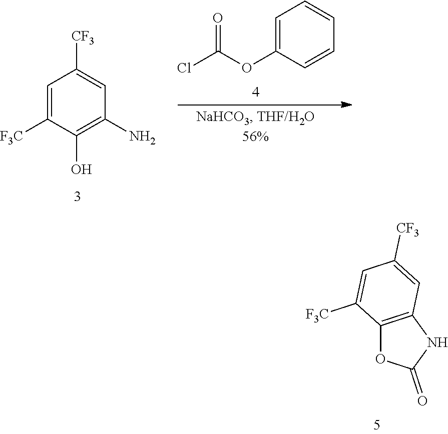 [SMILES: Nc1cc(C(F)(F)F)cc(C(F)(F)F)c1O ; O=C(Cl)Oc1ccccc1 ; O=c1[nH]c2cc(C(F)(F)F)cc(C(F)(F)F)c2o1]

To a solution of compound 3 (2.64 g, 10.77 mmol, 1.0 eq) in THF/H₂O (30 mL/10 mL) were added NaHCO₃ (1.08 mg, 12.9 mmol, 1.2 eq) and compound 4 (2.03 g, 12.9 mmol, 1.2 eq) at 0° C. The reaction mixture was stirred at rt for 4 h. After completion of the reaction (monitored by TLC (R_{f}=0.3, PE/EtOAc=3/1)), the reaction solution was poured into water (30 mL). The mixture was extracted with ethyl acetate (50 mL×2). The combined organic layer was washed with brine, dried over Na₂SO₄ and filtered. The filtration was concentrated under reduced pressure. The residue was purified by column chromatography (100-200 silica gel, 20% EtOAc in PE as eluent) to afford compound 5 as pale-yellow solid (1.65 g, 56%).

### Step 4

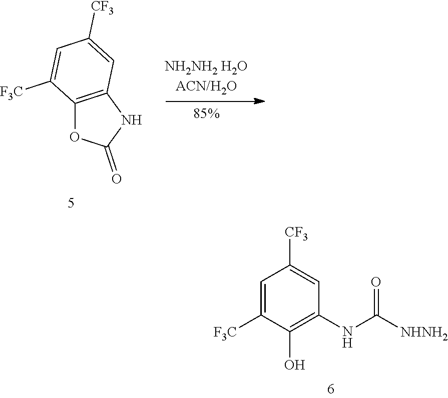 [SMILES: O=c1[nH]c2cc(C(F)(F)F)cc(C(F)(F)F)c2o1 ; NNC(=O)Nc1cc(C(F)(F)F)cc(C(F)(F)F)c1O]

To a solution of compound 5 (1.65 g, 6.37 mmol, 1.0 eq) in MeCN (5 mL/2 mL) was added NH₂NH₂·H₂O (2.17 g, 31.85 mmol, 5.0 eq, 80%) at rt. The reaction mixture was stirred at rt for 16 h. After completion of the reaction (monitored by TLC (R_{f}=0.5, DCM/MeOH=10/1)), the reaction solution was poured into water (30 mL). The mixture was extracted with EtOAc (50 mL×2). The combined organic layer was washed with brine, dried with Na₂SO₄, and filtered. The filtration was concentrated under reduced pressure. The residue was purified by column chromatography (100-200 silica gel, 5.5% MeOH in DCM as eluent) to afford compound 6 as pale-red solid (1.58 g, 85%).

### Step 5

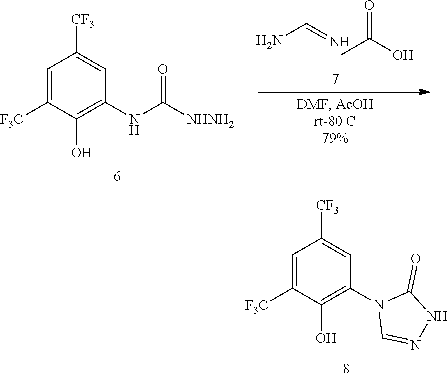

To a solution of compound 6 (1.58 g, 5.21 mmol, 1.0 eq) in DMF (20 mL) was added compound 7 (2.17 g, 20.8 mmol, 4.0 eq) at rt. The reaction mixture was stirred at rt for 0.5 h, followed by addition of AcOH (2 mL). The reaction mixture was stirred at 80° C. for 9 h. After completion (monitored by TLC (R_{f} 0.3, DCM/MeOH=10/1)), the reaction solution was poured into water (30 mL). The mixture was extracted with ethyl acetate (50 mL×2). The combined organic layer was washed with brine, dried over Na₂SO₄, and filtered. The filtration was concentrated under reduced pressure. The residue was purified by column chromatography (100-200 silica gel, 6.5% MeOH in DCM as eluent) to afford compound 8 as pale-yellow solid (1.3 g, 79%).

### Step 6

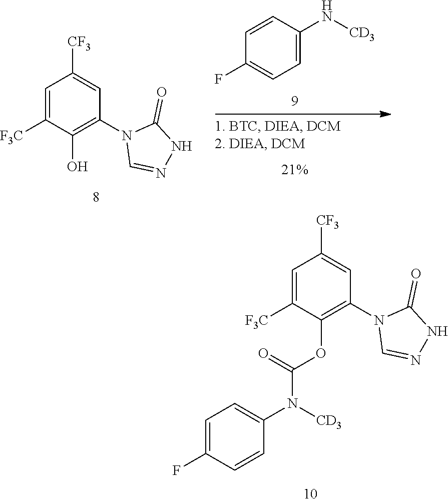 [SMILES: O=c1[nH]ncn1-c1cc(C(F)(F)F)cc(C(F)(F)F)c1O ; [2H]C([2H])([2H])Nc1ccc(F)cc1 ; [2H]C([2H])([2H])N(C(=O)Oc1c(-n2cn[nH]c2=O)cc(C(F)(F)F)cc1C(F)(F)F)c1ccc(F)cc1]

To a solution of compound 8 (100 mg, 0.32 mmol, 1.0 eq) in DCM (10 mL) were added DIEA (62 mg, 0.48 mmol, 1.5 eq) and triphosgene (48 mg, 0.16 mmol, 0.5 eq) in DCM (3 mL) at −10° C. The reaction mixture was stirred for 10 mins at rt under N₂ atmosphere. The reaction solution was concentrated under reduced pressure to remove solvent. Then DCM (3 mL) added to above residue. The solution was used for next step.

To a solution of compound 9 (81 mg, 0.48 mmol, 1.5 eq) in DCM (5 mL) was added the above residue solution at 0° C. The mixture was stirred for 1 h at rt under N₂ atmosphere. After completion (monitored by TLC (R_{f}=0.5, DCM/MeOH=10/1)), HCl (1 M, 15 mL) was added. Then the mixture was extracted with DCM (20 mL×2). The combined organic layer was washed with brine, dried over Na₂SO₄, and filtered. The filtration was concentrated under reduced pressure. The residue was purified Prep-HPLC to give the titled compound (AB38256: 31.3 mg, 21%) as white solid.

LCMS [M+H]⁺: 468.25

¹H NMR (400 MHz, CD₃OD) δ 8.01 (s, 1H), 7.89 (s, 1H), 7.73 (s, 1H), 7.28-7.33 (m, 2H), 7.09-7.13 (m, 2H).

### Example 35

### 2-{1-[2-hydroxy-3-(methylamino) propyl]-5-oxo-4,5-dihydro-1H-1,2,4-triazol-4-yl}-4,6-bis(trifluoromethyl)phenyl N-(4-fluorophenyl)-N-(methyl-d3)carbamate

### Step 1

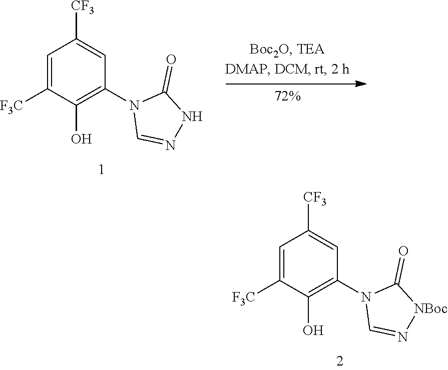 [SMILES: O=c1[nH]ncn1-c1cc(C(F)(F)F)cc(C(F)(F)F)c1O ; CC(C)(C)OC(=O)n1ncn(-c2cc(C(F)(F)F)cc(C(F)(F)F)c2O)c1=O]

To a solution of compound 1 (2.04 g, 6.52 mmol, 1.0 eq) in DMF (10 mL) were added TEA (790 mg, 7.82 mmol, 1.2 eq), DMAP (80 mg, 0.652 mmol, 0.1 eq) and Boc₂O (1.49 g, 6.84 mmol, 1.05 eq) at rt. The reaction was stirred for 2 h at rt. After completion of the reaction by TLC (R_{f}=0.4, PE/EA=3/1). The mixture was extracted with EA (50 mL×2). The combined organic layer was washed with brine, dried over Na₂SO₄ and filtered. The filtration was concentrated under reduced pressure. The residue was purified by column chromatography (100-200 silica gel, 35% EA in PE as eluent) to afford compound 2 (1.95 g, 72%) as off-white solid.

### Step 2

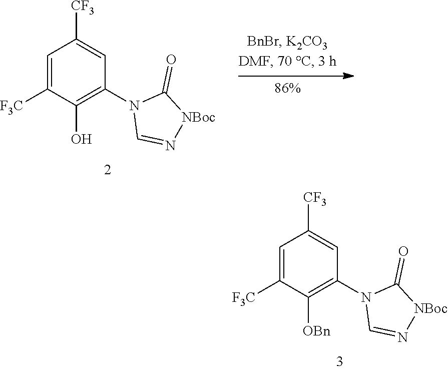 [SMILES: CC(C)(C)OC(=O)n1ncn(-c2cc(C(F)(F)F)cc(C(F)(F)F)c2O)c1=O ; CC(C)(C)OC(=O)n1ncn(-c2cc(C(F)(F)F)cc(C(F)(F)F)c2OCc2ccccc2)c1=O]

To a solution of compound 2 (1.95 g, 4.73 mmol, 1.0 eq) in DCM (10 mL) were added K₂CO₃ (1.65 g, 11.8 mmol, 2.5 eq) and BnBr (1.22 g, 7.09 mmol, 1.5 eq) at rt. The reaction was stirred at 70° C. for 3 h. After completion of the reaction by TLC (R_{f}=0.9, PE/EA=3/1), the reaction solution was poured into water (30 mL). The mixture was extracted with EA (50 mL×2). The combined organic layer was washed with brine, dried over Na₂SO₄ and filtered. The filtration was concentrated under reduced pressure. The residue was purified by column chromatography (100-200 silica gel, 5% EA in PE as eluent) to afford compound 3 as pale yellow solid (2.04 g, 86%).

### Step 3

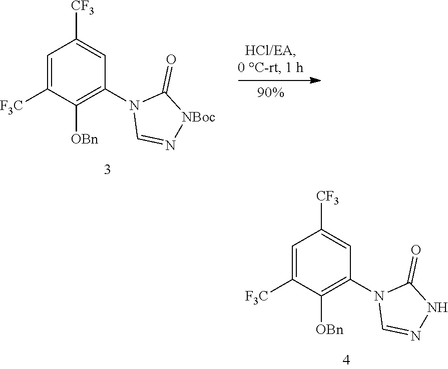 [SMILES: CC(C)(C)OC(=O)n1ncn(-c2cc(C(F)(F)F)cc(C(F)(F)F)c2OCc2ccccc2)c1=O ; O=c1[nH]ncn1-c1cc(C(F)(F)F)cc(C(F)(F)F)c1OCc1ccccc1]

Compound 3 (2.04 g, 4.05 mmol, 1.0 eq) was added into HCl/dioxane (4M, 5 mL) at 0° C. The reaction was stirred for 1 h. After completion of the reaction by TLC (R_{f}=0.3, PE/EA=3/1), the mixture was concentrated to give compound 4 (1.47 g, 90%) as off-white solid.

### Step 4

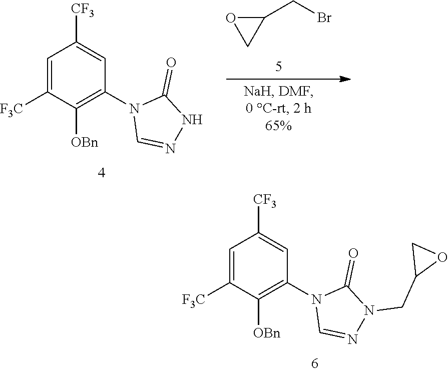 [SMILES: O=c1[nH]ncn1-c1cc(C(F)(F)F)cc(C(F)(F)F)c1OCc1ccccc1 ; BrCC1CO1 ; O=c1n(-c2cc(C(F)(F)F)cc(C(F)(F)F)c2OCc2ccccc2)cnn1CC1CO1]

To a solution of compound 4 (1.47 g, 3.35 mmol, 1.0 eq) in DMF (10 mL) was added 60% NaH (268 mg, 6.69 mmol, 2.0 eq) at 0° C. The reaction mixture was stirred for 30 mins at rt under N₂. Compound 5 (689 mg, 5.02 mmol, 1.4 eq) was added at 0° C. The reaction mixture was stirred for 2 h at rt under N₂. After completion of the reaction by TLC (R_{f}=0.4, PE/EA=3/1), the mixture was extracted with EA (50 mL×2). The combined organic layer was washed with brine, dried over Na₂SO₄ and filtered. The filtration was concentrated under reduced pressure. The residue was purified by column chromatography (100-200 silica gel, 40% EA in PE as eluent) to afford the product compound 6 (1.08 g, 65%) as colorless oil.

### Step 5

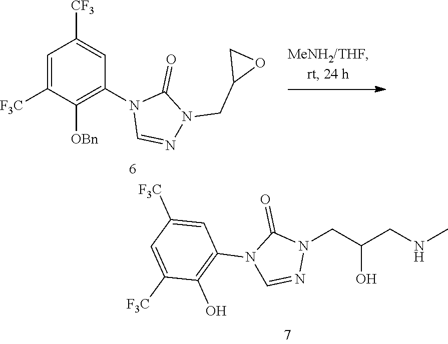

To a solution of compound 6 (1.08 g, 2.35 mmol, 1.0 eq) in THF (4 mL) was added MeNH₂/THF (4 mL) at rt. The reaction was stirred for 24 h at rt. After completion of the reaction by TLC (R_{f}=0.1, DCM/MeOH=10/1), the mixture was concentrated under reduced pressure to afford compound 7 as yellow oil (1.2 g, crude).

### Step 6

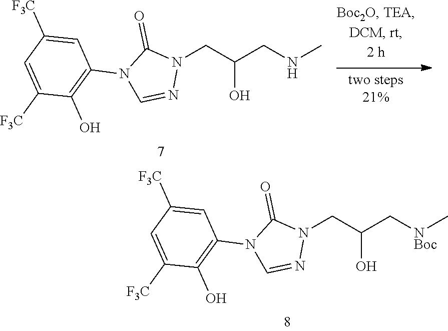 [SMILES: CNCC(O)Cn1ncn(-c2cc(C(F)(F)F)cc(C(F)(F)F)c2O)c1=O ; CN(CC(O)Cn1ncn(-c2cc(C(F)(F)F)cc(C(F)(F)F)c2O)c1=O)C(=O)OC(C)(C)C]

To a solution of compound 7 (1.2 g, 2.6 mmol, 1.0 eq) in DCM (10 mL) were added TEA (606 mg, 6.0 mmol, 2 eq) and Boc₂O (719 mg, 3.3 mmol, 1.1 eq) at rt. The reaction was stirred for 2 h at rt. After completion of the reaction by TLC (R_{f}=0.4, PE/EA=3/1). The mixture was extracted with EA (50 mL×2). The combined organic layer was washed with brine, dried over Na₂SO₄ and filtered. The filtration was concentrated under reduced pressure. The residue was purified by column chromatography (100-200 silica gel, 35% EA in PE as eluent) to afford compound 8 (250 mg, 21%) as off-white solid.

### Step 7

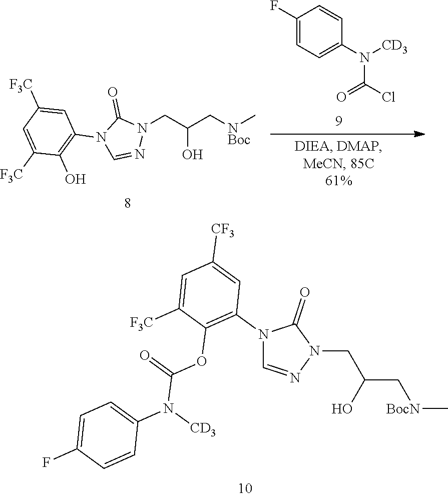 [SMILES: CN(CC(O)Cn1ncn(-c2cc(C(F)(F)F)cc(C(F)(F)F)c2O)c1=O)C(=O)OC(C)(C)C ; [2H]C([2H])([2H])N(C(=O)Cl)c1ccc(F)cc1 ; [2H]C([2H])([2H])N(C(=O)Oc1c(-n2cnn(CC(O)CN(C)C(=O)OC(C)(C)C)c2=O)cc(C(F)(F)F)cc1C(F)(F)F)c1ccc(F)cc1]

To a solution of compound 8 (250 mg, 0.5 mmol, 1.0 eq) in MeCN (5 mL) were added compound 9 (123 mg, 0.65 mmol, 1.3 eq), DIEA (162 mg, 1.25 mmol, 2.5 eq), DMAP (13 mg, 0.1 mmol, 0.2 eq) at rt. The reaction was heated for 2 h at 85° C. After completion of the reaction by TLC (R_{f}=0.4, PE/EA=1/1). The mixture was extracted with EA (50 mL×2). The combined organic layer was washed with brine, dried over Na₂SO₄ and filtered. The filtration was concentrated under reduced pressure. The residue was purified by column chromatography (100-200 silica gel, 65% EA in PE as eluent) to afford compound 10 (200 mg, 61%) as off-white solid.

### Step 8

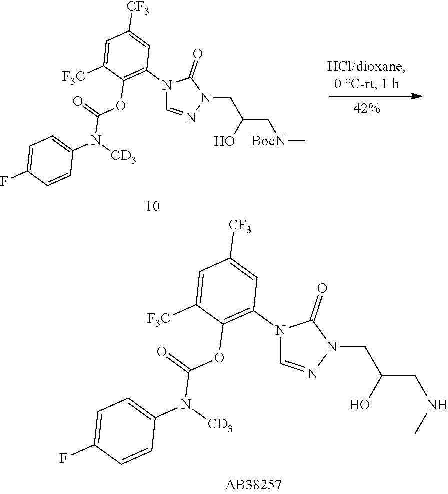 [SMILES: [2H]C([2H])([2H])N(C(=O)Oc1c(-n2cnn(CC(O)CN(C)C(=O)OC(C)(C)C)c2=O)cc(C(F)(F)F)cc1C(F)(F)F)c1ccc(F)cc1 ; [2H]C([2H])([2H])N(C(=O)Oc1c(-n2cnn(CC(O)CNC)c2=O)cc(C(F)(F)F)cc1C(F)(F)F)c1ccc(F)cc1]

Compound 10 (200 mg, 0.31 mmol, 1.0 eq) was added into HCl/dioxane (4M, 5 mL) at 0° C. The reaction was stirred for 1 h at rt. After completion of the reaction by TLC (R_{f}=0.3, DCM/MeOH=10/1), The mixture was extracted with EA (50 mL×2) and NaHCO₃ (sat. aq.) The combined organic layer was washed with brine, dried over Na₂SO₄ and filtered. The filtration was concentrated under reduced pressure. The residue was purified by column chromatography (100-200 silica gel, 12% MeOH in DCM as eluent) to afford the titled compound (AB38257: 70.5 mg, 42%) as off-white solid.

LCMS: 555.2 [M+H]⁺

¹H NMR (400 MHz, CD₃OD) δ 8.28-7.85 (m, 3H), 7.34-7.25 (m, 2H), 7.20-7.12 (m, 2H), 4.31-4.20 (m, 1H), 3.92 (br, 2H), 2.89-2.79 (m, 2H), 2.53 (s, 2H), 2.45 (s, 1H).

### Example 36

### 3-(2-(2-(4-(2-(((4-fluorophenyl)(methyl)carbamoyl)oxy)-3,5-bis(trifluoromethyl)phenyl)-1H-1,2,3-triazol-1-yl)ethoxy)ethoxy)propanoic Acid

### Step 1

### Synthesis of 2-(2,4-bis(trifluoromethyl)phenyl)-4,4,5,5-tetramethyl-1,3,2-dioxaborolane

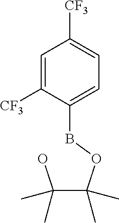

A solution of 1-bromo-2,4-bis(trifluoromethyl)benzene (3 g, 0.01 mol) in 1,4 dioxane (90 mL) was purged with N₂ in a sealed tube for 30 min and then added bispinacolato diboron (5.07 g, 0.02 mol), potassium acetate (1.96 g, 0.02 mol) and Pd(dppf)Cl₂·DCM (0.816 g, 0.001 mol) with stirring under inert atmosphere. The tightly closed sealed tube was heated to 100° C. in an oil bath for 6 h with stirring. Progress of the reaction was monitored by TLC (Rf=0.6, 10% EtOAc in Hexane). After completion of the reaction, the reaction mixture was concentrated under reduced pressure to afford the crude. Crude was washed with n-hexane (4×75 mL); washings collected was concentrated under reduced pressure to afford the yellow sticky solid (crude 6 g, Quantitative). The product was taken as such to next step without further purification.

### Step 2

### Synthesis of 2,4-bis(trifluoromethyl)phenol

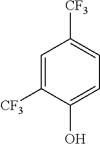 [SMILES: Oc1ccc(C(F)(F)F)cc1C(F)(F)F]

To a cold (0° C.) solution of 2-(2,4-bis(trifluoromethyl)phenyl)-4,4,5,5-tetramethyl-1,3,2-dioxaborolane (crude 6 g) in EtOH (120 mL), added hydrogen peroxide 30% aq. solution (6.0 mL) under inert atmosphere with stirring. The reaction mixture was stirred at ambient temperature for 16 h. After completion of the reaction confirmed by TLC (R_{f} 0.2, 10% EtOAc in Hexane), the reaction mixture was cooled to 0° C. and quenched with aq. sodium metabisulfite solution followed by extraction with EtOAc (2×100 mL). The organic layer collected dried over anhydrous Na₂SO₄, concentrated under reduced pressure to afford the crude. The crude was purified by column chromatography (100-200 silica gel, 10-15% EtOAc in Hexane as eluent) to afford the title compound as pale yellow liquid (1.5 g, 65% yield).

### Step 3

### Synthesis of 2-iodo-4,6-bis(trifluoromethyl)phenol

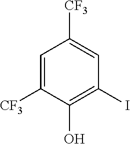 [SMILES: Oc1c(I)cc(C(F)(F)F)cc1C(F)(F)F]

A solution of 2,4-bis(trifluoromethyl)phenol (1.0 g, 0.0043 mol) in THF:H₂O (3:1, 20:6 mL) was cooled 0° C. in an ice bath with stirring. After 15 minutes, was added I₂ (1.43 g, 0.0056 mol) followed by Na2CO3 (0.68 g, 0.0064 mol) under inert atmosphere with stirring. Allowed the reaction mixture to stir at ambient temperature for 24 h. After completion of the reaction (TLC: R_{f}—0.4, 30% EtOAc in Hexane followed by 30% DCM in Hexane), the reaction mixture was cooled to 0° C. and then quenched with aq. sodium metabisulfite solution followed by extraction with EtOAc (2×100 mL). The organic layer collected dried over anhydrous Na₂SO4 filtered and concentrated under reduced pressure to afford the crude. The crude was purified by column chromatography (100-200 silica gel, 5-10% EtOAc in Hexane as eluent) to afford the title compound as pale yellow solid (0.98 g, 64% yield).

### Step 4

### Synthesis of (4-fluorophenyl)(methyl)carbamic Chloride

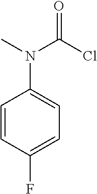

To a cold (0° C.) solution of triphosgene (1.6 g, 0.0128 mol) in DCM (40 mL) added a solution of N-methyl-4-fluoro aniline (1.89 g, 0.0064 mol) and pyridine (2.52 g, 2.6 mL, 0.032 mol) dropwise for over a period of 10 min. After that continued stirring at RT for 16 h. Progress of the reaction was monitored by TLC (R_{f}—31 0.7, 10% EtOAc in Hexane (4 times)). After completion of the reaction, quenched the reaction mixture with 1M aq.HCl (50 mL) and then extracted with DCM (2×50 mL). The DCM layer separated was dried over anhydrous Na₂SO₄, filtered, and concentrated under reduced pressure to afford the title compound as green solid (1.8 g, 75%).

### Step 5

### Synthesis of 2-iodo-4,6-bis(trifluoromethyl)phenyl (4-fluorophenyl)(methyl)carbamate

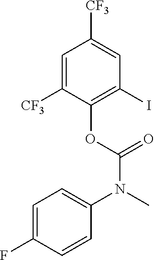 [SMILES: CN(C(=O)Oc1c(I)cc(C(F)(F)F)cc1C(F)(F)F)c1ccc(F)cc1]

To a stirred solution of 2-iodo-4,6-bis(trifluoromethyl)phenol, (0.5 g, 0.0014 mol) in Pyridine (10 mL) at RT added (4-fluorophenyl)(methyl)carbamic chloride (0.34 g, 0.0018 mol) and continued stirring at 80° C. for 4 hours. After completion of the reaction was confirmed by TLC (R_{f}—0.8, 20% EtOAc in Hexane) quenched the reaction mixture with 1M HCl (50 mL) and extracted with EtOAc (2×50 mL). The organic layer separated was combined, dried over anhydrous Na₂SO₄, filtered, and concentrated under reduced pressure to afford the crude. The crude obtained was purified by silica gel chromatography (100-200 and 5-10% EtOAc in Hexane as eluent) to afford the title compound as off-white solid (0.66 g, 93%).

### Step 6

### Synthesis of 2,4-bis(trifluoromethyl)-6-((trimethylsilyl)ethynyl)phenyl (4-fluorophenyl)(methyl)carbamate

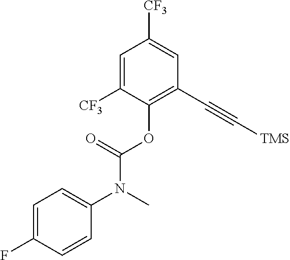 [SMILES: CN(C(=O)Oc1c(C#C[Si](C)(C)C)cc(C(F)(F)F)cc1C(F)(F)F)c1ccc(F)cc1]

2-iodo-4,6-bis(trifluoromethyl)phenyl (4-fluorophenyl)(methyl)carbamate (1.0 g, 0.0019 mol), trimethylsilylacetylene (0.78 mL 0.00552 mol) and), triethylamine (0.55 mL, 0.0039 mol) were dissolved in DMF (30 mL) purged with N₂ for 30 minutes. To the above mixture added copper (I)-iodide (0.075 g, 0.00039 mol) and PdCl₂(PPh₃)₂ (0.138 g, 0.00019 mol) degassed the mixture with N₂ for 15 minutes. The reaction mixture was heated at 60° C. in an oil-bath for 5 h. After completion of the reaction by TLC (R_{f} 0.6, 10% EtOAc in Hexane) the reaction mixture was quenched with ice cold water (150 mL) and extracted with EtOAc (2×50 mL) and dried over anhydrous Na₂SO₄, filtered and concentrated under reduced pressure. The residue obtained was purified by column chromatography (100-200 silica gel, 4-6% EtOAc in Hexane as eluent) to afford the title compound as pale yellow liquid (0.5 g, 53%).

### Step 7

### Synthesis of 2-ethynyl-4,6-bis(trifluoromethyl)phenyl (4-fluorophenyl)(methyl)carbamate

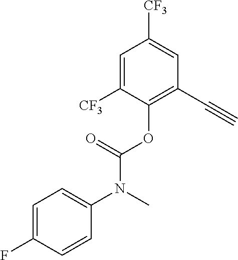 [SMILES: C#Cc1cc(C(F)(F)F)cc(C(F)(F)F)c1OC(=O)N(C)c1ccc(F)cc1]

To a stirred solution of 2,4-bis(trifluoromethyl)-6-((trimethylsilyl)ethynyl)phenyl (4-fluorophenyl)(methyl)carbamate (0.5 g, 0.0104 mol) in THF (25 mL) was added 1M TBAF solution in THF (0.45 mL, 0.0156 mol) at 0° C. The reaction mixture was stirred at RT for 1 h. After completion of the reaction confirmed by TLC (R_{f} 0.3, 10% EtOAc in Hexane) the reaction mixture was diluted with cold water (50 mL) followed by extraction with ethyl acetate (2×50 mL). The organic fractions separated were combined, dried over anhydrous Na₂SO₄, filtered and concentrated under reduced pressure to afford crude. The crude residue was purified by column chromatography (100-200 silica gel, 7-10% EtOAc in Hexane as eluent) to afford the title compound as colorless sticky liquid (0.4 g, 95%).

### Step 8

### Synthesis of tert-butyl 3-(2-(2-(4-(2-(((4-fluorophenyl)(methyl)carbamoyl)oxy)-3,5-bis(trifluoromethyl)phenyl)-1H-1,2,3-triazol-1-yl)ethoxy)ethoxy)propanoate

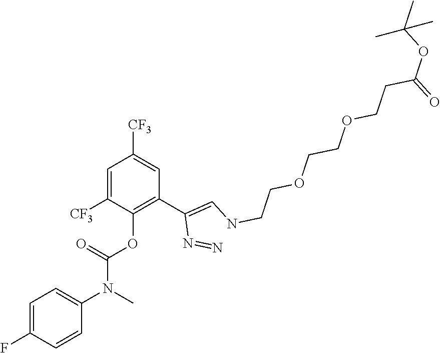 [SMILES: CN(C(=O)Oc1c(/C(=C/NCCOCCOCCC(=O)OC(C)(C)C)N=N)cc(C(F)(F)F)cc1C(F)(F)F)c1ccc(F)cc1]

A solution of 2-ethynyl-4,6-bis(trifluoromethyl)phenyl (4-fluorophenyl)(methyl)carbamate (0.1 g, 0.00024 mol) and triethylamine (0.038 mL, 0.00027 mol) in a mixture of ^{t}BuOH:H₂O (1:1, 6 mL) was stirred at 0° C. for 10 minute. To the above mixture at 0° C. added tert-butyl 3-(2-(2-azidoethoxy) ethoxy) propanoate (0.044 g, 0.0001 mol), CuSO₄·5H2O (0.018 g, 0.00007 mol) and sodium ascorbate (0.022 g, 0.00012 mol) and continued stirring at RT under N₂ atmosphere for 5 h. After completion of the reaction confirmed by TLC (R_{f} 0.3, 50% EtOAc in Hexane) the reaction mixture was diluted with cold water (50 mL) followed by extraction with ethyl acetate (2×50 mL). The organic fractions separated were combined, dried over anhydrous Na₂SO₄, filtered and concentrated under reduced pressure to afford crude. The crude residue was purified by column chromatography (100-200 silica gel, 30-40% EtOAc in Hexane as eluent) to afford the title compound as pale yellow sticky liquid (0.06 g, 36%).

### Step 9

### Synthesis of 3-(2-(2-(4-(2-(((4-fluorophenyl)(methyl)carbamoyl)oxy)-3,5-bis(trifluoromethyl)phenyl)-1H-1,2,3-triazol-1-yl)ethoxy)ethoxy)propanoic Acid

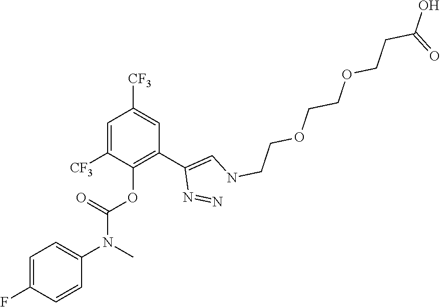 [SMILES: CN(C(=O)Oc1c(/C(=C/NCCOCCOCCC(=O)O)N=N)cc(C(F)(F)F)cc1C(F)(F)F)c1ccc(F)cc1]

A solution of tert-butyl 3-(2-(2-(4-(2-(((4-fluorophenyl)(methyl)carbamoyl)oxy)-3,5-bis(trifluoromethyl)phenyl)-1H-1,2,3-triazol-1-yl)ethoxy)ethoxy)propanoate (0.1 g, 0.00015 mol) in DCM (5 mL) was cooled to 0° C. with stirring for 10 minute. To the above cold solution added TFA (0.5 mL, 5 vol) and was stirring was continued at RT for 5 h. After completion of the reaction was confirmed by TLC (R_{f} 0.2, 5% MeOH in DCM) the reaction mixture was quenched with aq. NaHCO₃ solution at 0° C. followed by extraction with 10% MeOH in DCM (2×50 mL). The organic fractions separated were combined, dried over anhydrous Na₂SO₄, filtered and concentrated under reduced pressure to afford crude. The crude obtained was purified by preparative TLC (solid phase: merck, 20×20 cm, silicagel 60 GF₂₅₄, 1 mm, PLC glass plate, 60% EtOAc in Hexane as eluent) to afford the titled compound as pale yellow solid (20 mg, 22%).

¹H NMR (400 MHz, DMSO-d6) δ 12.14 (s, 1H), 8.56 (m 2H), 8.11 (d, J=30.4 Hz, 1H), 7.58 (dd, J=8.2 Hz, J=4.8 Hz, 1H), 7.33 (m 2H), 7.24 (t, J=8.4 Hz, 1H), 4.68 (s, 2H), 3.89 (m, 2H), 3.53 (d, J=6.9 Hz, 5H), 3.45 (s, 2H), 3.24 (s, 2H), 2.35 (m, 2H).

### Example 37

### 2-(1-(2-(2-(3-(((S)-1-((2S,4R)-4-hydroxy-2-((4-(4-methylthiazol-5-yl)benzyl)carbamoyl) pyrrolidin-1-yl)-3,3-dimethyl-1-oxobutan-2-yl)amino)-3-oxopropoxy)ethoxy)ethyl)-1H-1,2,3-triazol-4-yl)-4,6-bis(trifluoromethyl)phenyl (4-fluorophenyl)(methyl)carbamate

### Step 1

### Synthesis of 2-(2,4-bis(trifluoromethyl)phenyl)-4,4,5,5-tetramethyl-1,3,2-dioxaborolane

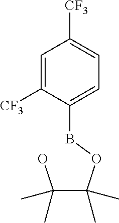

A solution of 1-bromo-2,4-bis(trifluoromethyl)benzene (3 g, 0.01 mol) in 1,4 dioxane (90 mL) was purged with N₂ in a sealed tube for 30 min and then added bispinacolato diboron (5.07 g, 0.02 mol), potassium acetate (1.96 g, 0.02 mol) and Pd(dppf)Cl₂·DCM (0.816 g, 0.001 mol) with stirring under inert atmosphere. The tightly closed sealed tube was heated to 100° C. in an oil bath for 6 h with stirring. Progress of the reaction was monitored by TLC (Rf=0.6, 10% EtOAc in Hexane). After completion of the reaction, the reaction mixture was concentrated under reduced pressure to afford the crude. Crude was washed with n-hexane (4×75 mL); washings collected was concentrated under reduced pressure to afford the yellow sticky solid (crude 6 g, Quantitative). The product was taken as such to next step without further purification.

### Step 2

### Synthesis of 2,4-bis(trifluoromethyl)phenol

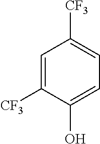 [SMILES: Oc1ccc(C(F)(F)F)cc1C(F)(F)F]

To a cold (0° C.) solution of 2-(2,4-bis(trifluoromethyl)phenyl)-4,4,5,5-tetramethyl-1,3,2-dioxaborolane (crude 6 g) in EtOH (120 mL), added hydrogen peroxide 30% aq. solution (6.0 mL) under inert atmosphere with stirring. The reaction mixture was stirred at ambient temperature for 16 h. After completion of the reaction confirmed by TLC (R_{f} 0.2, 10% EtOAc in Hexane), the reaction mixture was cooled to 0° C. and quenched with aq. sodium metabisulfite solution followed by extraction with EtOAc (2×100 mL). The organic layer collected dried over anhydrous Na₂SO₄, concentrated under reduced pressure to afford the crude. The crude was purified by column chromatography (100-200 silica gel, 10-15% EtOAc in Hexane as eluent) to afford the title compound as pale yellow liquid (1.5 g, 65% yield).

### Step 3

### Synthesis of 2-iodo-4,6-bis(trifluoromethyl)phenol

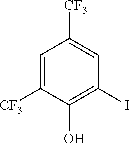 [SMILES: Oc1c(I)cc(C(F)(F)F)cc1C(F)(F)F]

A solution of 2,4-bis(trifluoromethyl)phenol (1.0 g, 0.0043 mol) in THF:H₂O (3:1, 20:6 mL) was cooled 0° C. in an ice bath with stirring. After 15 minutes, was added I₂ (1.43 g, 0.0056 mol) followed by Na2CO3 (0.68 g, 0.0064 mol) under inert atmosphere with stirring. Allowed the reaction mixture to stir at ambient temperature for 24 h. After completion of the reaction (TLC: R_{f}—0.4, 30% EtOAc in Hexane followed by 30% DCM in Hexane), the reaction mixture was cooled to 0° C. and then quenched with aq. sodium metabisulfite solution followed by extraction with EtOAc (2×100 mL). The organic layer collected dried over anhydrous Na₂SO₄, filtered and concentrated under reduced pressure to afford the crude. The crude was purified by column chromatography (100-200 silica gel, 5-10% EtOAc in Hexane as eluent) to afford the title compound as pale yellow solid (0.98 g, 64% yield).

### Step 4

### Synthesis of (4-fluorophenyl)(methyl)carbamic Chloride

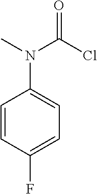

To a cold (0° C.) solution of triphosgene (1.6 g, 0.0128 mol) in DCM (40 mL) added a solution of N-methyl-4-fluoro aniline (1.89 g, 0.0064 mol) and pyridine (2.52 g, 2.6 mL, 0.032 mol) dropwise for over a period of 10 min. After that continued stirring at RT for 16 h. Progress of the reaction was monitored by TLC (R_{f}—0.7, 10% EtOAc in Hexane (4 times)). After completion of the reaction, quenched the reaction mixture with 1M aq.HCl (50 mL) and then extracted with DCM (2×50 mL). The DCM layer separated was dried over anhydrous Na₂SO₄, filtered, and concentrated under reduced pressure to afford the title compound as green solid (1.8 g, 75%).

### Step 5

### Synthesis of 2-iodo-4,6-bis(trifluoromethyl)phenyl (4-fluorophenyl)(methyl)carbamate

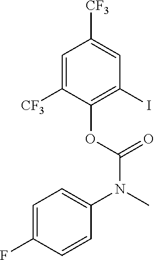 [SMILES: CN(C(=O)Oc1c(I)cc(C(F)(F)F)cc1C(F)(F)F)c1ccc(F)cc1]

To a stirred solution of 2-iodo-4,6-bis(trifluoromethyl)phenol, (0.5 g, 0.0014 mol) in Pyridine (10 mL) at RT added (4-fluorophenyl)(methyl)carbamic chloride (0.34 g, 0.0018 mol) and continued stirring at 80° C. for 4 hours. After completion of the reaction was confirmed by TLC (R_{f}—0.8, 20% EtOAc in Hexane) quenched the reaction mixture with 1M HCl (50 mL) and extracted with EtOAc (2×50 mL). The organic layer separated was combined, dried over anhydrous Na₂SO₄, filtered, and concentrated under reduced pressure to afford the crude. The crude obtained was purified by silica gel chromatography (100-200 and 5-10% EtOAc in Hexane as eluent) to afford the title compound as off-white solid (0.66 g, 93%).

### Step 6

### Synthesis of 2,4-bis(trifluoromethyl)-6-((trimethylsilyl)ethynyl)phenyl (4-fluorophenyl)(methyl)carbamate

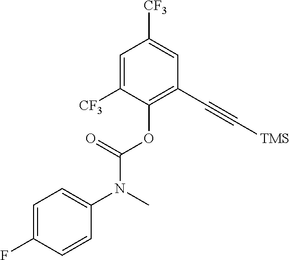 [SMILES: CN(C(=O)Oc1c(C#C[Si](C)(C)C)cc(C(F)(F)F)cc1C(F)(F)F)c1ccc(F)cc1]

2-iodo-4,6-bis(trifluoromethyl)phenyl (4-fluorophenyl)(methyl)carbamate (1.0 g, 0.0019 mol), trimethylsilylacetylene (0.78 mL 0.00552 mol) and), triethylamine (0.55 mL, 0.0039 mol) were dissolved in DMF (30 mL) purged with N₂ for 30 minutes. To the above mixture added copper (I)-iodide (0.075 g, 0.00039 mol) and PdCl₂(PPh₃)₂ (0.138 g, 0.00019 mol) degassed the mixture with N₂ for 15 minutes. The reaction mixture was heated at 60° C. in an oil-bath for 5 h. After completion of the reaction by TLC (R_{f} 0.6, 10% EtOAc in Hexane) the reaction mixture was quenched with ice cold water (150 mL) and extracted with EtOAc (2×50 mL) and dried over anhydrous Na₂SO₄, filtered and concentrated under reduced pressure. The residue obtained was purified by column chromatography (100-200 silica gel, 4-6% EtOAc in Hexane as eluent) to afford the title compound as pale yellow liquid (0.5 g, 53%).

### Step 7

### Synthesis of 2-ethynyl-4,6-bis(trifluoromethyl)phenyl (4-fluorophenyl)(methyl)carbamate

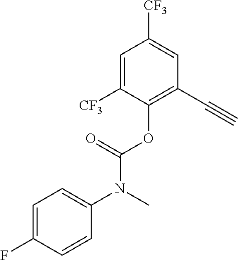 [SMILES: C#Cc1cc(C(F)(F)F)cc(C(F)(F)F)c1OC(=O)N(C)c1ccc(F)cc1]

To a stirred solution of 2,4-bis(trifluoromethyl)-6-((trimethylsilyl)ethynyl)phenyl (4-fluorophenyl)(methyl)carbamate (0.5 g, 0.0104 mol) in THF (25 mL) was added 1M TBAF solution in THF (0.45 mL, 0.0156 mol) at 0° C. The reaction mixture was stirred at RT for 1 h. After completion of the reaction confirmed by TLC (R_{f} 0.3, 10% EtOAc in Hexane) the reaction mixture was diluted with cold water (50 mL) followed by extraction with ethyl acetate (2×50 mL). The organic fractions separated were combined, dried over anhydrous Na₂SO₄, filtered and concentrated under reduced pressure to afford crude. The crude residue was purified by column chromatography (100-200 silica gel, 7-10% EtOAc in Hexane as eluent) to afford the title compound as colourless sticky liquid (0.4 g, 95%).

### Step 8

### Synthesis of tert-butyl 3-(2-(2-(4-(2-(((4-fluorophenyl)(methyl)carbamoyl)oxy)-3,5-bis(trifluoromethyl)phenyl)-1H-1,2,3-triazol-1-yl)ethoxy)ethoxy)propanoate

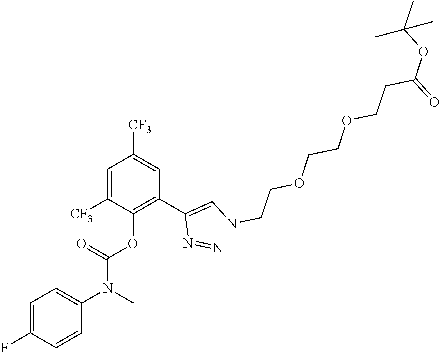 [SMILES: CN(C(=O)Oc1c(/C(=C/NCCOCCOCCC(=O)OC(C)(C)C)N=N)cc(C(F)(F)F)cc1C(F)(F)F)c1ccc(F)cc1]

A solution of 2-ethynyl-4,6-bis(trifluoromethyl)phenyl (4-fluorophenyl)(methyl)carbamate (0.1 g, 0.00024 mol) and triethylamine (0.038 mL, 0.00027 mol) in a mixture of ^{t}BuOH:H₂O (1:1, 6 mL) was stirred at 0° C. for 10 minute. To the above mixture at 0° C. added tert-butyl 3-(2-(2-azidoethoxy) ethoxy) propanoate (0.044 g, 0.0001 mol), CuSO₄·5H₂O (0.018 g, 0.00007 mol) and sodium ascorbate (0.022 g, 0.00012 mol) and continued stirring at RT under N₂ atmosphere for 5 h. After completion of the reaction confirmed by TLC (R_{f} 0.3, 50% EtOAc in Hexane) the reaction mixture was diluted with cold water (50 mL) followed by extraction with ethyl acetate (2×50 mL). The organic fractions separated were combined, dried over anhydrous Na₂SO₄, filtered and concentrated under reduced pressure to afford crude. The crude residue was purified by column chromatography (100-200 silica gel, 30-40% EtOAc in Hexane as eluent) to afford the title compound as pale yellow sticky liquid (0.06 g, 36%).

### Step 9

### Synthesis of 3-(2-(2-(4-(2-(((4-fluorophenyl)(methyl)carbamoyl)oxy)-3,5-bis(trifluoromethyl)phenyl)-1H-1,2,3-triazol-1-yl)ethoxy)ethoxy)propanoic Acid

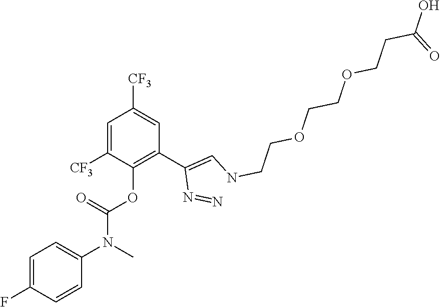 [SMILES: CN(C(=O)Oc1c(/C(=C/NCCOCCOCCC(=O)O)N=N)cc(C(F)(F)F)cc1C(F)(F)F)c1ccc(F)cc1]

A solution of tert-butyl 3-(2-(2-(4-(2-(((4-fluorophenyl)(methyl)carbamoyl)oxy)-3,5-bis(trifluoromethyl)phenyl)-1H-1,2,3-triazol-1-yl)ethoxy)ethoxy)propanoate (0.1 g, 0.00015 mol) in DCM (5 mL) was cooled to 0° C. with stirring for 10 minute. To the above cold solution added TFA (0.5 mL, 5 vol) and was stirring was continued at RT for 5 h. After completion of the reaction was confirmed by TLC (R_{f} 0.2, 5% MeOH in DCM) the reaction mixture was quenched with aq. NaHCO₃ solution at 0° C. followed by extraction with 10% MeOH in DCM (2×50 mL). The organic fractions separated were combined, dried over anhydrous Na₂SO₄, filtered and concentrated under reduced pressure to afford crude. The crude obtained was purified by preparative TLC (solid phase: merck, 20×20 cm, silicagel 60 GF₂₅₄, 1 mm, PLC glass plate, 60% EtOAc in Hexane as eluent) to afford the title compound as pale yellow solid (20 mg, 22%).

### Step 10

### Synthesis of 2-(1-(2-(2-(3-(((S)-1-((2S,4R)-4-hydroxy-2-((4-(4-methylthiazol-5-yl)benzyl)carbamoyl) pyrrolidin-1-yl)-3,3-dimethyl-1-oxobutan-2-yl)amino)-3-oxopropoxy)ethoxy)ethyl)-1H-1,2,3-triazol-4-yl)-4,6-bis(trifluoromethyl)phenyl (4-fluorophenyl)(methyl)carbamate

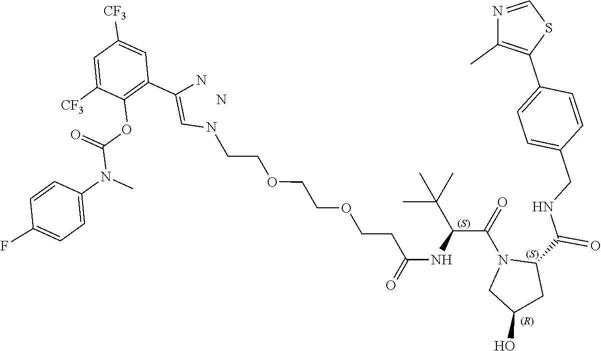

To a stirred solution of 3-(2-(2-(4-(2-(((4-fluorophenyl)(methyl)carbamoyl)oxy)-3,5-bis(trifluoromethyl)phenyl)-1H-1,2,3-triazol-1-yl)ethoxy)ethoxy)propanoic acid (0.03 g, 0.000049 mol) in DCM (3 mL) was cooled to 0° C. with stirring for 10 minute. To the above cold solution added Oxalyl chloride (0.15 mL, 5 vol) and stirring was continued at RT for 5 h. After completion of the reaction, the reaction mixture was concentrated under reduced pressure to afford the crude. The resultant crude was dissolved in DCM (3 mL) and added dropwise to a cold (0° C.) solution of triethylamine (0.02 mL, 0.000147 mol), (2S,4R)-1-((S)-2-amino-3,3-dimethylbutanoyl)-4-hydroxy-N-(4-(4-methylthiazol-5-yl)benzyl) pyrrolidine-2-carboxamide (0.04323 g, 0.000049 mol) in DCM (5 mL) with stirring. The reaction mixture was continued with stirring at RT for 12 h. After completion of the reaction was confirmed by TLC (R_{f} 0.4, 5% MeOH in DCM) the reaction mixture was quenched with ice cold water (20 mL) followed by extraction with DCM (2×50 mL). The organic fractions separated were combined, dried over anhydrous Na₂SO₄, filtered and concentrated under reduced pressure to afford crude. The crude obtained was purified by preparative TLC (solid phase: merck, 20×20 cm, silicagel 60 GF₂₅₄, 1 mm, PLC glass plate, 5% MeOH in DCM as eluent) to afford the title compound as pale yellow solid (17 mg, 34%).

¹H NMR (400 MHz, DMSO-d6) δ 8.98 (s, 1H), 8.67 (d, J=35.5 Hz, 1H), 8.57 (m, 2H), 8.43 (s, 1H), 8.11 (m, 1), 7.90 (m, 1H), 7.58 (m, 1H), 7.32 (m, 6H), 5.13 (d, J=3.6 Hz, 1H), 4.66 (m, 2H), 4.53 (d, J=9.3 Hz, 1H), 4.42 (m, 2H), 4.34 (d, J=1.5 Hz, 1H), 4.21 (dd, J=16.4 Hz, J=6.3 Hz, 1H), 3.89 (t, J=4.9 Hz, 2H), 3.60 (m, 3H), 3.24 (s, 2H), 2.44 (s, 3H), 2.02 (m, 2H), 1.91 (m, 2H), 1.24 (s, 5H), 0.90 (2, 9H).

### Biological Data

The following Cy5 fluorescence assay was used to measure the ability of compounds to inhibit Polq polymerase domain in vitro. The fluorescent based assay was performed as follows: 60 nM of the pre-annealed primer-template containing a 5′ Cy5 fluorophore conjugated template strand (SEQ ID NO 6: 5′-/5Cy5/ CACTGTGAGCTTAGTCACATTTCATCATGCA GGACAG-3′), a downstream complementary oligo conjugated with a 3′ Blackhole quencher (SEQ ID NO 7: 5′-CTAAGCTCACAGTG/3IAbRQSp/-3′) and a primer strand (SEQ ID NO 8: 5′-CTGTCCTGCATGATG-3′) was mixed with 50 uM 2′-deoxyribonucleoside triphosphates (dNTPs), 0.1 mg/mL Bovine serum albumin (BSA), 0.01% NP-40, 10% glycerol, 1 mM dithiothreitol (DTT), 10 mM MgCl₂, 25 mM TrisHCl pH 7.8 in the presence of 2.5% DMSO with or without various concentrations (7-point dilution series) of Polq small molecule inhibitors represented by Formula I at 37° C. in a volume of 40 μL. The reactions were initiated by the addition of 5 nM of purified recombinant human Polq polymerase domain (comprising amino acid residues 1792-2590 (SEQ ID NO: 1). The reactions were terminated by the addition of 20 mM EDTA after 18 min, and the Cy5 fluorescence intensity was measured using a Clariostar (BMG Labtech) plate reader. Reactions were performed in triplicate and the % inhibition at each concentration of the respective compound of Formula I was based on the mean. The compounds of Examples 1 to 10 were tested in the above Polq polymerase enzymatic activity assay. The IC50 of each compound represents the average concentration of compound that resulted in 50% inhibition of Polq polymerase enzymatic activity which was determined from a scatter plot (% inhibition versus compound concentration) curve generated by PRISM software for each compound inhibition data set.

### Polθ₁₇₉₂₋₂₅₉₀

### Amino Acid Sequence:

(SEQ ID NO: 1)
GFKDNSPISDTSFSLQLSQDGLQLTPASSSSESLSIIDVASDQNLFQTF
IKEWRCKKRFSISLACEKIRSLTSSKTATIGSRFKQASSPQEIPIRDDG
FPIKGCDDTLVVGLAVCWGGRDAYYFSLQKEQKHSEISASLVPPSLDPS
LTLKDRMWYLQSCLRKESDKECSVVIYDFIQSYKILLLSCGISLEQSYE
DPKVACWLLDPDSQEPTLHSIVTSFLPHELPLLEGMETSQGIQSLGLNA
GSEHSGRYRASVESILIFNSMNQLNSLLQKENLQDVFRKVEMPSQYCLA
LLELNGIGFSTAECESQKHIMQAKLDAIETQAYQLAGHSFSFTSSDDIA
EVLFLELKLPPNREMKNQGSKKTLGSTRRGIDNGRKLRLGRQFSTSKDV
LNKLKALHPLPGLILEWRRITNAITKVVFPLQREKCLNPFLGMERIYPV
SQSHTATGRITFTEPNIQNVPRDFEIKMPTLVGESPPSQAVGKGLLPMG
RGKYKKGFSVNPRCQAQMEERAADRGMPFSISMRHAFVPFPGGSILAAD
YSQLELRILAHLSHDRRLIQVLNTGADVFRSIAAEWKMIEPESVGDDLR
QQAKQICYGIIYGMGAKSLGEQMGIKENDAACYIDSFKSRYTGINQFMT
ETVKNCKRDGFVQTILGRRRYLPGIKDNNPYRKAHAERQAINTIVQGSA
ADIVKIATVNIQKQLETFHSTFKSHGHREGMLQSDQTGLSRKRKLQGMF
CPIRGGFFILQLHDELLYEVAEEDVVQVAQIVKNEMESAVKLSVKLKVK
VKIGASWGELKDFDV.

| Example | |
|---|---|
| number | Polq IC₅₀ (nM) |
| 1 | 4.8 |
| 2 | 4 |
| 3 | 3.3 |
| 4 | 5.5 |
| 5 | 4.6 |
| 6 | 4 |
| 7 | 5.8 |
| 8 | 4.2 |
| 9 | 2.4 |
| 10 | 2.9 |
| 11 | 2 |
| 12 | 2.2 |
| 13 | 4.6 |
| 14 | 2.3 |
| 15 | 1.9 |
| 16 | 1.5 |
| 17 | 1.5 |
| 18 | 2.6 |
| 19 | 295 |
| 20 | 439 |
| 21 | 3.2 |
| 22 | 24.5 |
| 23 | 46.8 |
| 24 | 13.6 |
| 25 | 24 |
| 26 | 2 |
| 27 | 1.8 |
| 28 | 3.2 |
| 29 | 3.4 |
| 30 | 1.6 |
| 31 | 5.6 |
| 32 | 18 |
| 33 | 2.9 |
| 34 | 51 |
| 35 | 104 |
| 36 | 3 |
| 37 | 4.6 |

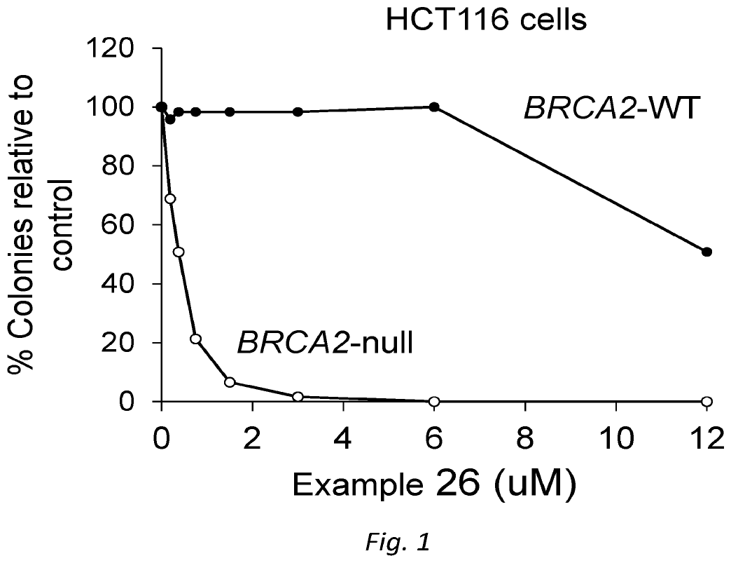

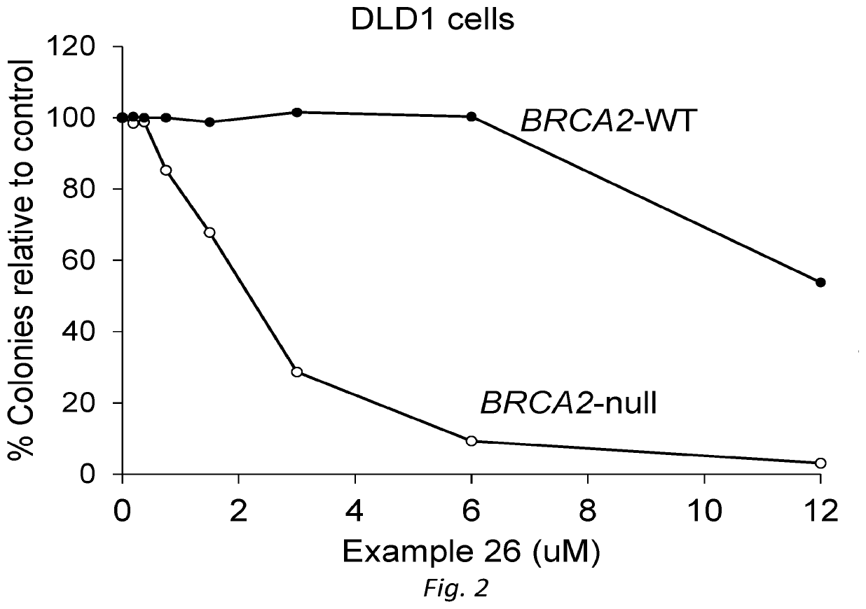

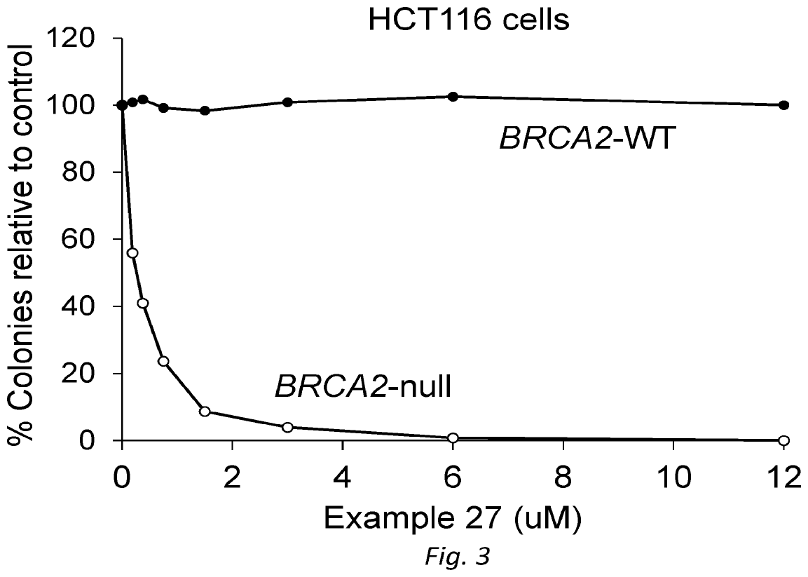

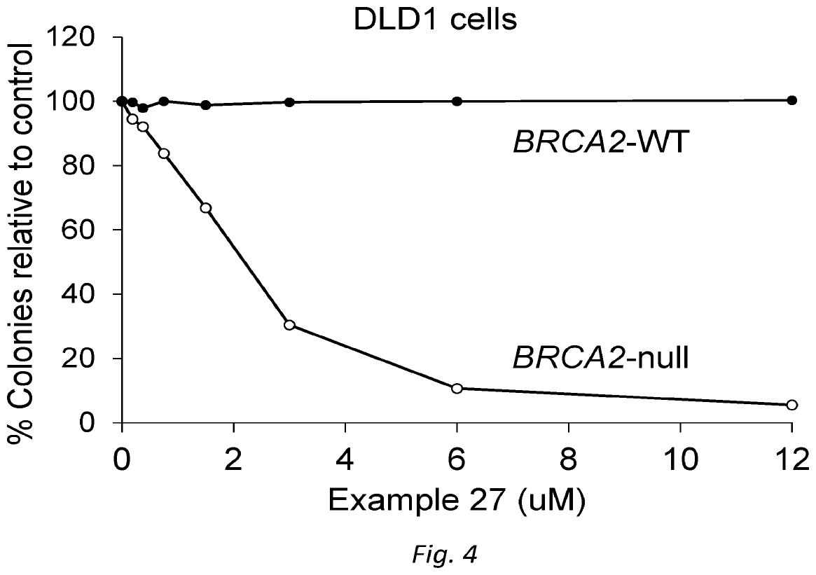

FIGS. 1 through 4, depict representative results from experiments showing that examples of Polq inhibitors (Polqi) selectively kill BRCA-mutant cancer cells. FIGS. 1 and 2 depict scatter plots showing that the survival of BRCA2-null HCT116 cells (FIG. 1) and BRCA2-null DLD1 cells (FIG. 2) is significantly reduced by treatment with the indicated Polqi (Example 26) as compared to BRCA2-WT HCT116 cells (FIG. 1) and BRCA2-WT DLD1 cells (FIG. 2), which are mostly resistant to Polqi. FIGS. 3 and 4 depict scatter plots showing that the survival of BRCA2-null HCT116 cells (FIG. 3) and BRCA2-null DLD1 cells (FIG. 4) is significantly reduced by treatment with the indicated Polqi (Example 27) as compared to BRCA2-WT HCT116 cells (FIG. 3) and BRCA2-WT DLD1 cells (FIG. 4) which are mostly resistant to Polqi. Data are represented as mean. n=3, +/−s.d.

What is claimed is:

1. A compound having the structure of Formula (I), or a tautomeric or a stereochemically isomeric form, a pharmaceutically acceptable salt or a solvate thereof: 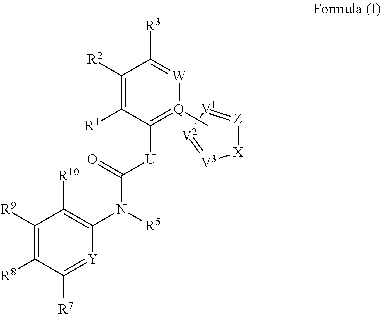
wherein:
W represents C(R⁴) or N;
U represents CH₂, O, S, or NR^{U};
Y represents C(R⁶) or N;
ring A represents an unsaturated 5-membered ring;
Q represents a carbon atom directly bonded to ring A;
R¹, R², R³, R₄, R⁷, R⁸, R⁹, R¹⁰, and RU independently represent hydrogen, deuterium, C₁₋₆ alkyl, C₂₋₆ alkenyl, alkynyl, hydroxy, thiol, C₁₋₆ alkoxy, halogen, haloC₁₋₆ alkyl, haloC₁₋₆ alkoxy, C₃₋₈ cycloalkyl, nitrile, NR^{X}R^{Y}, and combinations thereof; wherein two adjacent groups R¹ to R⁴ or R⁶ to R¹⁰ optionally join to form a 5- to 7-membered saturated or unsaturated ring optionally containing one or more heteroatoms selected from O, N or S;
R⁵ and R⁶ independently represent hydrogen, deuterium, C₁₋₆ alkyl, C₂₋₆ alkenyl, alkynyl, hydroxy, thiol, C₁₋₆ alkoxy, halogen, haloC₁₋₆ alkyl, haloC₁₋₆ alkoxy, C₃₋₈ cycloalkyl, nitrile, —NR^{X}R^{Y}, aryl, heteroaryl, heterocyclyl, amide, and combinations thereof;
Z represents CR^{Z} or N, V¹ is C, and the bond between V¹ and Z is a double bond; or
Z represents NR^{Z}, CR^{Z}R^{Z′}, C═S, or C═O, V¹ is N, and the bond between V¹ and Z is a single bond;
V² represents N or CR^{V2}
V³ represents N or CR^{V3}
X represents C(R¹⁵)(R¹⁶), N(R¹⁷), S, or O;
R¹⁵, R¹⁶, and R¹⁷ independently represent hydrogen, deuterium, C₁₋₂₀ alkyl, haloC₁₋₂₀ alkyl, —OR^{15a}, —SR^{15a}, nitrile, —COC₁₋₂₀ alkyl, —COOC₁₋₂₀ alkyl, hydroxy, C₁₋₂₀ alkoxy, C₁₋₂₀ alkanol, C₃₋₈ cycloalkyl, halogen, carbonyl, —NR^{V}R^{W}, —CH₂—NR^{V}R^{W}, —OSO₂NH₂, —P(O)OH₂, aryl, heteroaryl, heterocyclyl, and combinations thereof; wherein R¹⁵, R¹⁶, and R¹⁷ may further comprise one or more divalent linkers L selected from the group consisting alkylene, cycloalkylene, heteroalkylene, heterocycloalkylene, alkenylene, alkynylene, arylene, heteroarylene, silyl, amine, amide, ester, ether, carbonyl, carbamate, carbonate, sulfamate, sulfonic ester, sulfoximine, sulfonamide, thioether, thioester, disulfide, hydrazine, urea, thiourea, phosphate, phosphonate ester, poly (alkyl ether), heteroatom, and combinations thereof;
R^{Z}, R^{Z′}, R^{V2}, and R^{V3} independently represent hydrogen, deuterium, C₁₋₆ alkyl, hydroxy, C₁₋₆ alkoxy, C₁₋₆ alkanol, halogen, —OR^{15b}, CO₂H, CO₂R^{15b}, haloC₁₋₆ alkyl, and combinations thereof;
provided one of R^{Z}, R^{Z′}, R^{V2}, R^{V3}, R¹⁵, R¹⁶, and R¹⁷ represents a direct bond to Q;
each R^{15a} and R^{15b} independently represents hydrogen, deuterium, or C₁₋₆ alkyl;
R^{V}, R^{W}, R^{X} and R^{Y} independently represent hydrogen, deuterium, C₁₋₆ alkyl, haloC₁₋₆ alkyl, C₃₋₈ cycloalkyl, —COC₁₋₆ alkyl or heterocyclyl; wherein said alkyl groups may be optionally substituted with or more deuterium, hydroxy, amino or sulfone groups; and said heterocyclyl ring may be optionally substituted by one or more deuterium, oxo, hydroxy, C₁₋₆ alkanol or —COC₁₋₆ alkyl groups.

2. The compound of claim 1, wherein U is O.

3. The compound of claim 1, wherein U is CH₂.

4. The compound of claim 1, wherein U is S.

5. The compound of claim 1, wherein at least one of R¹ and R³ represents halogen or haloC₁₋₆ alkyl.

6. The compound of claim 1, wherein R¹ and R³ each independently represents halogen, haloC₁₋₆ alkyl, or CF₃.

7. The compound of claim 1, wherein R¹ and R³ each represent CF₃.

8. The compound of claim 1, wherein R² is selected from the group consisting of hydrogen, deuterium, halogen, C₁₋₆ alkyl, and haloC₁₋₆ alkyl.

9. The compound of claim 1, wherein R⁴ is selected from the group consisting of hydrogen, deuterium, halogen, nitrile, methyl, and ethynyl.

10. The compound of claim 1, wherein R⁵ represents CH₃ or CD₃.

11. The compound of claim 1, wherein R⁵ represents cyclopropyl or CH₂CN.

12. The compound of claim 1, wherein R⁵ represents: 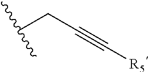
wherein R^{5′} is selected from the group consisting of hydrogen, deuterium, C₁₋₆ alkyl, C₂₋₆ alkenyl, alkynyl, hydroxy, thiol, C₁₋₆ alkoxy, halogen, haloC₁₋₆ alkyl, haloC₁₋₆ alkoxy, C₃₋₈ cycloalkyl, nitrile, —NR^{X}R^{Y}, aryl, heteroaryl, heterocyclyl, amide, and combinations thereof.

13. The compound of claim 1, wherein R⁵ or R⁶ represents one of the following substituents:

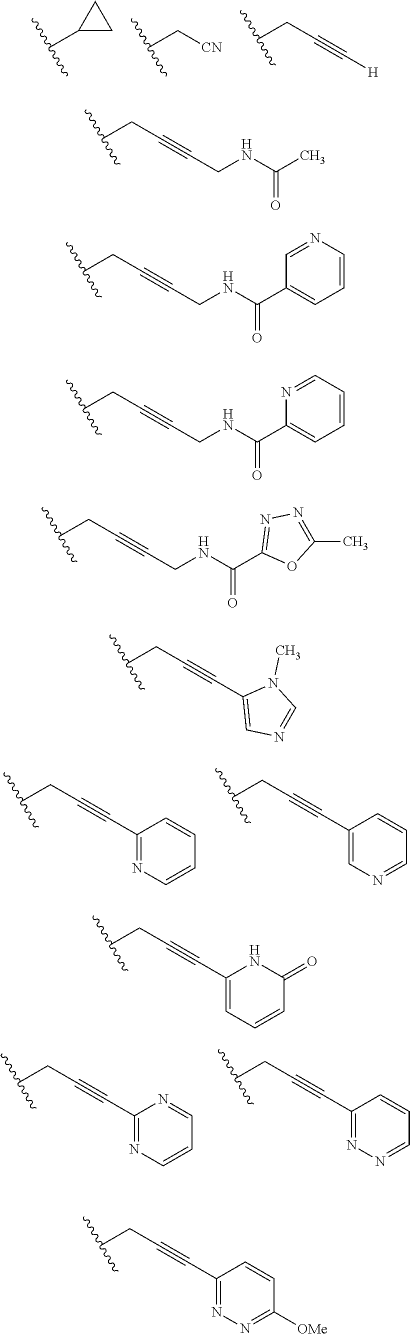

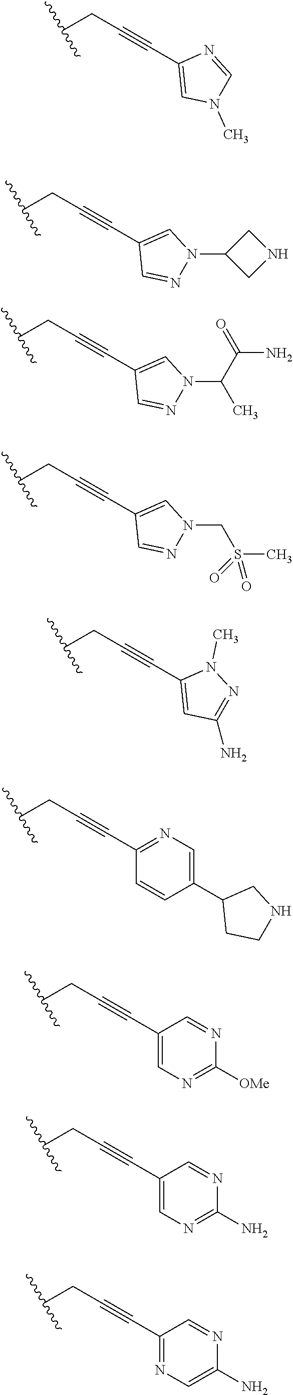

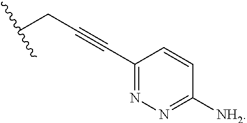 [SMILES: *CC#Cc1ccc(N)nn1]

14. The compound of claim 1, wherein R⁸, R⁹, and R¹⁰ each represent halogen.

15. The compound of claim 1, wherein R⁷, R⁸, and R¹⁰ each represent halogen.

16. The compound of claim 1, wherein R⁸ and R⁹ each represent halogen.

17. The compound of claim 1, wherein R⁸ represents fluorine.

18. The compound of claim 1, wherein R⁹ represents chlorine.

19. The compound of claim 1, wherein R¹⁰ represents fluorine.

20. The compound of claim 1, wherein Z represents CR^{Z} or N, V¹ is C, and the bond between V¹ and Z is a double bond.

21. The compound of claim 1, wherein Z represents NR^{Z}, CR^{Z}R^{Z′}, C═S, or C═O, V¹ is N, and the bond between V¹ and Z is a single bond.

22. The compound of claim 1, wherein Z represents C═O or C═S, V¹ is N, and X is NR¹⁷.

23. The compound of claim 1, wherein V¹ is C, Z is CH, V² and V³ are N, and X is NR¹⁷.

24. The compound of claim 1, wherein X represents NR¹⁷ and R¹⁷ is represented by: 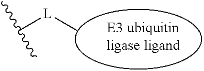
wherein L represents a divalent organic linker selected from the group consisting of alkylene, cycloalkylene, heteroalkylene, heterocycloalkylene, alkenylene, alkynylene, arylene, heteroarylene, silyl, amine, amide, ester, ether, carbonyl, carbamate, sulfamate, thioether, thioester, disulfide, hydrazine, urea, thiourea, phosphate, phosphonate ester, poly (alkyl ether), heteroatom, and combinations thereof; and
E3 ubiquitin ligase ligand represents pomalidomide, Von Hippel-Lindau (VHL), thalidomide, lenalidomide, iberdomide, avadomide, apremilast, Mouse double minute 2 homolog (MDM2), bestatin, or an MV1 derivative.

25. The compound of claim 1, wherein X represents NR¹⁷ and R¹⁷ represents one of the following substituents: 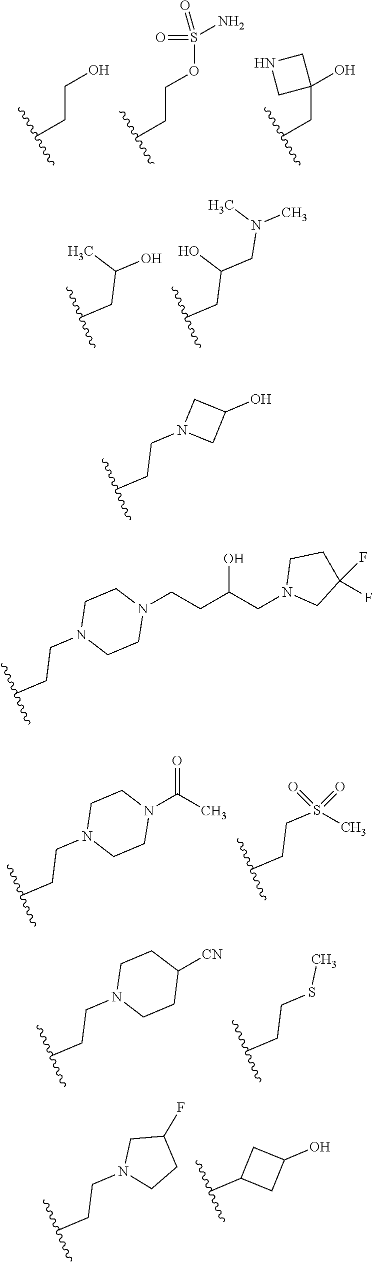 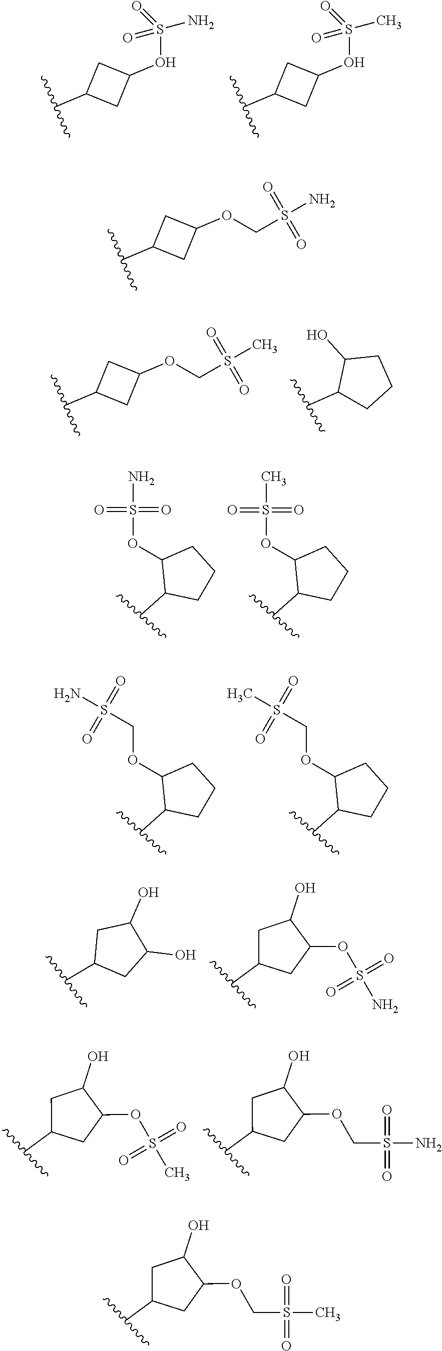 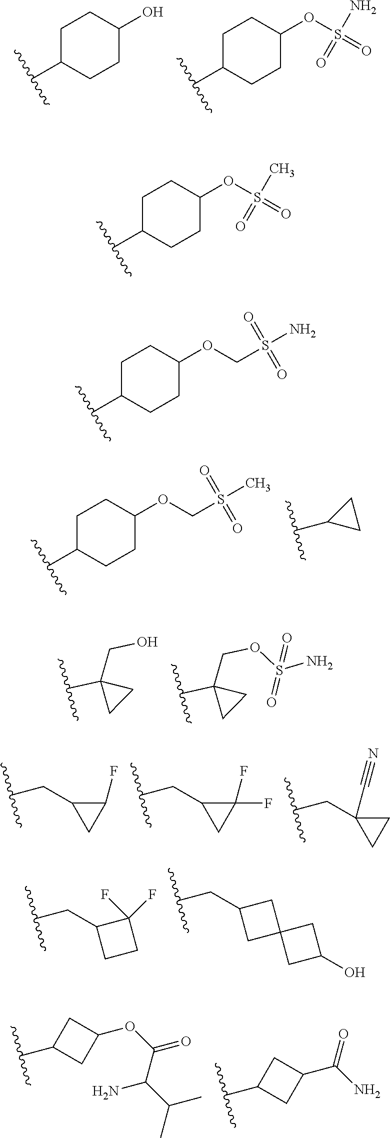 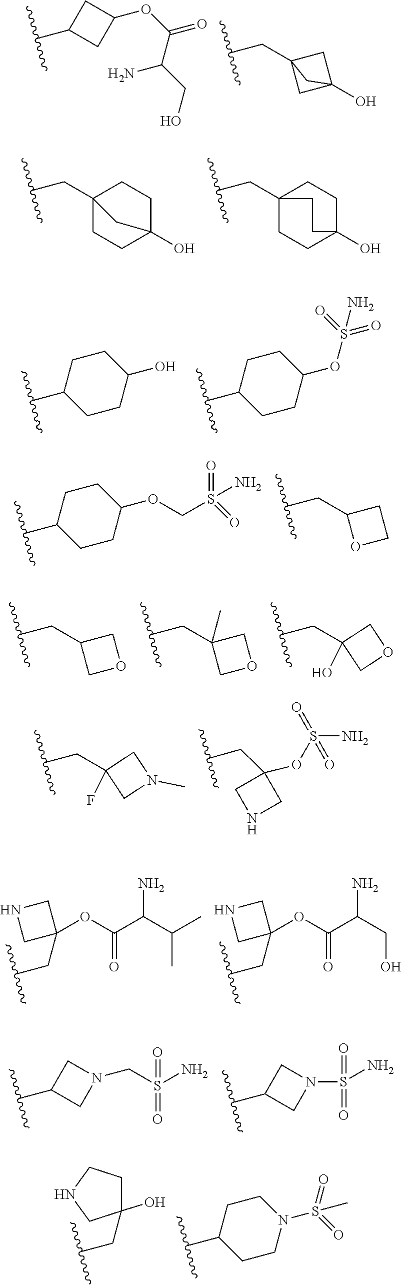 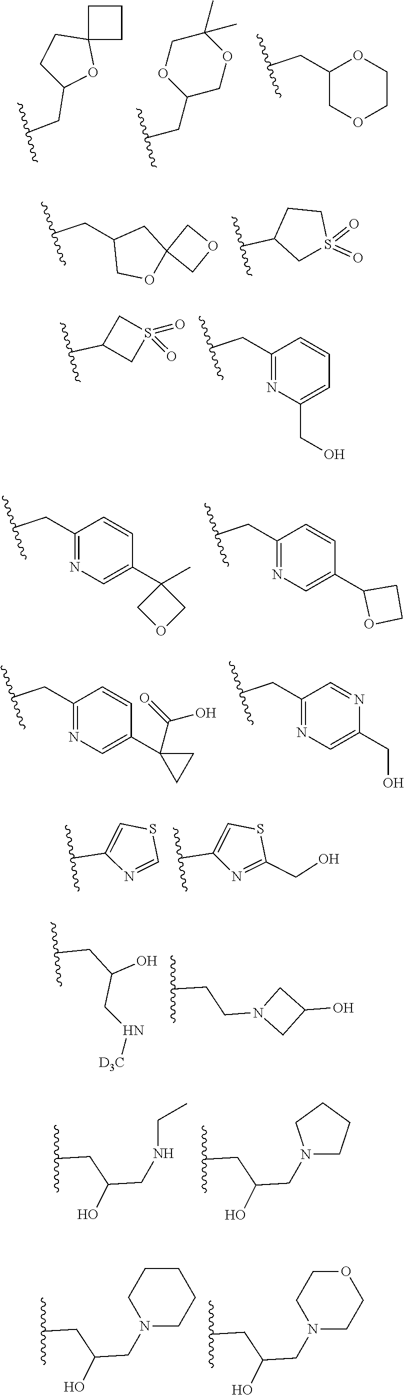 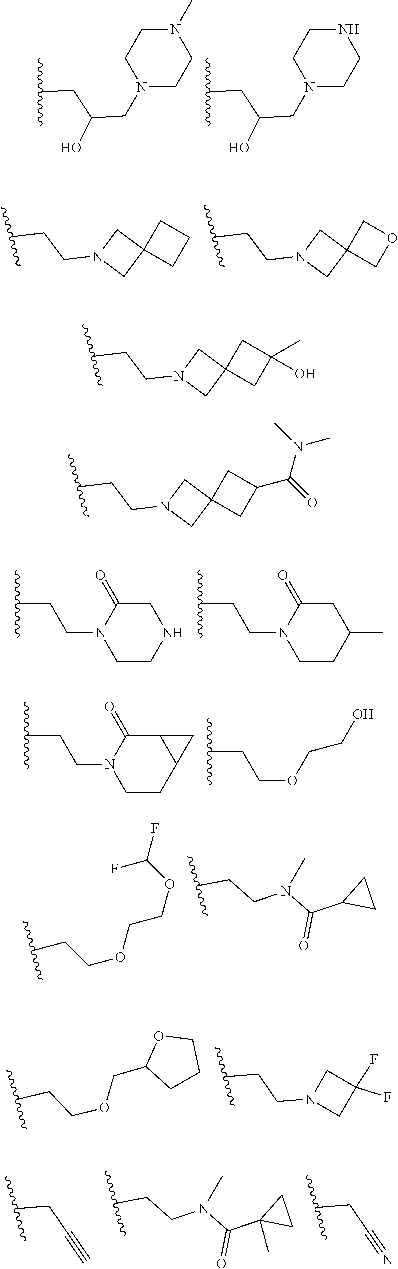 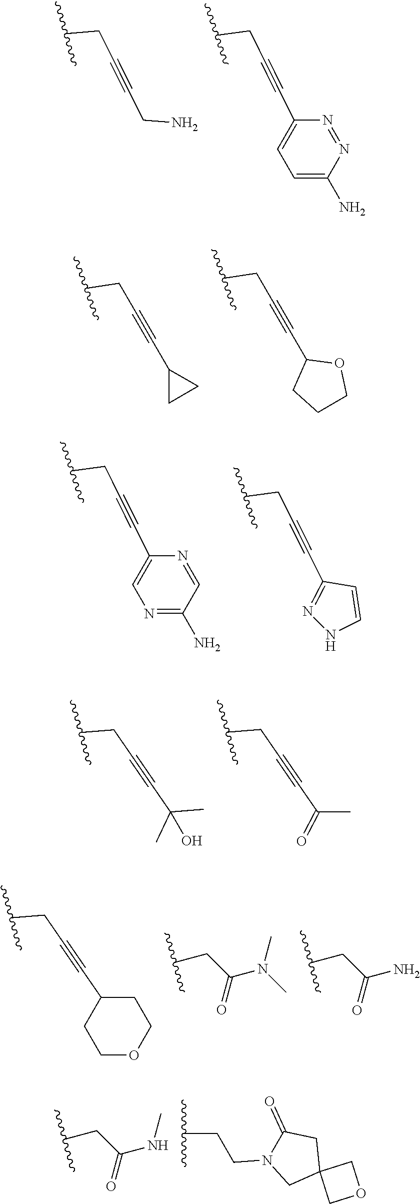 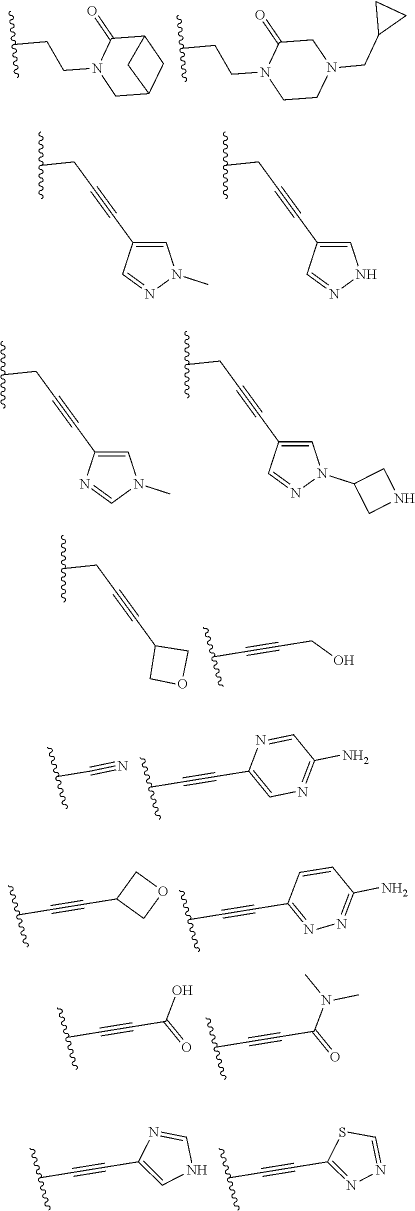 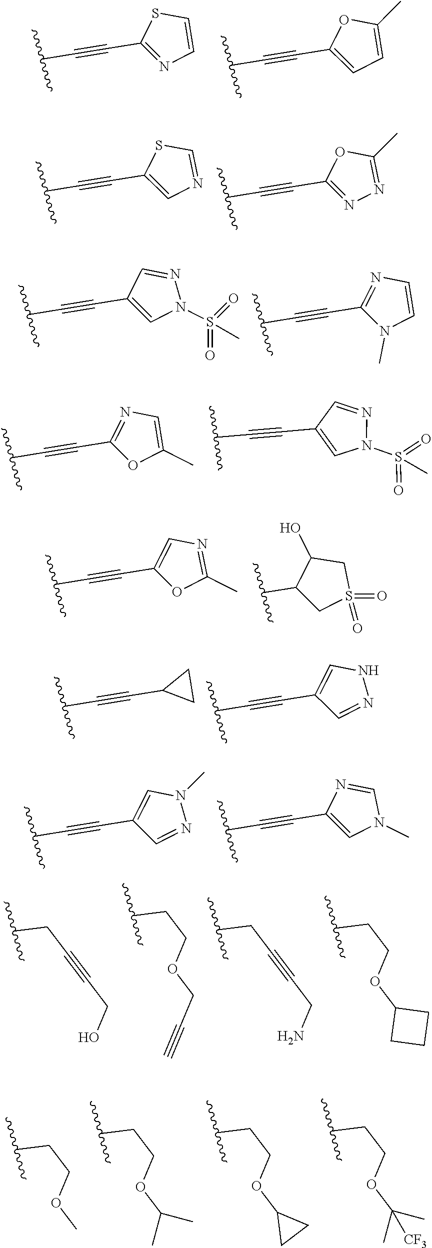 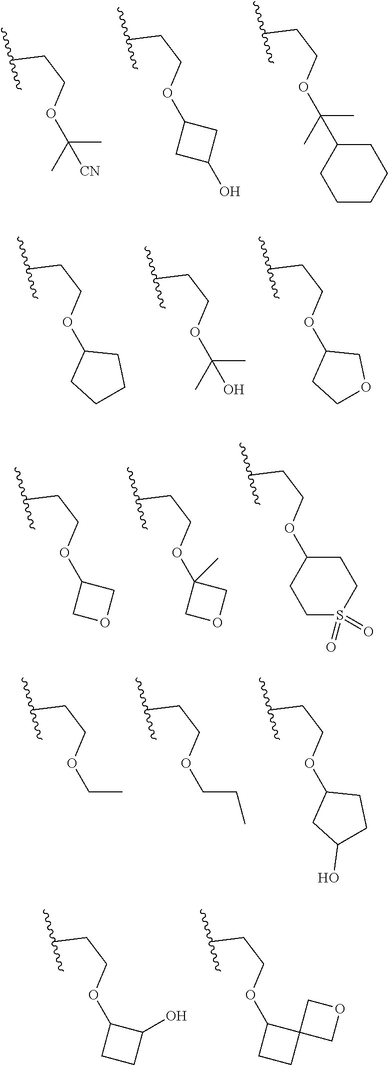 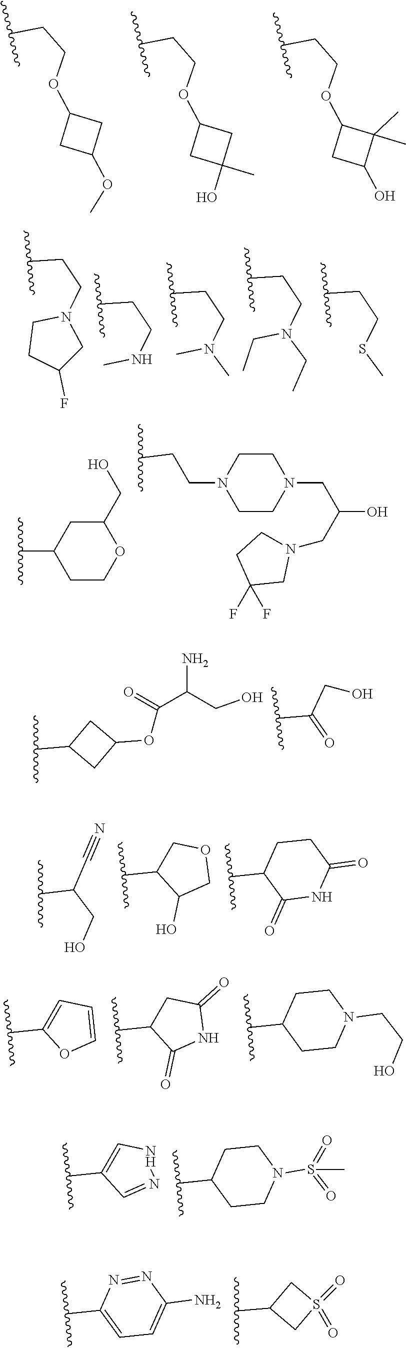 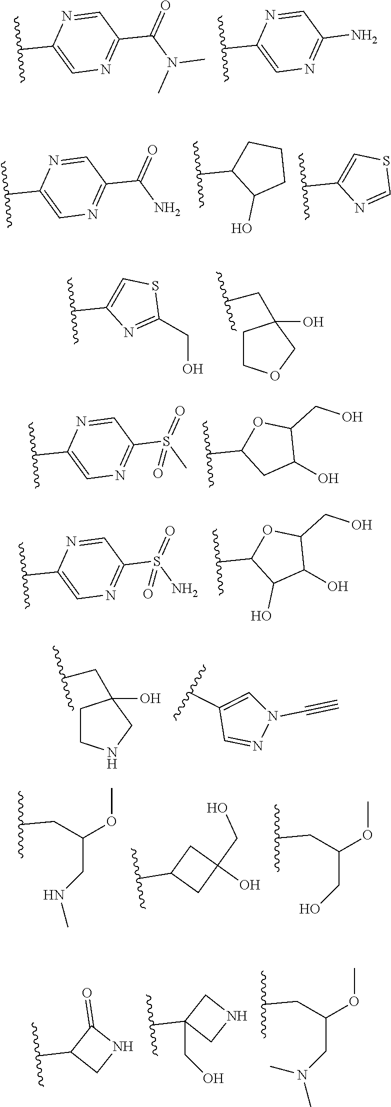 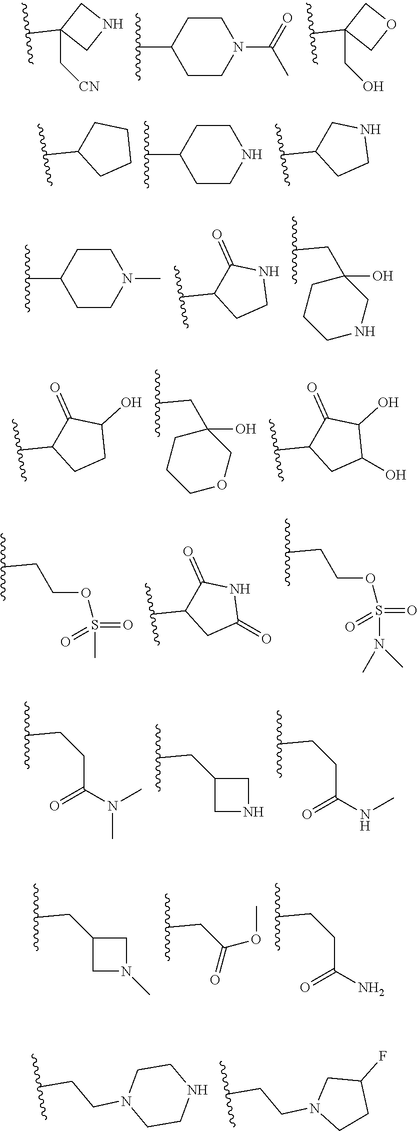 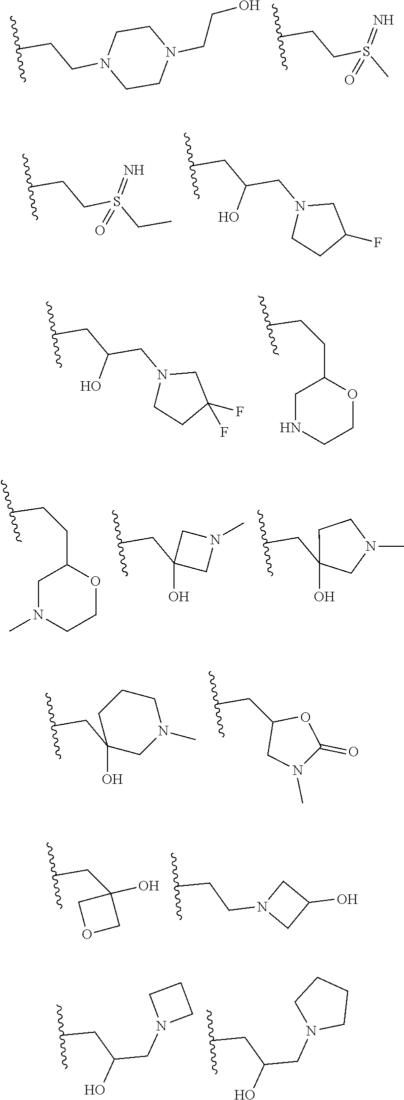 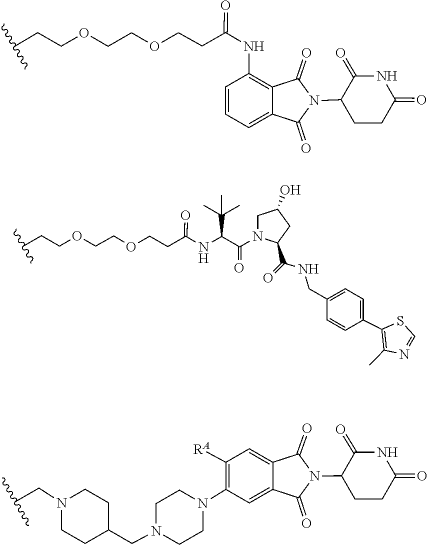
wherein R^{A} is H or halogen.

26. The compound of claim 1, wherein the compound of Formula (I) is represented by Formula (Ia):

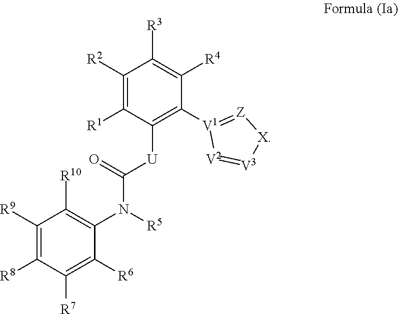

27. The compound of claim 1, wherein the compound of Formula (I) is represented by Formula (Ib):

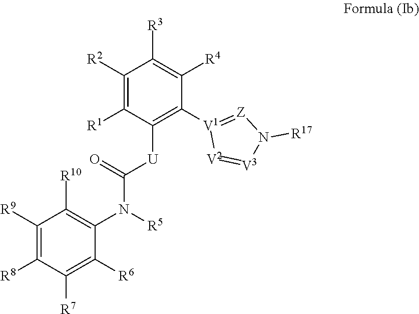

28. The compound of claim 1, wherein the compound of Formula (I) is represented by Formula (Ic) or Formula (Ic′):

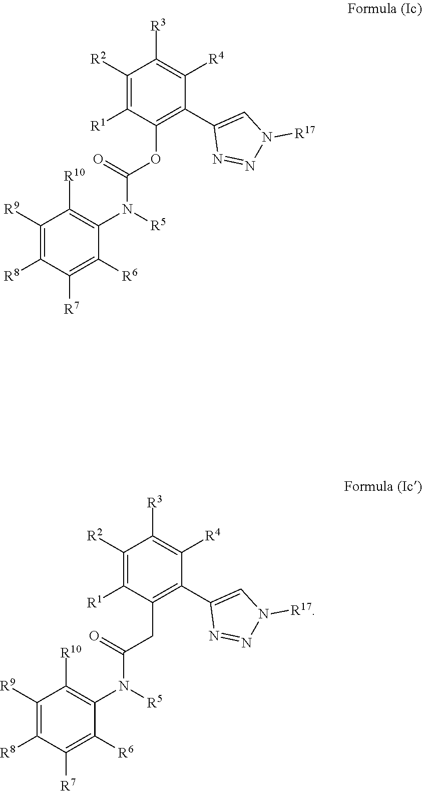

29. The compound of claim 1, wherein the compound of Formula (I) is represented by Formula (Id) or Formula (Id′):

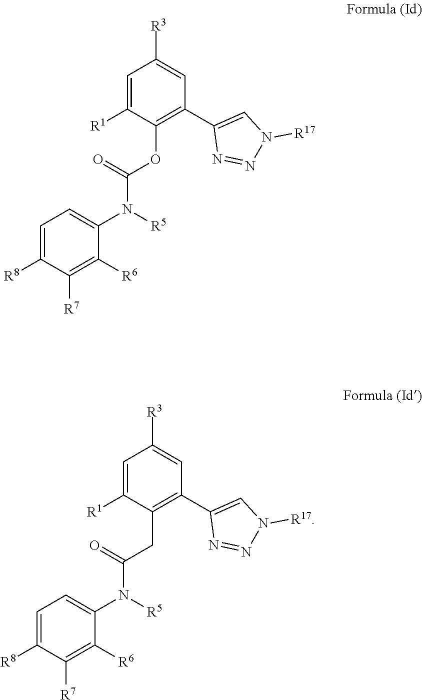

30. The compound of claim 1, wherein the compound of Formula (I) is represented by Formula (Ie) or Formula (Ie′):

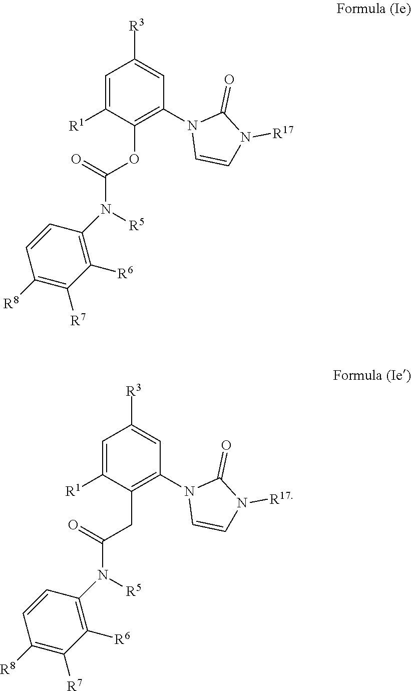

31. The compound of claim 1, wherein the compound of Formula (I) is represented by Formula (If) or Formula (If′):

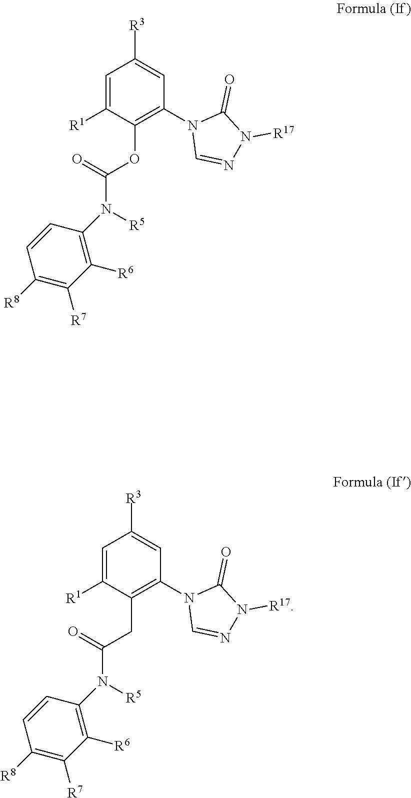

32. The compound of claim 1, wherein the compound of Formula (I) is represented by Formula (Ig) or Formula (Ig′):

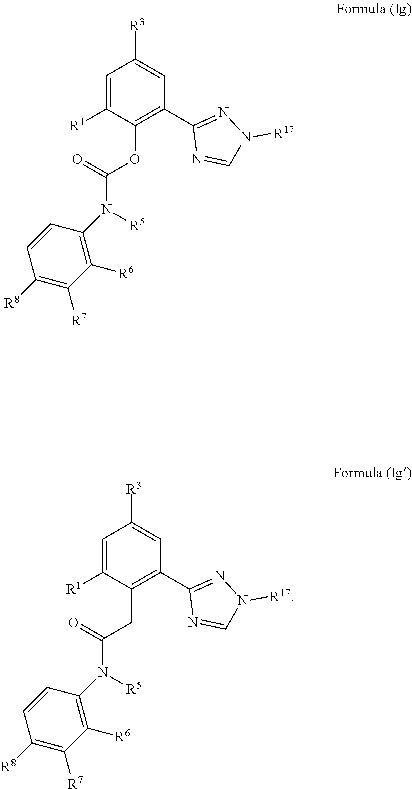

33. The compound of claim 1, wherein the compound of Formula (I) is represented by Formula (Ih) or Formula (Ih′):

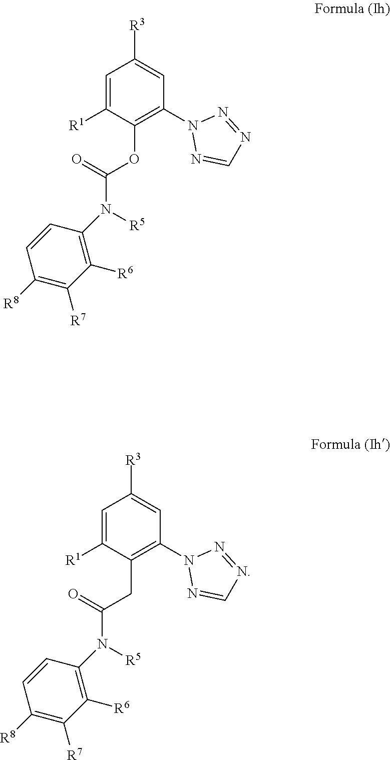

34. The compound of claim 1, wherein the compound of Formula (I) is represented by Formula (Ii) or Formula (Ii′):

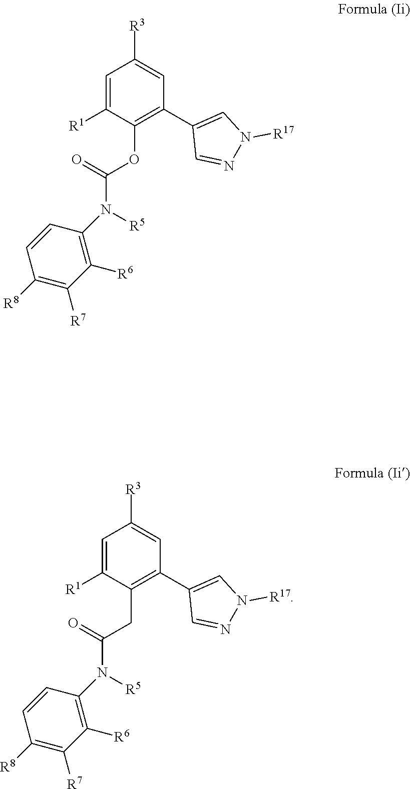

35. The compound of claim 1, wherein the compound of Formula (I) is represented by Formula (Ij) or Formula (Ij′):

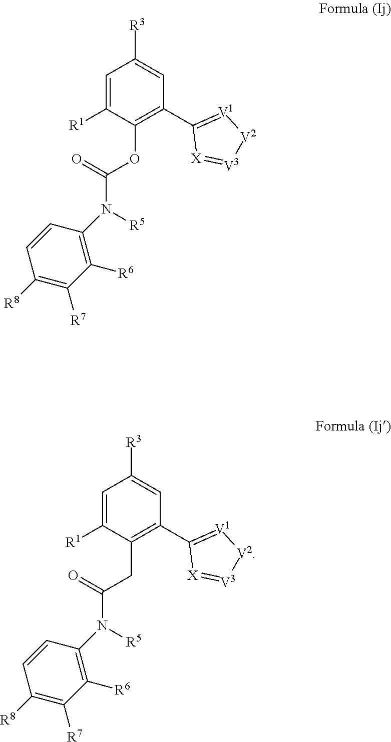

36. A method of inhibiting the expression or stability of DNA polymerase theta (Polq) in a subject, the method comprising administering to the subject a compound of claim 1 or a tautomeric or a stereochemically isomeric form, a pharmaceutically acceptable salt or a solvate thereof.

37. A method of treating cancer in a subject in need thereof, the method comprising administering to the subject a compound of Formula (I) or a tautomeric or a stereochemically isomeric form, a pharmaceutically acceptable salt or a solvate thereof: 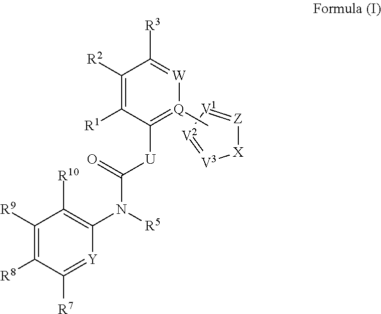
wherein:
W represents C(R⁴) or N;
U represents CH₂, O, S, or NR^{U};
Y represents C(R⁶) or N;
ring A represents an unsaturated 5-membered ring;
Q represents a carbon atom directly bonded to ring A;
R¹, R², R³, R⁴, R⁷, R⁸, R⁹, R¹⁰, and RU independently represent hydrogen, deuterium, C₁₋₆ alkyl, C₂₋₆ alkenyl, alkynyl, hydroxy, thiol, C₁₋₆ alkoxy, halogen, haloC₁₋₆ alkyl, haloC₁₋₆ alkoxy, C₃₋₈ cycloalkyl, nitrile, NR^{X}R^{Y}, and combinations thereof; wherein two adjacent groups R¹ to R⁴ or R⁶ to R¹⁰ optionally join to form a 5- to 7-membered saturated or unsaturated ring optionally containing one or more heteroatoms selected from O, N or S;
R⁵ and R⁶ independently represent hydrogen, deuterium, C₁₋₆ alkyl, C₂₋₆ alkenyl, alkynyl, hydroxy, thiol, C₁₋₆ alkoxy, halogen, haloC₁₋₆ alkyl, haloC₁₋₆ alkoxy, C₃₋₈ cycloalkyl, nitrile, —NR^{X}R^{Y}, aryl, heteroaryl, heterocyclyl, amide, and combinations thereof;
Z represents CR^{Z} or N, V¹ is C, and the bond between V¹ and Z is a double bond; or
Z represents NR^{Z}, CR^{Z}R^{Z′}, C═S, or C═O, V¹ is N, and the bond between V¹ and Z is a single bond;
V² represents N or CR^{V2}
V³ represents N or CR^{V3}
X represents C(R¹⁵)(R¹⁶), N(R¹⁷), S, or O;
R¹⁵, R¹⁶, and R¹⁷ independently represent hydrogen, deuterium, C₁₋₂₀ alkyl, haloC₁₋₂₀ alkyl, —OR^{15a}, —SR^{15a}, nitrile, —COC₁₋₂₀ alkyl, —COOC₁₋₂₀ alkyl, hydroxy, C₁₋₂₀ alkoxy, C₁₋₂₀ alkanol, C₃₋₈ cycloalkyl, halogen, carbonyl, —NR^{V}R^{W}, —CH₂—NR^{V}R^{W}, —OSO₂NH₂, —P(O)OH₂, aryl, heteroaryl, heterocyclyl, and combinations thereof; wherein R¹⁵, R¹⁶, and R¹⁷ may further comprise one or more divalent linkers L selected from the group consisting alkylene, cycloalkylene, heteroalkylene, heterocycloalkylene, alkenylene, alkynylene, arylene, heteroarylene, silyl, amine, amide, ester, ether, carbonyl, carbamate, carbonate, sulfamate, sulfonic ester, sulfoximine, sulfonamide, thioether, thioester, disulfide, hydrazine, urea, thiourea, phosphate, phosphonate ester, poly (alkyl ether), heteroatom, and combinations thereof;
R^{Z}, R^{Z′}, R^{V2}, and R^{V3} independently represent hydrogen, deuterium, C₁₋₆ alkyl, hydroxy, C₁₋₆ alkoxy, C₁₋₆ alkanol, halogen, —OR^{15b}, CO₂H, CO₂R^{15b}, haloC₁₋₆ alkyl, and combinations thereof;
provided one of R^{Z}, R^{Z′}, R^{V2}, R^{V3}, R¹⁵, R¹⁶, and R¹⁷ represents a direct bond to Q;
each R^{15a} and R^{15b} independently represents hydrogen, deuterium, or C₁₋₆ alkyl;
R^{V}, R^{W}, R^{X} and R^{Y} independently represent hydrogen, deuterium, C₁₋₆ alkyl, haloC₁₋₆ alkyl, C₃₋₈ cycloalkyl, —COC₁₋₆ alkyl or heterocyclyl; wherein said alkyl groups may be optionally substituted with or more deuterium, hydroxy, amino or sulfone groups; and said heterocyclyl ring may be optionally substituted by one or more deuterium, oxo, hydroxy, C₁₋₆ alkanol or —COC₁₋₆ alkyl groups.

38. The method of claim 37, further comprising the step of administering to the subject one or more PARP inhibitors, one or more topoisomerase inhibitors, or an anti-cancer radiotherapy.

39. A method of inhibiting the activity of DNA polymerase theta (Polq), the method comprising the step of contacting Polq with a compound of Formula (I) or a tautomeric or a stereochemically isomeric form, a pharmaceutically acceptable salt or a solvate thereof: 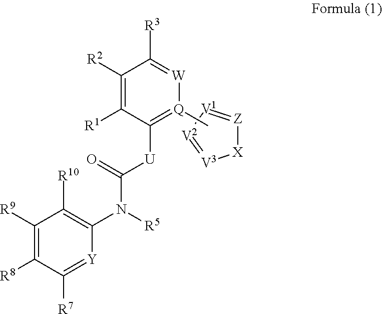
wherein:
W represents C(R⁴) or N;
U represents CH₂, O, S, or NR^{U};
Y represents C(R⁶) or N;
ring A represents an unsaturated 5-membered ring;
Q represents a carbon atom directly bonded to ring A;
R¹, R², R³, R⁴, R⁷, R⁸, R⁹, R¹⁰, and RU independently represent hydrogen, deuterium, C₁₋₆ alkyl, C₂₋₆ alkenyl, alkynyl, hydroxy, thiol, C₁₋₆ alkoxy, halogen, haloC₁₋₆ alkyl, haloC₁₋₆ alkoxy, C₃₋₈ cycloalkyl, nitrile, NR^{X}R^{Y}, and combinations thereof; wherein two adjacent groups R¹ to R⁴ or R⁶ to R¹⁰ optionally join to form a 5- to 7-membered saturated or unsaturated ring optionally containing one or more heteroatoms selected from O, N or S;
R⁵ and R⁶ independently represent hydrogen, deuterium, C₁₋₆ alkyl, C₂₋₆ alkenyl, alkynyl, hydroxy, thiol, C₁₋₆ alkoxy, halogen, haloC₁₋₆ alkyl, haloC₁₋₆ alkoxy, C₃₋₈ cycloalkyl, nitrile, —NR^{X}R^{Y}, aryl, heteroaryl, heterocyclyl, amide, and combinations thereof;
Z represents CR^{Z} or N, V¹ is C, and the bond between V¹ and Z is a double bond; or
Z represents NR^{Z}, CR^{Z}R^{Z′}, C═S, or C═O, V¹ is N, and the bond between V¹ and Z is a single bond;
V² represents N or CR^{V2}
V³ represents N or CR^{V3}
X represents C(R¹⁵)(R¹⁶), N(R¹⁷), S, or O;
R¹⁵, R¹⁶, and R¹⁷ independently represent hydrogen, deuterium, C₁₋₂₀ alkyl, haloC₁₋₂₀ alkyl, —OR^{15a}, —SR^{15a}, nitrile, —COC₁₋₂₀ alkyl, —COOC₁₋₂₀ alkyl, hydroxy, C₁₋₂₀ alkoxy, C₁₋₂₀ alkanol, C₃₋₈ cycloalkyl, halogen, carbonyl, —NR^{V}R^{W}, —CH₂—NR^{V}R^{W}, —OSO₂NH₂, —P(O)OH₂, aryl, heteroaryl, heterocyclyl, and combinations thereof; wherein R¹⁵, R¹⁶, and R¹⁷ may further comprise one or more divalent linkers L selected from the group consisting alkylene, cycloalkylene, heteroalkylene, heterocycloalkylene, alkenylene, alkynylene, arylene, heteroarylene, silyl, amine, amide, ester, ether, carbonyl, carbamate, carbonate, sulfamate, sulfonic ester, sulfoximine, sulfonamide, thioether, thioester, disulfide, hydrazine, urea, thiourea, phosphate, phosphonate ester, poly (alkyl ether), heteroatom, and combinations thereof;
R^{Z}, R^{Z′}, R^{V2}, and R^{V3} independently represent hydrogen, deuterium, C₁₋₆ alkyl, hydroxy, C₁₋₆ alkoxy, C₁₋₆ alkanol, halogen, —OR^{15b}, CO₂H, CO₂R^{15b}, haloC₁₋₆ alkyl, and combinations thereof;
provided one of R^{Z}, R^{Z′}, R^{V2}, R^{V3}, R¹⁵, R¹⁶, and R¹⁷ represents a direct bond to Q;
each R^{15a} and R^{15b} independently represents hydrogen, deuterium, or C₁₋₆ alkyl;
R^{V}, R^{W}, R^{X} and R^{Y} independently represent hydrogen, deuterium, C₁₋₆ alkyl, haloC₁₋₆ alkyl, C₃₋₈ cycloalkyl, —COC₁₋₆ alkyl or heterocyclyl; wherein said alkyl groups may be optionally substituted with or more deuterium, hydroxy, amino or sulfone groups; and said heterocyclyl ring may be optionally substituted by one or more deuterium, oxo, hydroxy, C₁₋₆ alkanol or —COC₁₋₆ alkyl groups.
